(19) United States
(12) Patent Application Publication
(10) Pub. No.: US 2026/0103474 A1
(43) Pub. Date: Apr. 16, 2026

(54) NOVEL COMPOUNDS FOR THE DIAGNOSIS OF TDP-43 PROTEINOPATHIES

(71) Applicant: AC IMMUNE SA, Lausanne (CH)

(72) Inventors: Nicolas Dreyfus, Lausanne (CH); Heiko Kroth, Lausanne (CH)

(21) Appl. No.: 19/116,853

(22) PCT Filed: Sep. 29, 2023
(86) PCT No.: PCT/EP2023/077098

§ 371 Date: Mar. 28, 2025

(30) Foreign Application Priority Data
Sep. 30, 2022 (EP) 22199297.7
Mar. 27, 2023 (EP) 23164491.5
Jul. 12, 2023 (EP) 23185134.6

(51) Int. Cl. C07D 513/04; A61K 51/04; C07B 59/00

(52) U.S. Cl. CPC C07D 513/04; A61K 51/0455; A61K 51/0459; C07B 59/002; A61K 2123/00; C07B 2200/05

(57) ABSTRACT

The present invention relates to compounds which are suitable for imaging TDP-43 (Transactive response (TAR) DNA binding protein 43 kDa) aggregates. The compounds can be used, for example, for diagnosing a disease, disorder or abnormality associated with TDP-43 aggregates or a TDP-43 proteinopathy, such as amyotrophic lateral sclerosis (ALS), Alzheimer's disease (AD), Frontotemporal dementia (FTD) and limbic-predominant age-related TDP-43 encephalopathy (LATE).

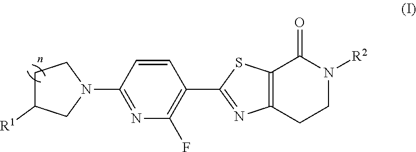

### FIELD OF THE INVENTION

The present invention relates to compounds which are suitable for imaging TDP-43 (Transactive response (TAR) DNA binding protein 43 kDa) aggregates. Said compounds can be used, for example, for diagnosing a disease, disorder or abnormality associated with TDP-43 aggregates or a TDP-43 proteinopathy, such as amyotrophic lateral sclerosis (ALS), Alzheimer's disease (AD), Frontotemporal dementia (FTD) and limbic-predominant age-related TDP-43 encephalopathy (LATE). The present invention also relates to processes for the preparation of said compounds, diagnostic compositions comprising said compounds, methods of using said compounds, kits comprising said compounds and their uses thereof.

### BACKGROUND OF THE INVENTION

Age-associated brain disorders characterized by pathological aggregation of proteins in the CNS (proteinopathies) and peripheral organs represent one of the leading causes of disability and mortality in the world. The best characterized protein that forms extracellular aggregates is amyloid beta (Abeta) in Alzheimer's disease (AD) and Abeta-related disorders. Other disease-associated, aggregation-prone proteins leading to neurodegeneration include but are not limited to Tau, alpha-synuclein (a-syn), huntingtin, fused in sarcoma (FUS), dipeptide repeat proteins (DPRs) produced by unconventional translation of the C9orf72 repeat expansion, superoxide dismutase 1 (SOD1), and TDP-43. Diseases involving TDP-43 aggregates are generally referred to as TDP-43 proteinopathies and include, but are not limited to, amyotrophic lateral sclerosis (ALS) and frontotemporal lobar degeneration (FTLD), including frontotemporal lobar dementia with TDP-43 pathology (FTLD-TDP, Frontotemporal lobar degeneration with TDP-43 inclusions) and limbic-predominant age-related TDP-43 encephalopathy (LATE).

### TDP-43 Introduction

Transactive response (TAR) DNA binding protein 43 kDa (TDP-43) is a 414-amino acid protein encoded by the TARDBP gene on chromosome 1p36.2 (ALS10). TARDBP is comprised of six exons (exon 1 is non-coding; exons 2-6 are protein-coding). TDP-43 belongs to the family of heterogeneous ribonucleoprotein (hnRNP) RNA binding proteins (Wang et al., Trends in Molecular Medicine, Vol. 14, No. 11, 2008, 479-485; Lagier-Tourenne et al., Human Molecular Genetics, 2010, Vol. 19, Review Issue 1 R46-R64). TDP-43 contains five functional domains (FIG. 1 in Warraich et al., The International Journal of Biochemistry & Cell Biology, 42 (2010) 1606-1609): two RNA recognition motifs (RRM1 and RRM2), which have two highly conserved hexameric ribonucleoprotein 2 (RNP2) and octameric ribonucleioprotein 1 (RNP1) regions, a nuclear export signal (NES) and a nuclear localization signal (NLS) enabling it to shuttle between the nucleus and the cytoplasm transporting bound mRNA, and a glycine rich domain at the C-terminal, which mediates protein-protein interactions. TDP-43 is involved in multiple aspects of RNA processing, including transcription, splicing, transport, and stabilization (Buratti and Baralle, FEBS Journal, 277 (2010) 2268-2281). It is a highly conserved, ubiquitously expressed protein with a tightly autoregulated expression level that shuttles continuously between the nucleus and cytoplasm, but is normally localized predominantly to the nucleus. In 2006, TDP-43 was identified as the protein that accumulates in the vast majority of cases of frontotemporal lobar degeneration (FTLD) with tau-negative, ubiquitin-positive inclusions (then referred to as FTLD-TDP), and in most cases of amyotrophic lateral sclerosis (ALS) (Arai et al., Biochemical and Biophysical Research Communications, 351 (2006) 602-611; Neumann et al., Science, 314, (2006), 130-133).

Thirty-eight negative-dominant mutations in TDP-43 have been identified in sporadic and familial ALS patients as well as in patients with inherited FTD (K263E, N267S), mainly located in the glycine-rich domain (FIG. 1; Lagier-Tourenne and Cleveland, Cell, 136, 2009, 1001-1004). TDP-43 is inherently aggregation-prone, as shown by sedimentation assays, and this propensity is increased by some ALS-associated TARDBP mutations (Ticozzi et al., CNS Neurol. Disord. Drug Targets, 2010, 9(3), 285-296.).

### TDP-43 in Neurodegeneration

TDP-43 aggregates have been identified in a growing list of pathological conditions (Lagier-Tourenne et al., Human Molecular Genetics, 2010, Vol. 19, Review Issue 1 R46-R64), including but not limited to: frontotemporal dementia (sporadic or familial with or without motor-neuron disease (MND), with progranulin (GRN) mutation, with TARDBP mutation, with valosine-containing protein (VCP) mutation, linked to chromosome 9p, corticobasal degeneration, frontotemporal lobar degeneration with ubiquitin-positive inclusions, argyrophilic grain disease, Pick's disease and the like), amyotrophic lateral sclerosis (sporadic ALS, with TARDBP mutation, with ANG mutation), Alzheimer's disease (sporadic and familial), Down syndrome, familial British dementia, polyglutamine diseases (Huntington's disease and SCA3), hippocampal sclerosis dementia and myopathies (sporadic inclusion body myositis, inclusion body myopathy with VCP mutation, oculo-pharyngeal muscular dystrophy with rimmed vacuoles, myofibrillar myopathies with MYOT or DES mutation).

Aggregated TDP-43 from patient brains shows a number of abnormal modifications, including hyperphosphorylation, ubiquitination, acetylation and C-terminal fragments through proteolytic cleavage (Arai et al., Biochemical and Biophysical Research Communications, 351 (2006) 602-611; Neumann et al., Science, 314, (2006), 130-133; Neumann et al., Acta Neuropathol., (2009) 117: 137-149; Hasegawa et al., Annals of Neurology, 2008, Vol 64 No 1, 60-70; Cohen et al., Nat Commun.; 2015, 6: 5845). Another characteristic feature of TDP-43 pathology is redistribution and accumulation of TDP-43 from nucleus to cytoplasm. The hallmark lesions of FTLD-TDP are neuronal and glial cytoplasmic inclusions (neuronal cytoplasmic inclusions (NCI) and glial cytoplasmic inclusions (GCI), respectively) and dystrophic neurites (DN) that are immunoreactive for TDP-43, as well as ubiquitin and p62, but negative for other neurodegenerative disease-related proteins. Differences in inclusion morphology and tissue distribution thereof are associated with specific mutations and/or clinical representations. Four types of TDP-43 pathology are described so far by histological methods (Mackenzie and Neumann, J. Neurochem., (2016), 138 (Suppl. 1), 54-70). FTLD-TDP type A cases are characterized by abundant short DN and compact oval or crescentic NCI, predominantly in layer II of the neocortex (FIG. 2f in Mackenzie et al., J. Neurochem., 2016, 138 (Suppl. 1), 54-70). Lentiform neuronal intranuclear inclusions (NII) are also usually present, but much less abundant. Cases with this pathology usually present clinically with either behavioral-variant frontotemporal dementia (bvFTD) or nonfluent/agrammatic variants of Primary Progressive Aphasia (nfvPPA) and are associated with progranulin (GRN) mutations. Neuropsychiatric manifestations are particularly common in those with an underlying GRN or C9orf72 mutation. Type A is the most common type in the majority of AD cases (Josephs et al., Acta Neuropathol. 2014, 127(3), 441-50, Arai et al., Acta neuropathologica. 2009, 117, 125-136). TDP-43 neuropathology in most cases of LATE is also similar to that of Type A, which is consistently found in FTD-GRN (Nelson et al., Brain, 2019, 142, 1503-1527). Type B cases show moderate numbers of compact or granular NCI in both superficial and deep cortical layers with relatively few DN and NII (FIG. 2g in Mackenzie et al., J. Neurochem., 2016, 138 (Suppl. 1), 54-70). Most of the NCI have a diffuse granular morphology, sometimes referred to as “pre-inclusions”. Importantly, some cases also have a background of delicate, small TDP-43 threads and dots. Most cases with coappearence of FTD and ALS symptoms are found to have FTLD-TDP type B pathology. Type C cases have an abundance of long tortuous neurites, predominantly in the superficial cortical laminae, with few or no NCI (FIG. 2j in Mackenzie et al., J. Neurochem., 2016, 138 (Suppl. 1), 54-70). This pathology is particularly found in cases presenting with svPPA (semantic variant of primary progressive aphasia). FTLD-TDP type D displays with abundant lentiform neuronal intranuclear inclusions (NII) and short DN in the neocortex with only rare NCI (FIG. 2k in Mackenzie et al., J. Neurochem., 2016, 138 (Suppl. 1), 54-70). This pattern of pathology is only found in cases with VCP in association with inclusion body myositis.

### TDP-43 in FTD

Frontotemporal dementia (FTD) is a clinical term that covers a wide spectrum of disorders based on the degeneration of frontal and temporal lobes—a pathological feature termed frontotemporal lobar degeneration (FTLD). FTD is the second most abundant cause of early degenerative dementias in the age group below 65 years (Le Ber, Revue Neurologique, 169 (2013), 811-819). FTD is presented by several syndromes including bvFTD which is characterized by changes in personality and behavior; semantic dementia (SD) and progressive nonfluent aphasia (PNFA) characterized by changes in the language function; corticobasal syndrome (CBS), progressive supranuclear palsy syndrome and motor neuron disease (FTD-MND) characterized by movement disfunction. Diagnosis of these syndromes is complicated and final conclusion can only be achieved through postmortem tissue analysis based on immunohistochemistry to detect aggregated protein and description of the affected brain regions. In terms of pathological, proteinaceaous inclusions, about 45% of cases show pathological accumulation of misfolded Tau, 45% of cases have pathological TDP-43 and a smaller subgroup has aggregates of FUS and other proteins. FTLD-TDP is a pathology term describing FTD cases with TDP-43 pathology found predominantly as cytoplasmic or neuritic protein aggregates in neurons and glia containing misfolded, insoluble, phosphorylated and truncated TDP-43.

### TDP-43 in ALS

Amyotrophic lateral sclerosis (ALS) is an adult-onset neurodegenerative disorder characterized by the premature loss of upper and lower motor neurons. The progression of ALS is marked by fatal paralysis and respiratory failure with a disease course from diagnosis to death of 1 to 5 years. In most cases of sporadic ALS the neuropathology is characterized by abnormal cytoplasmic accumulations of TDP-43 in neurons and glia of the primary motor cortex, brainstem motor nuclei, spinal cord and the associated white matter tracts. ALS with dementia involves accumulation of TDP-43 in extramotor neocortex and hippocampus. The role of phosphorylation of TDP-43 in ALS patients has been explored with the help of phospho-specific antibodies that strongly bind to nuclear and cytoplasmic TDP-43 inclusions. Amino acids S379, S403, S404, S409, and S410 have been identified as the major sites of phosphorylation of TDP-43 (Hasegawa et al., Ann Neurol., 2008; 64: 60-70; Neumann et al., Acta Neuropathol., 2009, 117: 137-149).

### TDP-43 in LATE

Limbic-predominant age-related TDP-43 encephalopathy (LATE) neuropathological change (LATE-NC) is defined by a stereotypical TDP-43 proteinopathy in older adults, with or without coexisting hippocampal sclerosis pathology. LATE-NC is a common TDP-43 proteinopathy, associated with an amnestic dementia syndrome that mimicked Alzheimer's-type dementia in retrospective autopsy studies. LATE is distinguished from frontotemporal lobar degeneration with TDP-43 pathology based on its epidemiology (LATE generally affects older subjects), and relatively restricted neuroanatomical distribution of TDP-43 proteinopathy. There is no molecule-specific biomarker for LATE. A discovery of a TDP-43 PET tracer may enable accurate, potentially earlier diagnosis as well as monitoring of disease progression to facilitate longitudinal drug efficacy measurements in patients during clinical trials (including as a potential exclusion criterion for Alzheimer's disease clinical trials) and longitudinal studies of the clinical and pathological progression of LATE (Nelson et al., Brain, 2019, Vol. 142; issue 6, 1503-1527).

### TDP-43 in AD and Other Diseases

TDP-43 pathology occurs in up to 57% of brains of patients with Alzheimer's disease (Josephs K A et al., Acta Neuropathol., 2014; 127(6): 811-824; Josephs K A et al., Acta Neuropathol., 2014; 127(3): 441-450; McAleese et al., Brain Pathol., 2017 July; 27(4): 472-479). TDP-43 aggregation is associated with cognitive decline, memory loss and medial temporal atrophy in AD. TDP-43 positive patients are 10-fold more likely to be cognitively impaired at death compared to TDP-43 negative subjects. It appears that TDP-43 represents a secondary or independent pathology that shares overlapping features with AD by targeting the medial temporal lobe. Pathologic TDP-43 follows a stereotypical pattern of deposition that was captured by the TDP-43 in AD (TAD) staging scheme: TDP-43 first deposits in the amygdala (stage 1) followed by hippocampus, limbic, temporal, and finally frontostriatum (stage V) (Josephs K A et al., Acta Neuropathol., 2014; 127(6): 811-824; Josephs K A et al., Acta Neuropathol., 2014; 127(3): 441-450).

### Diagnostics in FTD and ALS

The diagnosis of FTD based on clinical manifestations is insufficient since the clinical representation can overlap with other diseases, in particular, in the earlier stages. Therefore, the development of sensitive and specific biomarkers allowing the differentiation between types of pathology within the FTD spectrum is an urgent task. Such tools will allow better detecting and understanding the specific type of pathology causing neurodegeneration. Eventually this will lead to the development of diagnostic biomarkers enabling more efficient and precise patient selection for longitudinal monitoring in clinical studies, supporting the development of novel therapeutics for ALS and FTD.

A number of approaches aim at development of biochemical biomarkers to distinguish different types of FTD pathology. Some studies showed that TDP-43 concentration is increased in cerebrospinal fluid (CSF) of clinically defined FTD or FTD-MND populations, although there is a significant overlap with control or AD subjects and it remains unclear if such an approach will prove clinically useful (Foulds et al., Acta Neuropathol., 2008, 116: 141-146; Steinacker et al., Arch. Neurol., 2008; 65(11): 1481-1487). Levels of total Tau or Thr181-phosphorylated Tau do not discriminate FTLD-Tau from control. A possible diagnostic tool for the differentiation of FTLD-Tau and FTLD-TDP is the reduced CSF p-Tau181 to Tau ratio below a value of 0.37 (Hu et al., Neurology., 2013; 81(22): 1945-1952). Another study showed that CSF phosphorylated Tau levels are positively associated with cerebral Tau burden in FTD and might help to distinguish TDP-43 proteinopathy from tauopathy (Irwin et al., Ann. Neurol., 2017 August; 82(2):247-258).

In parallel to biochemical biomarkers the development of imaging biomarkers will enable early and specific detection of the pathology in FTD and ALS. The ability to image TDP-43 deposition in the brain will be a substantial achievement for diagnosis and drug development for FTD, ALS and other neurodegenerative disorders. Progressive TDP-43 accumulation in the CNS is associated with disease progression and represents an obvious target for development of novel therapeutics and diagnostic tools to study pharmacodynamics and disease progression. Given the relative novelty of TDP-43 as a target, the development of a PET-tracer targeting this protein is at its beginning. However, most of the compound's reported so far are not specific for TDP-43 and no direct binding to the target was demonstrated for any of these compounds.

A number of challenges are associated with the development of a TDP-43-specific PET-tracer including low abundance and heterogenic distribution of the target in the patient's brain as well as the lack of reference compounds. In order to reduce background signal interference resulting from non-specific, off-target binding and to reduce dosing requirements, TDP-43 imaging compounds should bind with high affinity and selectivity to the target. For imaging of TDP-43 aggregates associated with neurological disorders such as FTD and ALS, imaging compounds need to penetrate the blood brain barrier and pass into the relevant regions of the brain. For targeting intracellular amyloid-like inclusions such as TDP-43 aggregates, cell permeability is a further requirement of imaging compounds. A further prerequisite in order to avoid accumulation of the compound in the tissue, which may result in increased risk of unwanted side effects, is a fast compound wash-out from the brain (or other target organ).

It was an object of the present invention to provide compounds which are able to bind to TDP-43 aggregates. In particular, the compounds of the present invention should be useful for identification and differentiation of patients and patient groups with TDP-43 proteinopathies (such as FTD, FTLD-TDP, LATE and ALS) and for differentiating TDP-43 proteinopathies from other proteinopathies.

The present inventors have surprisingly found that compounds having the formula (I) can recognize and bind to TDP-43 aggregates. Moreover, it was found that compounds of the invention display high selectivity to TDP-43 aggregates over co-pathologies such as Abeta and Tau in AD brain homogenates, as well as over a-syn in PD brain homogenates. Furthermore, it was shown that compounds of the invention have a robust brain uptake and fast washout in non-human primates, satisfying the criteria for PET tracer further development for use in human subjects.

### SUMMARY OF THE INVENTION

The present invention is summarized in the appended claims. In particular, the present invention refers to a compound having the formula (I)

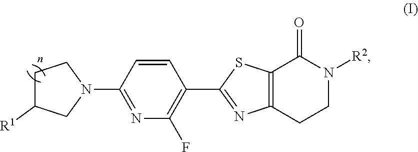

or a detectably labelled compound, stereoisomer, polymorph, racemic mixture, tautomer, pharmaceutically acceptable salt, prodrug, hydrate, or solvate thereof;
wherein
n is 1 or 2;
R¹ is H, hydroxy(C₁-C₄)alkyl or F; and
R² is a 5- or 6-membered carbocyclic or heterocyclic ring which can be optionally substituted with F, NH₂, CN and/or CH₃, wherein the heterocyclic ring contains one or more heteroatoms selected from N, O and S.

In another aspect, the present invention provides a diagnostic composition comprising a compound according to the definition of a compound of formula (I), or subformulae thereof, as defined herein, and optionally at least one physiologically acceptable carrier, diluent, adjuvant and/or excipient. Said compounds can be used for imaging of TDP-43 aggregates, particularly wherein the imaging is conducted by positron emission tomography or for diagnosing a disease, disorder or abnormality associated with TDP-43 aggregates, particularly wherein the diagnosis is conducted by positron emission tomography.

In another aspect, the invention provides a compound according to the definition of a compound of formula (I), or subformulae thereof, which can be used in the following methods:
method of diagnosing a disease, disorder or abnormality associated with TDP-43 aggregates, in a subject;
A method of positron emission tomography (PET) imaging of TDP-43 aggregates in a tissue of a subject;
A method for the detection and optionally quantification of TDP-43 aggregates in a tissue of a subject;
A method of the diagnostic imaging of the brain of a subject;
A method of collecting data for the diagnosis of a disease, disorder or abnormality associated with TDP-43 aggregates or for the diagnosis of a TDP-43 proteinopathy;
A method of collecting data for determining a predisposition to a disease, disorder or abnormality associated with TDP-43 aggregates or a TDP-43 proteinopathy;
A method of collecting data for monitoring the progression of a disease, disorder or abnormality associated with TDP-43 aggregates or for monitoring the progression of a TDP-43 proteinopathy in a patient; and
A method of collecting data for predicting responsiveness of a patient suffering from a disease, disorder or abnormality associated with TDP-43 aggregates to a treatment with a medicament.

In another aspect, the invention provides a compound according to the definition of a compound of formula (I), or subformulae thereof, that can also be used as a TDP-43 aggregates' biomarker or a TDP-43 proteinopathy biomarker, as a TDP-43 proteinopathy diagnostic agent or diagnostic tool or as an in vitro analytical reference or an in vitro screening tool.

Another aspect of the present invention provides a method of preparing a compound according to the definition of a compound of formula (I), or subformulae thereof.

In yet another aspect, the present invention relates to kit for preparing a radiopharmaceutical preparation, said kit comprising a precursor of a compound of formula (I), or subformulae thereof.

### BRIEF DESCRIPTION OF THE FIGURES

FIG. 1: Saturation binding curve on human FTD sarkosyl insoluble brain extracts. Each point represents the average of two independent experiments±standard error of the mean (SEM). FIGS. 1a-1e, human FTD type A sarkosyl insoluble brain extracts. FIG. 1a: [³H]-Compound 1; FIG. 1b: [³H]-Compound 7; FIG. 1c: [³H]-Compound 8; FIG. 1d: [³H]-Compound 9; FIG. 1e: [³H]-Compound 15; FIG. 1f: [³H]-Compound 16; FIG. 1g: [³H]-Compound 17. FIGS. 1h-1i, human FTD type B sarkosyl insoluble brain extracts. FIG. 1h: [³H]-Compound 1; FIG. 1i: [³H]-Compound 17

FIG. 2: Micro-autoradiography on FTLD-TDP brain tissue. Image of silver grain deposition (A, black arrows) on FTLD-TDP post-mortem human brain tissue colocalizing with pTDP-43 immuno-staining (B, white arrows). Scale bar is 10 μm (FIGS. 2a, 2b) or 20 μm (FIG. 2c, 2e). FIG. 2a: [³H]-Compound 1 on FTLD-TDP type A brain tissue; FIG. 2b: [³H]-Compound 9 on FTLD-TDP type A brain tissue; FIG. 2c: [³H]-Compound 17 on FTLD-TDP type A brain tissue. FIG. 2d: [³H]-Compound 1 on FTLD-TDP type B brain tissue. FIG. 2e: [³H]-Compound 17 on FTLD-TDP type B brain tissue.

FIG. 3: Classical autoradiography of [³H]-Compound 1 on FTLD-TDP or control brain tissue. FIG. 3a: Autoradiography images on control, FTLD-TDP type A or FTLD-TDP type B brain tissue. For each tissue type images of the total binding (Total) and non-specific binding (Self-block) are shown. FIG. 3b: Specific binding (total—non-specific) in control, FTLD-TDP type A and FTLD-TDP type B. Statistical analysis was performed using ordinary one-way ANOVA with Tukey's correction for multiple comparison. **p>0.01, *p<0.05. FIG. 3c: Saturation binding curve on FTLD-TDP type A by classical autoradiography. Specific binding of [³H]-Compound 1 is shown.

FIG. 4: Classical autoradiography of [³H]-Compound 17 on FTLD-TDP or control brain tissue. FIG. 4a: Autoradiography images on control, FTLD-TDP type A or FTLD-TDP type B brain tissue. For each tissue type images of the total binding (Total) and non-specific binding (Self-block) are shown. FIG. 4b: Specific binding (total—non-specific) in control, FTLD-TDP type A and FTLD-TDP type B. Statistical analysis was performed using ordinary one-way ANOVA with Tukey's correction for multiple comparison. **p>0.01, *p<0.05. FIG. 4c: Saturation binding curve on FTLD-TDP type A by classical autoradiography. Specific binding of [³H]-Compound 17 is shown.

FIG. 5: Binding specificity of [³H]-Compound 1 and [³H]-Compound 17 on human brain-derived extracts. FIGS. 5a-5b: Saturation binding curve of [³H]-Compound 1 (Figure Sa) or [³H]-Compound 17 (FIG. 5b) and [³H]-Abeta reference compound on AD brain homogenates. FIGS. 5c-5d: Saturation binding curve of [³H]-Compound 1 (FIG. 5c) or [³H]-Compound 17 (FIG. 5d) and a-syn reference compound on PD brain-derived insoluble fraction. FIGS. 5e-5f: Saturation binding curve of [³H]-Compound 1 (FIG. 5e) or [³H]-Compound 17 (FIG. 5f) and [³H]-Tau reference compound on AD brain-derived tau PHF.

FIG. 6: Target engagement on AD brain tissue sections by autoradiography. Classical autoradiography of [³H]-Compound 1 (FIG. 6a) and [³H]-Compound 17 (FIG. 6b) on Abeta- and tau-rich AD brain tissue sections. Scale bar is 2 mm. Micro-autoradiography of [³H]-Compound 1 (FIG. 6c) and [³H]-Compound 17 (FIG. 6d) on AD post-mortem human brain tissue containing pathological Tau aggregates.

FIG. 7: iv Non-Human Primate PK in whole monkey brain. FIG. 7a: using [¹⁸F]-Compound 1. FIG. 7b: using [¹⁸F]-Compound 17.

### DEFINITIONS

Unless defined otherwise, within the meaning of the present application the following definitions apply, and, when appropriate, a term used in the singular will also include the plural and vice versa:

Compounds of the invention may have one or more optically active carbons that can exist as racemates and racemic mixtures, stereoisomers (including diastereomeric mixtures and individual diastereomers, enantiomeric mixtures and single enantiomers, mixtures of conformers and single conformers), tautomers, atropoisomers, and rotamers. All isomeric forms are included in the present invention. Compounds described in this specification containing olefinic double bonds include E and Z geometric isomers. Also included in this invention are all salt forms, such as pharmaceutically acceptable salts, polymorphs, hydrates, solvates, prodrugs, and mixtures thereof. Unless specified otherwise, the terms “compound of formula (I)” or “compound of the (present) invention” refer to a “compound of formula (I), or a detectably labelled compound, stereoisomer, polymorph, racemic mixture, tautomer, pharmaceutically acceptable salt, prodrug, hydrate, or solvate thereof, or mixtures thereof”. Unless specified otherwise, the terms “compound of formula (I)” or “compound of the (present) invention” refers to compounds of formula (I), and subformulae thereof, and isotopically labelled compounds (including, but not limited to ¹⁸F and ³H substitutions). The terms “compound of formula (I)” or “compound of the (present) invention” refers to a compound as defined in any one of embodiments mentioned herein below.

The term “polymorphs” refers to the various crystalline structures of the compounds of the invention. This may include, but is not limited to, crystal morphologies (and amorphous materials) and all crystal lattice forms. Salts can also be crystalline and may exist as more than one polymorph.

Solvates, hydrates as well as anhydrous forms of the salt are also encompassed by the invention. The solvent included in the solvates is not particularly limited and can be any pharmaceutically acceptable solvent. Examples include C₁-C₄ alcohols (such as methanol or ethanol).

“Pharmaceutically acceptable salts” are defined as derivatives of the compounds of the present invention wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as, but not limited to, hydrochloric, hydrobromic, sulfuric, sulfamic, phosphoric, nitric and the like; and the salts prepared from organic acids such as, but not limited to, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. The pharmaceutically acceptable salts of the compound of formula (I) can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Organic solvents include, but are not limited to, nonaqueous media like ethers, ethyl acetate, ethanol, isopropanol, or acetonitrile. Lists of suitable salts can be found in Remington's Pharmaceutical Sciences, 18^{th} ed., Mack Publishing Company, Easton, PA, 1990, p. 1445, the disclosure of which is hereby incorporated by reference. Typically, the pharmaceutically acceptable salts are salts of amine residues in the compounds of the present invention.

The compound of the invention can also be provided in the form of a prodrug, namely a compound which is metabolized in vivo to the active metabolite.

The “patients” or “subjects” in the present invention are typically animals, particularly mammals, more particularly humans and mice. Even more particularly humans.

A “diagnostic composition” is defined in the present invention as a composition comprising the compound of the invention in a form suitable for administration to a patient, wherein the patient is e.g. a mammal such as a human.

“TDP-43 aggregates” are TDP-43-positive multimeric rich assemblies of TDP-43. They can be found in intracellular deposits in a range of diseases termed TDP-43 proteinopathies, in particular in amyotrophic lateral sclerosis (ALS), Alzheimer's disease (AD), Frontotemporal dementia (FTD) and limbic-predominant age-related TDP-43 encephalopathy (LATE). TDP-43 aggregates can be found in the following morphologies: compact oval or crescentic neuronal cytoplasmic inclusions (NCI), lentiform neuronal intranuclear inclusions (NII), glial cytoplasmic inclusions (GCI), dystrophic neurites (DN) and long tortous neurites. In pathological aggregates TDP-43 often displays a substantial increase in post-translational modifications such as phosphorylation, ubiquitination, acetylation, sumoylation and proteolytic cleavage to generate C-terminal fragments.

The “preclinical state” of disease is defined as the phase of disease where disease-associated changes on the molecular level are not leading to overt clinical representation in the patient.

The “clinical state” of a disease is defined as the phase of a disease where disease-associated changes on the molecular level led to overt clinical representation in the patient.

The terms “diagnosing” or “diagnosis” generally refer to the process or act of recognizing, deciding on or concluding on a disease or condition in a patient on the basis of symptoms and signs and/or from results of a diagnostic procedure.

A “normal control value” is determined by conducting the respective method with a plurality of healthy subjects, measuring the amount of the compound bound to the TDP-43 aggregates, if any, for each healthy subject and calculating an average thereof.

A “healthy control subject” or “healthy subject” is a person showing no clinical evidence of neurodegenerative disease. Said person needs to meet the following criteria:
Males and females' subjects which are healthy with no clinically relevant findings upon physical examination.
No family history of TDP-43 proteinopathy, TDP-43 aggregates formation, or other early-onset neurological diseases associated with dementia.
No personal history of clinically significant neurologic and/or psychiatric disorders.
No clinical signs or symptoms of a current neurological deficit such as cognitive impairment or motor deficit.

A “preclinical control value” is determined by conducting the respective method with a plurality of subjects who are in a preclinical state, measuring the amount of the compound bound to the TDP-43 aggregates, if any, for each subject and calculating an average thereof.

A “clinical control value” is determined by conducting the respective method with a plurality of subjects who are in a clinical state, measuring the amount of the compound bound to the TDP-43 aggregates, if any, for each subject and calculating an average thereof.

The terms “predicting” or “prediction” generally refer to an advance declaration, indication or foretelling of a disease or condition in a patient not having a disease, disorder or abnormality. For example, a prediction of a disease, disorder or abnormality in a patient may indicate a probability, chance or risk that the patient will contract the disease, disorder or abnormality, for example within a certain time period or by a certain age.

Detectable labels include suitable isotopes such as radioisotopes, in particular positron emitters or gamma emitters, and include ²H, ³H, ¹⁸F, ¹²³I, ¹²⁴I, ¹²⁵I, ¹³¹I, ¹¹C, ¹³N, ¹⁵O, ^{99m}Tc and ⁷⁷Br, preferably ²H, ³H, ¹¹C, ¹³N, ¹⁵O, and ¹⁸F, more preferably ²H, ³H and ¹⁸F, even more preferably ³H and ¹⁸F, most preferably ¹⁸F.

The term “Hal”, “halogen” or “halo” means F, Cl, Br or I, particularly Br or I, more particularly Br.

The term “carbocyclic” refers to a 5- or 6-membered carbocyclic ring which is not particularly limited and includes any 5- or 6-membered, saturated or unsaturated carbocyclic ring. Unsaturated carbocyclic rings include, but are not limited to, aromatic rings. Examples of 5- or 6-membered carbocyclic rings include, for instance, phenyl, cyclopentyl, cyclohexyl, cyclopentenyl, and cyclohexenyl. Phenyl being preferred.

The term “heterocyclic ring” refers to a stable 5- or 6-membered heterocyclic ring, is not particularly limited and includes any 5- or 6-membered, saturated or unsaturated heterocyclic ring. Unsaturated heterocyclic rings include, but are not limited to, aromatic rings. The heterocyclic ring contains one or more heteroatoms (for instance, one or two heteroatoms) selected from N, O and S. The heteroatom is/are preferably N or O, more preferably N. Examples of 5- or 6-membered heterocyclic rings include, for instance, pyridinyl, pyrimidinyl, pyrrolyl, pyrrolidinyl, furanyl, tetrahydrofuranyl, thiophenyl, imidazolidinyl, pyrazolidinyl, imidazolyl, pyrazolyl, oxathiolidinyl, isoxthiolidinyl, oxathiolyl, isoxathiolyl, thiazolidinyl, iosthiazolidinyl, thiazolyl, and isothiazolyl.

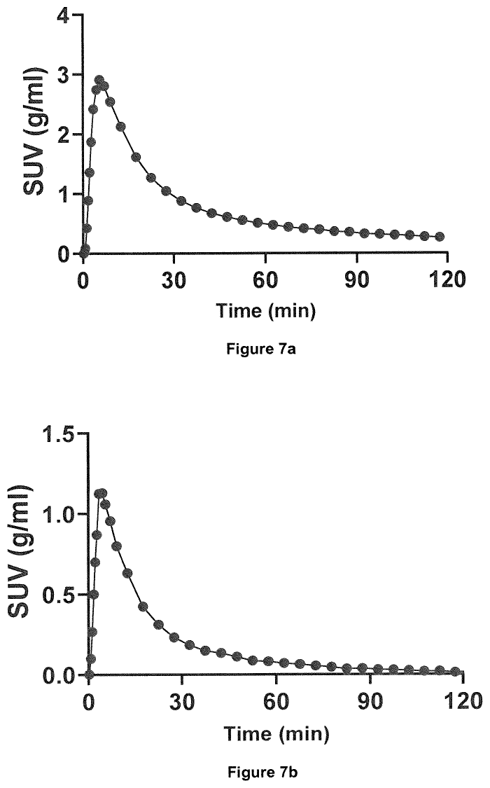

The term “leaving group” (LG) as employed herein is any leaving group and means an atom or group of atoms that can be replaced by another atom or group of atoms. Examples are given, e.g., in Synthesis (1982), p. 85-125, table 2, Carey and Sundberg, Organische Synthese, (1995), pages 279-281, table 5.8; or Netscher, Recent Res. Dev. Org. Chem., 2003, 7, 71-83, scheme 1, 2, 10 and 15 and others). (Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50, explicitly: scheme 4 pp. 25, scheme 5 pp 28, table 4 pp 30, FIG. 7 pp 33). Preferably, the “leaving group” (LG) is selected from C₁₋₄ alkyl sulfonate, C₆₋₁₀ aryl sulfonate or nitro. More preferably, the Leaving Group (LG) is mesylate, tosylate, nosylate or nitro. Even more preferably, the Leaving Group (LG) is mesylate or nitro.

The term “detecting” as used herein encompasses quantitative and/or qualitative detection.

The compounds of the present invention can be used as an analytical reference or an in vitro screening tool.

For example, the non-labelled compounds of formula (I) according to of the present invention can be used as an analytical reference for the quality control and release of a corresponding labelled compound of the present invention, for example a corresponding ¹⁸F labelled compound of Formula (I-F) or (I-F′). This quality control is conducted in an in vitro method.

The compounds of the present invention can be used as an in vitro screening tool for characterization of tissue with Tau pathology and for testing of compounds targeting Tau pathology on such tissue.

The preferred definitions given in the “Definition”-section apply to all of the embodiments described below unless stated otherwise. Various embodiments of the invention are described herein, it will be recognized that features specified in each embodiment may be combined with other specified features to provide further embodiments of the present invention.

### DETAILED DESCRIPTION OF THE INVENTION

Various embodiments of the invention are described herein, it will be recognized that features specified in each embodiment may be combined with other specified features to provide further embodiments of the present invention.

It is understood that all of the definitions which are given with respect to the formula (I) apply to all of the subgenera thereof, including the formulae (I-H), (I-F), (II), (III), and (IV).

In a first aspect, the present invention relates to a compound having the formula (I)

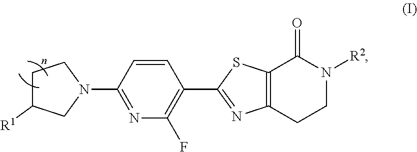

or a detectably labelled compound, stereoisomer, polymorph, racemic mixture, tautomer, pharmaceutically acceptable salt, prodrug, hydrate, or solvate thereof;
wherein
n is 1 or 2;
R¹ is H, hydroxy(C₁-C₄)alkyl or F; and
R² is a 5- or 6-membered carbocyclic or heterocyclic ring which can be optionally substituted with F, NH₂, CN and/or CH₃, wherein the heterocyclic ring contains one or more heteroatoms selected from N, O and S, preferably N.

In one embodiment, the present invention relates to a compound having the formula (I)

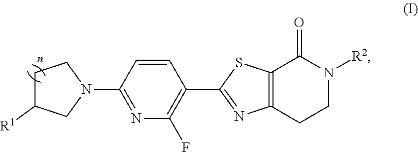

or a detectably labelled compound, stereoisomer, polymorph, racemic mixture, tautomer, pharmaceutically acceptable salt, prodrug, hydrate, or solvate thereof;
wherein
n is 1 or 2;
R¹ is H or F; and
R² is a 5- or 6-membered carbocyclic or heterocyclic ring which can be optionally substituted with F, NH₂, CN and/or CH₃, wherein the heterocyclic ring contains one or more heteroatoms selected from N, O and S, preferably N.

The present invention relates to compound of formula (I), wherein n is 1 or 2. In a preferred embodiment n is 1. In another preferred embodiment n is 2.

The present invention relates to a compound of formula (I), wherein R¹ is H, hydroxy(C₁-C₄)alkyl or F. In one embodiment, R¹ is H or F. In a preferred embodiment R¹ is H. In another preferred embodiment R¹ is F. In another preferred embodiment R¹ is hydroxy(C₁-C₄)alkyl. Preferred examples of hydroxy(C₁-C₄)alkyl are hydroxymethyl or hydroxyethyl, more preferably hydroxymethyl.

In a preferred embodiment R¹ is selected from the group consisting of F, H and —CH₂—OH.

The present invention refers to a compound of formula (I), wherein R² is a 5- or 6-membered carbocyclic or heterocyclic ring which can be optionally substituted with F, NH₂, CN and/or CH₃, wherein the heterocyclic ring contains one or more heteroatoms selected from N, O and S.

Preferably, R² is
a 5- or 6-membered carbocyclic ring, preferably an aryl ring, which can be optionally substituted with F, NH₂, CN and/or CH₃,
a 5-membered heteroaryl ring which can be optionally substituted with F, NH₂, CN and/or CH₃, wherein the 5-membered heteroaryl ring contains one or more heteroatoms selected from N, O and S, or
a 6-membered heteroaryl ring which can be optionally substituted with F, NH₂, CN and/or CH₃, wherein the 6-membered heteroaryl ring contains one or two heteroatoms selected from O, N and S.

Preferred examples of the ring of R² are given in the Definitions section above. Preferably, R² is phenyl, pyridyl, pyrimidinyl, pyrazolyl, pyridazinyl, thiazolyl or isothiazolyl (such as phenyl, pyridyl, pyrimidinyl, pyrazolyl), any of which can be optionally substituted with F, NH₂, CN and/or CH₃, e.g., by F, NH₂ and/or CH₃.

The ring of R² can be optionally substituted with F, NH₂, CN and/or CH₃ (e.g., F, NH₂ and/or CH₃) at any available position. In an embodiment, the 5- or 6-membered carbocyclic or heterocyclic ring is substituted with one or more of F, NH₂, CN and/or CH₃ (e.g., F, NH₂ and/or CH₃).

In one embodiment, the present invention relates to a compound of formula (I), wherein R² is a 5- or 6-membered carbocyclic or heterocyclic ring which can be optionally substituted with F, NH₂ and/or CH₃, wherein the heterocyclic ring contains one or more heteroatoms selected from N, O and S. In a preferred embodiment R² is a 5- or 6-membered carbocyclic or heterocyclic ring which can be optionally substituted with F, NH₂ and/or CH₃ at any available position. Preferably the 5- or 6-membered carbocyclic or heterocyclic ring is substituted with one or more of F, NH₂ and/or CH₃.

In one embodiment, the present invention relates to a compound of formula (I), wherein R² is a 5- or 6-membered carbocyclic or heterocyclic ring which can be optionally substituted with F, NH₂, CN, and/or CH₃, wherein the heterocyclic ring contains one or more heteroatoms selected from N, O and S. In a preferred embodiment R² is a 5- or 6-membered carbocyclic or heterocyclic ring which can be optionally substituted with F, NH₂, CN and/or CH₃ at any available position. In another embodiment, the 5- or 6-membered carbocyclic or heterocyclic ring is substituted with one or more of F, NH₂, CN and/or CH₃.

In one preferred embodiment, the phenyl can be optionally substituted by F, NH₂, and/or CN, preferably with CN and NH₂ or with F and NH₂. In another preferred embodiment, the pyrazolyl can be optionally substituted by CH₃. In another preferred embodiment, the pyridyl can be optionally substituted with F or NH₂. In another preferred embodiment, the pyridyl is unsubstituted. In another more preferred embodiment, the pyrimidinyl is unsubstituted. In another more preferred embodiment, the isothiazolyl or thiazolyl is unsubstituted. In another more preferred embodiment, the pyrazinyl is unsubstituted.

In a preferred embodiment, the present invention relates to a compound of formula (I), wherein
R² is

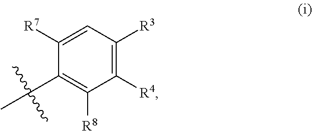

wherein

| | | | |
|---|---|---|---|
| R³ is F, | R⁴ is NH₂, | R⁷ is H, and | R⁸ is H, |
| R³ is NH₂, | R⁴ is F, | R⁷ is H, and | R⁸ is H, |
| R³ is CN, | R⁴ is NH₂, | R⁷ is H, and | R⁸ is H, |
| R³ is H, | R⁴ is NH₂, | R⁷ is H, and | R⁸ is CN, |
| R³ is H, | R⁴ is NH₂, | R⁷ is H, and | R⁸ is F, or |
| R³ is H, | R⁴ is NH₂, | R⁷ is H, and | R⁸ is CN. |

In another preferred embodiment, the present invention relates to a compound of formula (I), wherein
R² is

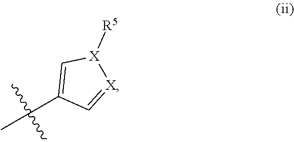

wherein X is N and R⁵ is H or CH₃. In a preferred embodiment R⁵ is CH₃.

In another preferred embodiment, the present invention relates to a compound of formula (I), wherein
R² is

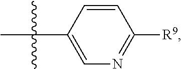

wherein R⁹ is selected from H, F, CH₃ and NH₂; preferably H;
or R² is

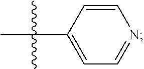 [SMILES: *c1ccncc1]

or R² is

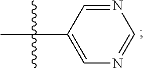 [SMILES: *c1cncnc1]

or R² is

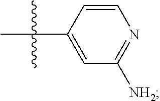 [SMILES: *c1ccnc(N)c1]

or R² is

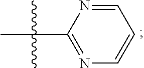 [SMILES: *c1ncccn1]

or R² is

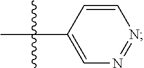 [SMILES: *c1ccnnc1]

or R² is

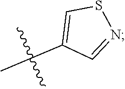 [SMILES: *c1cnsc1]

or R² is

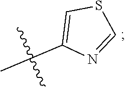 [SMILES: *c1cscn1]

or R² is

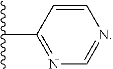 [SMILES: *c1ccncn1]

In one embodiment, the compound of formula (I) is defined as n is 1; R¹ is F; and R² is

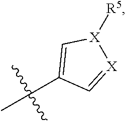

wherein X is N and R⁵ is CH₃ or H, preferably CH₃.

In one embodiment, the compound of formula (I) is defined as n is 1; R¹ is H; and R² is

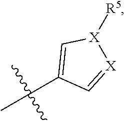

wherein X is N and R⁵ is CH₃ or H, preferably CH₃.

In one preferred embodiment, the compound of formula (I) is defined as n is 1; R¹ is F or H; and R² is

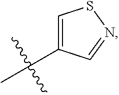 [SMILES: *c1cnsc1]

### or R² is

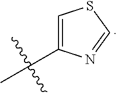 [SMILES: *c1cscn1]

In one embodiment, the compound of formula (I) is defined as n is 1; R¹ is H; and R² is

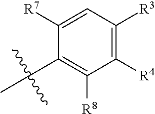

wherein R³ is F, R⁴ is —NH₂, R⁷ is H, R⁵ is H.

In one embodiment, the compound of formula (I) is defined as n is 1; R¹ is F; and R² is

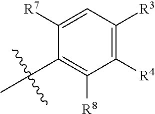

wherein R³ is F, R⁴ is —NH₂, R⁷ is H, R⁸ is H.

In one embodiment, the compound of formula (I) is defined as n is 1 or 2; R¹ is F; and R² is

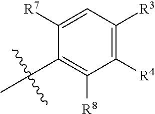

wherein R³ is H, R⁴ is NH₂, R⁷ is H, and R⁸ is CN.

In one embodiment, the compound of formula (I) is defined as n is 1; R¹ is H or F; and R² is

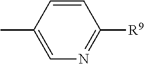

wherein R⁹ is selected from H, F, NH₂ or CH₃. In a preferred embodiment R⁹ is F. In one more preferred embodiment R⁹ is H.

In one embodiment, the compound of formula (I) is defined as n is 1; R¹ is H or F; and R² is

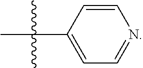 [SMILES: *c1ccncc1]

In another embodiment, the compound of formula (I) is defined as n is 1; R¹ is F; and R² is

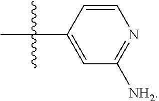 [SMILES: *c1ccnc(N)c1]

In another embodiment, the compound of formula (I) is defined as n is 2; R¹ is F; and R² is

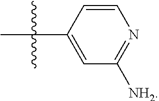 [SMILES: *c1ccnc(N)c1]

In one embodiment, the compound of formula (I) is defined as n is 1; R¹ is H or F; and R² is

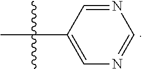 [SMILES: *c1cncnc1]

In one embodiment, the compound of formula (I) is defined as n is 1; R¹ is H or F; and R² is

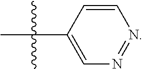 [SMILES: *c1ccnnc1]

In one embodiment, the compound of formula (I) is defined as n is 1; R¹ is H or F; and R² is

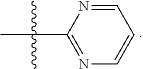 [SMILES: *c1ncccn1]

In one embodiment, the compound of formula (I) is defined as n is 1; R¹ is F; and R² is

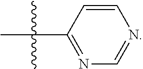 [SMILES: *c1ccncn1]

In one embodiment, the compound of formula (I) is defined as n is 2; R¹ is hydroxy(C₁-C₄)alkyl, preferably hydroxymethyl or hydroxyethyl, more preferably hydroxyethyl; and R² is

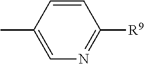

wherein R⁹ is selected from H, F, NH₂ or CH₃. In a preferred embodiment R⁹ is H.

Preferred compounds of formula (I) include:

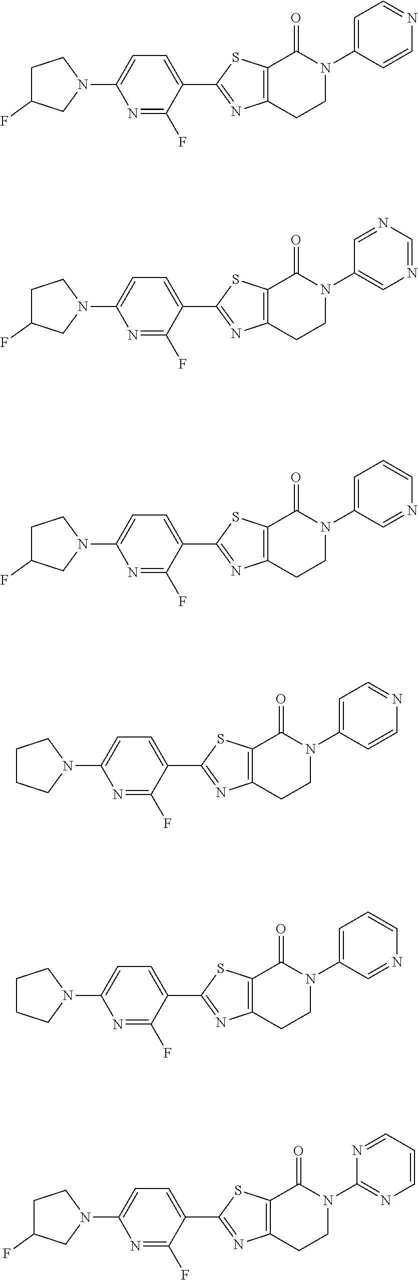 [SMILES: O=C1c2sc(-c3ccc(N4CCC(F)C4)nc3F)nc2CCN1c1ccncc1 ; O=C1c2sc(-c3ccc(N4CCC(F)C4)nc3F)nc2CCN1c1cncnc1 ; O=C1c2sc(-c3ccc(N4CCC(F)C4)nc3F)nc2CCN1c1cccnc1 ; O=C1c2sc(-c3ccc(N4CCCC4)nc3F)nc2CCN1c1ccncc1 ; O=C1c2sc(-c3ccc(N4CCCC4)nc3F)nc2CCN1c1cccnc1 ; O=C1c2sc(-c3ccc(N4CCC(F)C4)nc3F)nc2CCN1c1ncccn1]

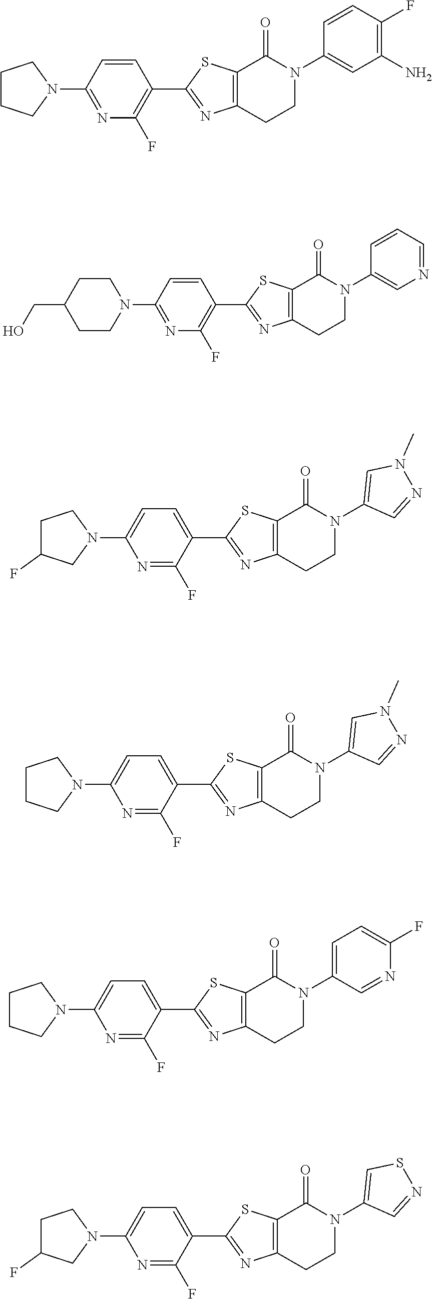 [SMILES: Nc1cc(N2CCc3nc(-c4ccc(N5CCCC5)nc4F)sc3C2=O)ccc1F ; O=C1c2sc(-c3ccc(N4CCC(CO)CC4)nc3F)nc2CCN1c1cccnc1 ; Cn1cc(N2CCc3nc(-c4ccc(N5CCC(F)C5)nc4F)sc3C2=O)cn1 ; Cn1cc(N2CCc3nc(-c4ccc(N5CCCC5)nc4F)sc3C2=O)cn1 ; O=C1c2sc(-c3ccc(N4CCCC4)nc3F)nc2CCN1c1ccc(F)nc1 ; O=C1c2sc(-c3ccc(N4CCC(F)C4)nc3F)nc2CCN1c1cnsc1]

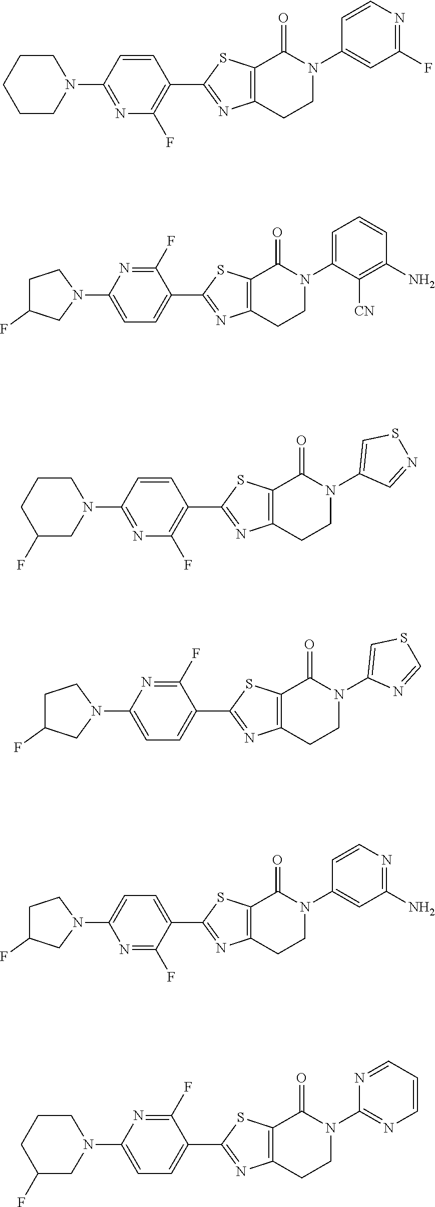 [SMILES: O=C1c2sc(-c3ccc(N4CCCCC4)nc3F)nc2CCN1c1ccnc(F)c1 ; N#Cc1c(N)cccc1N1CCc2nc(-c3ccc(N4CCC(F)C4)nc3F)sc2C1=O ; O=C1c2sc(-c3ccc(N4CCCC(F)C4)nc3F)nc2CCN1c1cnsc1 ; O=C1c2sc(-c3ccc(N4CCC(F)C4)nc3F)nc2CCN1c1cscn1 ; Nc1cc(N2CCc3nc(-c4ccc(N5CCC(F)C5)nc4F)sc3C2=O)ccn1 ; O=C1c2sc(-c3ccc(N4CCCC(F)C4)nc3F)nc2CCN1c1ncccn1]

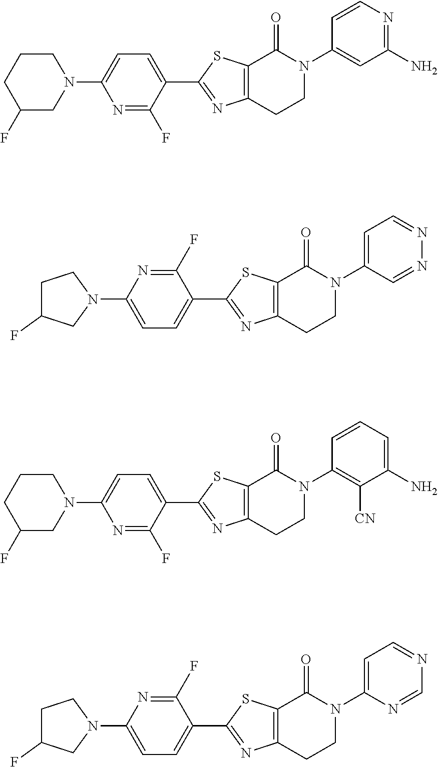 [SMILES: Nc1cc(N2CCc3nc(-c4ccc(N5CCCC(F)C5)nc4F)sc3C2=O)ccn1 ; O=C1c2sc(-c3ccc(N4CCC(F)C4)nc3F)nc2CCN1c1ccnnc1 ; N#Cc1c(N)cccc1N1CCc2nc(-c3ccc(N4CCCC(F)C4)nc3F)sc2C1=O ; O=C1c2sc(-c3ccc(N4CCC(F)C4)nc3F)nc2CCN1c1ccncn1]

In one embodiment preferred compounds of formula (I) can be selected from the following stereoisomers:

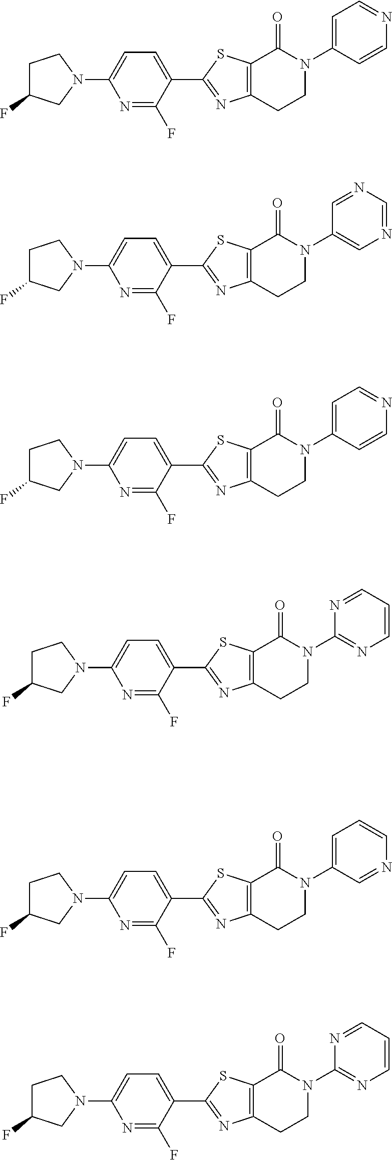 [SMILES: O=C1c2sc(-c3ccc(N4CC[C@H](F)C4)nc3F)nc2CCN1c1ccncc1 ; O=C1c2sc(-c3ccc(N4CC[C@@H](F)C4)nc3F)nc2CCN1c1cncnc1 ; O=C1c2sc(-c3ccc(N4CC[C@@H](F)C4)nc3F)nc2CCN1c1ccncc1 ; O=C1c2sc(-c3ccc(N4CC[C@H](F)C4)nc3F)nc2CCN1c1ncccn1 ; O=C1c2sc(-c3ccc(N4CC[C@H](F)C4)nc3F)nc2CCN1c1cccnc1 ; O=C1c2sc(-c3ccc(N4CC[C@H](F)C4)nc3F)nc2CCN1c1ncccn1]

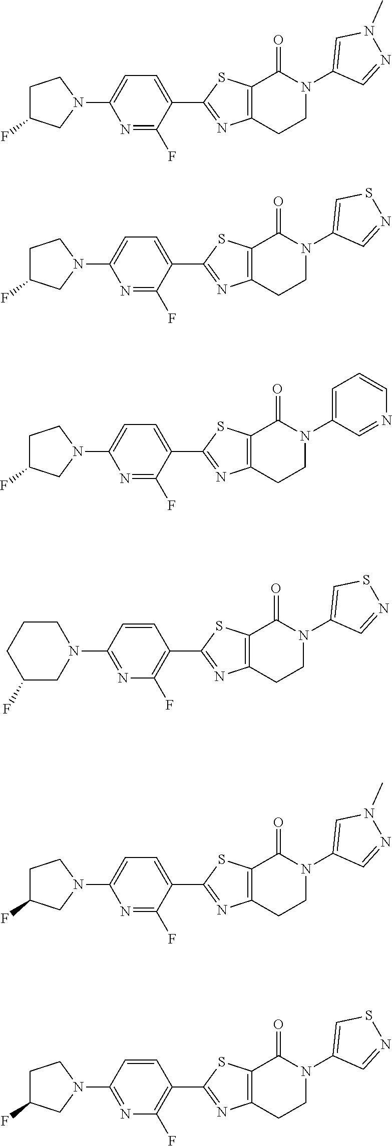 [SMILES: Cn1cc(N2CCc3nc(-c4ccc(N5CC[C@@H](F)C5)nc4F)sc3C2=O)cn1 ; O=C1c2sc(-c3ccc(N4CC[C@@H](F)C4)nc3F)nc2CCN1c1cnsc1 ; O=C1c2sc(-c3ccc(N4CC[C@@H](F)C4)nc3F)nc2CCN1c1cccnc1 ; O=C1c2sc(-c3ccc(N4CCC[C@@H](F)C4)nc3F)nc2CCN1c1cnsc1 ; Cn1cc(N2CCc3nc(-c4ccc(N5CC[C@H](F)C5)nc4F)sc3C2=O)cn1 ; O=C1c2sc(-c3ccc(N4CC[C@H](F)C4)nc3F)nc2CCN1c1cnsc1]

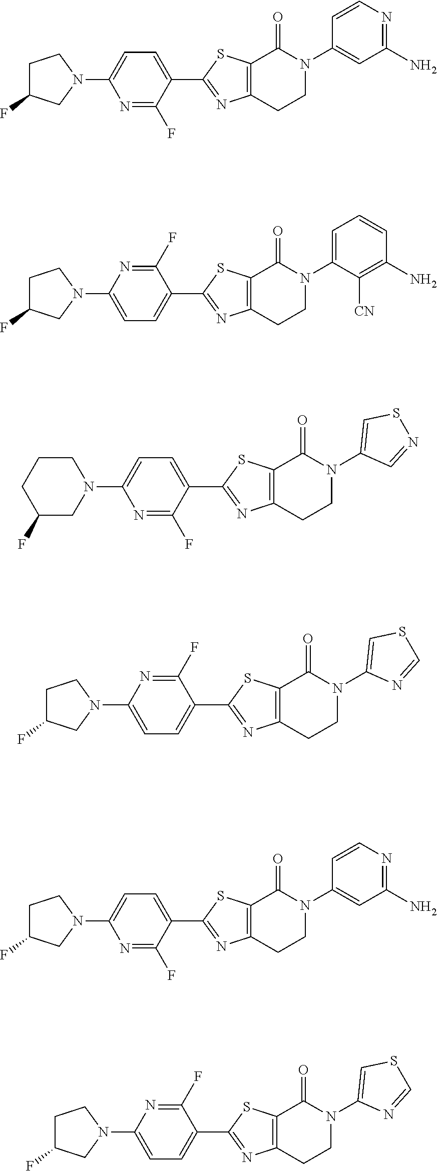 [SMILES: Nc1cc(N2CCc3nc(-c4ccc(N5CC[C@H](F)C5)nc4F)sc3C2=O)ccn1 ; N#Cc1c(N)cccc1N1CCc2nc(-c3ccc(N4CC[C@H](F)C4)nc3F)sc2C1=O ; O=C1c2sc(-c3ccc(N4CCC[C@H](F)C4)nc3F)nc2CCN1c1cnsc1 ; O=C1c2sc(-c3ccc(N4CC[C@@H](F)C4)nc3F)nc2CCN1c1cscn1 ; Nc1cc(N2CCc3nc(-c4ccc(N5CC[C@@H](F)C5)nc4F)sc3C2=O)ccn1 ; O=C1c2sc(-c3ccc(N4CC[C@@H](F)C4)nc3F)nc2CCN1c1cscn1]

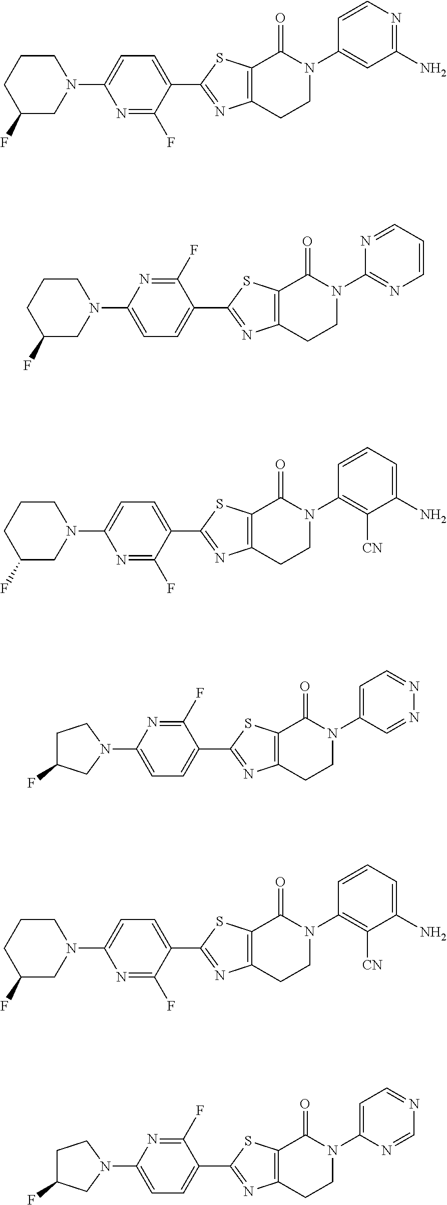 [SMILES: Nc1cc(N2CCc3nc(-c4ccc(N5CCC[C@H](F)C5)nc4F)sc3C2=O)ccn1 ; O=C1c2sc(-c3ccc(N4CCC[C@H](F)C4)nc3F)nc2CCN1c1ncccn1 ; N#Cc1c(N)cccc1N1CCc2nc(-c3ccc(N4CCC[C@@H](F)C4)nc3F)sc2C1=O ; O=C1c2sc(-c3ccc(N4CC[C@H](F)C4)nc3F)nc2CCN1c1ccnnc1 ; N#Cc1c(N)cccc1N1CCc2nc(-c3ccc(N4CCC[C@H](F)C4)nc3F)sc2C1=O ; O=C1c2sc(-c3ccc(N4CC[C@H](F)C4)nc3F)nc2CCN1c1ccncn1]

In one embodiment, the present invention relates to a compound of formula (I), which comprises a detectable label. Preferably, the compound of formula (I) comprises one or more detectable labels.

The type of the detectable label is not specifically limited and will depend on the detection method chosen. Examples of possible detectable labels include isotopes such as radioisotopes (namely radionuclides), in particular, positron emitters or gamma emitters. The detectable label such as the radioisotope, in particular, the positron emitter or gamma emitter, should be present in an amount, which is not identical to the natural amount of the respective isotope. Furthermore, the employed amount should allow detection thereof by the chosen detection method.

In a preferred embodiment, the detectable label is selected from ³H and ¹⁸F, most preferably ¹⁸F. The detectable label can be present at any available position. Typically, the detectable label is a radioactive isotope of one of the atoms which are present in the compound of formula (I). For instance, any reference to “F” in the present invention covers ¹⁹F (stable) or ¹⁸F (detectable label). Any reference to “H” covers ¹H (stable) or ³H (detectable label, so called tritium and represented herein as “T”).

Isotopic variations of the compounds of the invention can generally be prepared by conventional procedures such as by the illustrative methods or by the preparations described in the Examples and Preparative Examples hereinafter using appropriate isotopic variations of suitable reagents, commercially available or prepared by known synthetic techniques. Radioisotopes, in particular positron emitters or gamma emitters, can be included into the compounds of the invention by methods which are usual in the field of organic synthesis. Typically, they will be introduced by using a correspondingly labeled starting material. Illustrative methods of introducing detectable labels are described, for instance, in U.S. Pat. No. 8,932,557 which is incorporated herein by reference.

¹⁸F can be attached at any position which is suitable for attaching a fluorine. ¹⁸F-labeled compounds are particularly suitable for imaging applications such as positron emission tomography (PET). The corresponding compounds which include natural fluorine isotope ¹⁹F are also of particular interest as they can be used as analytical standards and references during manufacturing, quality control, release, and clinical use of their ¹⁸F-analogs.

In the compounds having the formula (I), ¹⁸F can be present, for instance, as the F substituent of R² or as R¹. Preferably it is present as R¹.

If ³H is employed as a detectable label it is preferably attached in the form of —CT₃ (T means ³H) at any position at which a CH₃ group can be attached. Substitution with radioisotopes such as ³H may afford certain diagnostic advantages resulting from greater metabolic stability by reducing, for example, defluorination, increasing in vivo half-life or reducing dosage requirements, while keeping or improving the original compound efficacy.

In one embodiment, the present invention relates to Tritium (³H) detectably labeled compounds having the formula (I), as described above, wherein at least one Hydrogen (H) is replaced by a detectable label selected from Tritium (³H). Tritium (³H) detectably labeled compounds having the formula (I) are preferably defined wherein 1 to 3 Hydrogens (H) are replaced by Tritium (³H). Tritium (³H) detectably labeled compounds having the formula (I) are more preferably defined wherein 2 or 3 Hydrogens (H) are replaced by Tritium (³H). Tritium (³H) detectably labeled compounds having the formula (I) are even more preferably defined wherein 3 Hydrogens (H) are replaced by Tritium (³H).

In one embodiment, the present invention provides a compound of formula (I), having a formula (I-T). In particular, the present invention relates to Tritium (³H) detectably labeled compounds having the formula (I-T)

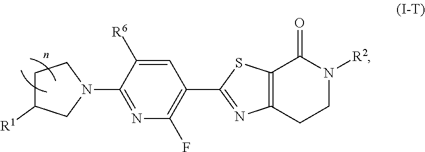

or a stereoisomer, polymorph, racemic mixture, tautomer, pharmaceutically acceptable salt, prodrug, hydrate, or solvate thereof, or mixtures thereof; wherein n, R¹, and R² are as defined herein with respect to compound of formula (I); R⁶ is T or H, and/or wherein R² is substituted by at least one CT₃ or at least one of the hydrogen atoms in R² is replaced by T, and/or wherein at least one of the hydrogen atoms in R¹ is replaced by T. In one embodiment, R⁶ is T or H, and/or wherein R² is substituted by at least one CT₃ or at least one of the hydrogen atoms in R² is replaced by T. T is ³H.

In one embodiment, R⁶ is T. In another embodiment, R² is substituted by at least one CT₃. In yet another embodiment, at least one of the hydrogen atoms in R² is replaced by T. In a preferred embodiment, R⁶ is T and at least one of the hydrogen atoms in R² is replaced by T. In yet another embodiment, at least one of the hydrogen atoms in R¹ is replaced by T.

In a preferred embodiment, the present invention relates to a compound of formula (I-T), wherein
n is 1 or 2; preferably n is 1;
R¹ is H or F;
R⁸ is T or H;
T is ³H; and
wherein
R² is

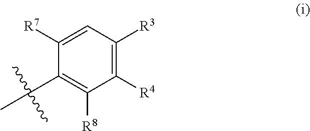

wherein R³ is F, R⁴ is —NH₂, and at least one of R⁷ and R⁸ is T and, if applicable, the other is H;
preferably R⁷ is T; and R⁸ is T; or
R² is

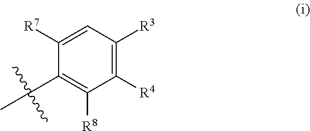

wherein R⁶ is CN, R⁴ is —NH₂, and at least one of R⁷ and R³ is T and, if applicable, the other is H; in one embodiment R⁷ is T; and R³ is T; in another embodiment R⁷ is T; and R³ is H.
R² is

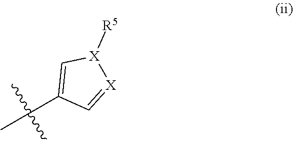

wherein X is N and R⁵ is tritiated CH₃ (CT₃); or
R² is

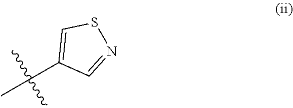 [SMILES: *c1cnsc1]

or
R² is

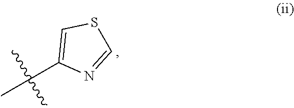 [SMILES: *c1cscn1]

or
R² is (iii) selected from

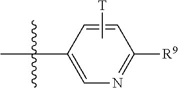

wherein R⁹ is selected from H, F, NH₂ or CH₃;

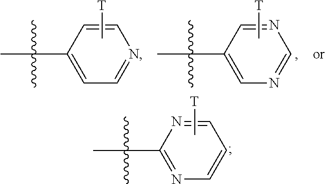

or
R² is

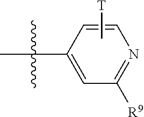

wherein R⁹ is selected from H, F, NH₂ or CH₃, preferably NH₂ or F;
or R² is

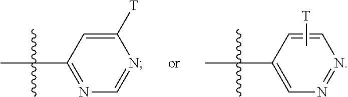

In an embodiment, the present invention relates to a compound of formula (I-T),

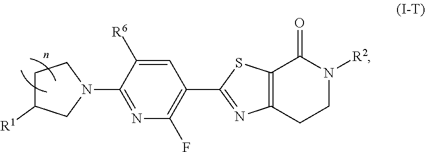

or a stereoisomer, polymorph, racemic mixture, tautomer, pharmaceutically acceptable salt, prodrug, hydrate, or solvate thereof;
wherein
n is 1 or 2;
R¹ is H, hydroxy(C₁-C₄)alkyl, or F;
T is ³H; and wherein
   R² is

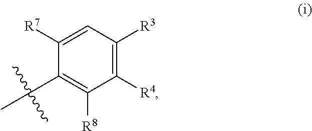

wherein R³ is F, R⁴ is NH₂, and at least one of R⁷ and R⁸ is T and, if applicable, the other is H; preferably R⁷ is T and R⁸ is T; and
R⁶ is T;
or wherein
R² is

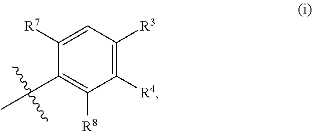

wherein R⁴ is NH₂, R⁸ is CN, and at least one of R³ or R⁷ is T and, if applicable, the other is H; preferably R⁷ is T and R³ is T; or R⁷ is T and R³ is H; and
R⁶ is T; or
wherein
R² is

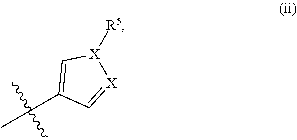

wherein X is N and R⁵ is CT₃; and
R⁶ is H; or wherein
R² is

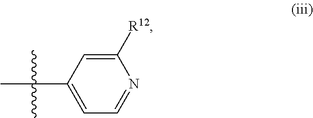

wherein R² is T;
or wherein
R² is

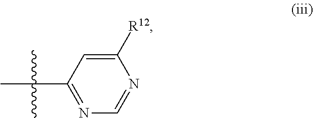

wherein R¹² is T.
or wherein
R² is

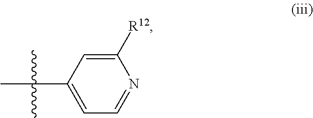

wherein R¹² is T.
or wherein
R² is

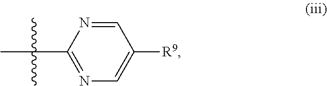

wherein R¹² is T.

In another embodiment, the present invention relates to a compound of formula (I-T), wherein
n is 1 or 2; preferably n is 2;
R¹ is H or F (preferably H);
R⁶ is T; and
R² is

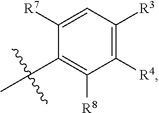

wherein R³ is F; R⁴ is NH₂; and at least one of R¹ and R⁸ is T and, if applicable, the other is H.

Preferably R⁷ is T; R⁸ is T;
or
R² is

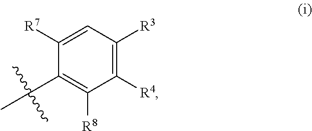

wherein R⁸ is CN, R⁴ is NH₂, and at least one of R⁷ and R³ is T and, if applicable, the other is H;

In one embodiment R⁷ is T; and R³ is T; or in another embodiment R⁷ is T; and R³ is H;
R² is

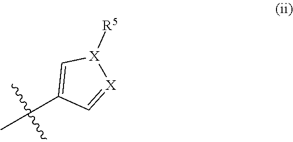

wherein X is N and R⁵ is tritiated CH₃ (CT₃); or
R² is

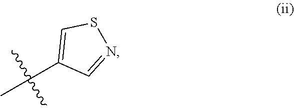 [SMILES: *c1cnsc1]

or
R² is

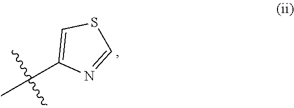 [SMILES: *c1cscn1]

or
R² is (iii) selected from

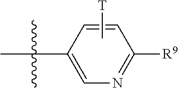

wherein R⁹ is selected from H, F, NH₂ or CH₃; from

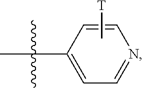

from

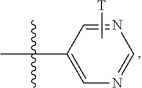

or R² is

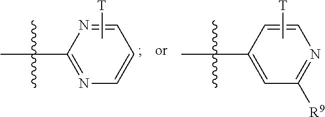

wherein R⁹ is selected from H, F, NH₂ or CH₃, preferably NH₂ or F; or R² is

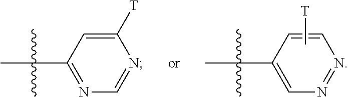

In one embodiment, the present invention relates to a compound of formula (I-T) wherein
n is 1 or 2; preferably n is 2;
R¹ is F;
R⁶ is H; and
R² is

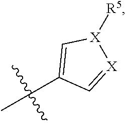

wherein X is N, and R⁵ is tritiated CH₃ (CT₃).

In one embodiment, the present invention relates to a compound of formula (I-T), wherein
n is 1 or 2; preferably n is 2;
R¹ is H or F;
R⁶ is T; and
R² is

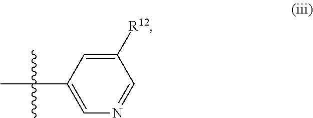

wherein R¹² is T.

In one embodiment, the present invention relates to a compound of formula (I-T), wherein
n is 1 or 2; preferably n is 2;
R¹ is H or F;
R⁶ is T; and
R² is

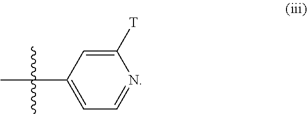

In one embodiment, the present invention relates to a compound of formula (I-T), wherein
n is 1 or 2; preferably n is 2;
R¹ is H or F;
R⁶ is T; and
R² is

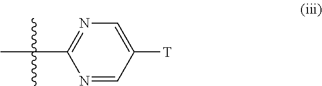

In another embodiment, the present invention relates to a compound of formula (I-T), wherein
n is 1 or 2;
R¹ is H or F, preferably H;
R⁶ is T; and
R² is

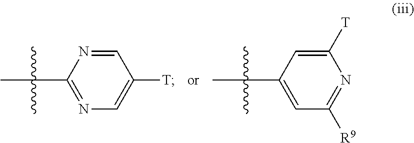

wherein R⁹ is selected from H, F, NH₂ or CH₃, preferably NH₂ or F; or R² is

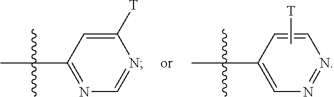

In a most preferred embodiment, the present invention relates to a compound of formula (I-T), wherein
n is 1 or 2; preferably n is 2;
R¹ is hydroxy(C₁-C₄)alkyl, preferably hydroxyethyl or hydroxymethyl, more preferably hydroxymethyl, wherein at least one of the hydrogen atoms in R¹ is replaced by T; preferably two of the hydrogen atoms in R¹ are replaced by T;
R⁶ is T or H;
T is ³H; and
wherein
R² is a 5- or 6-membered carbocyclic or heterocyclic ring which can be optionally substituted with F, NH₂, CN and/or CH₃, wherein the heterocyclic ring contains one or more heteroatoms selected from N, O and S, preferably N.

Preferred Tritium (³H) detectably labeled compounds of formula (I-T) according to present invention include (wherein T means ³H):

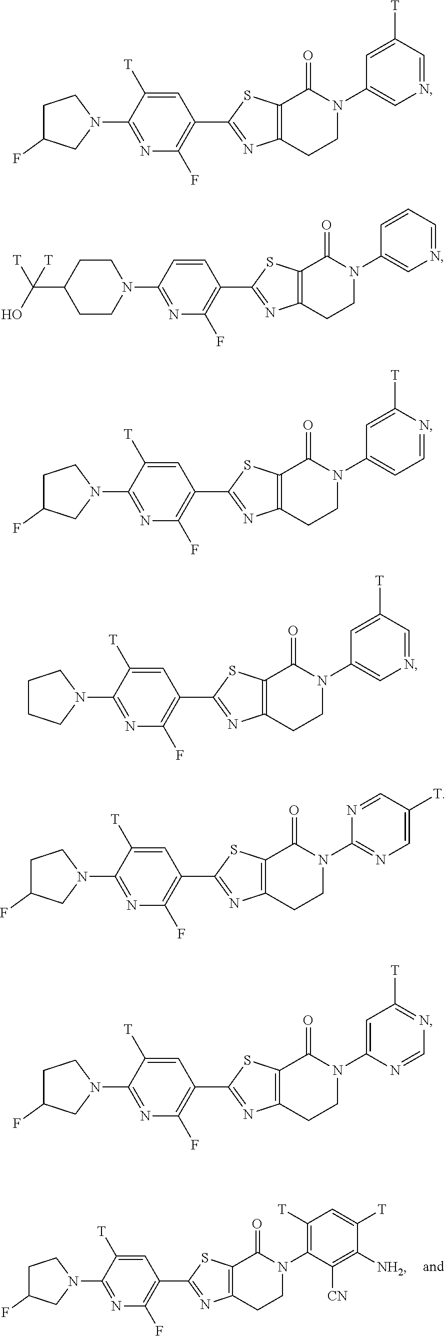

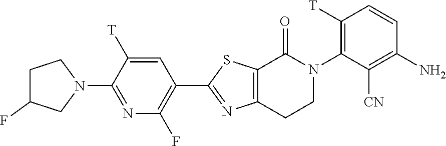

In a preferred embodiment, the Tritium (³H) detectably labeled compound for formula (I-T) according to the present invention can be the stereoisomer (wherein T means ³H)

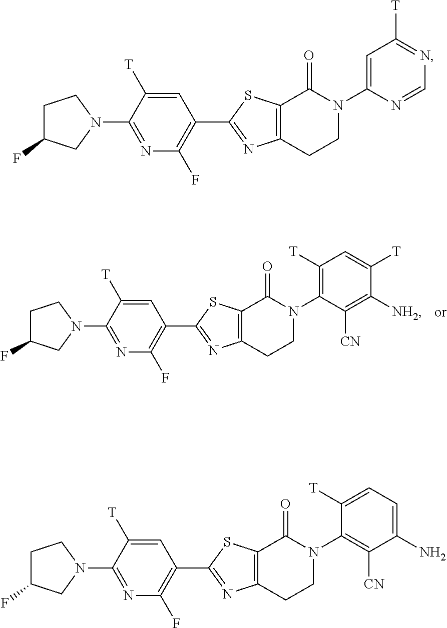

In a more preferred embodiment, the Tritium (³H) detectably labeled compound for formula (I-T) according to the present invention can be the stereoisomer (wherein T means ³H)

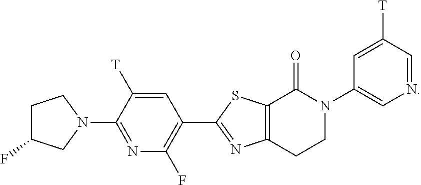

In another more preferred embodiment, the Tritium (³H) detectably labeled compound for formula (I-T) according to the present invention can be the compound (wherein T means ³H)

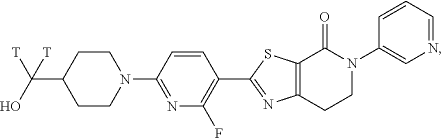

In one embodiment, the present invention provides a ¹⁸F detectably labeled compound of formula (I-F)

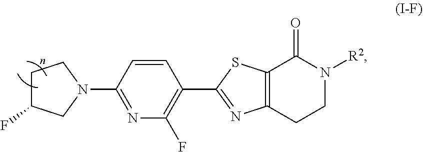

or a stereoisomer, polymorph, racemic mixture, tautomer, pharmaceutically acceptable salt, prodrug, hydrate, or solvate thereof, or mixtures thereof; wherein n, R¹, and R² are as defined herein with respect to compound of formula (I) and at least one F is ¹⁸F.

In a preferred embodiment, the present invention relates to a compound of formula (I-F), wherein R¹ is ¹⁸F (detectable label).

In a further preferred embodiment,
n is 1 or 2;
R¹ is ¹⁸F (detectable label); and
R² is

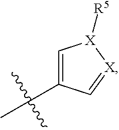

wherein X is N and R⁵ is CH₃ or H preferably CH₃; or
R² is

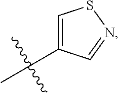 [SMILES: *c1cnsc1]

or R² is

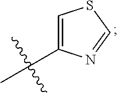 [SMILES: *c1cscn1]

or R² is

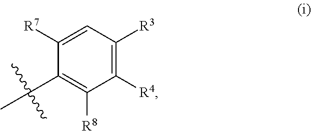

wherein

| | | | |
|---|---|---|---|
| R³ is F, | R⁴ is NH₂, | R⁷ is H, and | R⁸ is H; or |
| R³ is H, | R⁴ is NH₂, | R⁷ is H, and | R⁸ is CN; |

R² is

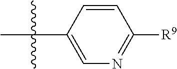

wherein R⁹ is selected from H, and F, NH₂ or CH₃; or
R² is

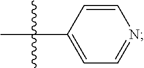 [SMILES: *c1ccncc1]

or
R² is

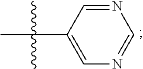 [SMILES: *c1cncnc1]

or
R² is

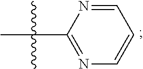 [SMILES: *c1ncccn1]

or
R² is

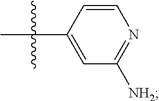 [SMILES: *c1ccnc(N)c1]

or
R² is

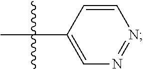 [SMILES: *c1ccnnc1]

or
R² is

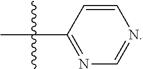 [SMILES: *c1ccncn1]

In a preferred embodiment, the present invention relates to a compound of formula (I-F), wherein
n is 1 or 2;
R¹ is ¹⁸F (detectable label);
R² is

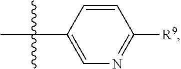

wherein R⁹ is H.

In a preferred embodiment n is 1. In another preferred embodiment n is 2.

In another embodiment, (I-F) is the following compound

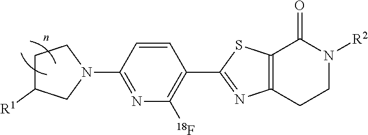

or a stereoisomer, polymorph, racemic mixture, tautomer, pharmaceutically acceptable salt, prodrug, hydrate, or solvate thereof, or mixtures thereof; wherein n, R¹, and R² are as defined herein with respect to compound of formula (I).

In another preferred embodiment, (I-F) is the following compound

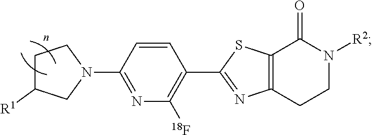

wherein n is 1 or 2;
R¹ is hydroxy(C₁-C₄)alkyl, preferably hydroxyethyl or hydroxymethyl, more preferably hydroxymethyl; and
R² is a 5- or 6-membered carbocyclic or heterocyclic ring which can be optionally substituted with F, NH₂ CN and/or CH₃, wherein the heterocyclic ring contains one or more heteroatoms selected from N, O and S, preferably N.

In a preferred embodiment
R² is

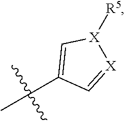

wherein X is N and R⁵ is CH₃ or H, preferably CH₃; or
R² is

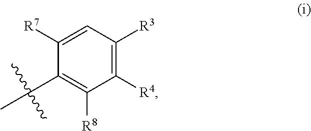

wherein

| | | | |
|---|---|---|---|
| R³ is F, | R⁴ is NH₂, | R⁷ is H, and | R⁸ is H; or |
| R³ is H, | R⁴ is NH₂, | R⁷ is H, and | R⁸ is CN; |

R² is

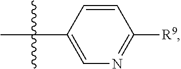

wherein R⁹ is selected from H, and F, NH₂ or CH₃; or
R² is

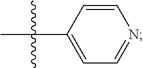 [SMILES: *c1ccncc1]

or
R² is

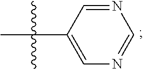 [SMILES: *c1cncnc1]

or
R² is

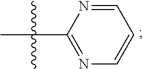 [SMILES: *c1ncccn1]

or
R² is

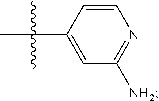 [SMILES: *c1ccnc(N)c1]

or
R² is

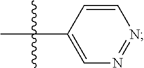 [SMILES: *c1ccnnc1]

or
R² is

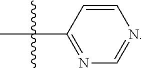 [SMILES: *c1ccncn1]

In one preferred embodiment n is 1 or 2, preferably 2;
R¹ is hydroxy(C₁-C₄)alkyl, preferably hydroxyethyl or hydroxymethyl, more preferably hydroxymethyl; and R² is

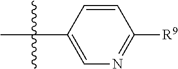

wherein R⁹ is selected from H, and F, NH₂ or CH₃, preferably H.

In an embodiment n is 1. In a preferred embodiment n is 2.

In one preferred embodiment, the present invention relates to a compound of formula (I-F),

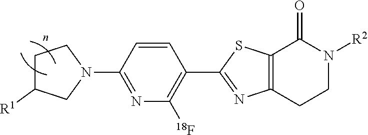

wherein
n is 1 or 2, preferably 2;
R¹ is hydroxy(C₁-C₄)alkyl, preferably hydroxyethyl or hydroxymethyl, more preferably hydroxymethyl; and
R² is

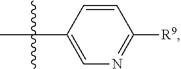

wherein R⁹ is H.

Preferred ¹⁸F detectably labeled compounds of formula (I-F) according to the present invention can be selected from

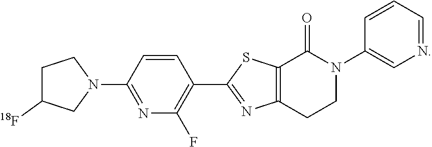 [SMILES: O=C1c2sc(-c3ccc(N4CCC([18F])C4)nc3F)nc2CCN1c1cccnc1]

More preferably, the ¹⁸F detectably labeled compound of formula (I-F) according to the present invention can be the stereoisomer

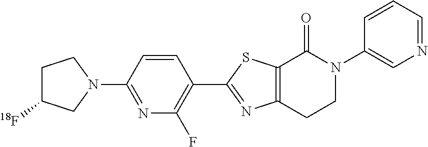 [SMILES: O=C1c2sc(-c3ccc(N4CC[C@@H]([18F])C4)nc3F)nc2CCN1c1cccnc1]

Other preferred ¹⁸F detectably labeled compounds of formula (I-F) according to the present invention can be

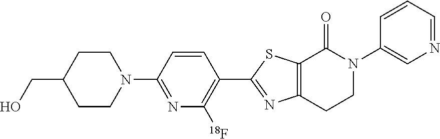 [SMILES: O=C1c2sc(-c3ccc(N4CCC(CO)CC4)nc3[18F])nc2CCN1c1cccnc1]

### Diagnostic Compositions

In a second aspect, the present invention relates to a diagnostic composition comprising a compound of formula (I), as described above, and optionally at least one physiologically acceptable carrier, diluent, adjuvant and/or excipient.

The compounds of the present invention are particularly suitable for imaging TDP-43 aggregates. The imaging can be conducted in mammals, preferably in humans. The imaging is preferably in vitro imaging, ex vivo imaging, or in vivo imaging. More preferably the imaging is in vivo imaging. Even more preferably, the imaging is brain imaging. The imaging can also be eye/retinal imaging or imaging of tissue of the central nervous system.

The compounds of the present invention are particularly suitable for use in diagnostics. The diagnostics can be conducted for mammals, preferably for humans. The tissue of interest on which the diagnostics is conducted can be brain tissue, tissue of the central nervous system, tissue of the eye (such as retinal tissue) or other tissues, or body fluids such as cerebrospinal fluid (CSF). The tissue is preferably brain tissue.

A “diagnostic composition” is defined in the present invention as a composition comprising one or more compounds of the present invention, in a form suitable for administration to a patient, (e.g., a mammal such as a human), and which is suitable for use in the diagnosis of the specific disease, disorder or abnormality at issue. In one embodiment, the diagnostic composition comprises a detectably labeled compound of the invention as described above and optionally at least one physiologically acceptable carrier, diluent, adjuvant and/or excipient.

Preferred detectably labeled compounds of the invention are of formula (I-T) or (I-F).

The diagnostic composition is suitable for use in the diagnosis of a disease, disorder or abnormality associated with TDP-43 aggregates or a TDP-43 proteinopathy, as defined herein below. Preferably a diagnostic composition further comprises, optionally, a physiologically acceptable excipient, carrier, diluent, or adjuvant. Administration is preferably carried out as defined below. More preferably by injection of the composition as an aqueous solution. The diagnostic composition may optionally contain further ingredients such as buffers; pharmaceutically acceptable solubilizers (e.g., cyclodextrins or surfactants such as Pluronic, Tween or phospholipids); and pharmaceutically acceptable stabilizers or antioxidants (such as ascorbic acid, gentisic acid or para-aminobenzoic acid). The dose of the compound of the invention will vary depending on the exact compound to be administered, the weight of the patient, and other variables as would be apparent to a physician skilled in the art.

While it is possible for the compounds of the invention to be administered alone, it is preferable to formulate them into a diagnostic composition in accordance with standard pharmaceutical practice. Thus, a diagnostic composition which comprises a diagnostically effective amount of a compound of the invention in combination with a pharmaceutically acceptable carrier, diluent, adjuvant and/or excipient is part of the invention. The preferred pharmaceutically acceptable carrier, diluent, adjuvant and/or excipient is one that is physiologically compatible with the diagnostic composition according to the present invention.

Pharmaceutically acceptable excipients are well known in the pharmaceutical art, and are described, for example, in Remington's Pharmaceutical Sciences, 18^{th} Ed. (Alfonso R. Gennaro, ed.; Mack Publishing Company, Easton, PA, 1990). The pharmaceutically acceptable excipient can be selected with regard to the intended route of administration and standard pharmaceutical practice. The excipient must be acceptable in the sense of being not deleterious to the recipient thereof.

Pharmaceutically useful excipients, carriers, adjuvants and diluents that may be used in the formulation of the diagnostic composition of the present invention may comprise, for example, solvents such as monohydric alcohols such as ethanol, isopropanol and polyhydric alcohols such as glycols and edible oils such as soybean oil, coconut oil, olive oil, safflower oil cottonseed oil, oily esters such as ethyl oleate, isopropyl myristate, binders, adjuvants, solubilizers, thickening agents, stabilizers, disintegrants, glidants, lubricating agents, buffering agents, emulsifiers, wetting agents, suspending agents, sweetening agents, colorants, flavors, coating agents, preservatives, antioxidants, processing agents, drug delivery modifiers and enhancers such as calcium phosphate, magnesium stearate, talc, monosaccharides, disaccharides, starch, gelatin, cellulose, methylcellulose, sodium carboxymethyl cellulose, dextrose, hydroxypropyl-β-cyclodextrin, polyvinylpyrrolidone, low melting waxes, and ion exchange resins.

The routes for administration (delivery) of the compounds of the invention include, but are not limited to, one or more of: intravenous, gastrointestinal, intraspinal, intraperitoneal, intramuscular, oral (e.g. as a tablet, capsule, or as an ingestible solution), topical, mucosal (e.g. as a nasal spray or aerosol for inhalation), nasal, parenteral (e.g. by an injectable form), intrauterine, intraocular, intradermal, intracranial, intratracheal, intravaginal, intracerebroventricular, intracerebral, subcutaneous, ophthalmic (including intravitreal or intracameral), transdermal, rectal, buccal, epidural and sublingual. Preferably, the route for administration (delivery) of the compounds of the invention is parenteral.

If the compounds of the present invention (for instance, detectably labeled compounds such as those with a ³H or ¹⁸F detectable label) are administered parenterally, then examples of such routes of administration include one or more of: intravenously, intraarterially, intraperitoneally, intrathecally, intraventricularly, intraurethrally, intrasternally, intracranially, intramuscularly or subcutaneously and/or using infusion techniques. For parenteral administration, the compounds are best used in the form of a sterile aqueous solution which may contain other excipients. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Typically, a physician will determine the actual dosage which will be most suitable for an individual patient. The dose of the compounds of the present invention (for instance, detectably labeled compounds such as those with a ³H or ¹⁸F detectable label) will vary depending on the exact compound to be administered, the weight of the patient, size and type of the sample, and other variables as would be apparent to a physician skilled in the art. Generally, the dose could preferably lie in the range 0.001 μg/kg to 10 μg/kg, preferably 0.01 μg/kg to 1.0 μg/kg. The radioactive dose can be, e.g., 100 to 600 MBq, more preferably 150 to 450 MBq.

Due to their design and their binding characteristics, the compounds of the present invention, as defined herein, can be use in the diagnosis of diseases, disorders and abnormalities associated with TDP-43 aggregates. The compounds of the present invention are particularly suitable for positron emission tomography imaging of TDP-43 aggregates.

The compounds of the present invention, as disclosed herein, are particularly suitable for use in the diagnosis of diseases, disorders or abnormalities associated with TDP-43 aggregates or the TDP-43 proteinopathy, such as disease, disorder or abnormality selected from, but not limited to, Frontotemporal dementia (FTD, such as Sporadic or familial with or without motor-neuron disease (MND), with progranulin (GRN) mutation, with C9orf72 mutations, with TARDBP mutation, with valosine-containing protein (VCP) mutation, linked to chromosome 9p, corticobasal degeneration, frontotemporal lobar degeneration (FTLD) including Frontotemporal lobar dementia TDP-43 or Frontotemporal lobar degeneration with ubiquitin-positive TDP-43 inclusions (FTLD-TDP), Argyrophilic grain disease, Pick's disease, semantic variant primary progressive aphasia (svPPA), behavioural variant FTD (bvFTD), Nonfluent Variant Primary Progressive Aphasia (such as nfvPPA), Amyotrophic lateral sclerosis (ALS, such as Sporadic ALS, with TARDBP mutation, with angiogenin (ANG) mutation), Alexander disease (AxD), limbic-predominant age-related TDP-43 encephalopathy (LATE), Chronic Traumatic Encephalopathy, Perry syndrome, Alzheimer's disease (AD, including sporadic and familial forms of AD), Down syndrome, Familial British dementia, Polyglutamine diseases (Huntington's disease and spinocerebellar ataxia type 3 (SCA3; also known under Machado Joseph Disease)), Hippocampal sclerosis dementia and Myopathies (Sporadic inclusion body myositis, Inclusion body myopathy with a mutation in the valosin-containing protein (VCP); also Paget disease of bone and frontotemporal dementia), Oculo-pharyngeal muscular dystrophy with rimmed vacuoles, Myofibrillar myopathies with mutations in the myotilin (MYOT) gene or mutations in the gene coding for desmin (DES), Traumatic Brain Injury (TBI), Dementia with Lewy Bodies (DLB) and Parkinson's disease (PD), preferably, the disease, the disorder or the abnormality associated with TDP-43 aggregates or the TDP-43 proteinopathy is selected from Frontotemporal dementia (FTD), amyotrophic lateral sclerosis (ALS), Alzheimer's disease (AD), Parkinson's disease (PD), Chronic Traumatic Encephalopathy (CTE), and limbic-predominant age-related TDP-43 encephalopathy (LATE).

In one embodiment, the diseases, disorders or abnormalities associated with TDP-43 aggregates or the TDP-43 proteinopathy is amyotrophic lateral sclerosis (ALS).

In one embodiment, the diagnosis of diseases, disorders or abnormalities associated with TDP-43 aggregates or the TDP-43 proteinopathy is Alzheimer's disease (AD).

In one embodiment, the diagnosis of diseases, disorders or abnormalities associated with TDP-43 aggregates or the TDP-43 proteinopathy is Frontotemporal dementia (FTD) including Frontotemporal lobar dementia TDP-43 or Frontotemporal lobar degeneration with TDP-43 inclusions (FTLD-TDP).

In one embodiment, the diagnosis of diseases, disorders or abnormalities associated with TDP-43 aggregates or the TDP-43 proteinopathy is limbic-predominant age-related TDP-43 encephalopathy (LATE).

### Methods and Uses

In a third aspect, the present invention relates to the methods and uses as listed below
A method of imaging a disease, disorder or abnormality associated with TDP-43 aggregates in a subject;
A method of positron emission tomography (PET) imaging of TDP-43 aggregates in a tissue of a subject;
A method for the detection and optionally quantification of TDP-43 aggregates in a tissue of a subject;
A method of the diagnostic imaging of the brain of a subject;
A method of determining an amount of TDP-43 aggregates in a sample or a specific body part or body area;
A method of diagnosing a disease, disorder or abnormality associated with TDP-43 aggregates or of diagnosing a TDP-43 proteinopathy;
A method of collecting data for the diagnosis of a disease, disorder or abnormality associated with TDP-43 aggregates or for the diagnosis of a TDP-43 proteinopathy;
A method of collecting data for determining a predisposition to a disease, disorder or abnormality associated with TDP-43 aggregates or a TDP-43 proteinopathy;
A method of collecting data for monitoring the progression of a disease, disorder or abnormality associated with TDP-43 aggregates or for monitoring the progression of a TDP-43 proteinopathy in a patient,
method of collecting data for predicting responsiveness of a patient suffering from a disease, disorder or abnormality associated with TDP-43 aggregates to a treatment with a medicament;
Use of a compound of the invention as a TDP-43 aggregates' biomarker or a TDP-43 proteinopathy biomarker,
Use of a compound of the invention as a TDP-43 proteinopathy diagnostic agent or diagnostic tool,
Use of a compound of the invention as an in vitro analytical reference or an in vitro screening tool.

Any of the compounds of the present invention (e.g. compound of formula (I), (I-T) or (I-F)) can be used in the above summarized methods. Preferably, said compounds are detectably labeled compounds (e.g. such as those with a ³H or ¹⁸F detectable label).

The methods of the invention can include the step of bringing a sample, a specific body part or a body area suspected to contain TDP-43 aggregates into contact with a compound of the invention.

The body is preferably of a mammal, more preferably of a human, including the full body or partial body area/part of the patient suspected to contain TDP-43 aggregates.

The sample can be selected from tissue or body fluids suspected to contain TDP-43 aggregates, the sample being obtained from the patient. Preferably, the tissue is selected from tissue of the central nervous system (CNS), eye tissue or brain tissue, more preferably brain tissue. Examples of body fluids include cerebrospinal fluid (CSF) or blood. The sample can be obtained from a mammal, more preferably a human. Preferably, the sample is an in vitro sample from a patient.

An in vitro sample or a specific body part or body area obtained from a patient can be brought into contact with a compound of the invention by direct incubation.

In an in vivo method, the specific body part or body area can be brought into contact with a compound of the invention by administering an effective amount of a compound of the invention to the patient. The effective amount of a compound of the invention is an amount which is suitable for allowing the presence or absence of TDP-43 aggregates in the specific body part or body area to be determined using the chosen analytical technique.

The step of allowing the compound of the invention to bind to the TDP-43 aggregates includes allowing sufficient time for said binding to happen. The amount of time required for binding will depend on the type of test (e.g., in vitro or in vivo) and can be determined by a person skilled in the field by routine experiments. In an in vitro method the amount of time will depend on the sample or specific body part or body area and can range, for instance, from about 30 min to about 120 min. In an in vivo method, the amount of time will depend on the time which is required for the compound of the invention to reach the specific body part or body area suspected to contain TDP-43 aggregates. The amount of time should not be too long to avoid washout and/or metabolism of the compound of the invention. The duration can range, for instance, from about 0 min to about 240 min (which is the duration of a PET scan during initial compound characterization (NHP PET and later FiH-study)).

The method of detecting the compound of the invention bound to the TDP-43 aggregates is not particularly limited and depends, among others, on the detectable label, the type of sample, specific body part or body area and whether the method is an in vitro or in vivo method. Possible detection methods include, but are not limited to, a fluorescence imaging technique or a nuclear imaging technique such as positron emission tomography (PET), single photon emission computed tomography (SPECT), magnetic resonance imaging (MRI), and contrast-enhanced magnetic resonance imaging (MRI). The fluorescence imaging technique and/or nuclear imaging technique can be employed for monitoring and/or visualizing the distribution of the compound of the invention within the sample or the body. The imaging system is such to provide an image of bound detectably label such as radioisotopes, in particular positron emitters or gamma emitters, as present in the tested sample, the tested specific body part or the tested body area. Preferably, the compound of the invention bound to the TDP-43 aggregates is detected by an imaging apparatus such as PET or a SPECT scanner. The amount of the compound bound to the TDP-43 can be determined by visual or quantitative analysis, for example, using PET scan images.

In one embodiment, the presence or absence of a compound of the invention bound with the TDP-43 aggregates can be correlated with the presence or absence of TDP-43 aggregates in the sample or specific body part or body area. The correlation can be qualitative or quantitative. In a preferred embodiment, this step comprises:
Determining the amount of the compound of the invention bound to the TDP-43 aggregates;
Correlating the amount of the compound of the invention bound to the TDP-43 aggregates with the amount of TDP-43 aggregates in the sample or specific body part or body area; and
Optionally comparing the amount of the compound bound with the TDP-43 aggregates in the sample or specific body part or body area to a normal control value in a healthy control subject.

The amount of the compound bound with the TDP-43 aggregates can be determined by any appropriate method. A preferred method is positron emission tomography (PET).

In another embodiment, the presence or absence of the compound of the invention bound to TDP-43 aggregates can be correlated with the disease, disorder or abnormality associated with TDP-43 aggregates or with the TDP-43 proteinopathy or a predisposition thereto. The correlation can be qualitative or quantitative. In a preferred embodiment, this step comprises:
Determining the amount of the compound of the invention bound to the TDP-43 aggregates;
Correlating the amount of the compound of the invention bound to the TDP-43 aggregates with the amount of TDP-43 aggregates in the sample or specific body part or body area; and
Optionally comparing the amount of the compound bound with the TDP-43 aggregates in the sample or specific body part or body area to a normal control value in a healthy control subject.

In any of the methods disclosed herein, steps (a) to (c) and, if present, optional step (d) can be repeated at least one time. The repetition of the steps is particularly useful in the method of collecting data for monitoring the progression and the method of collecting data for predicting responsiveness. In these methods, it may be expedient to monitor the patient over time and repeat the above steps after a certain period of time has elapsed. The time interval before the above-mentioned steps are repeated can be determined by a physician depending on the severity of the disease, disorder or abnormality associated with TDP-43 aggregates or the TDP-43 proteinopathy.

In one embodiment, the present invention relates to a method of detecting a neurological disease, disorder or abnormality associated with TDP-43 aggregates in a subject or a predisposition thereto, the method comprising the steps:
(a) Administering a compound of the invention; or a diagnostic composition comprising a compound of the invention as disclosed herein, to the subject;
(b) Allowing the compound to bind to the TDP-43 aggregates; and
(c) Detecting the compound which is bound to the TDP-43 aggregates.

In one embodiment, the present invention relates to a method (e.g. in vivo or in vitro method) for the detection and optionally quantification of TDP-43 aggregates in a tissue of a subject, the method comprising the steps:
(a) Administering a compound of the invention as disclosed herein; or a diagnostic composition comprising a compound of the invention as disclosed herein, to the subject;
(b) Allowing the compound to bind to the TDP-43 aggregates; and
(c) Detecting and optionally quantifying the compound bound to the TDP-43 aggregates using positron emission tomography.

In one embodiment, the present invention relates to a method of the diagnostic imaging of the brain of a subject, the method comprising the steps:
(a) Administering a compound of the invention; or a diagnostic composition comprising a compound of the invention as disclosed herein, to the subject;
(b) Allowing the compound to bind to the TDP-43 aggregates; and
(c) Detecting the compound bound to the TDP-43 aggregates by collecting a positron emission tomography (PET) image of the brain of the subject.

### Imaging:

The present invention relates to a method of imaging TDP-43 aggregates using the compounds of the invention. Imaging can be conducted, for example, using any of the above-mentioned methods, particularly by PET.

In one embodiment, the present invention relates to a method of imaging TDP-43 aggregates in a sample or a patient, in particular in a brain or a sample taken from the patient's brain, the method comprising the steps:
(a) Administering a compound of the invention; or a diagnostic composition comprising a compound of the invention as disclosed herein, to the subject;
(b) Allowing the compound to bind to the TDP-43 aggregates; and
(c) Detecting the compound bound to the TDP-43 aggregates.

In one embodiment, the present invention relates to a method of imaging or diagnosing a disease, disorder or abnormality associated with TDP-43 aggregates in a subject or a predisposition thereto, the method comprising the steps:
(a) Administering a compound of the invention; or a diagnostic composition comprising a compound of the invention as disclosed herein, to the subject;
(b) Allowing the compound to bind to the TDP-43 aggregates; and
(c) Detecting the compound bound to the TDP-43 aggregates in the brain of the subject.

In one embodiment, the present invention relates to a method of imaging or diagnosing a disease, disorder or abnormality associated with TDP-43 aggregates in a subject or a predisposition thereto, the method comprising the steps:
(a) Administering a compound of the invention; or a diagnostic composition comprising a compound of the invention as disclosed herein, to the subject;
(b) Allowing the compound to bind to the TDP-43 aggregates; and
(c) Detecting the compound bound to the TDP-43 aggregates.

In one embodiment, the present invention relates to a method of imaging or diagnosing a disease, disorder or abnormality associated with TDP-43 aggregates in a subject or a predisposition thereto, the method comprising the steps:
(a) Administering a compound of the invention; or a diagnostic composition comprising a compound of the invention as disclosed herein, to the subject;
(b) Allowing the compound to bind to the TDP-43 aggregates;
(c) Detecting the compound bound to the TDP-43 aggregates; and
(d) Generating an image representative of the location and/or amount of the compound bound to the TDP-43 aggregates.

In one embodiment, the present invention relates to a method of positron emission tomography (PET) imaging of TDP-43 aggregates in a tissue of a subject, the method comprising the steps:
(a) Administering a compound of the invention; or a diagnostic composition comprising a compound of the invention as disclosed herein, to the subject;
(b) Allowing the compound to bind to the TDP-43 aggregates; and
(c) Detecting the compound bound to the TDP-43 aggregates by collecting a positron emission tomography (PET) image of the tissue of the subject.

Preferably, the tissue is a tissue of the central nervous system (CNS), an eye tissue or a brain tissue.

More preferably, the tissue is brain tissue.

In one embodiment, the present invention relates to a method of imaging TDP-43 aggregates in a sample or a patient, the method comprises the steps:
(a) Bringing a sample, a specific body part or body area suspected to contain TDP-43 aggregates into contact with a compound of the invention; or with a diagnostic composition comprising a compound of the invention as disclosed herein;
(b) Allowing the compound to bind to the TDP-43 aggregates; and
(c) Detecting the compound bound to the TDP-43 aggregates by imaging the sample, the specific body part or the body area with an imaging system.

In one embodiment, the present invention relates to a method for imaging TDP-43 aggregates in an in vitro sample of a patient, the method comprising the steps:
(a) Bringing the in vitro sample suspected to contain TDP-43 aggregated into contact with a compound of the invention; or with a diagnostic composition comprising a compound of the invention as disclosed herein;
(b) Allowing the compound to bind to the TDP-43 aggregates; and
(c) Detecting the compound bound to the TDP-43 aggregates by imaging the in vitro sample with an imaging system.

In one embodiment, the present invention relates to a method of imaging TDP-43 aggregates in a patient or a specific body part or a body area of a patient, the method comprising the steps:
(a) Bringing a sample or a specific body part or body area suspected to contain TDP-43 aggregates into contact with a compound of the invention, preferably with a compound of formula (I-T) or of formula (I-F); or with diagnostic composition comprising a compound of the invention as disclosed herein, preferably a compound of formula (I-T) or of formula (I-F);
(b) Allowing the compound to bind to the TDP-43 aggregates; and
(c) Detecting the compound bound to the TDP-43 aggregates by imaging the sample or the specific body part or the body area of the patient with an imaging system.

The step of imaging the sample, the patient, the specific body part or the body area of the patient with an imaging system includes detecting the compound of the invention bound to the TDP-43 aggregates using an imaging system as disclosed herein. Detecting the compound of the invention bound to the TDP-43 aggregates allows to identify by imaging the distribution of TDP-43 aggregates in the tested sample, the patient, the specific body part or body area. The PET imaging should be conducted when the compound has penetrated the tissue and the compound has bound to the TDP-43 aggregates.

### Determining the Amount of TDP-43 Aggregates:

In one embodiment, the present invention relates to a method of determining the amount of TDP-43 aggregates in a sample, a specific body part or body area suspected to contain TDP-43 aggregates using a compound of the invention.

In one embodiment the present invention provides a method for determining the amount of TDP-43 aggregates in the sample, the specific body part or the body area suspected to contain TDP-43 aggregates, wherein the method comprises the steps of:
(a) Bringing a sample, a specific body part or body area suspected to contain TDP-43 aggregates into contact with a compound of the invention; or with a diagnostic composition comprising a compound of the invention as disclosed herein;
(b) Allowing the compound of the invention to bind to the TDP-43 aggregates;
(c) Detecting the compound of the invention bound to the TDP-43 aggregates;
(d) Determining the amount of compound of the invention bound to the TDP-43 aggregates; and
(e) Optionally calculating the amount of TDP-43 aggregates in the sample, the specific body part or body area.

A radioactive signal is observed when a detectably labelled compound of the invention, which comprises at least one radiolabeled atom (e.g. ³H, ²H, or ¹⁸F), is bound to the TDP-43 aggregates.

### Diagnosing:

In one embodiment, the present invention relates to a method of diagnosing a disease, disorder or abnormality associated with TDP-43 aggregates or TDP-43 proteinopathy or a predisposition thereto, the method comprising the steps of:
(a) Detecting the compound of the invention bound to the TDP-43 aggregates; and
(b) Correlating the presence or absence of the compound of the invention bound to TDP-43 aggregates with the disease, disorder or abnormality associated with TDP-43 aggregates or with the TDP-43 proteinopathy.

Preferably, the method of diagnosing the disease, disorder or abnormality associated with TDP-43 aggregates or the TDP-43 proteinopathy or a predisposition thereto comprises the steps of:
(a) Bringing a sample, a specific body part or body area suspected to contain TDP-43 aggregates into contact with a compound of the invention; or with a diagnostic composition comprising a compound of the invention as disclosed herein;
(b) Allowing the compound of the invention to bind to the TDP-43 aggregates;
(c) Detecting the compound of the invention bound to the TDP-43 aggregates; and
(d) Correlating the presence or absence of the compound of the invention bound to TDP-43 aggregates with the disease, disorder or abnormality associated with TDP-43 aggregates or with the TDP-43 proteinopathy.

In one embodiment, the present invention relates to a method of collecting data for the diagnosis of a disease, disorder or abnormality associated with TDP-43 aggregates or a TDP-43 proteinopathy or a predisposition thereto, the method comprising the following steps:
(a) Bringing a sample or a specific body part or body area suspected to contain TDP-43 aggregates into contact with a compound of the invention; or with a diagnostic composition comprising a compound of the invention as disclosed herein;
(b) Allowing the compound of the invention to bind to the TDP-43 aggregates;
(c) Detecting the compound of the invention bound to the TDP-43 aggregates; and
(d) Optionally correlating the presence or absence of the compound of the invention bound with the TDP-43 aggregates with the presence or absence of TDP-43 aggregates in the sample or specific body part or body area.

After the sample or a specific body part or body area has been brought into contact with the compound of the present invention, the compound is allowed to bind to the TDP-43 aggregates. The amount of time required for binding will depend on the type of test (e.g., in vitro or in vivo) and can be determined by a person skilled in the field by routine experiments. The compound which has bound to the TDP-43 aggregates can be subsequently detected by any appropriate method. The specific method chosen will depend on the detectable label which has been chosen. Examples of possible methods include, but are not limited to, a fluorescence imaging technique or a nuclear imaging technique such as positron emission tomography (PET), single photon emission computed tomography (SPECT), magnetic resonance imaging (MRI), and contrast-enhanced magnetic resonance imaging (MRI). The fluorescence imaging technique and/or nuclear imaging technique can be employed for monitoring and/or visualizing the distribution of the detectably labelled compound within the sample or a specific body part or body area.

The step of optionally correlating the presence or absence of the compound bound to the TDP-43 aggregates with the presence or absence of TDP-43 aggregates in the sample or specific body part or body area; as mentioned herein above, comprises the steps of
determining the amount of the compound bound to the TDP-43 aggregates;
correlating the amount of the compound bound to the TDP-43 aggregates with the amount of TDP-43 aggregates in the sample or specific body part or body area; and
optionally comparing the amount of the compound bound with the TDP-43 aggregates in the sample or specific body part or body area to a normal control value in a healthy control subject.

The amount of compound bound with the TDP-43 aggregates can be compared to a normal control value which has been determined in a sample or a specific body part or body area of a healthy subject, wherein an increase in the amount of the compound bound with the TDP-43 aggregates compared to a normal control value may indicate that the patient is suffering from or is at risk of developing a disease, disorder or abnormality associated with TDP-43 aggregates.

If the amount of the compound bound with the TDP-43 aggregates is higher than the normal control value, as defined herein, then it can be expected that the patient is suffering from or is likely to suffer from a disease, disorder or abnormality associated with TDP-43 aggregates or TDP-43 proteinopathy.

### Determining a Predisposition:

A further aspect of the present invention relates to a method of collecting data for determining a predisposition to a disease, disorder or abnormality associated with TDP-43 aggregates or a TDP-43 proteinopathy. The method comprises the steps:
(a) Bringing a sample or a specific body part or body area suspected to contain TDP-43 aggregates into contact with a compound of the invention; or with a diagnostic composition comprising a compound of the invention as disclosed herein;
(b) Allowing the compound of the invention to bind to the TDP-43 aggregates;
(c) Detecting the compound of the invention bound to the TDP-43 aggregates; and
(d) Optionally correlating the presence or absence of the compound of the invention bound with the TDP-43 aggregates with the presence or absence of TDP-43 aggregates in the sample or specific body part or body area.

The step of optionally correlating the presence or absence of the compound bound to the TDP-43 aggregates with the presence or absence of TDP-43 aggregates in the sample or specific body part or body area; as mentioned herein above, comprises the steps of
determining the amount of the compound bound to the TDP-43 aggregates;
correlating the amount of the compound bound to the TDP-43 aggregates with the amount of TDP-43 aggregates in the sample or specific body part or body area; and
optionally comparing the amount of the compound bound with the TDP-43 aggregates in the sample or specific body part or body area to a normal control value in a healthy control subject.

If the amount of the compound bound to the TDP-43 aggregates is higher than a normal control value of a healthy/reference subject this indicates that the patient/subject is suffering from or is at risk of developing a disease, disorder or abnormality associated with TDP-43 aggregates. In particular, if the amount of the compound bound to the TDP-43 aggregates is higher than what expected in a person showing no clinical evidence of neurodegenerative disease, it can be assumed that the patient has a disposition to a disease, disorder or abnormality associated with TDP-43 aggregates or with a TDP-43 proteinopathy.

### Monitoring Disease Progression:

In one embodiment, the present invention relates to a method of monitoring the progression of a disease, disorder or abnormality associated with TDP-43 aggregates or a TDP-43 proteinopathy in a patient. Typically, the patient is or has been undergoing treatment of the disease, disorder or abnormality associated with TDP-43 aggregates or is or with TDP-43 proteinopathy. In particular, the treatment can involve administration of an anti-TDP-43 medicament.

The method of collecting data for monitoring the progression of a disease, disorder or abnormality associated with TDP-43 aggregates or for monitoring the progression of a TDP-43 proteinopathy in a patient comprises the steps:
(a) Bringing a sample, a specific body part or body area suspected to contain TDP-43 aggregates into contact with the compound of the invention;
(b) Allowing the compound of the invention to bind to the TDP-43 aggregates;
(c) Detecting the compound of the invention bound to the TDP-43 aggregates;
(d) Optionally correlating the presence or absence of the compound of the invention bound with the TDP-43 aggregates with the presence or absence of TDP-43 aggregates in the sample or specific body part or body area; and
(e) Optionally repeating steps (a) to (c) and, if present, optional step (d) at least one time.

In order to monitor the progression over time of the disease, disorder or abnormality associated with TDP-43 aggregates or the TDP-43 proteinopathy, steps (a) to (c) and optional step (d) (if present) can be repeated one or more times. Preferably, the steps should be repeated until no further progression of the disease is observed in the patient.

The step of optionally correlating the presence or absence of the compound bound to the TDP-43 aggregates with the presence or absence of TDP-43 aggregates in the sample or specific body part or body area; as mentioned herein above, comprises the steps of
Determining the amount of the compound bound to the TDP-43 aggregates;
Correlating the amount of the compound bound to the TDP-43 aggregates with the amount of TDP-43 aggregates in the sample or specific body part or body area; and
Optionally comparing the amount of the compound bound with the TDP-43 aggregates in the sample or specific body part or body area to a normal control value in a healthy control subject.

In the method of monitoring progression over time the amount of the compound of the invention bound to the TDP-43 aggregates can be optionally compared at various points of time during the treatment, for instance, before and after onset of the treatment and/or at various points of time after the onset of the treatment. A change, especially a decrease, in the amount of the compound of the invention bound to the TDP-43 aggregates may indicate that the disease is not progressing.

### Predicting Responsiveness:

In one embodiment, the present invention relates to a method of predicting responsiveness of a patient suffering from a disease, disorder or abnormality associated with TDP-43 aggregates, or suffering from a TDP-43 proteinopathy to a treatment of said disease, disorder or abnormality associated with TDP-43 aggregates or TDP-43 proteinopathy.

The method can be used to predict the treatment which is most suitable for the patient. In particular, the treatment can involve administration of an anti-TDP-43 medicament.

The method for predicting responsiveness of a patient suffering from a disease, disorder or abnormality associated with TDP-43 aggregates or suffering from a TDP-43 proteinopathy to a treatment of said disease, disorder or abnormality associated with TDP-43 aggregates or TDP-43 proteinopathy can comprise the steps of:
(a) Bringing a sample, a specific body part or body area suspected to contain TDP-43 aggregates into contact with a compound of the invention; or with a diagnostic composition comprising a compound of the invention as disclosed herein;
(b) Allowing the compound of the invention to bind to the TDP-43 aggregates;
(c) Detecting the compound of the invention bound to the TDP-43 aggregates;
(d) Optionally correlating the presence or absence of the compound of the invention bound with the TDP-43 aggregates with the presence or absence of TDP-43 aggregates in the sample or specific body part or body area; and
(e) Optionally repeating steps (a) to (c) and, if present, optional step (d) at least one time.

Typically, the patient is/has been undergoing treatment of the disease, disorder or abnormality associated with TDP-43 aggregates or the TDP-43 proteinopathy. In particular, the treatment can involve administration of a medicament which is suitable for treating the disease, disorder or abnormality associated with TDP-43 aggregates.

The present method allows to predict the responsiveness of a patient to a certain treatment. In one embodiment, the responsiveness can be estimated, e.g., by repeating steps (a) to (c) and, if present, optional step (d) and monitoring the amount of the compound of the invention bound with the TDP-43 aggregates over a period of time during which the patient is undergoing treatment of the disease, disorder or abnormality associated with TDP-43 aggregates or with TDP-43 proteinopathy. If the amount changes over time, the skilled practioner can deduce whether the patient is responsive to the treatment. Typically, if the amount of the compound of the invention bound with the TDP-43 aggregates decreases over time, it can be assumed that the patient is responsive to the treatment.

Typically, if the amount of the compound bound with the TDP-43 aggregates is essentially constant or increases over time, it can be assumed that the patient is non-responsive to the treatment.

Alternatively, the responsiveness can be estimated by determining the amount of the compound of the invention bound to the TDP-43 aggregates. The amount of the compound bound to the TDP-43 aggregates can be compared to a control value such as a normal control value, a preclinical control value or a clinical control value. The control value may refer to the control value of healthy control subjects. Alternatively, the control value may refer to the control value of subjects known to be responsive to a certain therapy, or to the control value may refer to the control value of subjects known to be non-responsive to a certain therapy. The outcome with respect to responsiveness can either be “responsive” to a certain therapy, “non-responsive” to a certain therapy or “response undetermined” to a certain therapy. Response to the therapy may be different for the respective patients.

The step of optionally correlating the presence or absence of the compound bound to the TDP-43 aggregates with the presence or absence of TDP-43 aggregates in the sample or specific body part or body area; as mentioned herein above, comprises the steps of
Determining the amount of the compound bound to the TDP-43 aggregates;
Correlating the amount of the compound bound to the TDP-43 aggregates with the amount of TDP-43 aggregates in the sample or specific body part or body area; and
Optionally comparing the amount of the compound bound with the TDP-43 aggregates in the sample or specific body part or body area to a normal control value in a healthy control subject.

The control value can be, e.g., a normal control value, a preclinical control value and/or a clinical control value. A “healthy control subject” or “healthy subject” is a person showing no clinical evidence of neurodegenerative disease.

If in any of the above summarized methods the amount of the compound bound with the TDP-43 aggregates is higher than the normal control value, then it can be expected that the patient is suffering from or is likely to suffer from a disease, disorder or abnormality associated with TDP-43 aggregates or a TDP-43 proteinopathy.

Any of the compounds of the present invention can be used in the above summarized methods. Preferably detectably labeled compounds of the present invention, as disclosed herein, are employed in the above summarized methods.

In a fourth aspect, the present invention relates to the use of the compounds of the invention as a TDP-43 aggregates' diagnostic agent or diagnostic tool. In one embodiment, the present invention relates to the use of the compounds of the invention as an in vitro analytical reference or an in vitro screening tool. Said compounds of the invention are also useful in in vivo diagnostic methods. In such instances, the compounds of the invention may be detectably labeled compounds or contain cold isotopes.

In another embodiment, the present invention further relates to the use of the compounds of the present invention, more specifically detectably labelled compounds of the invention as defined herein, as diagnostic biomarkers enabling more efficient and precise patient selection, e.g., for longitudinal monitoring in clinical studies, or for supporting the development of novel therapeutics for treating TDP-43 proteinopathies. In another embodiment, the present invention further relates to the use of the compounds of the present invention, more specifically detectably labelled compounds of the invention as defined herein, as a TDP-43 aggregates' biomarker or a TDP-43 proteinopathy biomarker.

In another embodiment, the compounds of the invention may be employed for research use, in particular, as an analytical tool or reference molecule. Said compounds may also be used in detecting TDP-43 aggregates in vitro or in vivo. The compounds of the invention may be used to stain TDP-43 aggregates. For example, compounds of the invention may be used for histochemical detection in postmortem tissue such as brain tissue. The compounds of the invention are preferably detectably labelled compounds and may be directly or indirectly labelled as discussed herein.

### Kit of Parts

In a fifth aspect, the present invention relates further to a kit for use in one or more of the methods of the invention, wherein the kit comprises a compound of the invention as described herein. The kit typically comprises a container holding the compound of the invention and instructions for using the compound of the invention. Preferably, the kit comprises a compound of formula (I), as disclosed herein. More preferably, the compound of the invention is a detectably labelled compound (e.g. compound of formula (I-T) or (I-F)).

The term “kit” refers in general to any diagnostic kit known in the art. More specifically, the latter term refers to a diagnostic kit as described in Zrein et al., Clin. Diagn. Lab. Immunol., 1998, 5, 45-49.

The dose of the detectably labelled compounds of the present invention will vary depending on the exact compound to be administered, the weight of the patient, size and type of the sample, and other variables as would be apparent to a physician skilled in the art. Generally, the dose could preferably lie in the range 0.001 μg/kg to 10 μg/kg, preferably 0.01 μg/kg to 1.0 μg/kg. The radioactive dose can be, e.g., 100 to 600 MBq, more preferably 150 to 450 MBq.

In particular, such kits may be useful for performing the methods of the invention (which include, for example, but not limited to, imaging, diagnosing, and monitoring methods), e.g., for diagnosing of a disease, disorder or abnormality associated with TDP-43 aggregates or a TDP-43 proteinopathy. Such kits may comprise all necessary components for performing the herein provided methods. Typically, each component is stored separately in a single overall packaging. Suitable additional components for inclusion in the kits are, for example, buffers, detectable dyes, laboratory equipment, reaction containers, instructions and the like. Instructions for use may be tailored to the specific method for which the kit is to be employed.

The present invention relates further to a kit for the preparation of a detectably labeled compound of the invention, wherein in particular the detectable label is a radioisotope. Thus, the kit comprises a precursor of the detectably labeled compound of the formula (I) and a labeling agent which reacts with the precursor to introduce a detectable (e.g., radioactive) label. Preferred precursors are compounds of the formulae (II), (III) and (IV). The labeling agent which reacts with the precursor can be an agent which introduces a detectable (e.g., radioactive) label such as ¹⁸F or ³H. The labeling agent can be a ¹⁸F-fluorination agent.

### Method for Preparing a Compound of the Invention

In a sixth aspect, the present invention relates further to a method for preparing a compound of formula (I).

### Cold Isotope Compounds:

In one embodiment, the present invention relates to a method for preparing a compound of formula (I), as described above, the method comprising the step of:

Reacting a compound of formula (II) with R¹⁰ to provide a compound of formula (I)

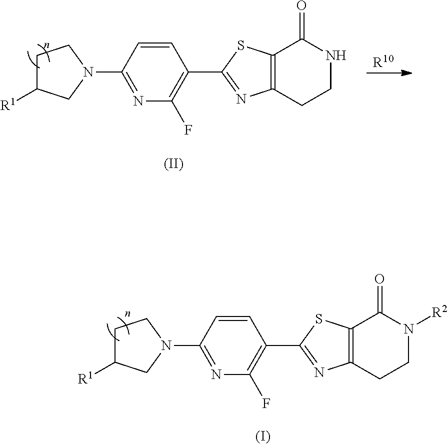

wherein
n, R¹, and R², are as defined above;
R¹⁰ is a 5- or 6-membered carbocyclic or heterocyclic ring which can be optionally substituted with F, NH₂, CN and/or CH₃, wherein the heterocyclic ring contains one or more heteroatoms selected from N, O and S, wherein the 5- or 6-membered carbocyclic or heterocyclic ring is substituted with Br or I.

In a preferred embodiment, R¹⁰ is selected from the following groups:

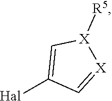

wherein X is N and R⁵ is CH₃ or H; and wherein Hal is Br or I; or

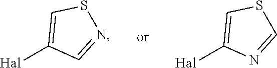

wherein Hal is Br;
R¹⁰ is

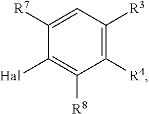

wherein Hal is Br or I; and

| | | | |
|---|---|---|---|
| R³ is F, | R⁴ is NH₂, | R⁷ is H, and | R⁸ is H; |
| R³ is NH₂, | R⁴ is F, | R⁷ is H, and | R⁸ is H, |
| R³ is CN, | R⁴ is NH₂, | R⁷ is H, and | R⁸ is H, |
| R³ is H, | R⁴ is NH₂, | R⁷ is H, and | R⁸ is CN, |
| R³ is H, | R⁴ is NH₂, | R⁷ is H, and | R⁸ is F, or |
| R³ is H, | R⁴ is NH₂, | R⁷ is H, and | R⁸ is CN; |

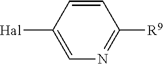

N wherein R⁹ is selected from H, F, NH₂ or CH₃, wherein Hal is Br or I;

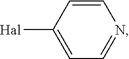

wherein Hal is Br;

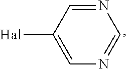

wherein Hal is Br;

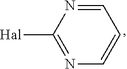

wherein Hal is Br;

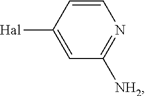

wherein Hal is Br;

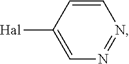

wherein Hal is Br; and

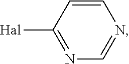

wherein Hal is Br.

The method of reacting the compound having the formula (II) with R¹¹ can be conducted by any suitable method. In one option, the reaction can be conducted in the presence of a diamine chelator such as DMEDA, a base such as potassium carbonate, a catalyst such as CuI, and an aprotic solvent such as dioxane. In another option, the reaction can be conducted under Pd-coupling conditions, in the presence of a Pd catalyst such as Pd[P(Ph)₃]₄ or Pd(OAc)₂, XantPhos.

### Tritium (³H) Detectably Labeled Compounds

In one embodiment, the present invention provides a method for preparing the Tritium (³H) detectably labeled compounds of the invention having the formula (I-T), said method comprises the step of radiolabeling a precursor of the compound having the formula (I-T) with a radioisotope, wherein at least one leaving group of the precursor of the compound having the formula (I-T) is replaced by Tritium (³H).

Tritium (³H) detectably labeled compounds having the formula (I-T) are preferably defined wherein at least 1 to 3 Hydrogen (H) are each replaced by Tritium (³H). Tritium (³H) detectably labeled compounds having the formula (I-T) are more preferably defined wherein 2 or 3 Hydrogen (H) are replaced by Tritium (³H). Tritium (³H) detectably labeled compounds having the formula (I) are even more preferably defined wherein 3 Hydrogen (H) are replaced by Tritium (³H).

In another embodiment, the present invention provides a method for preparing a compound of formula (I-T), said method comprises the step of:

Radiolabeling a precursor compound having the formula (III) with T (i.e., ³H) by either exchange of Br or I with T utilizing T₂ and a suitable catalyst or introduction of a CT₃-group

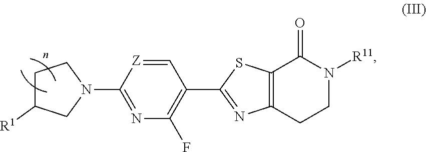

or a stereoisomer, polymorph, racemic mixture, tautomer, pharmaceutically acceptable salt, prodrug, hydrate, or solvate thereof, or mixtures thereof,
wherein
Z is selected from C—Br, C—I, and C—H;
n is 1 or 2;
R¹ is H, hydroxy(C₁-C₄)alkyl, or F, preferably R¹ is H, or F;
R¹¹ is a 5- or 6-membered carbocyclic or heterocyclic ring which can be optionally substituted with Br, I, F, NH₂, CN and/or CH₃, wherein the heterocyclic ring contains one or more heteroatoms selected from N, O and S;
and either at least one of Z and/or R¹¹ comprises Br or I;
wherein the at least one Br or I is replaced by or T; or
R¹¹ comprises a NH moiety;
wherein the NH-moiety is substituted with N-CT₃,
and wherein T is ³H.

In a preferred embodiment, Z is C—Br or C—I and the Br or I is replaced by T.

In another preferred embodiment, R¹¹ comprises Br or I and the Br or I is replaced by T.

In one preferred embodiment Z is C—Br or C—I and the Br or I is replaced by T; and R¹¹ comprises Br or I and the Br or I is replaced by T.

In another embodiment Z is C—H and R¹¹ is

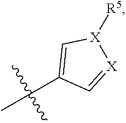

wherein R⁵ is H or CH₃ and at least one H on R¹¹ is replaced by CT₃.

In another embodiment, the present invention provides a method for preparing a compound of formula (I-T) comprising radiolabeling a precursor compound having the formula (III) with T (i.e., ³H)

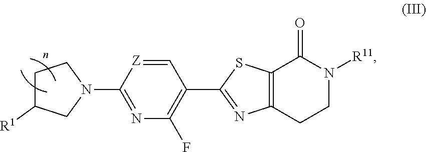

wherein
n is 1 or 2; preferably 2
R¹ is —COOR^{A}, wherein R^{A} is (C₁-C₄)alkyl, preferably hydroxyethyl or hydroxymethyl, more preferably hydroxymethyl, wherein at least one of the hydrogen atoms in R⁷ is replaced by T; preferably two of the hydrogen atoms in R¹ are replaced by T;
R⁶ is T or H;
T is ³H; and
wherein
R¹¹ is a 5- or 6-membered carbocyclic or heterocyclic ring which can be optionally substituted with F, NH₂, CN and/or CH₃, wherein the heterocyclic ring contains one or more heteroatoms selected from N, O and S, preferably N.

Preferably, the Tritium (³H) detectably labeled compounds having the formula (I-T), according to the present invention include (wherein T means ³H)

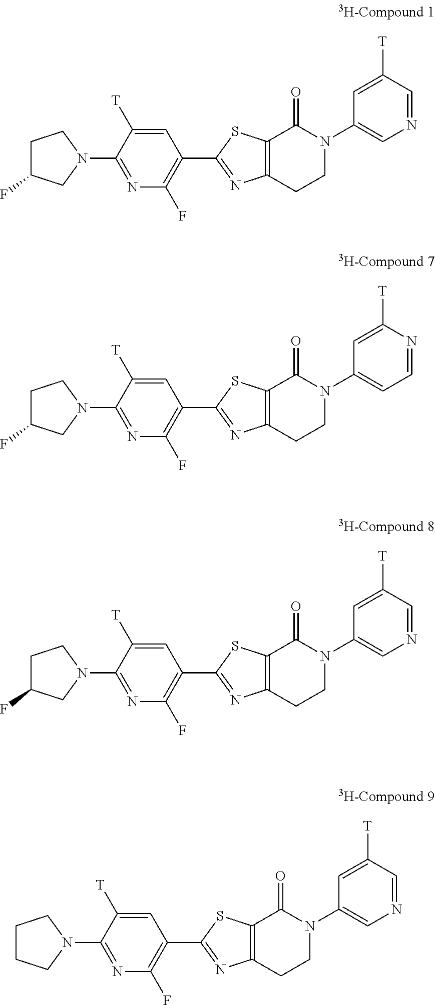

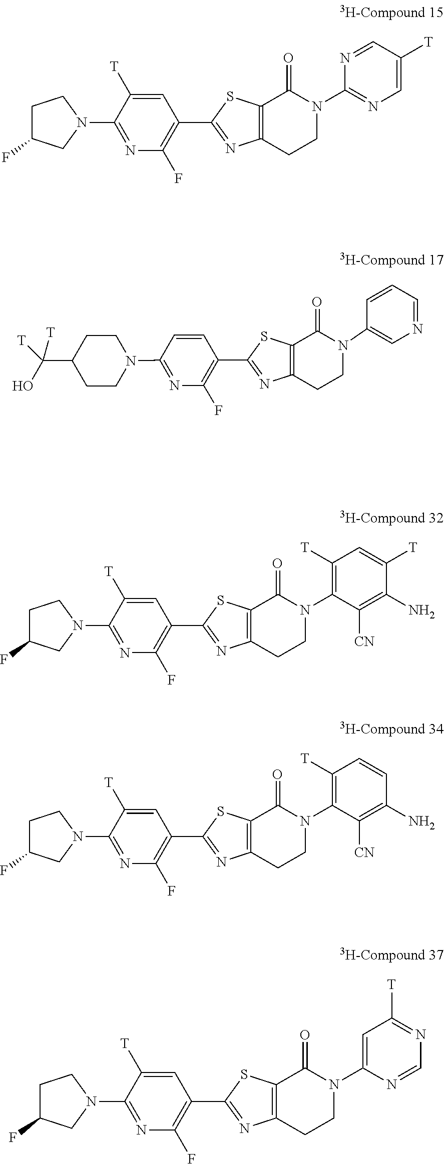

Preferably, the precursors having the formula (III) according to the present invention, can be selected from

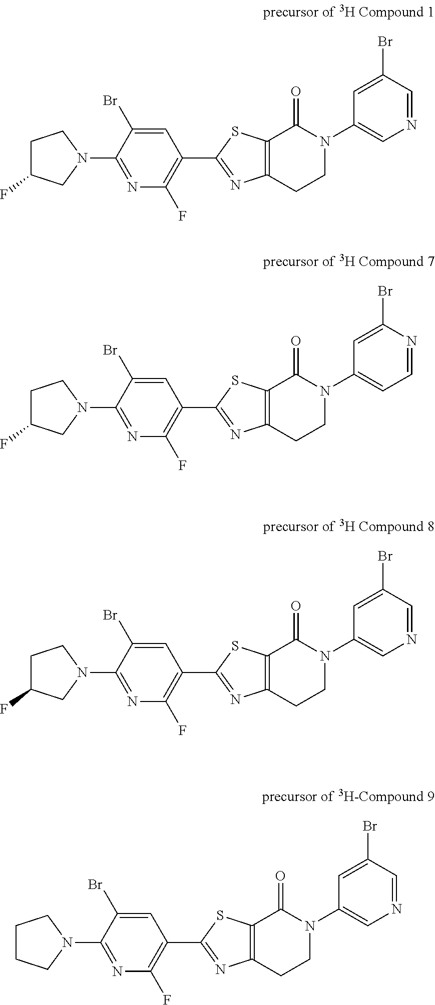 [SMILES: O=C1c2sc(-c3cc(Br)c(N4CC[C@@H](F)C4)nc3F)nc2CCN1c1cncc(Br)c1 ; O=C1c2sc(-c3cc(Br)c(N4CC[C@@H](F)C4)nc3F)nc2CCN1c1ccnc(Br)c1 ; O=C1c2sc(-c3cc(Br)c(N4CC[C@H](F)C4)nc3F)nc2CCN1c1cncc(Br)c1 ; O=C1c2sc(-c3cc(Br)c(N4CCCC4)nc3F)nc2CCN1c1cncc(Br)c1]

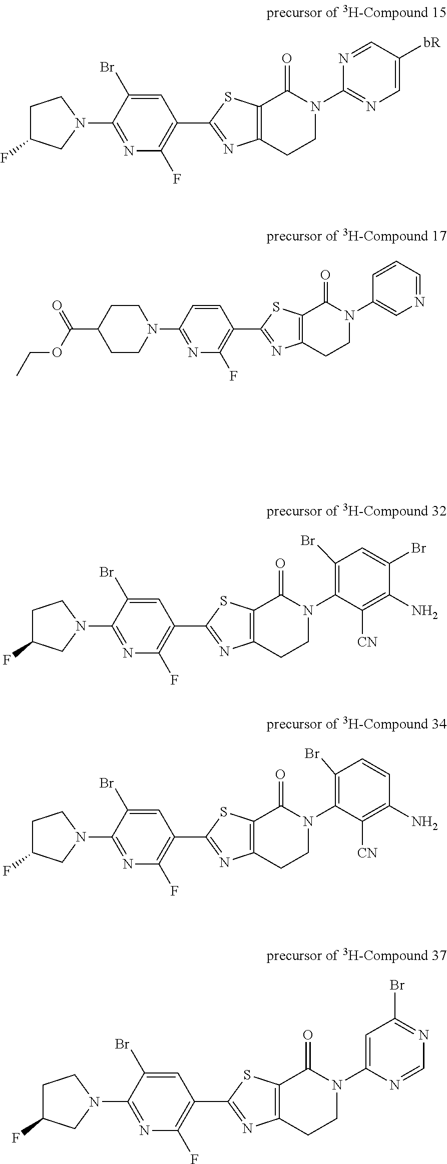

The methods used for introducing a radioisotope such as ³H are well known in the art and include the methods described below.

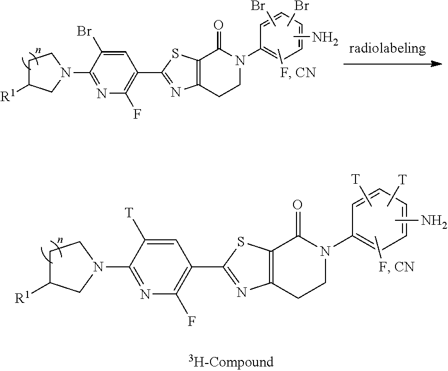

In this scheme, the substituents Br, NH₂, F and CN are just shown as an example. The definitions of formula (I-T) apply in this respect.

A further example is shown in the following scheme:

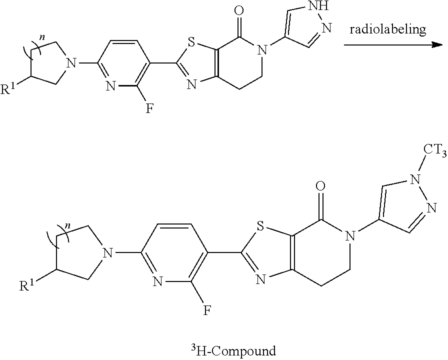

A further example is shown in the following scheme:

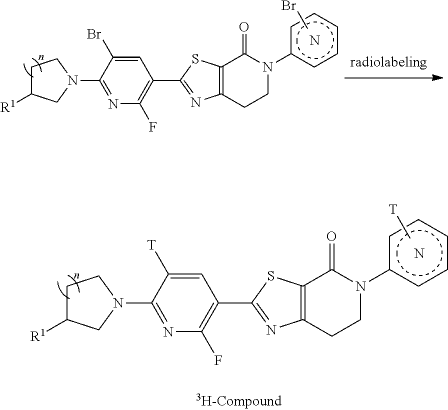

[symbol] means that the nitrogen atom can be present at any available position in the ring.

A further example is shown in the following scheme:

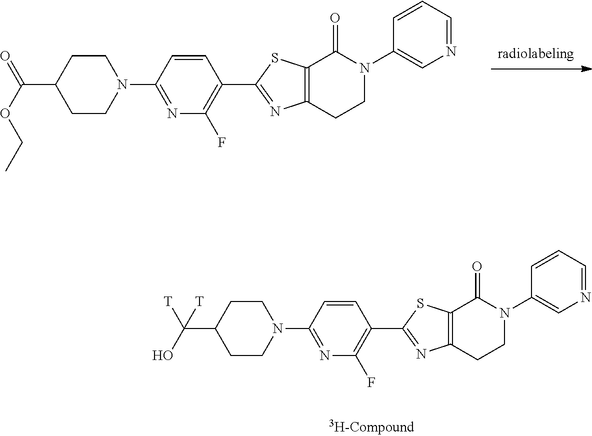

For the introduction of T (tritium), the ³H radiolabeling agent can be tritium gas. The method can be conducted in the presence of a catalyst such as palladium on carbon (Pd/C) or Lindlar's catalyst, a solvent such as N,N-dimethylformamide (DMF) and a base such as N,N-diisopropylethylamine (DIEA). Alternatively, the ³H radiolabeling agent can be LiT, prepared from n-BuLi and tritium gas in the presence of TMEDA, in the presence of AlCl₃ and a solvent such as THF.

In one embodiment, the present invention relates to a method for preparing a precursor compound of formula (III), as described above, the method comprising the step of:

Reacting a compound of formula (II) as defined above with R¹⁰ to provide a compound of formula (Ia) followed by either NBS bromination or acid cleavage of a trimethylsilylethoxymethyl (SEM)-protecting group. The following examples are given as an illustration:

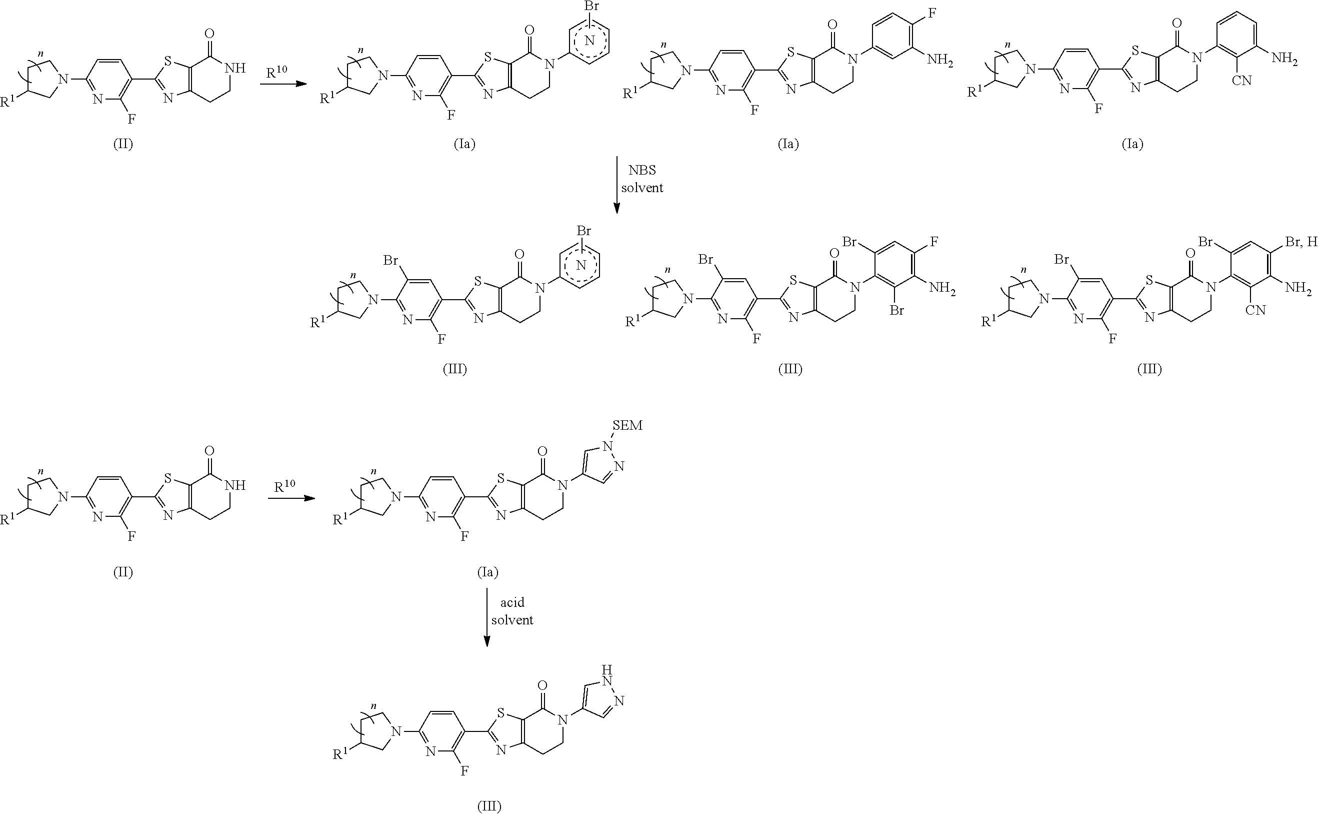

wherein
n and R¹, are as defined above;
R¹⁰ is a 5- or 6-membered carbocyclic or heterocyclic ring which can be optionally substituted with F, NH₂, CN and/or CH₃, wherein the heterocyclic ring contains one or more heteroatoms selected from N, O and S, wherein the 5- or 6-membered carbocyclic or heterocyclic ring is substituted with Br or I and may be optionally substituted with SEM.

In one preferred embodiment, R¹⁰ is selected from the following groups:

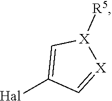

wherein X is N and R⁵ is SEM; and wherein Hal is Br or I;

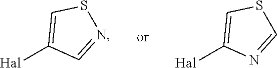

wherein Hal is Br;
R¹⁰ is

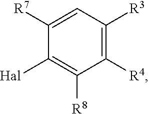

wherein Hal is Br or I; and

| | | | |
|---|---|---|---|
| R³ is F, | R⁴ is NH₂, | R⁷ is H, and | R⁸ is H: |
| R³ is NH₂, | R⁴ is F, | R⁷ is H, and | R⁸ is H, |
| R³ is CN, | R⁴ is NH₂. | R⁷ is H, and | R⁸ is H, |
| R³ is H, | R⁴ is NH₂, | R⁷ is H, and | R⁸ is CN, or |
| R³ is H, | R⁴ is NH₂, | R⁷ is H, and | R⁸ is F; |
| R³ is H, | R⁴ is NH₂, | R⁷ is H, and | R⁸ is CN; |

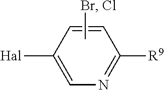

wherein R⁹ is selected from H, F, NH₂ or CH₃, wherein Hal is Br if Cl is present or Hal is I if Br is present;

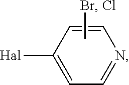

wherein Hal is Br if Cl is present or Hal is I if Br is present; nd

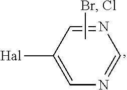

wherein Hal is Br if Cl is present or Hal is I if Br is present.

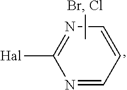

wherein Hal is Br if Cl is present or Hal is I if Br is present;

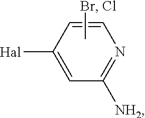

wherein Hal is Br if Cl is present or Hal is I if Br is present;

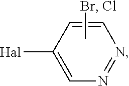

wherein Hal is Br if Cl is present or Hal is I if Br is present; and

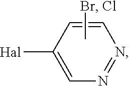

wherein Hal is Br if Cl is present or Hal is I if Br is present.

The method of reacting the compound having the formula (II) with R¹⁰ can be conducted by any suitable method. In one option, the reaction can be conducted in the presence of a diamine chelator such as DMEDA, a base such as potassium carbonate, a catalyst such as CuI, and an aprotic solvent such as dioxane. In another option, the reaction can be conducted under Pd-coupling conditions, in the presence of a Pd catalyst such as Pd[P(Ph)₃]₄ or Pd(OAc)₂, XantPhos.

### Fluorine (¹⁸F) Detectably Labeled Compounds:

In one embodiment, the present invention provides a method for preparing the Fluorine (¹⁸F) detectably labeled compounds of the invention, said method comprises radiolabeling a precursor having the formula (IV) with a radioisotope [¹⁸F]:

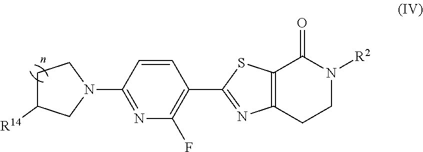

wherein
n, and R² are as defined above, and
R¹⁴ is a leaving group (LG) which is replaced by ¹⁸F in the radiolabeling step.

In one preferred embodiment, the present invention provides a method for preparing the fluorine (¹⁸F) detectably labeled compounds of the invention, said method comprises radiolabeling a precursor having the formula (IV) with a radioisotope [¹⁸F]:

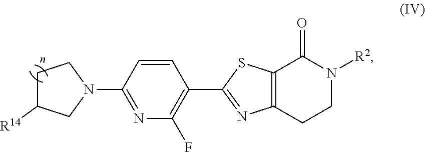

wherein
n is 1 or 2, preferably 1;
R² is

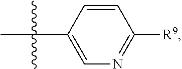

wherein R⁹ is H; and
R¹⁴ is a leaving group which is replaced by ¹⁸F in the radiolabeling step.

In another embodiment, the present invention provides a method for preparing the Fluorine (¹⁸F) detectably labeled compounds of the invention, said method comprises radiolabeling a precursor having the formula (V) with a radioisotope [¹⁸F]:

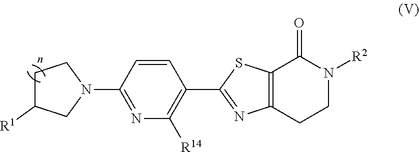

wherein n, R¹, and R² are as defined herein with respect to compound of formula (I) and
R¹⁴ is a leaving group (LG) which is replaced by ¹⁸F in the radiolabeling step.

In one preferred embodiment, the present invention provides a method for preparing the fluorine (¹⁸F) detectably labeled compounds of the invention, said method comprises radiolabeling a precursor having the formula (V) with a radioisotope [¹⁸F]:

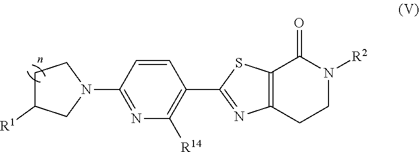

wherein n is 1 or 2, preferably 2;
R¹ is H or hydroxy(C₁-C₄)alkyl, preferably hydroxyethyl or hydroxymethyl, more preferably hydroxymethyl; and
R² is a 5- or 6-membered carbocyclic or heterocyclic ring which can be optionally substituted with F, NH₂, CN and/or CH₃, wherein the heterocyclic ring contains one or more heteroatoms selected from N, O and S, preferably N, preferably R² is

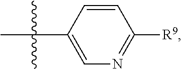

wherein R⁹ is H; and
R¹⁴ is a leaving group (LG) which is replaced by ¹⁸F in the radiolabeling step.

The fluorination can be conducted in the presence of a ¹⁸F-fluorination agent which can be selected from K[¹⁸F], Cs¹⁸F, Na¹⁸F, Rb¹⁸F, Kryptofix[222]K¹⁸F, tetra(C₁₋₆alkyl) ammonium salt of ¹⁸F, and tetrabutylammonium [¹⁸F]fluoride.

Preferably, the Leaving Group (LG) is C₁₋₄ alkyl sulfonate or C₆₋₁₀ aryl sulfonate or nitro. More preferably, the Leaving Group (LG) is mesylate, tosylate or nosylate or nitro. Even more preferably, the Leaving Group (LG) is mesylate or nitro.

Suitable solvents for the ¹⁸F-fluorination step are known to a skilled person. The solvent can be, for example, selected from the group consisting of DMF, DMSO, acetonitrile, DMA, or mixtures thereof. Preferably, the solvent is acetonitrile or DMSO.

Preferably, the method for preparing the Fluorine (¹⁸F) detectably labeled Compound 1 comprises a radiolabeling step in which the Leaving Group (LG), which in this case is mesylate, of the precursor L1 is replaced with a Fluorine (¹⁸F) in the presence of the ¹⁸F-fluorinating agent, such as K[¹⁸F] or [¹⁸F]TBAF, as shown below:

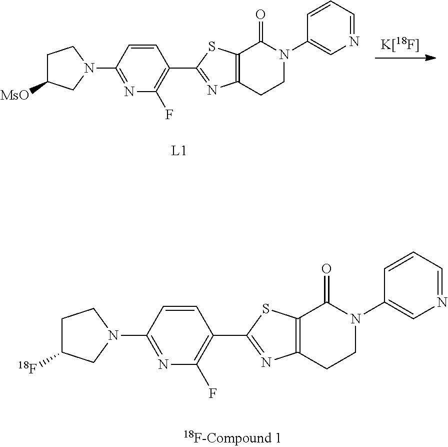

Preferably, the method for preparing the fluorine (¹⁸F) detectably labeled Compound 17 comprises a radiolabeling step in which the Leaving Group (LG), which in this case is nitro, of the precursor 20 is replaced with a fluorine (¹⁸F) in the presence of the ¹⁸F-fluorinating agent, such as K[¹⁸F] or [¹⁸F]TBAF, as shown below:

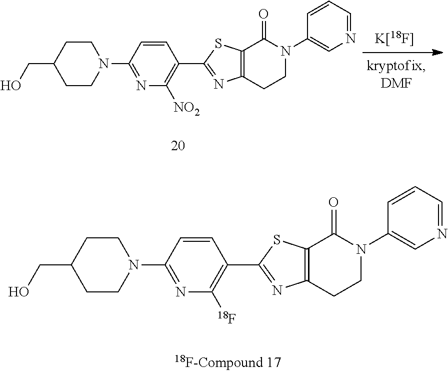

The compounds of the invention can be prepared by one of the general methods shown in the following schemes. These methods are only given for illustrative purposes and should not be construed as limiting.

The precursor compounds having the formulae (II), (III), (IV), or (V) as defined above or the stereoisomer, the polymorph, the racemic mixture, the tautomer, the pharmaceutically acceptable salt, the prodrug, the hydrate, or the solvate thereof are part of the invention.

### Abbreviations

Boc₂O Di-tert.-butyldicarbonate
CH₃CN Acetonitrile
CCl₄ Carbon tetrachloride
Cs₂CO₃ Cesium carbonate
CsF Cesium fluoride
CuBr₂ Copper bromide
CuC Copper(I)-chloride
CuI Copper iodide
DCM Dichloromethane
DIEA N,N-Diisopropyethyllamine
DMA N,N-Dimethylacetamide
DMAP Dimethylaminopyridine
DMEDA 1,2-Dimethylethylenediamine
DMF N,N-Dimethylformamide
DMSO Dimethylsulfoxide
EtOH Ethanol
dppf 1,1′-Ferrocenediyl-bis(diphenylphosphine)
EtOAc Ethyl acetate
h Hour
HCl Hydrochloric acid
K₂CO₃ Potassium carbonate
LCMS Liquid chromatography-mass spectrometry
n-BuOH n-BuOH
NMR Nuclear magnetic resonance
NBS N-Bromosuccinimide
Pd(PPh₃)₄ Palladium tetrakistriphenylphosphine
Pd(dppf)Cl₂×CH₂Cl₂ 1,1′-Bis(diphenylphosphino)ferrocene]-dichloropalladium (II) dichloromethane complex
Pd(OAc)₂ Palladium(II) acetate
RCP Radiochemical purity
RT Room Temperature (approx. 25° C.)
TBDMSCl Tert-butyldimethylsilylchloride
^{t}BuONO Tert-butyl nitrite
TEA Triethylamine
TFA Trifluoroacetic acid
XantPhos 4,5-Bis(diphenylphosphino)-9,9-dimethylxanthene

### General Synthetic Schemes

### Synthetic Scheme for the Preparation of 2-[2-fluoro-6-[(3R)-3-fluoropyrrolidin-1-yl]-3-pyridyl]-5-(3-pyridyl)-6,7-dihydrothiazolo[5,4-c]pyridin-4-one (Compound 1)

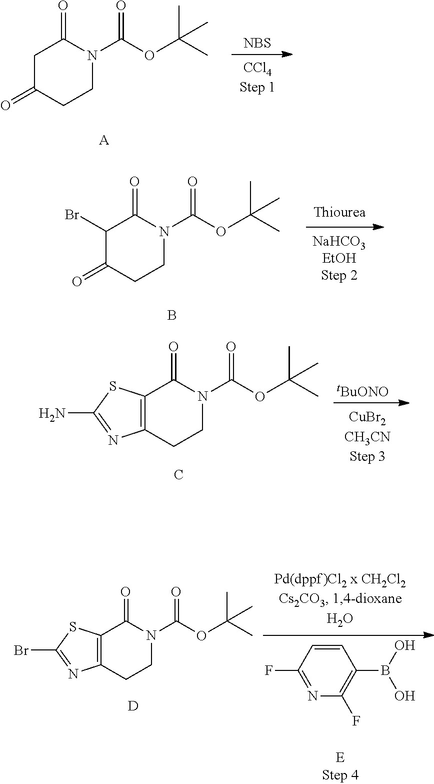

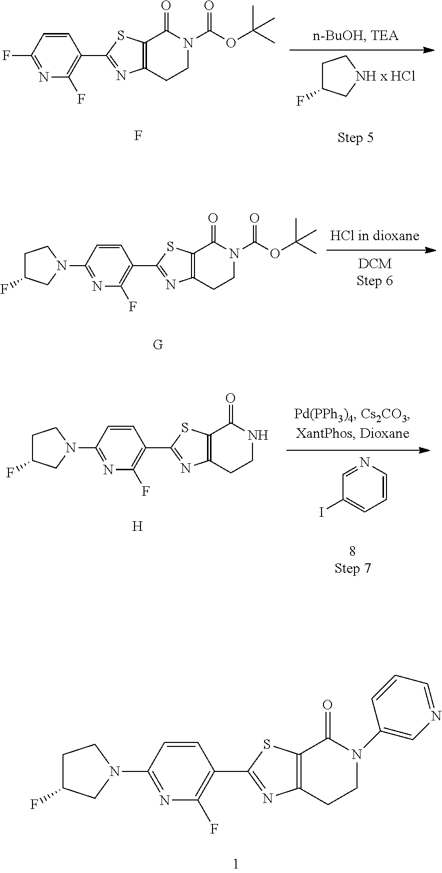

### Synthetic Scheme for the Preparation of 2-(2-fluoro-6-(4-(hydroxymethyl)piperidin-1-yl)pyridin-3-yl)-5-(pyridin-3-yl)-6,7-dihydrothiazolo[5,4-c]pyridin-4(5H)-one (Compound 17)

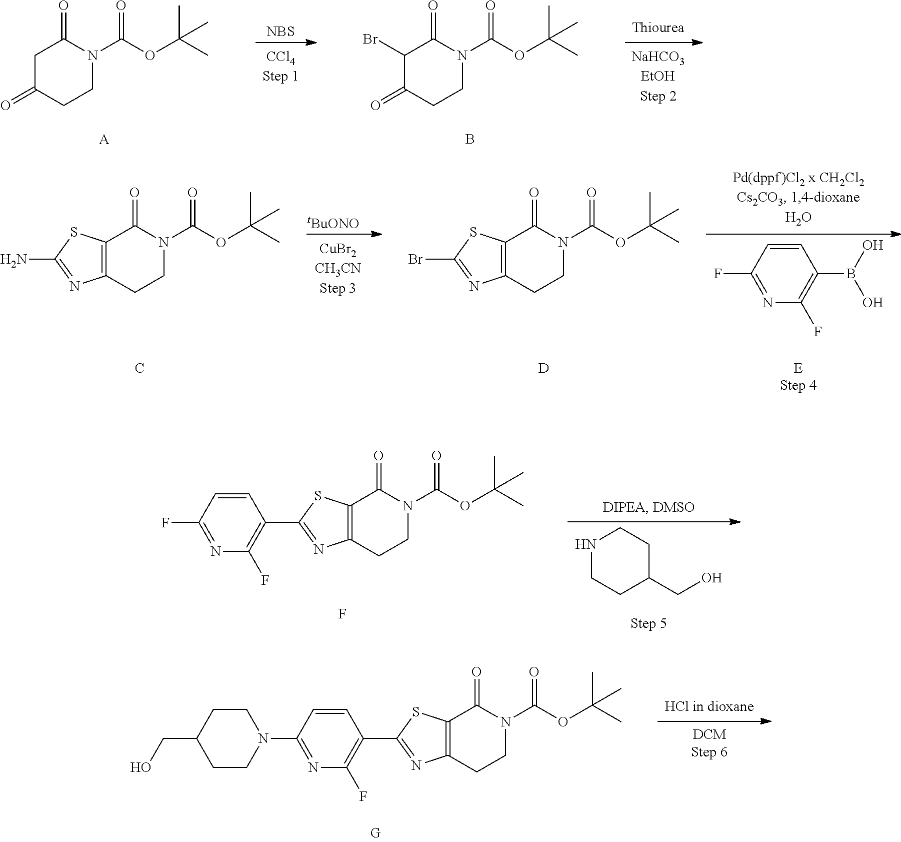

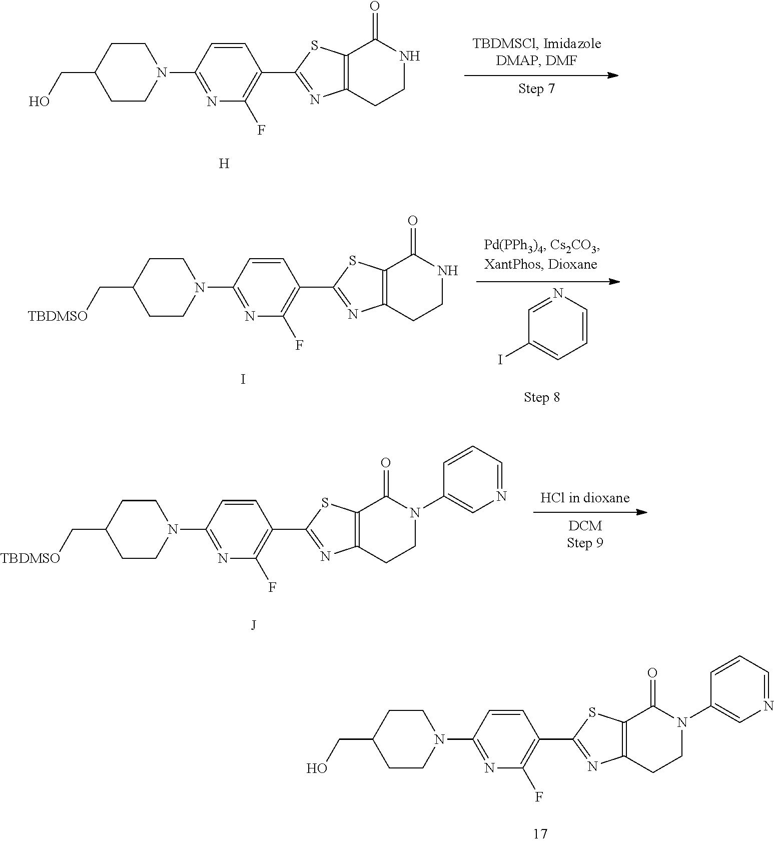

### Synthetic Scheme for the Preparation of [³H] Precursor Compounds

### Synthetic Scheme for the Preparation of 5-(3-amino-2,6-dibromo-4-fluorophenyl)-2-(5-bromo-6-(Pyrrolidin-1-yl)pyridin-3-vi)-6,7-dihydrothiazolo[5,4-c]pyridin-4(5H)-one (precursor of ³H Compound 1)

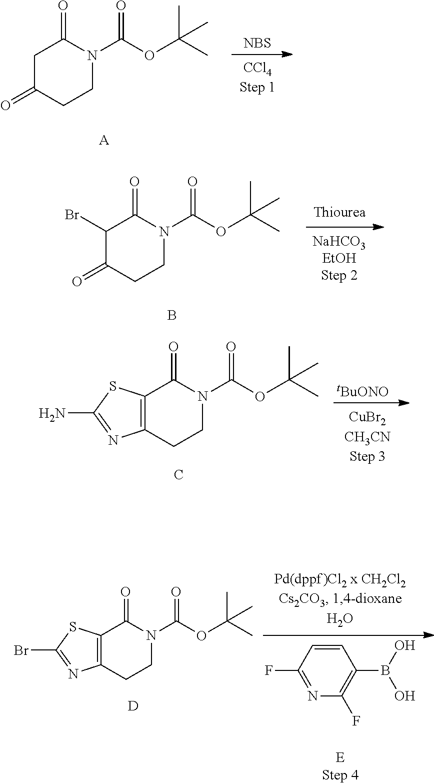

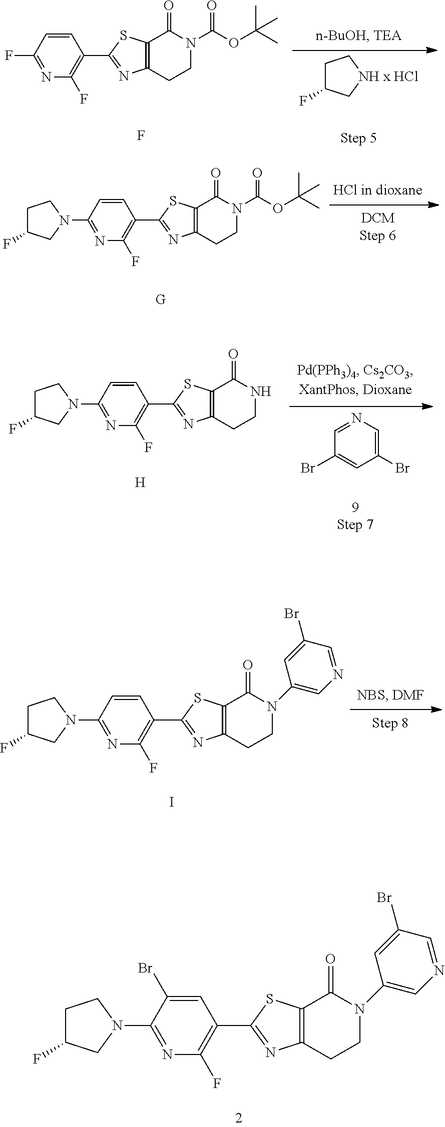

### Synthetic Scheme for the Preparation of ethyl 1-(3-bromo-5-(5-(5-bromopyridin-3-yl)-4-oxo-4,5,6,7-tetrahydrothiazolo[5,4-c]pyridin-2-yl)-6-fluoropyridin-2-yl)piperidine-4-carboxylate (Precursor of ³H Compound 17)

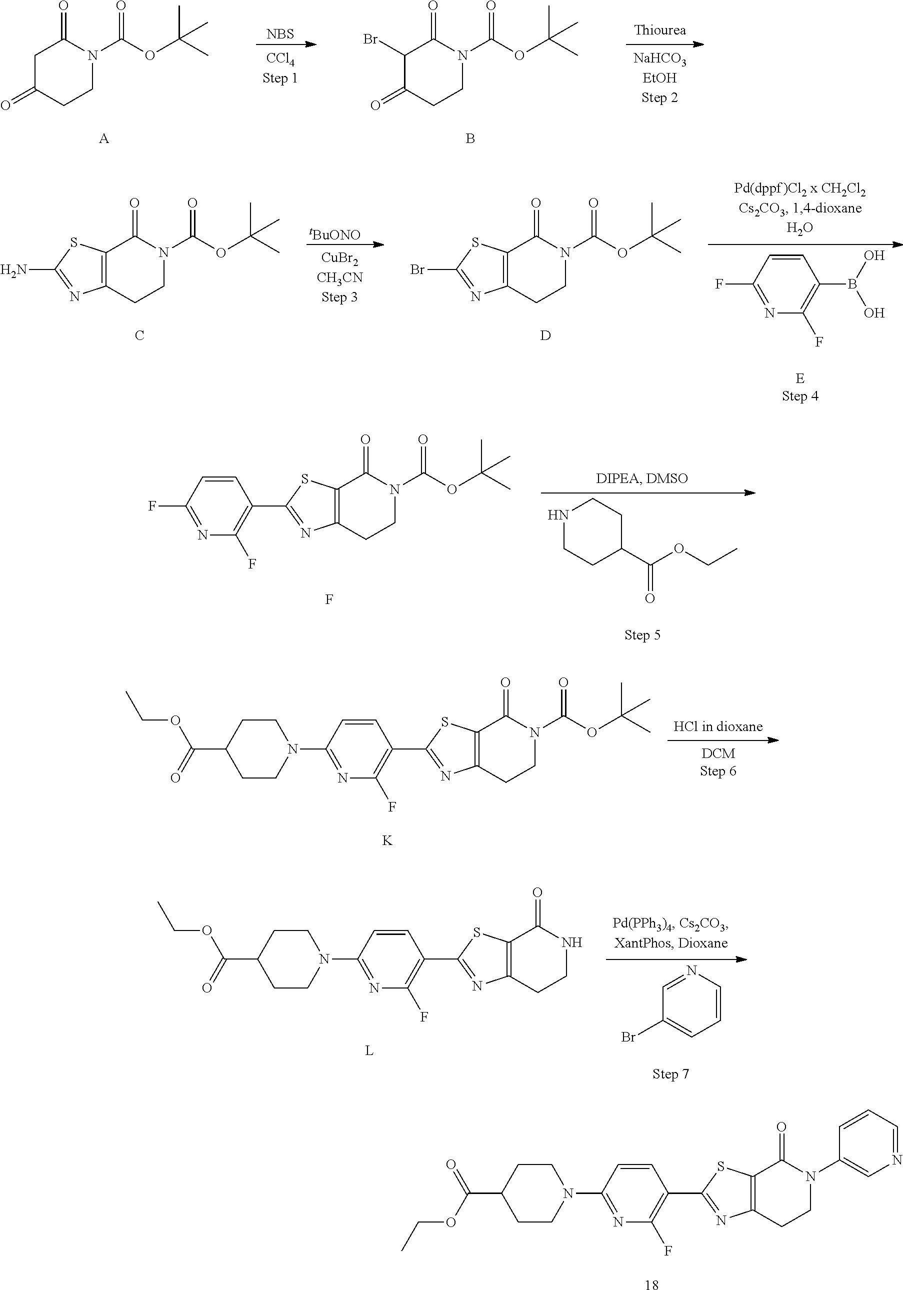

³H labeled compounds can be prepared from a suitable precursor compound containing halogen atoms by catalytic tritiodehalogenation with tritium gas (M. Saljoughian Synthesis (2002), 1781-1801), or from a suitable precursor compound containing a NH moiety by methylation with methyl iodide [³H], or from a suitable precursor compound with a moiety susceptible to be reduced by a [³H]-containing reducing agent. (Y. Chen, Chemistry 25 (2019):3405-3439). Preferably, the solvents used in the ³H-labeling are DMF or DMA, preferably the solvent is DMF.

### Synthetic Schemes for the Preparation of Precursors for ¹⁸F-Labeling

### Synthetic Scheme for the Preparation of [(3S)-1-[6-fluoro-5-[4-oxo-5-(3-pyridyl)-6,7-dihydrothiazolo[5,4-c]pyridin-2-yl]-2-pyridyl]pyrrolidin-3-yl]methanesulfonate (Precursor of ¹⁸F Compound 1)

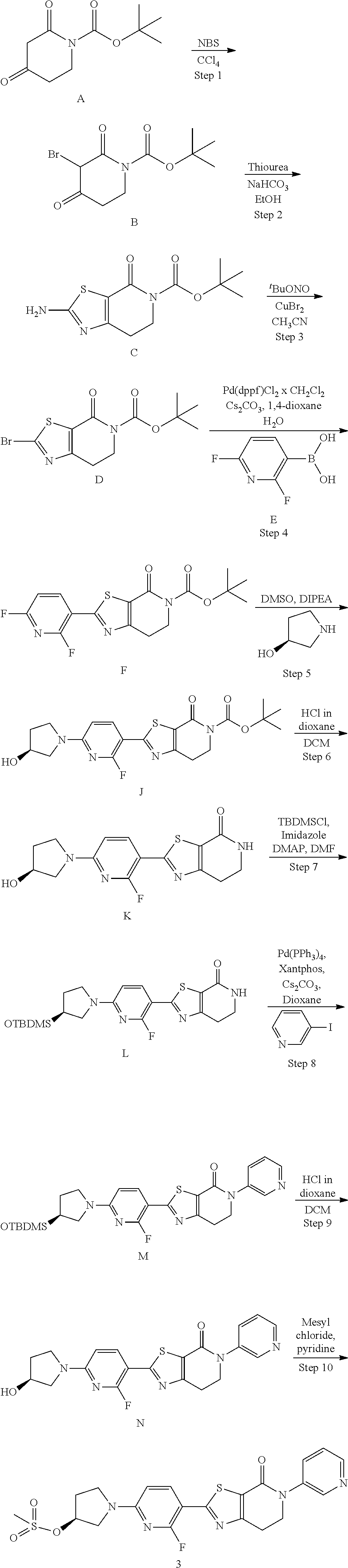

### Synthetic Scheme for the Preparation of 2-(6-(4-(hydroxymethyl)piperidin-1-yl)-2-nitropyridin-3-yl)-5-(pyridin-3-yl)-6,7-dihydrothiazolo[5,4-c]pyridin-4(5H)-one (Precursor of ¹⁸F Compound 17)

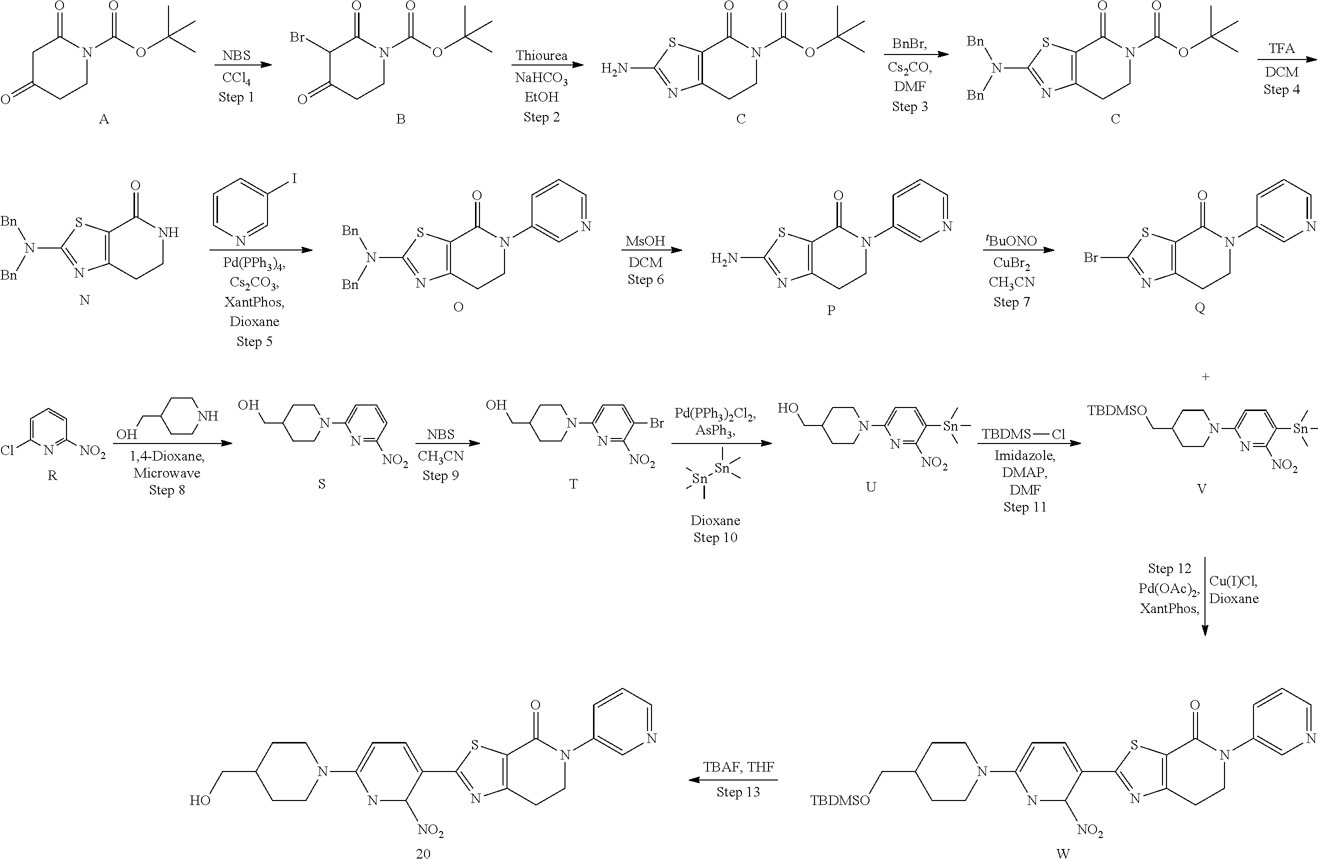

The reactions take place in the presence of a fluorinating agent and typically a solvent.

¹⁸F labeled compounds can be prepared by reacting the precursor compounds containing a LG with an ¹⁸F-fluorinating agent, so that the LG is replaced by ¹⁸F. The ¹⁸F-fluorinating agent can be a tetraalkylammonium salt of ¹⁸F (such as tetra(C₁₋₆ alkyl) ammonium salt of ¹⁸F, e.g., tetrabutylammonium [¹⁸F]fluoride), a tetraalkylphosphonium salt of ¹⁸F (such as tetra(C₁₋₆ alkyl) phosphonium salt of ¹⁸F), K[¹⁸F], Cs¹⁸F, Na¹⁸F, Rb¹⁸F, or Kryptofix[222]K¹⁸F. Preferably, the ¹⁸F-fluorination agent is Cs¹⁸F, K¹⁸F, or tetrabutylammonium [¹⁸F]fluoride. The reagents, solvents and conditions which can be used for the ¹⁸F-fluorination are well-known to a skilled person in the field (L. Cai, S. Lu, V. Pike, Eur. J. Org. Chem. 2008, 2853-2873; J. Fluorine Chem., 27 (1985):177-191; Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50). Preferably, the solvents used in the ¹⁸F-fluorination are DMF, DMSO, acetonitrile, DMA, or mixtures thereof, preferably the solvent is acetonitrile or DMSO.

Although the reaction is shown above with respect to ¹⁸F as a radioactive label, other radioactive labels can be introduced following similar procedures.

The invention is illustrated by the following examples which, however, should not be construed as limiting.

### EXAMPLES

All reagents and solvents were obtained from commercial sources and were used without further purification. Proton (¹H) NMR spectra were recorded on a JEOL-500 MHz NMR spectrometer. Mass spectra (MS) were recorded on an UPLC H-Class Plus with Photodiode Array detector and a QDa Mass spectrometer. Chromatography was performed using silica gel (Fluka: Silica gel 60, 0.063-0.2 mm) and suitable solvents as indicated in the specific examples. Flash purification was conducted with a Biotage Isolera One flash purification system using HP-Sil or KP-NH SNAP cartridges (Biotage) and the solvent gradient indicated in the specific examples. Thin layer chromatography (TLC) was carried out on silica gel plates with UV detection.

Although some of the present examples do not indicate that the respective compounds were detectably labeled, it is understood that corresponding detectably labeled compounds are intended and can be easily prepared, e.g., by using detectably labeled starting materials, such as starting materials containing C(³H)₃, (¹¹C)H₃ or ¹⁸F.

### Example 1 Synthesis of 2-[2-fluoro-6-[(3R)-3-fluoropyrrolidin-1-yl]-3-pyridyl]-5-(3-pyridyl)-6,7-dihydrothiazolo[5,4-c]pyridin-4-one

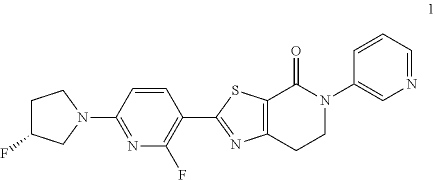 [SMILES: O=C1c2sc(-c3ccc(N4CC[C@@H](F)C4)nc3F)nc2CCN1c1cccnc1]

### Step-1: Synthesis of tert-butyl 3-bromo-2,4-dioxopiperidine-1-carboxylate (B)

Tert-butyl 2,4-dioxopiperidine-1-carboxylate A (10 g, 46.9 mmol) was dissolved in carbon tetrachloride (125 mL) and cooled to 0 to 5° C. To the above solution, N-bromo succinimide (8.35 g, 46.9 mmol) was added portionwise and stirring was continued at 28° C. for 1 hour. The reaction mixture was diluted with two runs of ethyl acetate (500 mL) and water (200 mL). The organic phase was separated, dried over Na₂SO₄, filtered and the solvents were removed under reduced pressure to obtain the title compound B as a white solid (10 g, 73%).

¹H NMR (500 MHz, DMSO-d₆): δ 8.1 (s, 2H), 3.89 (t, 2H), 2.76 (t, 2H), 1.45 (s, 9H). LCMS (ESI) 292.04 m/z [M+H]⁺.

### Step-2: Synthesis of tert-butyl 2-amino-4-oxo-6,7-dihydrothiazolo[5,4-c]pyridine-5(4H)-carboxylate (C)

Title compound B (10 g, 34.36 mmol), thiourea (2.61 g, 34.36 mmol) and sodium bicarbonate (2.88 g, 34.36 mmol) were dissolved in ethanol (160 mL) and heated in an oil bath at 80° C. for 2.5 hours. The reaction mixture was diluted with two runs of ethyl acetate (500 mL) and water (200 mL). The organic phase was separated, dried over Na₂SO₄, filtered and the solvents were removed under reduced pressure. The solid obtained was recrystallized from ethanol to obtain the title compound C as a white solid (6.6 g, 71%).

¹H NMR (500 MHz, DMSO-d₆): δ 8.1 (s, 2H), 3.89 (t, 2H), 2.76 (t, 2H), 1.45 (s, 9H). LCMS (ESI) 270.3 m/z [M+H]⁺.

### Step-3: Synthesis of tert-butyl 2-bromo-4-oxo-6,7-dihydrothiazolo[5,4-c]pyridine-5(4H)-carboxylate (D)

Title compound C (6.6 g, 24.53 mmol) was dissolved in acetonitrile (82 mL) and cooled to −10° C. in an ice bath with stirring. Tert-butyl nitrite (4.3 mL, 36.4 mmol) was added to the above solution and stirring was continued at −10° C. for 1 hour. Copper(II)-bromide (6.5 g, 29.43 mmol) was added to the above mixture and it was allowed to stir at 28° C. for 1 hour. The reaction mixture was basified with aqueous saturated sodium bicarbonate solution to pH 8 to 9 and filtered. The filtrate collected was diluted with three runs of ethyl acetate (300 mL) and water (100 mL). The organic phase was separated, dried over Na₂SO₄, filtered and the solvents were removed under reduced pressure. The residue obtained was purified by column chromatography on silica gel (100 to 200 mesh) using ethyl acetate/n-hexane gradient (0/100->10/90) to afford the title compound D as a white solid (4.73 g, 58%).

¹H NMR (500 MHz DMSO-d₆): δ 4.12 (t, 2H), 3.10 (t, 2H), 1.5 (s, 9H). LCMS (ESI) 279 m/z [M+H-C₄H₈]⁺.

### Step-4: Synthesis of tert-butyl 2-(2,6-difluoropyridin-3-yl)-4-oxo-6,7-dihydrothiazolo[5,4-c]pyridine-5(4H)-carboxylate (F)

Tert-butyl 2-bromo-4-oxo-6,7-dihydrothiazolo[5,4-c]pyridine-5(4H)-carboxylate D (0.3 g, 0.9 mmol) was dissolved in 1,4-dioxane (15 mL) and degassed by passing a stream of nitrogen through the mixture. Then [1,1′-bis(diphenyl-phosphino)ferrocene]-dichloropalladium(II), complex with dichloromethane (0.07 g, 0.09 mmol), title compound E (0.21 g, 1.35 mmol) and potassium phosphate tribasic (0.28 g, 1.35 mmol) were added, and the reaction mixture was heated at 100° C. in an oil-bath for 8 h. The reaction mixture was concentrated under reduced pressure. The residue was purified by column chromatography on silica gel (basic, 100-200 mesh) using ethyl acetate/n-hexane gradient (0/100->10/90->15/85) to obtain title compound F as a pale yellow solid (0.2 g, 60%).

¹H NMR (500 MHz DMSO-d₆): δ 8.91 (q, 1H), 7.43 (dd, 1H), 4.1 (t, 2H), 3.19 (t, 2H), 1.5 (s, 9H). LCMS (ESI) 311.95 m/z [M+H-C₄H₈]⁺.

### Step-5: Synthesis of tert-butyl (R)-2-(2-fluoro-6-(3-fluoropyrrolidin-1-yl)pyridin-3-yl)-4-oxo-6,7-dihydrothiazolo[5,4-c]pyridine-5(4H)-carboxylate (G)

Title compound F (0.3 g, 0.86 mmol), (R)-3-fluoropyrrolidine hydrochloric acid salt (0.213 g, 1.7 mmol) and triethylamine (0.33 mL, 2.36 mmol) were suspended in n-butanol (8 mL) using a microwave vial. The sealed vial was then heated at 160° C. for 1 hour using a CEM microwave. The reaction mixture was concentrated under reduced pressure. The residue obtained was suspended in water (20 mL) and filtered over Whatmann filter paper. The solid was washed with water (20 mL) followed by n-hexane (20 mL) and dried under reduced pressure to obtain the title compound G as an off-white solid (0.27 g, 98%).

¹H NMR (500 MHz, DMSO-d₆): δ 8.75 (d, 1H), 8.06 (dd, 1H), 7.81 (s, 1H), 6.64 (d, 1H), 5.48 (d, 1H), 3.9-3.7 (m, 3H), 3.51-3.48 (m, 3H), 2.98 (t, 2H), 2.34.2.13 (m, 2H). LCMS (ESI) 319.00 m/z [M+H]⁺.

### Step-6: Synthesis of (R)-2-(2-fluoro-6-(3-fluoropyrrolidin-1-yl)pyridin-3-yl)-6,7-dihydrothiazolo[5,4-c]pyridin-4(5H)-one (H)

Title compound G (0.188 g, 0.011 mmol) was dissolved in DCM (4 mL) and cooled to 0° C. in an ice bath with stirring. 4M HCl in 1,4-dioxane (2 mL) was added to above solution and stirring continued at RT for 3 h. After completion of the reaction, solvent was removed under reduced pressure. The residue obtained was dissolved in ice cold water and basified with aqueous saturated sodium bicarbonate solution to pH 8-9. The solid precipitated was filtered and dried to afford the title compound H as a pale yellow solid (0.135 g, 92%).

¹H NMR (500 MHz DMSO-d₆): δ 7.81 (d, 2H), 7.75 (s, 1H), 6.66 (d, 2H), 5.48 (d, 1H), 3.52 (m, 6H), 2.96 (t, 2H), 2.24 (m, 2H). LCMS (ESI) 318 [M+H]⁺.

### Step-7: Synthesis of (R)-2-(2-fluoro-6-(3-fluoropyrrolidin-1-yl)pyridin-3-yl)-5-(pyridin-3-yl)-6,7-dihydrothiazolo[5,4-c]pyridin-4(5H)-one (1)

Compound H (0.05 g, 0.14 mmol) and 3-iodopyridine 8 (0.09 g, 0.44 mmol) were dissolved in degassed 1,4-dioxane (7.5 mL). After the addition of Pd[PPh₃]₄ (0.008 g, 0.0074 mmol), XantPhos (0.01 g, 0.014 mmol), and Cs₂CO₃ (0.14 g, 0.44 mmol), the reaction mixture was heated at ˜100° C. in a sand bath for 24 h. The reaction mixture was concentrated under reduced pressure to afford the crude residue. The crude residue was purified by column chromatography on silica gel (100-200 mesh) using CH₂Cl₂/MeOH gradient (100/0->99.5/0.5->99/1) to afford 1 as a yellow solid (0.025 g, 41%).

¹H NMR (400 MHz DMSO-d₆): δ 8.67 (d, 1H), 8.43 (m, 2H), 7.87 (m, 1H), 7.5 (dd, 1H), 6.64 (dd, 1H), 5.49 (d, 1H), 4.17 (t, 2H), 3.72 (m, 3H), 3.52 (m, 1H), 3.26 (t, 2H), 2.22 (m, 2H). LCMS (ESI) 414.1 m/z [M+H]⁺

### Example 2 Synthesis of 5-(3-amino-2,6-dibromo-4-fluorophenyl)-2-(5-bromo-6-(pyrrolidin-1-yl)pyridin-3-yl)-6,7-dihydrothiazolo[5,4-c]pyridin-4(5H)-one

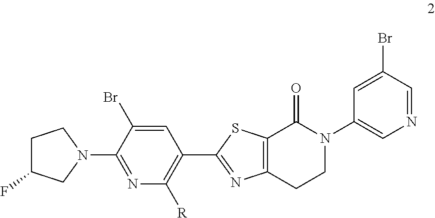

### Step-1: Synthesis of tert-butyl 3-bromo-2,4-dioxopiperidine-1-carboxylate (B)

Tert-butyl 2,4-dioxopiperidine-1-carboxylate A (10 g, 46.9 mmol) was dissolved in carbon tetrachloride (125 mL) and cooled to 0 to 5° C. To the above solution, N-bromo succinimide (8.35 g, 46.9 mmol) was added portionwise and stirring was continued at 28° C. for 1 hour. The reaction mixture was diluted with two runs of ethyl acetate (500 mL) and water (200 mL). The organic phase was separated, dried over Na₂SO₄, filtered and the solvents were removed under reduced pressure to obtain the title compound B as a white solid (10 g, 73%).

¹H NMR (500 MHz, DMSO-d₆): δ 8.1 (s, 2H), 3.89 (t, 2H), 2.76 (t, 2H), 1.45 (s, 9H). LCMS (ESI) 292.04 m/z [M+H]⁺.

### Step-2: Synthesis of tert-butyl 2-amino-4-oxo-6,7-dihydrothiazolo[5,4-c]pyridine-5(4H)-carboxylate (C)

Title compound B (10 g, 34.36 mmol), thiourea (2.61 g, 34.36 mmol) and sodium bicarbonate (2.88 g, 34.36 mmol) were dissolved in ethanol (160 mL) and heated in an oil bath at 80° C. for 2.5 hours. The reaction mixture was diluted with two runs of ethyl acetate (500 mL) and water (200 mL). The organic phase was separated, dried over Na₂SO₄, filtered and the solvents were removed under reduced pressure. The solid obtained was recrystallized from ethanol to obtain the title compound C as a white solid (6.6 g, 71%).

¹H NMR (500 MHz, DMSO-d₆): δ 8.1 (s, 2H), 3.89 (t, 2H), 2.76 (t, 2H), 1.45 (s, 9H). LCMS (ESI) 270.3 m/z [M+H]⁺.

### Step-3: Synthesis of tert-butyl 2-bromo-4-oxo-6,7-dihydrothiazolo[5,4-c]pyridine-5(4H)-carboxylate (D)

Title compound C (6.6 g, 24.53 mmol) was dissolved in acetonitrile (82 mL) and cooled to −10° C. in an ice bath with stirring. Tert-butyl nitrite (4.3 mL, 36.4 mmol) was added to the above solution and stirring was continued at −10° C. for 1 hour. Copper(II)-bromide (6.5 g, 29.43 mmol) was added to the above mixture, and it was allowed to stir at 28° C. for 1 hour. The reaction mixture was basified with aqueous saturated sodium bicarbonate solution to pH 8 to 9 and filtered. The filtrate collected was diluted with three runs of ethyl acetate (300 mL) and water (100 mL). The organic phase was separated, dried over Na₂SO₄, filtered and the solvents were removed under reduced pressure. The residue obtained was purified by column chromatography on silica gel (100 to 200 mesh) using ethyl acetate/n-hexane gradient (0/100->10/90) to afford the title compound D as a white solid (4.73 g, 58%).

¹H NMR (500 MHz DMSO-d₆): δ 4.12 (t, 2H), 3.10 (t, 2H), 1.5 (s, 9H). LCMS (ESI) 279 m/z [M+H-C₄H₈]⁺.

### Step-4: Synthesis of tert-butyl 2-(2,6-difluoropyridin-3-yl)-4-oxo-6,7-dihydrothiazolo[5,4-c]pyridine-5(4H)-carboxylate (3) (F)

Tert-butyl 2-bromo-4-oxo-6,7-dihydrothiazolo[5,4-c]pyridine-5(4H)-carboxylate D (0.3 g, 0.9 mmol) was dissolved in 1,4-dioxane (15 mL) and degassed by passing a stream of nitrogen through the mixture. Then [1,1′-bis(diphenyl-phosphino)ferrocene]-dichloropalladium(II), complex with dichloromethane (0.07 g, 0.09 mmol), title compound E (0.21 g, 1.35 mmol) and potassium phosphate tribasic (0.28 g, 1.35 mmol) were added, and the reaction mixture was heated at 100° C. in an oil-bath for 8 h. The reaction mixture was concentrated under reduced pressure. The residue was purified by column chromatography on silica gel (basic, 100-200 mesh) using ethyl acetate/n-hexane gradient (0/100->10/90->15/85) to obtain title compound F as a pale yellow solid (0.2 g, 60%).

¹H NMR (500 MHz DMSO-d₆): δ 8.91 (q, 1H), 7.43 (dd, 1H), 4.1 (t, 2H), 3.19 (t, 2H), 1.5 (s, 9H). LCMS (ESI) 311.95 m/z [M+H-C₄H₈]⁺.

### Step-5: Synthesis of tert-butyl (R)-2-(2-fluoro-6-(3-fluoropyrrolidin-1-yl)pyridin-3-yl)-4-oxo-6,7-dihydrothiazolo[5,4-c]pyridine-5(4H)-carboxylate (G)

Title compound F (0.3 g, 0.86 mmol), (R)-3-fluoropyrrolidine hydrochloric acid salt (0.213 g, 1.7 mmol) and triethylamine (0.33 mL, 2.36 mmol) were suspended in n-butanol (8 mL) using a microwave vial. The sealed vial was then heated at 160° C. for 1 hour using a CEM microwave. The reaction mixture was concentrated under reduced pressure. The residue obtained was suspended in water (20 mL) and filtered over Whatmann filter paper. The solid was washed with water (20 mL) followed by n-hexane (20 mL) and dried under reduced pressure to obtain the title compound G as an off-white solid (0.27 g, 98%).

¹H NMR (500 MHz, DMSO-d₆): δ 8.75 (d, 1H), 8.06 (dd, 1H), 7.81 (s, 1H), 6.64 (d, 1H), 5.48 (d, 1H), 3.9-3.7 (m, 3H), 3.51-3.48 (m, 3H), 2.98 (t, 2H), 2.34.2.13 (m, 2H). LCMS (ESI) 319.00 m/z [M+H]⁺.

### Step-6: Synthesis of (R)-2-(2-fluoro-6-(3-fluoropyrrolidin-1-yl)pyridin-3-yl)-6,7-dihydrothiazolo[5,4-c]pyridin-4(5H)-one (H)

Title compound G (0.188 g, 0.011 mmol) was dissolved in DCM (4 mL) and cooled to 0° C. in an ice bath with stirring. 4M HCl in 1,4-dioxane (2 mL) was added to above solution and stirring was continued at RT for 3 h. After completion of the reaction, solvent was removed under reduced pressure. The residue obtained was dissolved in ice cold water and basified with aqueous saturated sodium bicarbonate solution to pH 8-9, filtered the solid precipitated and dried to afford the title compound H as a pale yellow solid (0.135 g, 92%).

¹H NMR (500 MHz DMSO-d₆): δ 7.81 (d, 2H), 7.75 (s, 1H), 6.66 (d, 2H), 5.48 (d, 1H), 3.52 (m, 6H), 2.96 (t, 2H), 2.24 (m, 2H). LCMS (ESI) 318 [M+H]⁺.

### Step-7: Synthesis of (R)-5-(5-bromopyridin-3-yl)-2-(2-fluoro-6-(3-fluoropyrrolidin-1-yl)pyridin-3-yl)-6,7-dihydrothiazolo[5,4-c]pyridin-4(5H)-one (1)

(R)-2-(2-fluoro-6-(3-fluoropyrrolidin-1-yl)pyridin-3-yl)-6,7-dihydrothiazolo[5,4-c]pyridin-4(5H)-one H (0.05 g, 0.14 mmol) and 3,5-dibromopyridine 7 (0.1 g, 0.44 mmol) were dissolved in degassed 1,4-dioxane (5 mL). After the addition of Pd[PPh₃]₄ (0.008 g, 0.0074 mmol), XantPhos (0.08 g, 0.014 mmol), and Cs₂CO₃ (0.1 g, 0.3 mmol), the reaction mixture was heated at ˜100° C. in a oil-bath for 24 h. The reaction mixture was concentrated under reduced pressure to afford the crude residue. The crude residue was purified by column chromatography on silica gel (100-200 mesh) using CH₂Cl₂/MeOH gradient (100/0->99/1->98.8/1.2) to afford I as a yellow solid (0.05 g, 68%).

¹H NMR (400 MHz DMSO-d₆): δ 8.67 (d, 1H), 8.58 (d, 1H), 8.41 (dd, 1H), 6.64 (dd, 1H), 5.48 (d, 1H), 4.18 (t, 2H), 3.74 (m, 3H), 3.52 (m, 1H), 3.25 (t, 2H), 2.22 (m, 2H). LCMS (ESI) 492.0 m/z [M+H]⁺

### Step-8: Synthesis of (R)-2-(5-bromo-2-fluoro-6-(3-fluoropyrrolidin-1-yl)pyridin-3-yl)-5-(5-bromopyridin-3-yl)-6,7-dihydrothiazolo[5,4-c]pyridin-4(5H)-one (2)

Title compound I (0.14 g, 0.28 mmol) was dissolved in DMF (5 mL) and cooled to −50° C. NBS (0.1 g, 0.56 mmol) was added portionwise. The reaction mixture was allowed to warm up to 20° C. during a period of 1 h. The reaction mixture was diluted with cold water (50 mL) and the solid was filtered. The crude solid was purified by preparative TLC plates using dichloromethane/methanol (98.5/1.5) as mobile phase to obtain the title compound 2 as a pale yellow solid (0.083 g, 52%).

¹H NMR (400 MHz DMSO-d₆): δ 8.67 (d, 1H), 8.59 (d, 1H), 8.57 (d, 1H), 8.18 (t, 1H), 5.44 (d, 1H), 4.19 (t, 2H), 3.99 (m, 4H), 3.26 (t, 2H), 2.17 (m, 2H). LCMS (ESI) 571.55 m/z [M+2H]⁺.

### Example 3 Synthesis of (S)-1-(6-fluoro-5-(4-oxo-5-(pyridin-3-yl)-4,5,6,7-tetrahydrothiazolo[5,4-c]pyridin-2-yl)pyridin-2-yl)pyrrolidin-3-yl methanesulfonate

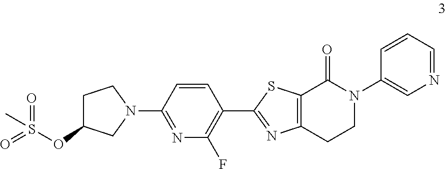 [SMILES: CS(=O)(=O)O[C@H]1CCN(c2ccc(-c3nc4c(s3)C(=O)N(c3cccnc3)CC4)c(F)n2)C1]

### Step-1: Synthesis of tert-butyl 3-bromo-2,4-dioxopiperidine-1-carboxylate (B)

Tert-butyl 2,4-dioxopiperidine-1-carboxylate A (10 g, 46.9 mmol) was dissolved in carbon tetrachloride (125 mL) and cooled to 0 to 5° C. To the above solution, N-bromo succinimide (8.35 g, 46.9 mmol) was added portion wise and stirring was continued at 28° C. for 1 hour. The reaction mixture was diluted with two runs of ethyl acetate (500 mL) and water (200 mL). The organic phase was separated, dried over Na₂SO₄, filtered and the solvents were removed under reduced pressure to obtain the title compound B as a white solid (10 g, 73%).

¹H NMR (500 MHz, DMSO-d₆): δ 8.1 (s, 2H), 3.89 (t, 2H), 2.76 (t, 2H), 1.45 (s, 9H). LCMS (ESI) 292.04 m/z [M+H]⁺.

### Step-2: Synthesis of tert-butyl 2-amino-4-oxo-6,7-dihydrothiazolo[5,4-c]pyridine-5(4H)-carboxylate (C)

Title compound B (10 g, 34.36 mmol), thiourea (2.61 g, 34.36 mmol) and sodium bicarbonate (2.88 g, 34.36 mmol) were dissolved in ethanol (160 mL) and heated in an oil bath at 80° C. for 2.5 hours. The reaction mixture was diluted with two runs of ethyl acetate (500 mL) and water (200 mL). The organic phase was separated, dried over Na₂SO₄, filtered and the solvents were removed under reduced pressure. The solid obtained was recrystallized from ethanol to obtain the title compound C as a white solid (6.6 g, 71%).

¹H NMR (500 MHz, DMSO-d₆): δ 8.1 (s, 2H), 3.89 (t, 2H), 2.76 (t, 2H), 1.45 (s, 9H). LCMS (ESI) 270.3 m/z [M+H]⁺.

### Step-3: Synthesis of tert-butyl 2-bromo-4-oxo-6,7-dihydrothiazolo[5,4-c]pyridine-5(4H)-carboxylate (D)

Title compound C (6.6 g, 24.53 mmol) was dissolved in acetonitrile (82 mL) and cooled to −10° C. in an ice bath with stirring. Tert-butyl nitrite (4.3 mL, 36.4 mmol) was added to the above solution and stirring was continued at −10° C. for 1 hour. Copper(II)-bromide (6.5 g, 29.43 mmol) was added to the above mixture, and it was allowed to stir at 28° C. for 1 hour. The reaction mixture was basified with aqueous saturated sodium bicarbonate solution to pH 8 to 9 and filtered. The filtrate collected was diluted with three runs of ethyl acetate (300 mL) and water (100 mL). The organic phase was separated, dried over Na₂SO₄, filtered and the solvents were removed under reduced pressure. The residue obtained was purified by column chromatography on silica gel (100 to 200 mesh) using ethyl acetate/n-hexane gradient (0/100->10/90) to afford the title compound D as a white solid (4.73 g, 58%).

¹H NMR (500 MHz DMSO-d₆): δ 4.12 (t, 2H), 3.10 (t, 2H), 1.5 (s, 9H). LCMS (ESI) 279 m/z [M+H-C₄H₈]⁺.

### Step-4: Synthesis of tert-butyl 2-(2,6-difluoropyridin-3-yl)-4-oxo-6,7-dihydrothiazolo[5,4-c]pyridine-5(4H)-carboxylate (3) (F)

Tert-butyl 2-bromo-4-oxo-6,7-dihydrothiazolo[5,4-c]pyridine-5(4H)-carboxylate D (0.3 g, 0.9 mmol) was dissolved in 1,4-dioxane (15 mL) and degassed by passing a stream of nitrogen through the mixture. Then [1,1′-bis(diphenyl-phosphino)ferrocene]-dichloropalladium(II), complex with dichloromethane (0.07 g, 0.09 mmol), title compound E (0.21 g, 1.35 mmol) and potassium phosphate tribasic (0.28 g, 1.35 mmol) were added, and the reaction mixture was heated at 100° C. in an oil-bath for 8 h. The reaction mixture was concentrated under reduced pressure. The residue was purified by column chromatography on silica gel (basic, 100-200 mesh) using ethyl acetate/n-hexane gradient (0/100->10/90->15/85) to obtain title compound F as a pale-yellow solid (0.2 g, 60%).

¹H NMR (500 MHz DMSO-d₆): δ 8.91 (q, 1H), 7.43 (dd, 1H), 4.1 (t, 2H), 3.19 (t, 2H), 1.5 (s, 9H). LCMS (ESI) 311.95 m/z [M+H-C₄H₈]⁺.

### Step-5: Synthesis of tert-butyl (S)-2-(2-fluoro-6-(3-hydroxypyrrolidin-1-yl)pyridin-3-yl)-4-oxo-6,7-dihydrothiazolo[5,4-c]pyridine-5(4H)-carboxylate (J)

Title compound 3 (0.2 g, 0.54 mmol), (S)-pyrrolidin-3-ol hydrochloride F (0.068 g, 0.54 mmol), N,N-diisopropylethylamine (0.09 mL, 1.06 mmol) were suspended in DMSO (20 mL) and stirring was continued at ambient temperature for 3 h. The reaction mixture was quenched with cold water (200 mL), and the solid precipitated was filtered over Whatmann filter paper and dried to obtain title compound J as a pale yellow solid (0.22 g, 92%).

### Step-6: Synthesis of (S)-2-(2-fluoro-6-(3-hydroxypyrrolidin-1-yl)pyridin-3-yl)-6,7-dihydrothiazolo[5,4-c]pyridin-4(5H)-one (K)

Title compound J (0.22 g, 0.5 mmol) was dissolved in DCM (4.4 mL) and cooled to 0° C. in an ice bath with stirring. 4M HCl in 1,4-dioxane (2.2 mL) was added to above solution and stirring was continued at RT for 2 h. After completion of the reaction, solvent was removed under reduced pressure. The residue obtained was dissolved in ice cold water and basified with aqueous saturated sodium bicarbonate solution to pH 8-9, the solid precipitated was filtered and dried to afford the title compound K as a pale yellow solid (0.155 g, 92%).

¹H NMR (500 MHz DMSO-d₆): δ 8.33 (t, 1H), 7.8 (s, 1H), 6.55 (d, 1H), 5.06 (s, 1H), 4.41 (s, 1H), 3.54 (m, 6H), 2.99 (t, 2H), 1.99 (d, 2H). LCMS (ESI) 334.9 [M+H]⁺.

### Step-7: Synthesis of (S)-2-(6-(3-((tert-butyldimethylsilyl)oxy)pyrrolidin-1-yl)-2-fluoropyridin-3-yl)-6,7-dihydrothiazolo[5,4-c]pyridin-4(5H)-one (L)

Title compound K (0.15 g, 0.44 mmol) and imidazole (0.3 g, 4.5 mmol) dissolved in DMF (3 mL) was cooled to 0° C. in an ice bath. tert-Butyl dimethylsilyl chloride (0.5 g, 3.35 mmol) was added portionwise. The reaction mixture was allowed to stir at ambient temperature for 36 h. The reaction mixture was diluted with cold water (150 mL), the solid precipitated was filtered and dried to obtain title compound L as a pale yellow solid (0.177 g, 88%).

¹H NMR (400 MHz, DMSO-d₆): δ 8.34 (t, 1H), 7.81 (s, 1H), 6.57 (dd, 1H), 4.59 (s, 1H), 3.63 (dd, 1H), 3.5 (m, 4H), 3.36 (m, 2H), 3.09 (s, 3H), 2.99 (t, 2H), 2.01 (d, 2H), 0.86 (s, 9H), 0.1 (d, 6H). LCMS (ESI) 449.25 m/z [M+H]⁺

### Step-8: Synthesis of (S)-2-(6-(3-((tert-butyldimethylsilyl)oxy)pyrrolidin-1-yl)-2-fluoropyridin-3-yl)-5-(pyridin-3-yl)-6,7-dihydrothiazolo[5,4-c]pyridin-4(5H)-one (M)

Compound L (0.17 g, 0.37 mmol) and 3-iodopyridine 8 (0.23 g, 1.1 mmol) were dissolved in degassed 1,4-dioxane (17 mL). After the addition of Pd[PPh₃]₄ (0.043 g, 0.03 mmol), XantPhos (0.043 g, 0.075 mmol), and Cs₂CO₃ (0.37 g, 1.13 mmol), the reaction mixture was heated at ˜100° C. in a oil-bath for 24 h. The reaction mixture was concentrated under reduced pressure to afford the crude residue. The crude residue was purified by column chromatography on silica gel (100-200 mesh) using CH₂Cl₂/MeOH gradient (100/0->99/1->98/2) to afford M as a yellow solid (0.17 g, 89%).

¹H NMR (500 MHz, DMSO-d₆): δ 8.75 (d, 1H), 8.54 (q, 1H), 8.48 (t, 1H), 7.94 (m, 1H), 7.52 (m, 1H), 6.7 (dd, 1H), 4.7 (s, 1H), 4.25 (t, 2H), 3.74 (dd, 1H), 3.63 (t, 2H), 3.35 (t, 2H), 3.2 (m, 1H), 2.21 (s, 1H), 2.02 (s, 1H), 0.96 (s, 9H), 0.2 (d, 6H). LCMS (ESI) 526 m/z [M+H]⁺.

### Step-9: Synthesis of (S)-2-(2-fluoro-6-(3-hydroxypyrrolidin-1-yl)pyridin-3-yl)-5-(pyridin-3-yl)-6,7-dihydrothiazolo[5,4-c]pyridin-4(5H)-one (N)

Title compound M (0.14 g, 0.26 mmol) was dissolved in DCM (2.8 mL) and cooled to 0° C. in an ice bath with stirring. 4M HCl in 1,4-dioxane (1.4 mL) was added to above solution and stirring was continued at RT for 3 h. After completion of the reaction, solvent was removed under reduced pressure. The residue obtained was dissolved in ice cold water and basified with aqueous saturated sodium bicarbonate solution to pH 8-9, the solid precipitated was filtered and dried to afford the title compound N as a pale yellow solid (0.08 g, 72%).

¹H NMR (500 MHz, DMSO-d₆): δ 8.66 (d, 1H), 8.44 (q, 1H), 8.38 (t, 1H), 7.84 (m, 1H), 7.47 (m, 1H), 6.7 (d, 1H), 6.58 (d, 1H), 5.08 (s, 1H), 4.42 (s, 1H), 4.16 (t, 2H), 3.55 (m, 4H), 3.25 (t, 2H), 2.0 (d, 2H). LCMS (ESI) 411.9 m/z [M+H]⁺.

### Step-10: Synthesis of ((S)-1-(6-fluoro-5-(4-oxo-5-(pyridin-3-yl)-4,5,6,7-tetrahydrothiazolo[5,4-c]pyridin-2-yl)pyridin-2-yl)pyrrolidin-3-yl methanesulfonate (3)

Title compound N (0.07 g, 0.17 mmol) was dissolved in pyridine (2.3 mL) and cooled to −50° C. in a dry ice bath with stirring. Methanesulfonylchloride (0.46 g, 4.08 mmol) was added to above solution and stirring was continued at 20° C. for 1 h. The reaction mixture was diluted with water (35 mL), the solid precipitated was filtered, washed with water (2×15 mL) and dried to obtain title compound 3 as a pale yellow solid (0.06 g, 72%).

¹H NMR (500 MHz, DMSO-d₆): δ 8.66 (d, 1H), 8.43 (m, 2H), 7.84 (m, 1H), 7.47 (q, 1H), 6.65 (dd, 1H), 5.45 (s, 1H), 4.16 (t, 2H), 3.76 (m, 3H), 3.55 (dd, 1H), 3.26 (m, 5H), 2.34 (s, 2H). LCMS (ESI) 489.85 m/z [M+H]⁺.

### Example 4 Synthesis of tritiated 2-[2-fluoro-6-[(3R)-3-fluoropyrrolidin-1-yl]-3-pyridyl]-5-(3-pyridyl)-6,7-dihydrothiazolo[5,4-c]pyridin-4-one ³H-Compound 1

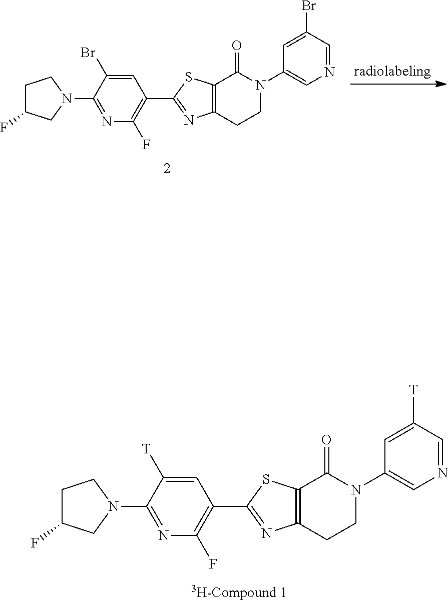

T means ³H.

2.49 mg of the brominated precursor 2, 50 μL DIPEA and 33.09 mg of Lindlar's catalyst were suspended in 0.3 ml DMF. The suspension was degassed three times at the high vacuum manifold and stirred under an atmosphere of tritium gas (772 mbar, 9.1 Ci) over night at room temperature. The solvent was removed in vacuo, and labile tritium was exchanged by adding 0.3 ml of methanol, stirring the solution, and removing the solvent again in vacuo. This process was repeated twice. The well dried solid was extracted with 5 ml ethanol/DMF (4:1) and the suspension was filtered through a 0.2 μm nylon membrane obtaining a clear solution. For the purification of the compound, the following HPLC conditions were found to be suitable: Waters Sunfire C18, 10×250 mm; solvents A: water+0.1% TFA); B: acetonitrile+0.1% TFA. The purified product ³H-Compound 1 was obtained (SA 48.5 Ci/mmol, 99% purity).

### Example 5 Synthesis of fluorinated 2-[2-fluoro-6-[(3R)-3-fluoropyrrolidin-1-yl]-3-pyridyl]-5-(3-pyridyl)-6,7-dihydrothiazolo[5,4-c]pyridin-4-one ¹⁸F Compound 1

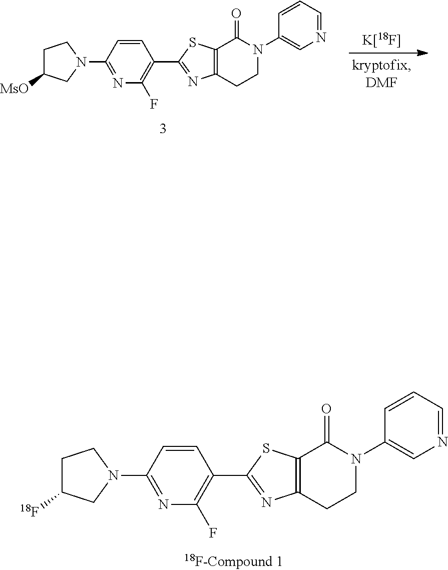

[¹⁸F]Fluoride in a shipping vial (U-18 enriched water obtained from a commercial cyclotron facility) was transferred onto and trapped on a Chromafix PS-HCO₃ cartridge (activated with KOTf (0.2 M, 6 mL), followed by water (6 mL). It was then eluted with a solution of TBAOTf (6 mg in 0.6 mL) into the reaction vessel of the TRACERlab® module. The solution was first heated at 70° C. for 3.5 min under vacuum and helium flow and the heat treatment was continued for an additional 2.5 min at 95° C. under the same conditions. The reactor was then cooled to 60° C. A solution of (S)-1-(6-fluoro-5-(4-oxo-5-(pyridin-3-yl)-4,5,6,7-tetrahydrothiazolo[5,4-c]pyridin-2-yl)pyridin-2-yl)pyrrolidin-3-yl methanesulfonate 3 (0.5 mg in 1 mL anhydrous acetonitrile) was added to the reaction vial and the vessel was heated at 100° C. for 10 min. The reaction vessel was cooled to 60° C. and the mixture was diluted with HPLC buffer (4 mL). Purification was performed by HPLC using a semi-preparative Phenomenex Gemini C18 column (5 μm, 250×10 mm) and elution with a mixture of acetonitrile/ammonium acetate solution (20 mM, 42/58, v/v) at a flow rate of 4 mL/min. The radiolabelled product was eluted from the SPE cartridge with 1.0 mL of ethanol into the formulation flask, pre-loaded with 10 mL of formulation base (ascorbic acid (10 mg) in saline). The resulting solution was passed through a sterilizing 0.2 μm membrane filter into a sterile, filter-vented vial. The final product ¹⁸F-Compound 1 was assayed, and sample was removed for QC testing. Confirmation of the identity of the product was determined by co-injection with a sample of the ¹⁹F-reference compound.

### Example Compounds 6 to 9 and 12 to 16

Following the coupling procedure as reported for the preparation of Example 1 and utilizing the building blocks of Example 1 Step 6, Example Compound 10 Step 2, Example 15 Step 2 and halogen derivatives indicated in Table 1, the following compounds were prepared.

TABLE 1

| | | | | 1. Yield |
|---|---|---|---|---|
| | | Halogen | | 2. ¹H NMR |
| Comp. | Building block | derivative | Product | 3. MH⁺ (ESI) |
| 6 | 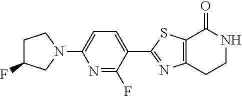 [SMILES: O=C1NCCc2nc(-c3ccc(N4CC[C@H](F)C4)nc3F)sc21] | 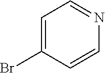 | 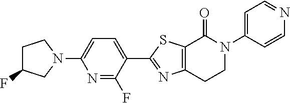 [SMILES: O=C1c2sc(-c3ccc(N4CC[C@H](F)C4)nc3F)nc2CCN1c1ccncc1] | 1. 88% 2. ¹H NMR (400 MHz, DMSO-d₆): δ 8.66 (d, 2H), 8.42 (dd, 1H), 7.7 (d, 2H), 6.65 (m, 1H), 5.49 (d, 1H), 4.26 (t, 2H), 3.73 (m, 3H), 3.53 |
| | | | | (m, 1H), 3.27 (t, 2H), |
| | | | | 2.22 (m, 2H). |
| | | | | 3. 414.15 |
| 7 | 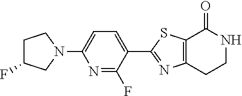 [SMILES: O=C1NCCc2nc(-c3ccc(N4CC[C@@H](F)C4)nc3F)sc21] | 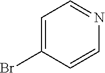 | 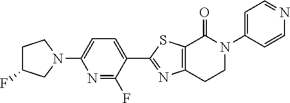 [SMILES: O=C1c2sc(-c3ccc(N4CC[C@@H](F)C4)nc3F)nc2CCN1c1ccncc1] | 1. 83% 2. ¹H NMR (400 MHz, DMSO-d₆): δ 8.62 (d, 2H), 8.42 (dd, 1H), 7.6 (d, 2H), 6.65 (m, 1H), 5.48 (d, 1H), 4.24 (t, 2H), 3.73 (m, 3H), 3.52 |
| | | | | (m, 1H), 3.26 (t, 2H), |
| | | | | 2.22 (m, 2H). |
| | | | | 3. 414.10 |
| 8 | 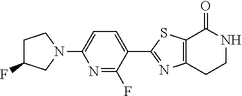 [SMILES: O=C1NCCc2nc(-c3ccc(N4CC[C@H](F)C4)nc3F)sc21] | 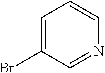 | 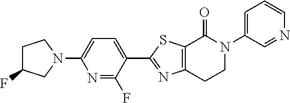 [SMILES: O=C1c2sc(-c3ccc(N4CC[C@H](F)C4)nc3F)nc2CCN1c1cccnc1] | 1. 73% 2. ¹H NMR (400 MHz, DMSO-d₆): δ 8.67 (d, 1H), 8.43 (m, 2H), 7.87 (d, 1H), 7.5 (dd, 1H), 6.64 (dd, 1H), 5.49 (d, 1H), 4.17 (t, 2H), 3.73 |
| | | | | (m, 3H), 3.52 (m, 1H), |
| | | | | 3.26 (t, 2H), 2.22 (m, |
| | | | | 2H). |
| | | | | 3. 414.15 |
| 9 | 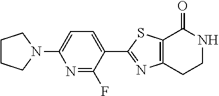 [SMILES: O=C1NCCc2nc(-c3ccc(N4CCCC4)nc3F)sc21] | 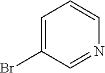 | 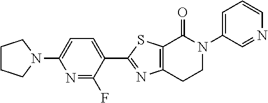 [SMILES: O=C1c2sc(-c3ccc(N4CCCC4)nc3F)nc2CCN1c1cccnc1] | 1. 50% 2. ¹H NMR (400 MHz, DMSO-d₆): δ 8.66 (s, 1H), 8.45 (d, 1H), 8.38 (t, 1H), 7.85 (d, 1H), 7.48 (dd, 1H), 6.58 (dd, 1H), 4.16 (t, 2H), 3.48 |
| | | | | (br-s, 4H), 3.25 (t, 2H), |
| | | | | 1.98 (br-s, 4H). |
| | | | | 3. 396.25 |
| 12 | 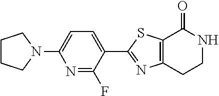 [SMILES: O=C1NCCc2nc(-c3ccc(N4CCCC4)nc3F)sc21] | 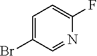 | 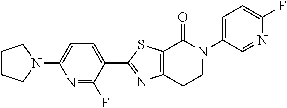 [SMILES: O=C1c2sc(-c3ccc(N4CCCC4)nc3F)nc2CCN1c1ccc(F)nc1] | 1. 16.8% 2. ¹H NMR (400 MHz, DMSO-d₆): δ 8.37 (dd, 1H), 8.31 (s, 1H), 8.06 (m, 1H), 7.27 (dd, 1H), 6.57 (dd, 1H), 4.14 (t, 2H), 3.48 (s, 4H), 3.25 |
| | | | | (t, 2H), 1.98 (s, 4H). |
| | | | | 3. 414.2 |
| 13 | 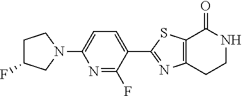 [SMILES: O=C1NCCc2nc(-c3ccc(N4CC[C@@H](F)C4)nc3F)sc21] | 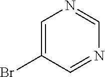 | 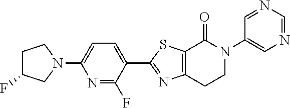 [SMILES: O=C1c2sc(-c3ccc(N4CC[C@@H](F)C4)nc3F)nc2CCN1c1cncnc1] | 1. 80% 2. ¹H NMR (400 MHz, DMSO-d₆): δ 9.04 (s, 1H), 8.93 (s, 2H), 8.42 (t, 1H), 6.65 (d, 1H), 5.49 (d, 1H), 4.23 (t, 2H), 3.75 (m, 3H), 3.53 |
| | | | | (m, 2H), 3.28 (t, 2H), |
| | | | | 2.25 (s, 2H). |
| | | | | 3. 414.9 |
| 14 | 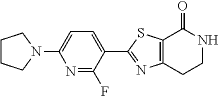 [SMILES: O=C1NCCc2nc(-c3ccc(N4CCCC4)nc3F)sc21] | 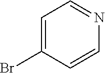 | 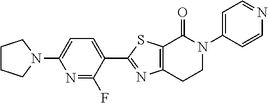 [SMILES: O=C1c2sc(-c3ccc(N4CCCC4)nc3F)nc2CCN1c1ccncc1] | 1. 25% 2. ¹H NMR (400 MHz DMSO-d₆): δ 8.56 (d, 2H), 8.37 (dd, 1H), 7.48- 7.46 (m, 2H), 6.58 (dd, 1H), 4.20 (t, 2H), 3.48 (br-s, 4H), 3.24 (t, 2H), |
| | | | | 1.98 (br-s, 4H) |
| | | | | 3. 414.9 |
| 15 | 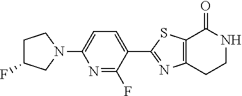 [SMILES: O=C1NCCc2nc(-c3ccc(N4CC[C@@H](F)C4)nc3F)sc21] | 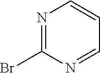 | 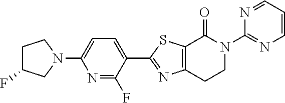 [SMILES: O=C1c2sc(-c3ccc(N4CC[C@@H](F)C4)nc3F)nc2CCN1c1ncccn1] | 1. 49% 2. ¹H NMR (400 MHz DMSO-d₆): δ 8.86 (d, 2H), 8.46 (dd, 1H), 7.39 (t, 1H), 6.64-6.72 (m, 1H), 5.6 (s, 1H), 4.33 (t, 2H), 3.9-3.73 (m, 4H), |
| | | | | 3.27-3.2 (m, 2H), 2.4- |
| | | | | 2.1 (m, 2H) |
| | | | | 3. 415.3 |
| 16 | 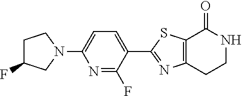 [SMILES: O=C1NCCc2nc(-c3ccc(N4CC[C@H](F)C4)nc3F)sc21] | 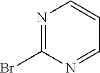 | 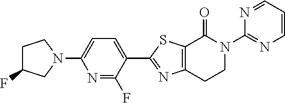 [SMILES: O=C1c2sc(-c3ccc(N4CC[C@H](F)C4)nc3F)nc2CCN1c1ncccn1] | 1. 82% 2. ¹H NMR (400 MHz DMSO-d₆): δ 8.81 (d, 2H), 8.42 (dd, 1H), 7.35 (t, 1H), 6.64 (dd, 1H), 5.49 (s, 1H), 4.29 (t, 2H), 3.94-3.62 (m, 3H), |
| | | | | 3.22(t, 2H), 2.23 (d, 2H) |
| | | | | 3. 415.3 |

### Example Compounds 11, 21, 22, 33

Following the coupling procedure as reported for the preparation of Example 15 (Step 3) and utilizing the building blocks from Example 1 Step 6, Example Compound 10 Step 2 and halogen derivatives indicated in Table 2, the following compounds were prepared.

TABLE 2

| | | | | 1. Yield |
|---|---|---|---|---|
| Com- | | Halogen | | 2. 1H NMR |
| pound | Building block | derivative | Product | 3. MH⁺ (ESI) |
| 11 | 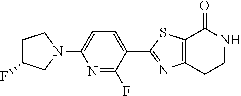 [SMILES: O=C1NCCc2nc(-c3ccc(N4CC[C@@H](F)C4)nc3F)sc21] | 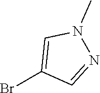 | 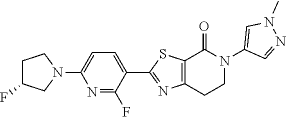 [SMILES: Cn1cc(N2CCc3nc(-c4ccc(N5CC[C@@H](F)C5)nc4F)sc3C2=O)cn1] | 1. 15% 2. ¹H NMR (400 MHz, DMSO-d₆): δ 8.4 (dd, 1H), 8.05 (s, 1H), 7.68 (s, 1H), 6.63 (dd, 1H), 5.48 (d, 1H), 4.08 (t, 2H), 3.84 (s, |
| | | | | 3H), 3.62 (m, 4H), |
| | | | | 3.22 (t, 2H), 2.22 |
| | | | | (d, 2H). |
| | | | | 3. 416.8 |
| 21 | 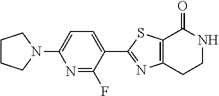 [SMILES: O=C1NCCc2nc(-c3ccc(N4CCCC4)nc3F)sc21] | 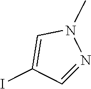 | 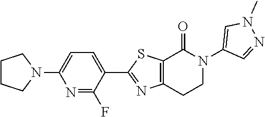 [SMILES: Cn1cc(N2CCc3nc(-c4ccc(N5CCCC5)nc4F)sc3C2=O)cn1] | 1. 12% 2. ¹H NMR (400 MHz, DMSO-d₆): δ 8.35 (dd, 1H), 8.05 (s, 1H), 7.68 (s, 1H), 6.56 (dd, 1H), 4.07 (t, 2H), 3.83 (s, 3H), 3.47 (s, |
| | | | | 4H), 3.21 (t, 2H), |
| | | | | 1.99 (s, 4H). |
| | | | | 3. 399.35 |
| 22 | 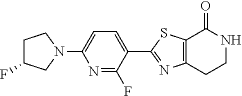 [SMILES: O=C1NCCc2nc(-c3ccc(N4CC[C@@H](F)C4)nc3F)sc21] | 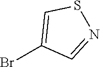 | 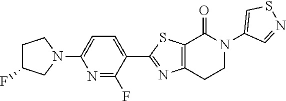 [SMILES: O=C1c2sc(-c3ccc(N4CC[C@@H](F)C4)nc3F)nc2CCN1c1cnsc1] | 1. 40% 2. ¹H NMR (500 MHz, DMSO-d₆): δ 8.95 (s, 1H), 8.89 (s, 1H), 8.41 (t, 1H), 6.64 (d, 1H), 5.48 (d, 1H), 4.22 |
| | | | | (t, 2H), 3.70 (dd, |
| | | | | 3H), 3.52 (td, 1H), |
| | | | | 3.27 (t, 2H) 2.4-2.2 |
| | | | | (m, 2H). |
| | | | | 3. 420.8 |
| 33 | 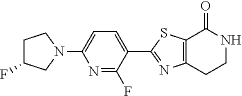 [SMILES: O=C1NCCc2nc(-c3ccc(N4CC[C@@H](F)C4)nc3F)sc21] | 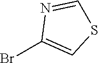 | 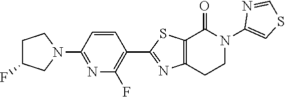 [SMILES: O=C1c2sc(-c3ccc(N4CC[C@@H](F)C4)nc3F)nc2CCN1c1cscn1] | 1. 12% 2. ¹H NMR (400 MHz, DMSO-d₆): δ 9.01 (d, 1H), 8.39- 8.34 (m, 1H), 7.82 (d, 1H), 6.60-6.57 |
| | | | | (m, 1H), 5.43 (d, |
| | | | | 1H), 4.46 (t, 2H), |
| | | | | 3.71-3.42 (m, 4H), |
| | | | | 3.19 (t, 2H), 2.24- |
| | | | | 2.10 (m, 2H). |
| | | | | 3. 420.30 |

### Synthesis of 5-(3-amino-4-fluorophenyl)-2-(2-fluoro-6-(pyrrolidin-1-yl)pyridin-3-yl)-6,7-dihydrothiazolo[5,4-c]pyridin-4(5H)-one (Example Compound 10)

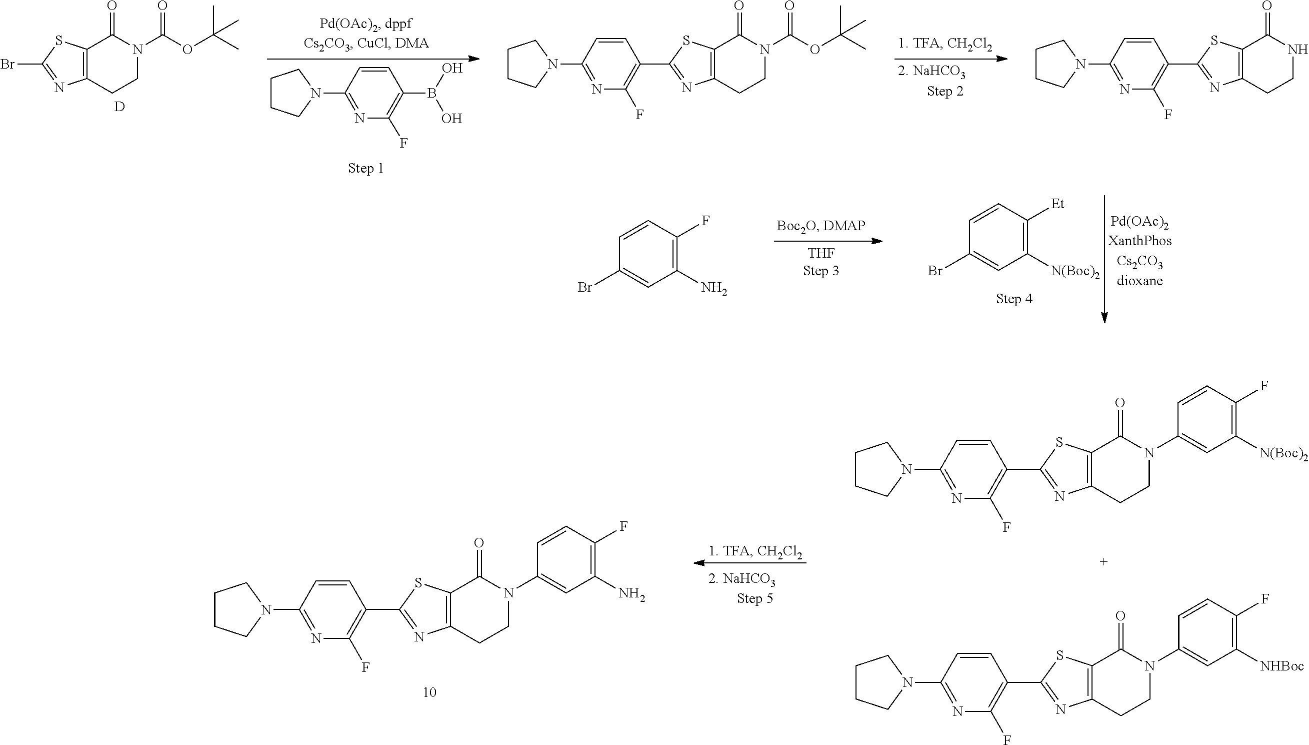

### Step-1: Synthesis of tert-butyl 2-(2-fluoro-6-(pyrrolidin-1-yl)pyridin-3-yl)-4-oxo-6,7-dihydrothiazolo[5,4-c]pyridine-5(4H)-carboxylate

In a 20 ml microwave vial, title compound D (0.3 g, 0.9 mmol) and commercially available (2-fluoro-6-(pyrrolidin-1-yl)pyridin-3-yl)boronic acid (0.38 g, 1.8 mmol) were dissolved in degassed DMA (10 mL). After the addition of Pd(OAc)₂ (0.01 g, 0.046 mmol), dppf (0.05 g, 0.092 mmol), CuCl (0.089 g, 0.9 mmol), and Cs₂CO₃ (0.588 g, 1.66 mmol), the reaction mixture was heated at ˜100° C. in a sand bath for 5 h. The above reaction was repeated one more time. The reaction mixture of both runs was diluted with CH₂Cl₂ (300 mL) and water/brine (140 mL; 1/1). The organic phase was separated, washed with brine (80 mL), dried with Na₂SO₄, filtered, and concentrated under reduced pressure. The dark residue was purified by chromatography on silica (100 g, HP-Ultra) using a Biotage Isolera system employing a CH₂Cl₂/EtOAc gradient (100/0->95/5->90/10->90/10) to afford the crude coupling product. The crude coupling product was further purified by chromatography on silica (100 g, HP-Ultra) using a Biotage Isolera system employing a CH₂Cl₂/EtOAc gradient (100/0->95/5->90/10->90/10) to afford the coupling product as a pale-yellow solid (0.473 g, 63%).

¹H NMR (400 MHz, DMSO-d₆/CDCl₃): δ 8.33 (t, 1H), 6.46 (dd, 1H), 4.06 (t, 2H), 3.08 (t, 2H), 2.00 (br-s, 4H), 1.50 (s, 9H). LCMS (ESI) 419/383.15/319.11 m/z [M+H]⁺.

### Step-2: Synthesis of 2-(2-fluoro-6-(pyrrolidin-1-yl)pyridin-3-yl)-6,7-dihydrothiazolo[5,4-c]pyridin-4(5H)-one

The starting material tert-butyl 2-(2-fluoro-6-(pyrrolidin-1-yl)pyridin-3-yl)-4-oxo-6,7-dihydrothiazolo[5,4-c]pyridine-5(4H)-carboxylate (0.473 g, 1.13 mmol) was dissolved in CH₂Cl₂ (25 mL) and the mixture was cooled to 0° C. At 0° C. TFA (4.4 mL) was added, and the reaction mixture was stirred at room temperature for 1 h. The solvents were evaporated under reduced pressure, and the residue was dissolved in CH₂Cl₂ (300 mL) and saturated NaHCO₃ (80 mL). The organic phase was separated, washed with brine (80 mL), dried with Na₂SO₄, filtered, and concentrated under reduced pressure to afford the deprotection product as a pale-yellow solid (0.349 g, 97%).

¹H NMR (400 MHz, DMSO-d₆): δ 8.33 (dd, 1H), 7.80 (br-s, 1H), 6.54 (dd, 1H), 3.53-3.43 (m, 6H), 2.99 (t, 2H), 1.97 (br-s, 4H). LCMS (ESI) 319.15 m/z [M+H]⁺.

### Step-3: Synthesis of tert-butyl (5-bromo-2-fluorophenyl)(tert-butoxycarbonyl)carbamate

Commercially available 5-bromo-2-fluoroaniline (0.5 g, 2.6 mmol) was dissolved in THF (15 mL) and Boc₂O (2.29 g, 10.5 mmol) was added. After the addition of DMAP (0.482 g, 3.95 mmol), the reaction mixture was stirred at room temperature for 3 days. The reaction mixture was diluted with EtOAc (150 mL) and water/brine (80 mL; 1/1). The organic phase was separated, dried with Na₂SO₄, filtered, and concentrated under reduced pressure. The dark residue was purified by chromatography on silica (50 g, HP-Ultra) using a Biotage Isolera system employing an EtOAc/n-heptane gradient (5/95->10/90->20/80->20/80) to afford the bis-Boc protected product as a colorless oil which becomes a white solid when stored at room temperature (0.452 g, 44%).

¹H NMR (400 MHz, DMSO-d₆): δ 7.74-7.70 (m, 1H), 7.63-7.59 (m, 1H), 7.33 (t, 1H), 1.39 (s, 18H).

### Step-4: Synthesis of tert-butyl (tert-butoxycarbonyl)(2-fluoro-5-(2-(2-fluoro-6-(pyrrolidin-1-yl)pyridin-3-yl)-4-oxo-6,7-dihydrothiazolo[5,4-c]pyridin-5(4H)-yl)phenyl)carbamate and tert-butyl (2-fluoro-5-(2-(2-fluoro-6-(pyrrolidin-1-yl)pyridin-3-yl)-4-oxo-6,7-dihydrothiazolo[5,4-c]pyridin-5(4H)-yl)phenyl)carbamate

In a 5 ml microwave vial Pd(OAc)₂ (0.002 g, 0.01 mmol), and XantPhos (0.016 g, 0.02 mmol) were dissolved in degassed 1,4 dioxane (6 mL). The reaction mixture was heated at ˜120° C. in a sand bath for ˜2 min to form the catalyst. After the addition of 2-(2-fluoro-6-(pyrrolidin-1-yl)pyridin-3-yl)-6,7-dihydrothiazolo[5,4-c]pyridin-4(5H)-one (0.035 g, 0.11 mmol), tert-butyl (5-bromo-2-fluorophenyl)(tert-butoxycarbonyl)carbamate (0.107 g, 0.275 mmol), and Cs₂CO₃ (0.163 g, 0.5 mmol), the reaction mixture was heated at ˜120° C. in a sand bath for 18 h. The reaction mixture was diluted with CH₂Cl₂ (50 mL) and water/brine (20 mL; 1/1). The organic phase was separated, washed with brine (20 mL), dried with Na₂SO₄, filtered, and concentrated under reduced pressure. The dark residue was purified by chromatography on silica (12 g, puriFlash, Interchim) using a Biotage Isolera system employing an EtOAc/n-heptane gradient (0/95->40/60->80/20->100/0) to afford a mixture of the bis-Boc protected and mono-Boc protected coupling products a yellow solid (0.0081 g, 11%).

LCMS (ESI) 628.44/528.32 (bis-Boc) and 528.30 (mono-Boc) m/z [M+H]⁺.

### Step-5: Synthesis of 5-(3-amino-4-fluorophenyl)-2-(2-fluoro-6-(pyrrolidin-1-yl)pyridin-3-yl)-6,7-dihydrothiazolo[5,4-c]pyridin-4(5H)-one (Compound 10)

The mixture of bis-Boc protected and mono-Boc protected coupling products (0.0225 g, 0.036 mmol) was dissolved in CH₂Cl₂ (5 mL) and the mixture was cooled to 0° C. At 0° C. TFA (0.39 mL) was added, and the reaction mixture was stirred at room temperature for 3 h. The solvents were evaporated under reduced pressure, and the residue was dissolved in CH₂Cl₂ (100 mL) and saturated NaHCO₃ (30 mL). The organic phase was separated, washed with brine (20 mL), dried with Na₂SO₄, filtered, and concentrated under reduced pressure. The residue was purified by chromatography on silica (12 g, puriFlash, Interchim) using a Biotage Isolera system employing an EtOAc/n-heptane gradient (5/95->40/60->80/20->80/20) to elute impurities, followed by EtOAc/CH₂Cl₂ (50/50) to afford Compound 10 as a pale-amber solid (0.0084 g, 54%).

¹H NMR (400 MHz, DMSO-d₆): δ 8.36 (t, 1H), 7.01 (dd, 1H), 6.77 (dd, 1H), 6.56 (dd, 1H), 6.52-6.48 (m, 1H), 5.24 (br-s, 2H), 3.99 (t, 2H), 3.48 (br-s, 4H), 3.19 (t, 2H), 1.99 (br-s, 4H). LCMS (ESI) 428.21 m/z [M+H]⁺.

### Example Compounds 26, 28 to 32, 34

Following the coupling procedure as reported for the preparation of Example 10 (Steps 4-5) and utilizing the building blocks from Example 1 Step 6, Example 15 Step 2, Example 16 Step 2, Example 17 Step 2 and halogen derivatives indicated in Table 3, the following compounds were prepared.

TABLE 3

| | | | | 1. Yield |
|---|---|---|---|---|
| Com- | | Halogen | | 2. ¹H NMR |
| pound | Building block | derivative | Product | 3. MH⁺ (ESI) |
| 26 | 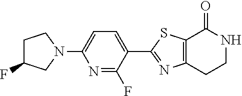 [SMILES: O=C1NCCc2nc(-c3ccc(N4CC[C@H](F)C4)nc3F)sc21] | 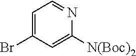 | 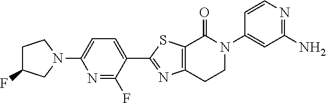 [SMILES: Nc1cc(N2CCc3nc(-c4ccc(N5CC[C@H](F)C5)nc4F)sc3C2=O)ccn1] | 1. 75% 2. ¹H NMR (400 MHz, DMSO-d₆): δ 8.41 (t, 1H), 7.91 (s, 1H), 6.6-6.4 (m, 3H), 5.97 (s, 2H), 5.48 (d, 1H), |
| | | | | 4. 08 (t, 2H), 3.9- |
| | | | | 3.6 (m, 3H), 3.6- |
| | | | | 3.5 (m, 1H), |
| | | | | 3.20(t, 2H), 2.4- |
| | | | | 2.1 (m, 2H). |
| | | | | 3. 529 |
| 28 | 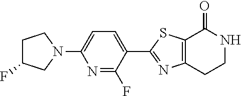 [SMILES: O=C1NCCc2nc(-c3ccc(N4CC[C@@H](F)C4)nc3F)sc21] | 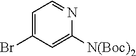 | 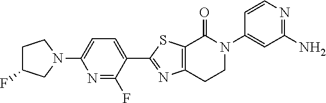 [SMILES: Nc1cc(N2CCc3nc(-c4ccc(N5CC[C@@H](F)C5)nc4F)sc3C2=O)ccn1] | 1. 32% 2. ¹H NMR (400 MHz, DMSO-d₆): δ 8.41(dd, 1H), 7.88 (d, 1H), 6.6 4(dd, 1H), 6.56 (dd, 1H), 6.49 (d, 1H), 5.96 |
| | | | | (s, 2H), 5.48 (d, |
| | | | | 1H), 4.1-4.06 (m, |
| | | | | 2H), 3.9-3.58 (m, |
| | | | | 3H), 3.56-3.46 |
| | | | | (m, 1H), 3.24-3.16 |
| | | | | (m, 2H), 2.4-2.1 |
| | | | | (m, 2H). |
| | | | | 3. 429.35 |
| 29 | 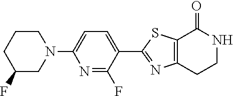 [SMILES: O=C1NCCc2nc(-c3ccc(N4CCC[C@H](F)C4)nc3F)sc21] | 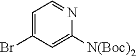 | 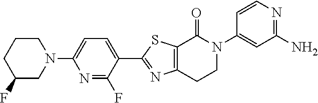 [SMILES: Nc1cc(N2CCc3nc(-c4ccc(N5CCC[C@H](F)C5)nc4F)sc3C2=O)ccn1] | 1. 89% 2. ¹H NMR (400 MHz, DMSO-d₆): δ 8.37 (t, 1H), 7.90 (s, 1H), 6.97 (d, 1H), 6.54 (d, 2H), 5.96 (s, 2H), 4.86 |
| | | | | (d, 1H), 4.26-4.16 |
| | | | | (m, 1H), 4.08 (t, |
| | | | | 2H), 4.01 (d, 1H), |
| | | | | 3.72-3.56 (m, |
| | | | | 1H), 3.38 (d, 1H), |
| | | | | 3.20 (t, 2H), 2.04- |
| | | | | 1.82 (m, 2H), 1.75, |
| | | | | (dd, 1H), 1.59, |
| | | | | (dd, 1H). |
| | | | | 3. 443.35 |
| 30 | 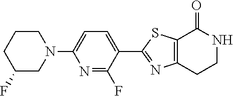 [SMILES: O=C1NCCc2nc(-c3ccc(N4CCC[C@@H](F)C4)nc3F)sc21] | 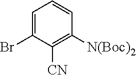 | 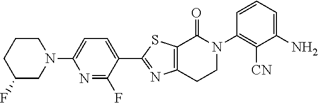 [SMILES: N#Cc1c(N)cccc1N1CCc2nc(-c3ccc(N4CCC[C@@H](F)C4)nc3F)sc2C1=O] | 1. 27% 2. ¹H NMR (400 MHz, DMSO-d₆): δ 8.38 (dd, 1H), 7.35 (t, 1H), 6.98 (dd, 1H), 6.75 (d, 1H), 6.63 (d, 1H), 6.20 |
| | | | | (s, 2H), 4.85 (dt, |
| | | | | 1H), 4.3-4.1 (m, |
| | | | | 1H), 4.15-3.85 |
| | | | | (m, 3H), 3.63 |
| | | | | (ddd, 1H), 3.22 (t, |
| | | | | 2H), 2.0-1.85 (m, |
| | | | | 2H), 1.8-1.7 (m, |
| | | | | 1H), 1.65-1.55 (m, |
| | | | | 1H). |
| | | | | 3. 467.35 |
| 31 | 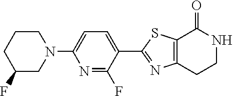 [SMILES: O=C1NCCc2nc(-c3ccc(N4CCC[C@H](F)C4)nc3F)sc21] | 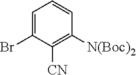 | 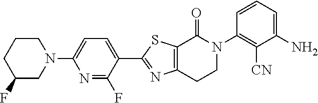 [SMILES: N#Cc1c(N)cccc1N1CCc2nc(-c3ccc(N4CCC[C@H](F)C4)nc3F)sc2C1=O] | 1. 52% 2. ¹H NMR (400 MHz, DMSO-d₆): δ 8.38(m, 1H), 7.35 (t, 1H), 6.97 (dd, 1H), 6.75 (d, 1H), 6.64 (d, 1H), |
| | | | | 6.19(s, 2H), 4.85 |
| | | | | (d, 1H), 4.25-4.1 |
| | | | | (m, 1H), 4.01-3.8 |
| | | | | (m, 3H), 3.64 (m, |
| | | | | 1H), 3.75-3.55 (m, |
| | | | | 1H), 3.23 (t, 2H), |
| | | | | 2.05-1.85 (m, 2H) |
| | | | | 1.8-1.7 (m, 1H), |
| | | | | 1.65-1.5 (m, 1H). |
| | | | | 3. 467.35 |
| 32 | 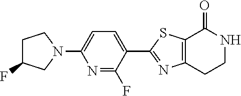 [SMILES: O=C1NCCc2nc(-c3ccc(N4CC[C@H](F)C4)nc3F)sc21] | 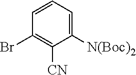 | 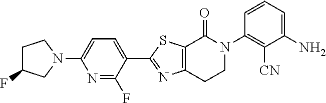 [SMILES: N#Cc1c(N)cccc1N1CCc2nc(-c3ccc(N4CC[C@H](F)C4)nc3F)sc2C1=O] | 1. 29% 2. ¹H NMR (400 MHz, DMSO-d₆): δ 8.43-8.38 (m, 1H), 7.35 (t, 1H), 6.76-6.74 (m, 1H), 6.63 (d, 2H), 6.19 |
| | | | | (s, 2H), 5.48 (d, |
| | | | | 1H), 4.01 (s, 2H), |
| | | | | 3.81-3.65 (m, 3H), |
| | | | | 3.53-3.48 (m, 1H), |
| | | | | 3.23 (t, 2H), 2.32- |
| | | | | 2.15 (m, 2H). |
| | | | | 3. 453.40 |
| 34 | 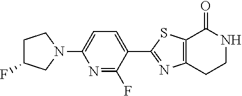 [SMILES: O=C1NCCc2nc(-c3ccc(N4CC[C@@H](F)C4)nc3F)sc21] | 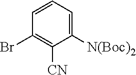 | 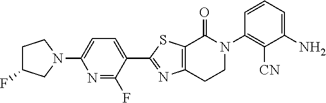 [SMILES: N#Cc1c(N)cccc1N1CCc2nc(-c3ccc(N4CC[C@@H](F)C4)nc3F)sc2C1=O] | 1. 30% 2. ¹H NMR (400 MHz, DMSO-d₆): δ 8.43-8.39 (m, 1H), 7.37-7.33 (m, 1H), 6.75 (d, 1H), 6.64 (d, 2H), 6.19 (s, |
| | | | | 1H), 5.49 (d, 1H), |
| | | | | 4.02 (s, 2H), 3.81- |
| | | | | 3.67 (m, 3H), |
| | | | | 3.55-3.49 (m, 1H), |
| | | | | 3.23 (t, 2H), 2.30- |
| | | | | 2.26 (m, 2H). |
| | | | | 3. 453.35 |

### Example Compounds 35-37

Following the coupling procedure as reported for the preparation of Example 1 and utilizing the building blocks of Example 15 Step 2, Example 16 Step 2, and halogen derivatives indicated in Table 4, the following compounds were prepared.

TABLE 4

| | | | | 1. Yield |
|---|---|---|---|---|
| Com- | | Halogen | | 2. ¹H NMR |
| pound | Building block | derivative | Product | 3. MH⁺ (ESI) |
| 35 | 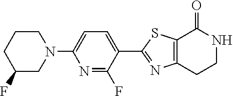 [SMILES: O=C1NCCc2nc(-c3ccc(N4CCC[C@H](F)C4)nc3F)sc21] | 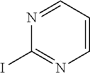 | 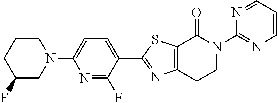 [SMILES: O=C1c2sc(-c3ccc(N4CCC[C@H](F)C4)nc3F)nc2CCN1c1ncccn1] | 1. 78% 2. ¹H NMR (400 MHz, DMSO-d₆): δ 8.81 (d, 2H), 8.40-8.35 (m, 1H), 7.34 (t, 1H), 6.99- 6.96 (m, 1H), |
| | | | | 4.92-4.89 (m, 1H), |
| | | | | 4.29 (t, 2H), 4.20- |
| | | | | 4.17 (m, 1H), |
| | | | | 4.02-3.99 (m, 1H), |
| | | | | 3.69-3.60 (m, 1H), |
| | | | | 3.37-3.36 (m, 1H), |
| | | | | 3.22 (t, 2H), 1.96- |
| | | | | 1.87 (m, 2H), |
| | | | | 1.74-1.73 (m, 1H), |
| | | | | 1.63-1.56 (m, 1H). |
| | | | | 3. 429.35 |
| 36 | 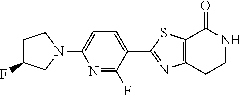 [SMILES: O=C1NCCc2nc(-c3ccc(N4CC[C@H](F)C4)nc3F)sc21] | 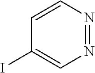 | 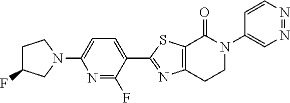 [SMILES: O=C1c2sc(-c3ccc(N4CC[C@H](F)C4)nc3F)nc2CCN1c1ccnnc1] | 1. 62% 2. ¹H NMR (400 MHz, DMSO-d₆): δ 9.44-9.43 (m, 1H), 9.20-9.18 (m, 1H), 8.44-8.39 (m, 1H), 7.71-7.68 (m, |
| | | | | 1H), |
| | | | | 6.66-6.63 (m, 1H), |
| | | | | 5.49 (d, 1H), 4.25 |
| | | | | (t, 2H), 3.76-3.67 |
| | | | | (m, 3H), 3.54-3.50 |
| | | | | (m, 1H), 3.28-3.25 |
| | | | | (m, 2H), 2.34-2.14 |
| | | | | (m, 2H). |
| | | | | 3. 415.35 |
| 37 | 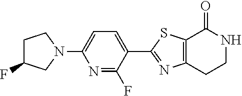 [SMILES: O=C1NCCc2nc(-c3ccc(N4CC[C@H](F)C4)nc3F)sc21] | 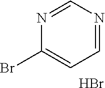 | 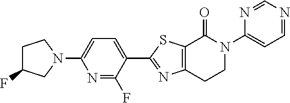 [SMILES: O=C1c2sc(-c3ccc(N4CC[C@H](F)C4)nc3F)nc2CCN1c1ccncn1] | 1. 9% 2. ¹H NMR (500 MHz, DMSO-d₆): δ 9.05 (d, 1H), 8.69 (d, 1H), 8.44-8.40 (m, 1H), 8.06-8.05 (m, 1H), 6.65 (d, |
| | | | | 1H), 5.49 (d, 1H), |
| | | | | 4.50 (t, 2H), 3.79- |
| | | | | 3.64 (m, 3H), |
| | | | | 3.53-3.50 (m, 1H), |
| | | | | 3.22 (t, 2H), 2.33- |
| | | | | 2.19 (m, 2H). |
| | | | | 3. 415.20 |

### Example 6 Synthesis of 2-(2-fluoro-6-(4-(hydroxymethyl)piperidin-1-yl)pyridin-3-yl)-5-(pyridin-3-yl)-6,7-dihydrothiazolo[5,4-c]pyridin-4(5H)-one (Example Compound 17)

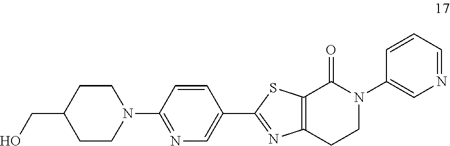 [SMILES: O=C1c2sc(-c3ccc(N4CCC(CO)CC4)nc3)nc2CCN1c1cccnc1]

### Step-1: Synthesis of tert-butyl 3-bromo-2,4-dioxopiperidine-1-carboxylate (B)

Tert-butyl 2,4-dioxopiperidine-1-carboxylate A (10 g, 46.9 mmol) was dissolved in CCl₄ (125 mL) and the mixture was cooled to 0 to 5° C. Then NBS (8.35 g, 46.9 mmol) was added portion wise and stirring was continued at 28° C. for 1 h. The reaction mixture was diluted with ethyl acetate (2×500 mL) and water (200 mL). The organic phase was separated, dried over Na₂SO₄, filtered and the solvents were removed under reduced pressure to obtain the title compound B as a white solid (10 g, 73%).

¹H NMR (500 MHz, DMSO-d₆): δ 8.1 (s, 2H), 3.89 (t, 2H), 2.76 (t, 2H), 1.45 (s, 9H). LCMS (ESI) 292.04 m/z [M+H]⁺.

### Step-2: Synthesis of tert-butyl 2-amino-4-oxo-6,7-dihydrothiazolo[5,4-c]pyridine-5(4H)-carboxylate (C)

Title compound B (10 g, 34.36 mmol), thiourea (2.61 g, 34.36 mmol) and sodium bicarbonate (2.88 g, 34.36 mmol) were dissolved in EtOH (160 mL) and heated in an oil bath at 80° C. for 2.5 h. The reaction mixture was diluted with ethyl acetate (2×500 mL) and water (200 mL). The organic phase was separated, dried over Na₂SO₄, filtered and the solvents were removed under reduced pressure. The solid was recrystallized from EtOH to obtain the title compound C as a white solid (6.6 g, 71%).

¹H NMR (500 MHz, DMSO-d₆): δ 8.1 (s, 2H), 3.89 (t, 2H), 2.76 (t, 2H), 1.45 (s, 9H). LCMS (ESI) 270.3 m/z [M+H]+.

### Step-3: Synthesis of tert-butyl 2-bromo-4-oxo-6,7-dihydrothiazolo[5,4-c]pyridine-5(4H)-carboxylate (D)

Title compound C (6.6 g, 24.53 mmol) was dissolved in CH₃CN (82 mL) and cooled to −10° C. in an ice bath with stirring. Tert-butyl nitrite (4.3 mL, 36.4 mmol) was added and stirring was continued at −10° C. for 1 h. Copper(II)-bromide (6.5 g, 29.43 mmol) was added, the ice bath was removed, and the reaction mixture was allowed to stir at 28° C. for 1 h. The reaction mixture was basified with aqueous saturated sodium bicarbonate solution to pH 8 to 9 and filtered. The collected filtrate was diluted ethyl acetate (3×300 mL) and water (100 mL). The organic phase was separated, dried over Na₂SO₄, filtered and the solvents were removed under reduced pressure. The residue obtained was purified by column chromatography on silica gel (100 to 200 mesh) using an ethyl acetate/n-hexane gradient (0/100->10/90) to afford the title compound D as a white solid (4.73 g, 58%).

¹H NMR (500 MHz DMSO-d₆): δ 4.12 (t, 2H), 3.10 (t, 2H), 1.5 (s, 9H). LCMS (ESI) 279 m/z [M+H-C₄H₈]⁺.

### Step-4: Synthesis of tert-butyl 2-(2,6-difluoropyridin-3-yl)-4-oxo-6,7-dihydrothiazolo[5,4-c]pyridine-5(4H)-carboxylate (F)

Title compound D (0.3 g, 0.9 mmol) was dissolved in 1,4-dioxane (15 mL) and degassed by passing a stream of nitrogen through the mixture. Then Pd(dppf)Cl₂×CH₂Cl₂ (0.07 g, 0.09 mmol), title compound E (0.21 g, 1.35 mmol) and potassium phosphate tribasic (0.28 g, 1.35 mmol) were added, and the reaction mixture was heated at 100° C. in an oil-bath for 8 h. The reaction mixture was concentrated under reduced pressure. The residue was purified by column chromatography on silica gel (basic, 100-200 mesh) using an ethyl acetate/n-hexane gradient (0/100->10/90->15/85) to obtain title compound F as a pale-yellow solid (0.2 g, 60%).

¹H NMR (500 MHz DMSO-d₆): δ 8.91 (q, 1H), 7.43 (dd, 1H), 4.1 (t, 2H), 3.19 (t, 2H), 1.5 (s, 9H).

LCMS (ESI) 311.95 m/z [M+H-C₄HB]⁺.

### Step-5: Synthesis of tert-butyl 2-(2-fluoro-6-(4-(hydroxymethyl)piperidin-1-yl)pyridin-3-yl)-4-oxo-6,7-dihydrothiazolo[5,4-c]pyridine-5(4H)-carboxylate (G)

Title compound F (0.15 g, 0.41 mmol), piperidin-4-ylmethanol (0.045 g, 0.41 mmol) and DIPEA (0.09 g, 0.82 mmol) were dissolved in DMSO (15 mL) and stirred at ambient temperature for 2 h. After completion of the reaction, the reaction mixture was quenched with ice cold water, filtered, and the solid was dried under reduced pressure to afford title compound G as an off-white solid (0.18 g, 98%).

¹H NMR (500 MHz, DMSO-d₆): δ 8.32 (t, 1H), 6.91 (dd, 1H), 4.51 (t, 1H), 4.37 (d, 2H), 4.05 (t, 2H), 3.28 (t, 3H), 3.09 (t, 2H), 2.98 (t, 2H), 2.54 (s, 3H), 1.73 (m, 3H), 1.49 (s, 9H), 1.12 (m, 2H). LCMS (ESI) 463.20 m/z [M+H]⁺.

### Step-6: Synthesis of 2-(2-fluoro-6-(4-(hydroxymethyl)piperidin-1-yl)pyridin-3-yl)-6,7-dihydrothiazolo[5,4-c]pyridin-4(5H)-one (H)

Title compound G (0.18 g, 0.38 mmol) was dissolved in DCM (4 mL) and cooled to 0° C. in an ice bath with stirring. Then 4M HCl in 1,4-dioxane (2 mL) was added and stirring was continued at RT for 3 h. After completion of the reaction, the solvent was removed under reduced pressure. The residue was dissolved in ice cold water and basified with aqueous saturated sodium bicarbonate solution to pH 8-9. The solid was collected by filtration and dried under reduced pressure to afford the title compound H as a pale-yellow solid (0.13 g, 92%).

¹H NMR (500 MHz, DMSO-d₆): δ 8.32 (t, 1H), 7.82 (s, 1H), 6.91 (dd, 1H), 4.51 (t, 1H), 4.36 (d, 2H), 3.5 (m, 2H), 3.27 (t, 2H), 2.97 (m, 4H), 1.73 (m, 3H), 1.12 (m, 2H). LCMS (ESI) 362.90 m/z [M+H]⁺.

### Step-7: Synthesis of 2-(6-(4-(((tert-butyldimethylsilyl)oxy)methyl)piperidin-1-yl)-2-fluoropyridin-3-yl)-6,7-dihydrothiazolo[5,4-c]pyridin-4(5H)-one (1)

Title compound H (0.13 g, 0.35 mmol) and imidazole (0.24 g, 3.6 mmol) were dissolved in DMF (3 mL) and the reaction mixture was cooled to 0° C. in an ice bath. Then tert-butyl dimethylsilyl chloride (0.26 g, 1.8 mmol) was added portionwise, and the reaction mixture was allowed to stir at RT for 24 h. The reaction mixture was diluted with cold water (50 mL), the precipitate was collected by filtration, and dried under reduced pressure. The residue was purified by column chromatography on silica gel (100-200 mesh) using a CH₂Cl₂/MeOH gradient (10010->99.5/0.5->99/1) to afford the title compound I as a yellow solid (0.15 g, 88%).

¹H NMR (500 MHz, DMSO-d₆): δ 8.32 (t, 1H), 7.82 (s, 1H), 6.90 (dd, 1H), 4.37 (d, 2H), 3.48 (m, 4H), 2.97 (m, 4H), 1.75 (d, 3H), 1.16 (m, 3H), 0.86 (s, 9H), 0.029 (s, 6H). LCMS (ESI) 477.00 m/z [M+H]⁺.

### Step-8: Synthesis of 2-(6-(4-(((tert-butyldimethylsilyl)oxy)methyl)piperidin-1-yl)-2-fluoropyridin-3-yl)-5-(pyridin-3-yl)-6,7-dihydrothiazolo[5,4-c]pyridin-4(5H)-one (J)

Title compound I (0.1 g, 0.21 mmol) and 3-iodopyridine (0.12 g, 0.63 mmol) were dissolved in degassed 1,4-dioxane (10 mL). After the addition of Pd[PPh₃]₄ (0.024 g, 0.021 mmol), XantPhos (0.024 g, 0.04 mmol), and Cs₂CO₃ (0.2 g, 0.63 mmol), the reaction mixture was heated at ˜100° C. in a sand bath for 24 h. The reaction mixture was concentrated under reduced pressure. The residue was purified by column chromatography on silica gel (100-200 mesh) using a CH₂Cl₂/MeOH gradient (100/0->99.5/0.5->99/1) to afford J as a yellow solid (0.085 g, 73%).

¹H NMR (500 MHz, DMSO-d₆): δ 8.32 (t, 1H), 7.82 (s, 1H), 6.90 (dd, 1H), 4.37 (d, 2H), 3.48 (m, 4H), 2.97 (m, 4H), 1.75 (d, 3H), 1.16 (m, 3H), 0.86 (s, 9H), 0.029 (s, 6H). LCMS (ESI) 477.00 m/z [M+H]⁺.

### Step-9: Synthesis of 2-(2-fluoro-6-(4-(hydroxymethyl)piperidin-1-yl)pyridin-3-yl)-5-(pyridin-3-yl)-6,7-dihydrothiazolo[5,4-c]pyridin-4(5H)-one (Compound 17)

Title compound J (0.08 g, 0.14 mmol) was dissolved in DCM (2 mL) and the mixture was cooled to 0° C. in an ice bath with stirring. Then 4M HCl in 1,4-dioxane (1 mL) was added and stirring was continued at RT for 3 h. After completion of the reaction, the solvent was removed under reduced pressure. The residue was dissolved in ice cold water and basified with aqueous saturated sodium bicarbonate solution to pH 8-9. The precipitated was collected by filtration and dried under reduced pressure to afford the title compound 4 as a pale-yellow solid (0.058 g, 92%).

¹H NMR (500 MHz, DMSO-d₆): δ 8.66 (d, 1H), 8.45 (q, 1H), 8.36 (dd, 1H), 7.84 (dt, 1H), 7.47 (q, 1H), 6.93 (dd, 1H), 4.52 (t, 1H), 4.38 (d, 2H), 4.16 (t, 2H), 3.26 (m, 4H), 2.98 (m, 2H), 1.72 (m, 3H), 1.13 (m, 2H). LCMS (ESI) 439.90 m/z [M+H]⁺.

### Example 7 Synthesis of ethyl 1-(6-fluoro-5-(4-oxo-5-(pyridin-3-yl)-4,5,6,7-tetrahydrothiazolo[5,4-c]pyridin-2-yl)pyridin-2-yl)piperidine-4-carboxylate (³H-Precursor of Compound 17)

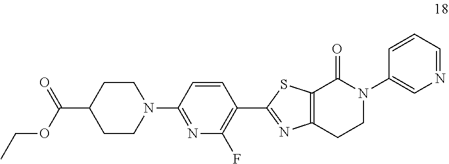 [SMILES: CCOC(=O)C1CCN(c2ccc(-c3nc4c(s3)C(=O)N(c3cccnc3)CC4)c(F)n2)CC1]

### Step-1: Synthesis of tert-butyl 3-bromo-2,4-dioxopiperidine-1-carboxylate (B)

Tert-butyl 2,4-dioxopiperidine-1-carboxylate A (10 g, 46.9 mmol) was dissolved in carbon tetrachloride (125 mL) and cooled to 0 to 5° C. To the above solution, N-bromo succinimide (8.35 g, 46.9 mmol) was added portionwise and stirring was continued at 28° C. for 1 hour. The reaction mixture was diluted with two runs of ethyl acetate (500 mL) and water (200 mL). The organic phase was separated, dried over Na₂SO₄, filtered and the solvents were removed under reduced pressure to obtain the title compound B as a white solid (10 g, 73%).

¹H NMR (500 MHz, DMSO-d₆): δ 8.1 (s, 2H), 3.89 (t, 2H), 2.76 (t, 2H), 1.45 (s, 9H). LCMS (ESI) 292.04 m/z [M+H]⁺.

### Step-2: Synthesis of tert-butyl 2-amino-4-oxo-6,7-dihydrothiazolo[5,4-c]pyridine-5(4H)-carboxylate (C)

Title compound B (10 g, 34.36 mmol), thiourea (2.61 g, 34.36 mmol) and sodium bicarbonate (2.88 g, 34.36 mmol) were dissolved in ethanol (160 mL) and heated in an oil bath at 80° C. for 2.5 hours. The reaction mixture was diluted with two runs of ethyl acetate (500 mL) and water (200 mL). The organic phase was separated, dried over Na₂SO₄, filtered and the solvents were removed under reduced pressure. The solid obtained was recrystallized from ethanol to obtain the title compound C as a white solid (6.6 g, 71%).

¹H NMR (500 MHz, DMSO-d₆): δ 8.1 (s, 2H), 3.89 (t, 2H), 2.76 (t, 2H), 1.45 (s, 9H). LCMS (ESI) 270.3 m/z [M+H]⁺.

### Step-3: Synthesis of tert-butyl 2-bromo-4-oxo-6,7-dihydrothiazolo[5,4-c]pyridine-5(4H)-carboxylate (D)

Title compound C (6.6 g, 24.53 mmol) was dissolved in acetonitrile (82 mL) and cooled to −10° C. in an ice bath with stirring. Tert-butyl nitrite (4.3 mL, 36.4 mmol) was added to the above solution and stirring was continued at −10° C. for 1 hour. Copper(II)-bromide (6.5 g, 29.43 mmol) was added to the above mixture, and it was allowed to stir at 28° C. for 1 hour. The reaction mixture was basified with aqueous saturated sodium bicarbonate solution to pH 8 to 9 and filtered. The filtrate collected was diluted with three runs of ethyl acetate (300 mL) and water (100 mL). The organic phase was separated, dried over Na₂SO₄, filtered and the solvents were removed under reduced pressure. The residue obtained was purified by column chromatography on silica gel (100 to 200 mesh) using ethyl acetate/n-hexane gradient (0/100->10/90) to afford the title compound D as a white solid (4.73 g, 58%).

¹H NMR (500 MHz DMSO-d₆): δ 4.12 (t, 2H), 3.10 (t, 2H), 1.5 (s, 9H). LCMS (ESI) 279 m/z [M+H-C₄H₈]⁺.

### Step-4: Synthesis of tert-butyl 2-(2,6-difluoropyridin-3-yl)-4-oxo-6,7-dihydrothiazolo[5,4-c]pyridine-5(4H)-carboxylate (F)

Tert-butyl 2-bromo-4-oxo-6,7-dihydrothiazolo[5,4-c]pyridine-5(4H)-carboxylate D (0.3 g, 0.9 mmol) was dissolved in 1,4-dioxane (15 mL) and the mixture was degassed by passing a stream of nitrogen through the mixture. Then [1,1′-bis(diphenyl-phosphino)ferrocene]-dichloropalladium(II), complex with dichloromethane (0.07 g, 0.09 mmol), title compound E (0.21 g, 1.35 mmol) and potassium phosphate tribasic (0.28 g, 1.35 mmol) were added, and the reaction mixture was heated at 100° C. in an oil-bath for 8 h. The reaction mixture was concentrated under reduced pressure. The residue was purified by column chromatography on silica gel (basic, 100-200 mesh) using ethyl acetate/n-hexane gradient (0/100->10/90->15/85) to obtain title compound F as a pale-yellow solid (0.2 g, 60%).

¹H NMR (500 MHz DMSO-d6): δ 8.91 (q, 1H), 7.43 (dd, 1H), 4.1 (t, 2H), 3.19 (t, 2H), 1.5 (s, 9H). LCMS (ESI) 311.95 m/z [M+H-C4H8]+

### Step-5: Synthesis of tert-butyl 2-(6-(4-(ethoxycarbonyl)piperidin-1-yl)-2-fluoropyridin-3-yl)-4-oxo-6,7-dihydrothiazolo[5,4-c]pyridine-5(4H)-carboxylate (K)

Title compound F (0.3 g, 0.82 mmol), ethyl piperidine-4-carboxylate (0.13 g, 0.82 mmol) and DIPEA (0.2 g, 1.63 mmol) were dissolved in DMSO (30 mL) and the reaction mixture was stirred at ambient temperature for 2 hours. After completion of the reaction the reaction mixture was quenched with ice cold water, the solid which precipitated was filtered and dried to afford the title compound K as an off-white solid (0.37 g, 90%).

¹H NMR (500 MHz, DMSO-d₆): δ 8.35 (t, 1H), 6.93 (dd, 1H), 4.26 (d, 2H), 4.07 (m, 4H), 3.15 (m, 2H), 3.10 (t, 2H), 2.69 (m, 1H), 1.93 (dd, 2H), 1.58 (m, 2H), 1.49 (s, 9H), 1.19 (t, 3H). LCMS (ESI) 505.35 m/z [M+H]⁺.

### Step-6: Synthesis of ethyl 1-(6-fluoro-5-(4-oxo-4,5,6,7-tetrahydrothiazolo[5,4-c]pyridin-2-yl)pyridin-2-yl)piperidine-4-carboxylate (L)

Title compound K (0.37 g, 0.73 mmol) was dissolved in DCM (11 mL) and the mixture was cooled to 0° C. in an ice bath with stirring. 4M HCl in 1,4-dioxane (4.0 mL) was added to the above solution and stirring was continued at RT for 2 h. After completion of the reaction, solvent was removed under reduced pressure. The residue obtained was dissolved in ice cold water and basified with aqueous saturated sodium bicarbonate solution to pH 8-9, the solid which precipitated was filtered and dried to afford the title compound L as a pale-yellow solid (0.26 g, 89%).

¹H NMR (500 MHz, DMSO-d₆): δ 8.34 (t, 1H), 7.83 (s, 1H), 6.92 (dd, 1H), 4.25 (d, 2H), 4.08 (q, 2H), 3.50 (m, 2H), 3.12 (m, 2H), 2.99 (t, 2H), 2.69 (m, 1H), 1.93 (m, 2H), 1.57 (m, 2H), 1.21 (m, 3H). LCMS (ESI) 405.5 m/z [M+H]⁺.

### Step-7: Synthesis of ethyl 1-(6-fluoro-5-(4-oxo-5-(pyridin-3-yl)-4,5,6,7-tetrahydrothiazolo[5,4-c]pyridin-2-yl)pyridin-2-yl)piperidine-4-carboxylate (18)

Title compound L (0.15 g, 0.37 mmol) and 3-bromopyridine (0.26 g, 1.11 mmol) were dissolved in degassed 1,4-dioxane (15 mL). After the addition of Pd[PPh₃]₄ (0.021 g, 0.018 mmol), XantPhos (0.021 g, 0.037 mmol), and Cs₂CO₃ (0.24 g, 0.74 mmol), the reaction mixture was heated at ˜100° C. in a oil-bath for 24 h. The reaction mixture was concentrated under reduced pressure to afford the crude residue. The crude residue was purified by column chromatography on silica gel (100-200 mesh) using CH₂Cl₂/MeOH gradient (100/0->99.5/0.5->99/1) to afford compound 18 as a yellow solid (0.15 g, 75%).

¹H NMR (500 MHz, DMSO-d₆): δ 8.67 (d, 1H), 8.58 (d, 1H), 8.38 (dd, 1H), 8.17 (t, 1H), 6.95 (dd, 1H), 4.26 (d, 2H), 4.18 (t, 2H), 4.08 (q, 2H), 3.25 (t, 2H), 3.16 (m, 2H), 2.72 (m, 1H), 1.94 (dd, 1H), 1.55 (m, 2H), 1.19 (t, 3H). LCMS (ESI) 560.30 m/z [M+H]⁺.

### Example 8 Synthesis of 2-(6-(4-(hydroxymethyl)piperidin-1-yl)-2-nitropyridin-3-yl)-5-(pyridin-3-yl)-6,7-dihydrothiazolo[5,4-c]pyridin-4(5H)-one

### (¹⁸F-Precursor of Compound 17)

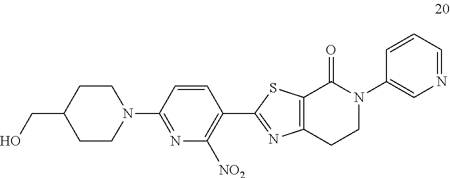 [SMILES: O=C1c2sc(-c3ccc(N4CCC(CO)CC4)nc3[N+](=O)[O-])nc2CCN1c1cccnc1]

### Step-1: Synthesis of tert-butyl 3-bromo-2,4-dioxopiperidine-1-carboxylate (B)

Tert-butyl 2,4-dioxopiperidine-1-carboxylate A (10 g, 46.9 mmol) was dissolved in carbon tetrachloride (125 mL) and the mixture was cooled to 0 to 5° C. To the above solution, N-bromosuccinimide (8.35 g, 46.9 mmol) was added portionwise and stirring was continued at 28° C. for 1 hour. The reaction mixture was diluted with two runs of ethyl acetate (500 mL) and water (200 mL). The organic phase was separated, dried over Na₂SO₄, filtered and the solvents were removed under reduced pressure to obtain the title compound B as a white solid (10 g, 73%).

¹H NMR (500 MHz, DMSO-d₆): δ 8.1 (s, 2H), 3.89 (t, 2H), 2.76 (t, 2H), 1.45 (s, 9H). LCMS (ESI) 292.04 m/z [M+H]⁺.

### Step-2: Synthesis of tert-butyl 2-amino-4-oxo-6,7-dihydrothiazolo[5,4-c]pyridine-5(4H)-carboxylate (C)

Title compound B (10 g, 34.36 mmol), thiourea (2.61 g, 34.36 mmol) and sodium bicarbonate (2.88 g, 34.36 mmol) were dissolved in ethanol (160 mL) and heated in an oil bath at 80° C. for 2.5 hours. The reaction mixture was diluted with two runs of ethyl acetate (500 mL) and water (200 mL). The organic phase was separated, dried over Na₂SO₄, filtered and the solvents were removed under reduced pressure. The solid obtained was recrystallized from ethanol to obtain the title compound C as a white solid (6.6 g, 71%).

¹H NMR (500 MHz, DMSO-d₆): δ 8.1 (s, 2H), 3.89 (t, 2H), 2.76 (t, 2H), 1.45 (s, 9H). LCMS (ESI) 270.3 m/z [M+H]⁺.

### Step-3: Synthesis of tert-butyl 2-(dibenzylamino)-4-oxo-6,7-dihydrothiazolo[5,4-c]pyridine-5(4H)-carboxylate (M)

Title compound C (0.1 g, 0.37 mmol) and cesium bicarbonate (0.84 g, 2.6 mmol) were suspended in DMF (5 mL) and the mixture was cooled to 0° C. in an ice bath while stirring. Benzyl bromide (0.26 g, 1.5 mmol) was added to the above solution and stirring was continued at RT for 7 h. The reaction mixture was quenched water (50 mL) and extracted with two runs of ethyl acetate (25 mL). The organic phase was separated, dried over Na₂SO₄, filtered and the solvents were removed under reduced pressure to afford the crude residue. The crude residue was purified by column chromatography on basic silica gel (100-200 mesh) using hexane/EtOAc gradient (100/0->98/2->96/4->94/6->92/8) to obtain the title compound M as an off-white sticky solid (0.09 g, 71%).

¹H NMR (400 MHz, DMSO-d₆): δ 7.33 (m, 10H), 4.8 (s, 4H), 3.93 (t, 2H), 2.85 (t, 2H), 1.44 (s, 9H). LCMS (ESI) 448 m/z [M−H]⁺.

### Step-4: Synthesis of 2-(dibenzylamino)-6,7-dihydrothiazolo[5,4-c]pyridin-4(5H)-one (N)

Title compound M (0.09 g, 0.2 mmol) was dissolved in DCM (2 mL) and cooled to 0° C. in an ice bath with stirring. TFA (0.45 mL) was added to the above solution and stirring was continued at RT for 2 h. After completion of the reaction, the solvent was removed under reduced pressure. The residue obtained was dissolved in ice cold water and basified with aqueous saturated sodium bicarbonate solution to pH 8-9. The mixture was extracted with two runs of ethyl acetate (25 mL). The organic phase was separated, dried over Na₂SO₄, filtered and the solvents were removed under reduced pressure to afford the title compound N as an off white solid (0.065 g, 93%).

¹H NMR (400 MHz, DMSO-d₆): δ 7.32 (m, 10H), 4.76 (s, 4H), 3.37 (m, 2H), 2.73 (t, 2H). LCMS (ESI) 350.4 m/z [M−H]⁺.

### Step-5: Synthesis of 2-(dibenzylamino)-5-(pyridin-3-yl)-6,7-dihydrothiazolo[5,4-c]pyridin-4(5H)-one(O)

Title compound N (1.0 g, 2.86 mmol) and 3-iodopyridine (1.75 g, 8.5 mmol) were dissolved in degassed 1,4-dioxane (50 mL). After the addition of Pd[PPh₃]₄ (0.16 g, 0.14 mmol), XantPhos (0.16 g, 0.28 mmol), and Cs₂CO₃ (2.78 g, 8.5 mmol), the reaction mixture was heated at ˜100° C. in a oil-bath for 24 h. The reaction mixture was concentrated under reduced pressure to afford the crude residue. The crude residue was purified by column chromatography on silica gel (100-200 mesh) using CH₂Cl₂/MeOH gradient (100/0->99.5/0.5->99/1->98/2) to afford O as an off white solid (0.75 g, 61%).

¹H NMR (400 MHz, DMSO-d₆): δ 8.57 (d, 1H), 8.37 (dd, 1H), 7.74 (m, 1H), 7.39 (m, 5H), 7.31 (m, 6H), 4.81 (s, 4H), 4.02 (t, 2H), 3.0 (t, 2H). LCMS (ESI) 424.8 m/z [M−H]⁺.

### Step-6: Synthesis of 2-amino-5-(pyridin-3-yl)-6,7-dihydrothiazolo[5,4-c]pyridin-4(5H)-one (P)

Title compound O (0.75 g, 1.7 mmol) was suspended in toluene (75 mL) and cooled to 0° C. in an ice bath with stirring. Methane sulfonic acid (7.5 mL) was added to the above solution and stirring was continued at 100° C. for 6 h. After completion of the reaction, two layers were separated. The toluene phase was decanted to obtain a crude brown liquid. The crude liquid was cooled to 0° C. in an ice bath and quenched with aqueous saturated sodium bicarbonate solution to pH 8-9, extracted with two volumes of 10% of MeOH in DCM (75 mL). The organic phase was separated, dried over Na₂SO₄, filtered and the solvents were removed under reduced pressure to afford the title compound P as an off white solid (0.22 g, 51%).

¹H NMR (400 MHz, DMSO-d₆): δ 8.58 (d, 1H), 8.37 (d, 1H), 7.93 (s, 2H), 7.74 (m, 1H), 7.41 (d, 1H), 3.99 (t, 2H), 2.9 (t, 2H). LCMS (ESI) 244.8 m/z [M−H]⁺.

### Step-7: Synthesis of 2-bromo-5-(pyridin-3-yl)-6,7-dihydrothiazolo[5,4-c]pyridin-4(5H)-one(Q)

Title compound P (0.5 g, 2.03 mmol) was dissolved in acetonitrile (25 mL) and cooled to 0° C. in an ice bath with stirring. Tert-butyl nitrite (0.7 mL, 6.0 mmol) was added to the above solution and stirring was continued at 0° C. for 30 min. Copper (II)-bromide (0.9 g, 4.06 mmol) was added to the above mixture, and it was allowed to stir at 28° C. for 3 hours. The reaction mixture was basified with aqueous saturated sodium bicarbonate solution to pH 8 to 9 and filtered. The filtrate collected was diluted with two runs of 10% MeOH in DCM (100 mL). The organic phase was separated, dried over Na₂SO₄, filtered and the solvents were removed under reduced pressure. The residue obtained was purified by column chromatography on silica gel (100 to 200 mesh) using CH₂Cl₂/MeOH gradient (100/0->99.5/0.5->99/1->98/2) to afford the title compound Q as off white solid (0.22 g, 35%).

¹H NMR (400 MHz, DMSO-d₆): δ 8.64 (m, 1H), 8.46 (dd, 1H), 7.83 (m, 1H), 7.48 (m, 1H), 4.13 ((t, 2H), 3.24 (t, 2H). LCMS (ESI) 307.7 m/z [M−H]⁺.

### Step-8: Synthesis of (1-(6-nitropyridin-2-yl)piperidin-4-yl)methanol (S)

Compound R (2.5 g, 31.64 mmol), piperidin-4-ylmethanol (9.4 g, 81.7 mmol) and DIPEA (12.26 mL, 94.8 mmol) were suspended in dioxane (120 mL) using a microwave vial. The sealed vial was then heated at 100° C. for 3 hours using a CEM microwave. The reaction mixture was concentrated under reduced pressure to afford a crude residue. The crude residue was purified by column chromatography on basic silica gel (100-200 mesh) using a hexane/EtOAc gradient (100/0->90/10->80/20->70/30->60/40->50/50) to obtain the title compound S as a yellow oil (4.0 g, 53%).

¹H NMR (500 MHz, DMSO-d₆): δ 7.83 (t, 1H), 7.38 (d, 1H), 7.27 (d, 1H), 4.5 (t, 1H), 4.37 (d, 2H), 3.28 (t, 2H), 2.9 (m, 2H), 1.75 (d, 2H), 1.66 (m, 1H), 1.12 (m, 2H). LCMS (ESI) 238.1 m/z [M+H]⁺.

### Step-9: Synthesis of (1-(5-bromo-6-nitropyridin-2-yl)piperidin-4-yl)methanol (T)

Title compound S (2.1 g, 8.8 mmol) was dissolved in ACN (42 mL) and cooled to −30° C. NBS (2.39 g, 13.29 mmol) was added portionwise. The reaction mixture was allowed to warm up to 0° C. during a period of 2 h. The reaction mixture was quenched water (50 mL) and extracted with two runs of ethyl acetate (100 mL). The organic phase was separated, dried over Na₂SO₄, filtered and the solvents were removed under reduced pressure to afford the crude residue. The crude residue was purified by column chromatography on basic silica gel (100-200 mesh) using a hexane/EtOAc gradient (100/0->90/10->80/20->70/30->60/40->50/50) to obtain the title compound T as a pale-yellow oil (1.5 g, 54%).

¹H NMR (400 MHz, DMSO-d₆): δ 7.96 (d, 1H), 7.11 (d, 1H), 4.5 (t, 1H), 4.23 (d, 2H), 3.26 (t, 2H), 2.88 (m, 2H), 1.68 (m, 3H), 1.11 (m, 2H). LCMS (ESI) 313.9 m/z [M−H]⁺.

### Step-10: Synthesis of (1-(6-nitro-5-(trimethylstannyl)pyridin-2-yl)piperidin-4-yl)methanol (U)

Title compound T (1.4 g, 4.4 mmol) was dissolved in degassed 1,4-dioxane (28 mL) in a microwave vial. After the addition of Pd[PPh₃)₂Cl₂ (0.3 g, 0.44 mmol), hexamethyldistannane (1.3 mL, 1.95 g, 6.0 mmol), and triphenylarsine (0.135 g, 0.4 mmol), the vial was sealed and heated at 100° C. in a CEM microwave for 1 h. The reaction mixture was concentrated under reduced pressure to afford the crude residue. The crude residue was purified by column chromatography on basic silica gel (100-200 mesh) using a hexane/EtOAc gradient (100/0->90/10->80/20->70/30->60/40->50/50) to afford U as yellow oil (0.7 g, 41%).

¹H NMR (400 MHz, DMSO-d₆): δ 7.8 (d, 1H), 7.24 (d, 1H), 4.49 (t, 1H), 4.36 (d, 2H), 3.26 (t, 2H), 2.88 (m, 2H), 1.7 (m, 3H), 1.14 (m, 2H), 0.26 (s, 9H). LCMS (ESI) 402.1 m/z [M+H]⁺.

### Step-11: Synthesis of 6-(4-(((tert-butyldimethylsilyl)oxy)methyl)piperidin-1-yl)-2-nitro-3-(trimethylstannyl)pyridine (V)

Title compound U (0.55 g, 1.37 mmol) and imidazole (1.3 g, 13.7 mmol) which had been dissolved in DMF (11 mL) was cooled to 0° C. in an ice bath. DMAP (0.033 g, 0.27 mmol) and tert-butyl dimethylsilyl chloride (1.0 g, 6.8 mmol) were added portionwise. The reaction mixture was allowed to stir at ambient temperature for 2 h. The reaction mixture was quenched water (100 mL) and extracted with two runs of ethyl acetate (100 mL). The organic phase was separated, dried over Na₂SO₄, filtered and the solvents were removed under reduced pressure to afford the crude residue. The crude residue was purified by column chromatography on basic silica gel (100-200 mesh) using a hexane/EtOAc gradient (100/0->99.5/0.5->99/1->98.5/1.5->98/2) to obtain title compound V as a yellow oil (0.32 g, 43%).

¹H NMR (400 MHz, DMSO-d₆): δ 7.81 (d, 1H), 7.25 (d, 1H), 4.38 (d, 2H), 3.45 (d, 2H), 2.89 (t, 2H), 1.73 (d, 3H), 1.15 (m, 2H), 0.85 (s, 9H), 0.26 (s, 6H). LCMS (ESI) 514.7 m/z [M]⁺.; 517.3 m/z [M+2H]⁺.

### Step-12: 2-(6-(4-(((tert-butyldimethylsilyl)oxy)methyl)piperidin-1-yl)-2-nitropyridin-3-yl)-5-(pyridin-3-yl)-6,7-dihydrothiazolo[5,4-c]pyridin-4(5H)-one (W)

Compound Q (0.3 g, 0.96 mmol) and compound V (0.74 g, 1.4 mmol) were dissolved in degassed 1,4-dioxane (45 mL). After the addition of Pd(OAc)₂ (0.02 g, 0.1 mmol), XantPhos (0.055 g, 0.1 mmol), and Cu(I)CI (0.095 g, 0.96 mmol), the reaction mixture was heated at 80° C. in an oil-bath for 4 h. The reaction mixture was concentrated under reduced pressure to afford the crude residue. The crude residue was purified by column chromatography on silica gel (100-200 mesh) using CH₂Cl₂/MeOH gradient (100/0->99/1->98/2) to afford impure solid. The impure solid was further purified by preparative TLC plates using dichloromethane/methanol (98/2) as a mobile phase to obtain the compound W as a yellow solid (0.15 g, 26%).

¹H NMR (400 MHz, DMSO-d₆): δ 8.65 (d, 1H), 8.45 (dd, 1H), 8.17 (d, 1H), 7.83 (m, 1H), 7.48 (dd, 1H), 7.23 (d, 1H), 4.39 (d, 2H), 4.13 (t, 2H), 3.46 (d, 2H), 3.18 (t, 2H), 3.01 (t, 2H), 1.76 (d, 3H), 1.17 (m, 2H), 0.86 (s, 9H), 0.031 (s, 6H). LCMS (ESI) 581.5 m/z [M+H]⁺.

### Step-13: 2-(6-(4-(hydroxymethyl)piperidin-1-yl)-2-nitropyridin-3-yl)-5-(pyridin-3-yl)-6,7-dihydrothiazolo[5,4-c]pyridin-4(5H)-one (20)

Compound W (0.1 g, 0.17 mmol) was dissolved in THF (10 mL) and cooled to 0° C. in an ice bath with stirring. Then a 1 M solution of TBAF in THF (1 mL) was added and stirring was continued at RT for 2 h. The reaction was quenched with aqueous saturated sodium bicarbonate solution (pH 8-9) and extracted with 5% MeOH in DCM (2×25 mL). The organic phase was separated, dried over Na₂SO₄, filtered and the solvents were removed under reduced pressure. The residue was purified by column chromatography on basic silica gel (100-200 mesh) using a CH₂Cl₂/MeOH gradient (100/0->99/1->98/2) to afford the desired compound containing some impurities. The impure material was further purified by preparative TLC plates using dichloromethane/ethanol (95/5) as mobile phase to obtain the title compound 20 as a yellow solid (0.026 g, 34%). ¹H NMR (500 MHz, DMSO-d₆): δ 8.65 (d, 1H), 8.45 (dd, 1H), 8.17 (d, 1H), 7.84-7.82 (m, 1H), 7.48 (dd, 1H), 7.23 (d, 1H), 4.53 (t, 1H), 4.38 (d, 2H), 4.14 (t, 2H), 3.28 (t, 2H), 3.19 (t, 2H), 3.00 (t, 2H), 1.78-1.67 (m, 3H). LCMS (ESI) 467.7 m/z [M+H]⁺.

### Example 9 Synthesis of 2-(2-fluoro-6-(4-(hydroxymethyl-t₂)piperidin-1-yl)pyridin-3-yl)-5-(pyridin-3-yl)-6,7-dihydrothiazolo[5,4-c]pyridin-4(5H)-one (³H-Compound 17)

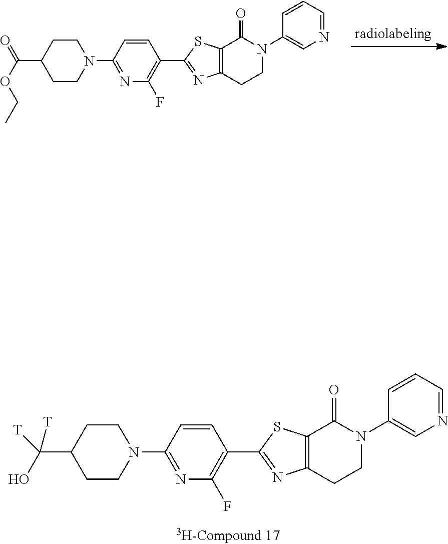

T means ³H.

A reaction vessel was charged with 150 μL n-BuLi (0.24 mmol, 1.6 M in hexane) and 50 μL TMEDA.

The solution was degassed three times at a high vacuum manifold and stirred under an atmosphere of tritium gas (1343 mbar, 9.2 Ci) at room temperature for 40 min. This process was repeated twice (2^{nd} run: 1097 mbar, 6.6 Ci and 3^{rd} run: 912 mbar, 4.8 Ci). At the end of the reaction (LiT formation), a pressure of 333 mbar was obtained. The solvent was removed under vacuum and an AlCl₃ solution (6.7 mg in 0.2 mL THF) was added under stirring. The mixture was stirred for 10 min at room temperature. A suspension of the precursor compound (3.3 mg in 0.5 mL THF) was added at −78° C. and the reaction mixture was stirred for 3 h at −78° C. The reaction vessel was cooled down to −196° C. and 0.2 mL H₂O was added. Warm up to room temperature was carried out very slowly due to the strong pressure increase. The solvent was removed in vacuo, and labile tritium was exchanged by adding 0.3 ml of methanol, stirring the solution, and removing the solvent again in vacuo. This process was repeated twice. Finally, the well dried solid was dissolved in 5 ml EtOH. The radiochemical purity of the crude material was determined to be approx. 44% using the following HPLC system: Waters Sunfire C18, 5 μm, 4.6×250 mm; solvents A: water+0.05% TFA, B: acetonitrile+0.05% TFA; 0 min 20% B; 10-14.5 min 95% B, 15 min 20% B; 254 nm; 1.0 ml/min; 30° C. The product had a retention time of 5.82 min. For the purification of the compound, the following HPLC conditions were used: Waters Sunfire C18, 10×250 mm; solvents A: water+0.1% TFA; B: acetonitrile+0.1%); 27% B isocratic; 4.7 ml/min; 25° C. The target compound eluted at 8.8-9.6 min. The solvent was removed in vacuum and the product was dissolved in 5 ml ethanol. The purified product ³H-Compound 17 was obtained with a specific activity of 55.4 Ci/mmol, (97% purity).

### Example 10 Synthesis of Tritiated (R)-2-(2-fluoro-6-(3-fluoropyrrolidin-1-yl)pyridin-3-yl)-5-(pyridin-4-yl)-6,7-dihydrothiazolo[5,4-c]pyridin-4(5H)-one ³H-Compound 7

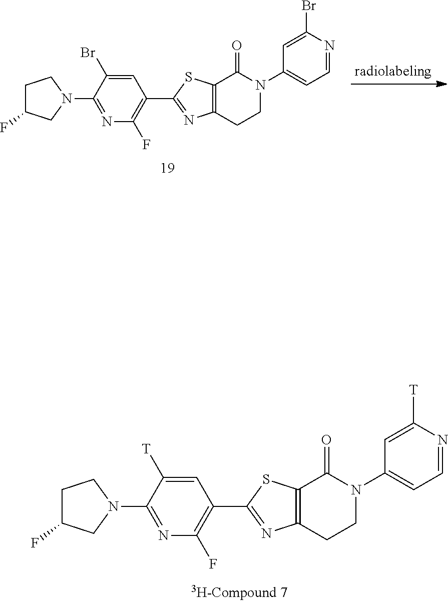

T means ³H.

2.29 mg of the brominated precursor 19 (prepared according to the synthetic scheme for [³H] precursor described in Example 2 above and utilizing the corresponding building block for compound 7 indicated in Table 1), 50 μL DIPEA and 31.7 mg of Lindlar's catalyst were suspended in 0.3 ml DMF. The suspension was degassed three times at a high vacuum manifold and stirred under an atmosphere of tritium gas (401 mbar, 11.2 Ci) overnight at room temperature. The solvent was removed in vacuo, and labile tritium was exchanged by adding 0.3 ml of methanol, stirring the solution, and removing the solvent again in vacuo. This process was repeated twice. The well dried solid was extracted with 5 ml ethanol/DMF (4:1) and the suspension was filtered through a 0.2 μm nylon membrane obtaining a clear solution. For the purification of the compound, the following HPLC conditions were found to be suitable: Waters Sunfire C18, 10×250 mm; solvents A: water+0.1% TFA); B: acetonitrile+0.1% TFA. The purified product ³H-Compound 7 was obtained (SA 50.8 Ci/mmol, 99% purity).

### Example 11: Synthesis of tritiated (S)-2-(2-fluoro-6-(3-fluoropyrrolidin-1-yl)pyridin-3-yl)-5-(pyridin-3-yl)-6,7-dihydrothiazolo[5,4-c]pyridin-4(5H)-one (³H-Compound 8)

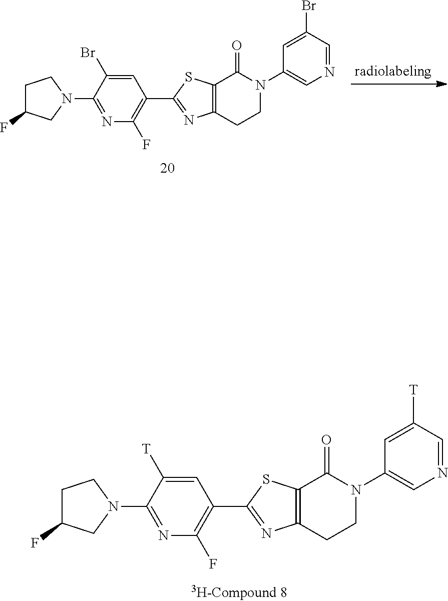

T means ³H.

2.45 mg of the brominated precursor 20 (prepared according to the synthetic scheme for [³H] precursor described in Example 2 above and utilizing the corresponding building block for compound 8 indicated in Table 1), 50 μL DIPEA and 33.5 mg of Lindlar's catalyst were suspended in 0.3 ml DMF. The suspension was degassed three times at the high vacuum manifold and stirred under an atmosphere of tritium gas (498 mbar, 4.3 Ci) overnight at room temperature. The solvent was removed in vacuo, and labile tritium was exchanged by adding 0.3 ml of methanol, stirring the solution, and removing the solvent again in vacuo. This process was repeated twice. The well dried solid was extracted with 5 ml ethanol/DMF (4:1) and the suspension was filtered through a 0.2 μm nylon membrane obtaining a clear solution. For the purification of the compound, the following HPLC conditions were found to be suitable: Waters Sunfire C18, 10×250 mm; solvents A: water+0.1% TFA); B: acetonitrile+0.1% TFA. The purified product ³H-Compound 8 was obtained (SA 48.5 Ci/mmol, 99% purity).

### Example 12 Synthesis of Tritiated 2-(2-fluoro-6-(pyrrolidin-1-yl)pyridin-3-yl)-5-(pyridin-3-yl)-6,7-dihydrothiazolo[5,4-c]pyridin-4(5H)-one ³H-Compound 9

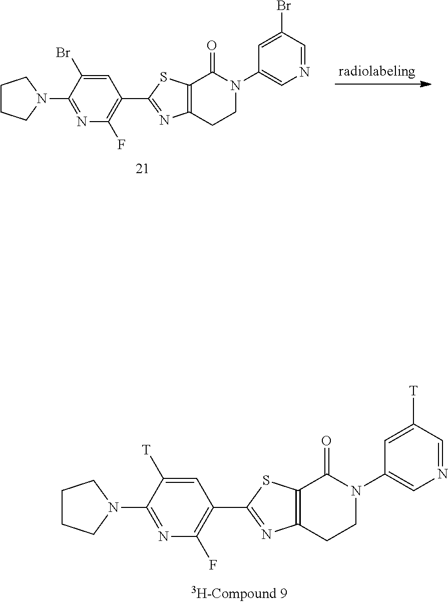

T means ³H.

1.7 mg of the brominated precursor 21 (prepared according to the synthetic scheme for [³H] precursor described in Example 2 above and utilizing the corresponding building block for compound 9 indicated in Table 1), 20 μL DIPEA and 7.5 mg of Pd on carbon (10% metal) were suspended in 0.3 ml DMF. The suspension was degassed three times at a high vacuum manifold and stirred under an atmosphere of tritium gas (670 mbar, 8.4 Ci) overnight at room temperature. The solvent was removed in vacuo, and labile tritium was exchanged by adding 0.3 ml of methanol, stirring the solution, and removing the solvent again in vacuo. This process was repeated twice. The well dried solid was extracted with 5 ml ethanol/DMF (4:1) and the suspension was filtered through a 0.2 μm nylon membrane obtaining a clear solution. For the purification of the compound, the following HPLC conditions were found to be suitable: Waters Sunfire C18, 10×250 mm; solvents A: water+0.1% TFA); B: acetonitrile+0.1% TFA. The purified product ³H-Compound 9 was obtained (SA 57.6 Ci/mmol, 99% purity).

### Example 13 Synthesis of Tritiated 2-[2-fluoro-6-[(3R)-3-fluoropyrrolidin-1-yl]-3-pyridyl]-5-pyrimidin-2-yl-6,7-dihydrothiazolo[5,4-c]pyridine-4-one ³H-Compound 15

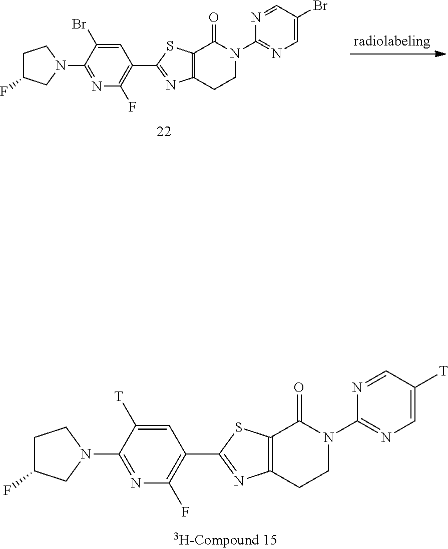

T means ³H.

Di-brominated precursor 22 (prepared according to the synthetic scheme for [³H] precursor described in Example 2 above and utilizing the corresponding building block for compound 15 indicated in Table 1) (2.45 mg, supplied by the customer), DIPEA (50 μL, Acros A0408500) and Lindlar's catalyst (15.5 mg) were suspended in DMF (0.4 ml, Acros 34831000). The suspension was degassed two times at the high vacuum manifold and stirred under an atmosphere of tritium gas (569 mbar, 7.7 Ci) overnight at room temperature. At the end of the reaction, a pressure of 531 mbar at room temperature was observed. The solvent was removed in vacuo, and labile tritium was exchanged by adding 0.3 ml of methanol, stirring the solution, and removing the solvent again in vacuo. This process was repeated twice. Finally, the well dried solid was extracted with 5 ml DCM and the suspension was filtered obtaining a clear solution. For the purification of the compound, the following HPLC conditions were found to be suitable: Waters Sunfire C18, 10×250 mm; solvents A: water (J. T. Baker HPLC Gradient Grade 4218)+0.1% TFA (Sigma-Aldrich T6508); B: acetonitrile (MACRON ChromAR HPLC Super Gradient 2856-25)+0.1% TFA (Sigma-Aldrich T6508); 55% B isocratic; 4.7 ml/min; 25° C. The purified product ³H-Compound 15 was obtained (SA 52 Ci/mmol, 99% purity).

### Example 14 Synthesis of Fluorinated 2-(2-(fluoro-¹⁸F)-6-(4-(hydroxymethyl)piperidin-1-yl)pyridin-3-yl)-5-(pyridin-3-yl)-6,7-dihydrothiazolo[5,4-c]pyridin-4(5H)-one

### ¹⁸F Compound 17

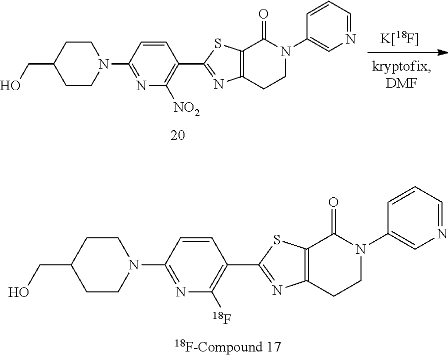

[¹⁸F]Fluoride in a shipping vial (0-18 enriched water obtained from a commercial cyclotron facility) was transferred onto and trapped on a Chromafix PS-HCO₃ cartridge (activated with KOTf (0.2 M, 6 mL), followed by water (6 mL). It was then eluted with a solution of K₂₂₂/K₂CO₃ (15/3 mg in 0.3 mL) into the reaction vessel of the TRACERlab® module. The solution was first heated at 70° C. for 3.5 min under vacuum and helium flow and the heat treatment was continued for an additional 2.5 min at 95° C. under the same conditions. The reactor was then cooled to 60° C. A solution of 2-(6-(4-(hydroxymethyl)piperidin-1-yl)-2-nitropyridin-3-yl)-5-(pyridin-3-yl)-6,7-dihydrothiazolo[5,4-c]pyridin-4(5H)-one 20 (3.35 mg in 0.3 mL DMSO) was added to the reaction vial and the vessel was heated at 160° C. for 10 min. The reaction vessel was cooled to 60° C. and the mixture was diluted with HPLC buffer (4 mL). Purification was performed by HPLC using a semi-preparative Xbridge C18 column (5 μm, 250×10 mm) and elution with a mixture of acetonitrile/ammonium acetate solution (20 mM, 35/65, v/v) at a flow rate of 5 mL/min. The radiolabelled product was eluted from the SPE cartridge with 1.0 mL of ethanol into the formulation flask, pre-loaded with 10 mL of formulation base (ascorbic acid (10 mg) in saline). The resulting solution was passed through a sterilizing 0.2 μm membrane filter into a sterile, filter-vented vial. The final product ¹⁸F-Compound 17 was assayed, and sample was removed for QC testing. Confirmation of the identity of the product was determined by co-injection with a sample of the ¹⁹F-reference compound 17.

### Synthetic Scheme for (S)-2-(2-fluoro-6-(3-fluoropyrrolidin-1-yl)pyridin-3-yl)-5-(isothiazol-4-yl)-6,7-dihydrothiazolo[5,4-c]pyridin-4(5H)-one (compound 25)

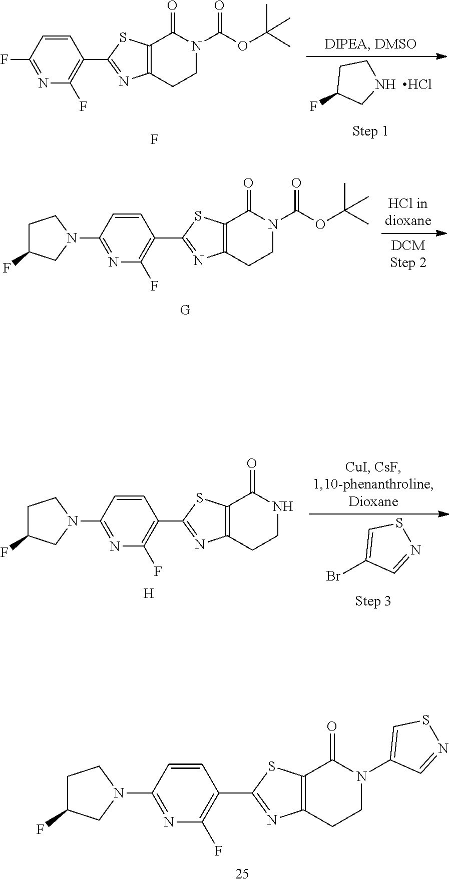

### Example 15 Synthesis of (S)-2-(2-fluoro-6-(3-fluoropyrrolidin-1-yl)pyridin-3-yl)-5-(isothiazol-4-yl)-6,7-dihydrothiazolo[5,4-c]pyridin-4(5H)-one

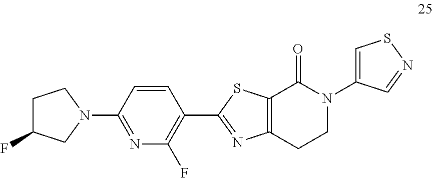 [SMILES: O=C1c2sc(-c3ccc(N4CC[C@H](F)C4)nc3F)nc2CCN1c1cnsc1]

### Step-1: Synthesis of tert-butyl (S)-2-(2-fluoro-6-(3-fluoropyrrolidin-1-yl)pyridin-3-yl)-4-oxo-6,7-dihydrothiazolo[5,4-c]pyridine-5(4H)-carboxylate (G)

Title compound F (0.26 g, 0.7 mmol), (S)-3-fluoropyrrolidine hydrochloric acid salt (0.088 g, 0.7 mmol) and N,N-diisopropylethylamine (0.2 mL, 1.4 mmol) were suspended in DMSO (26 mL) and stirred at RT for 2 h. After completion, the reaction mixture was suspended in water (260 mL) and the precipitated solid was filtered over Whatmann filter paper. The solid was washed with water (20 mL) and dried under reduced pressure to afford the title compound G as a pale-yellow solid (0.27 g, 91%). ¹H NMR (500 MHz, DMSO-d₆): δ 8.39 (t, 1H), 6.63 (d, 1H), 5.48 (d, 1H), 4.06 (t, 2H), 3.70 (dd, 3H), 3.54-3.49 (m, 1H), 3.10 (t, 2H), 2.30-2.27 (m, 2H), 1.49 (S, 9H). LCMS (ESI) 437.10 m/z [M+H]⁺.

### Step-2: Synthesis of (S)-2-(2-fluoro-6-(3-fluoropyrrolidin-1-yl)pyridin-3-yl)-6,7-dihydrothiazolo[5,4-c]pyridin-4(5H)-one (H)

Title compound G (0.27 g, 0.6 mmol) was dissolved in DCM (6 mL) and cooled to 0° C. in an ice bath with stirring, then 4M HCl in 1,4-dioxane (2.7 mL) was added to the above solution and stirring was continued at RT for 2 h. After completion of the reaction, solvent was removed under reduced pressure. The residue obtained was dissolved in ice-cold water and basified with aqueous saturated sodium bicarbonate solution to pH 8-9. The precipitated solid was filtered and dried to afford the title compound H as a pale-yellow solid (0.19 g, 91%). 1H NMR (500 MHz DMSO-d6): δ 8.39-8.35 (m, 1H), 7.82 (s, 1H), 6.61-6.60 (m, 1H), 5.48 (d, 1H), 3.80-3.63 (m, 4H), 3.53-3.48 (m, 2H), 2.99 (t, 2H), 2.30-2.18 (m, 2H). LCMS (ESI) 337 [M+H]⁺.

### Step-3: Synthesis of (S)-2-(2-fluoro-6-(3-fluoropyrrolidin-1-yl)pyridin-3-yl)-5-(isothiazol-4-yl)-6,7-dihydrothiazolo[5,4-c]pyridin-4(5H)-one (1)

Compound H (0.05 g, 0.14 mmol) and 4-bromoisothiazole (0.073 g, 0.44 mmol) were dissolved in degassed 1,4-dioxane (5 mL). After the addition of CuI (0.014 g, 0.074 mmol), 1,10-phenanthroline (0.026 g, 0.014 mmol), and CsF (0.045 g, 0.29 mmol), the reaction mixture was heated at ˜100° C. in a sand bath for 48 h. The reaction mixture was concentrated under reduced pressure to afford the crude residue. The crude residue was purified by column chromatography on silica gel (100-200 mesh) using CH₂Cl₂/MeOH gradient (100/0->99.5/0.5->99/1) to afford the pure compound 25 as a pale-yellow solid (0.029 g, 46%). ¹H NMR (400 MHz DMSO-d₆): δ 8.95 (s, 1H), 8.88 (s, 1H), 8.43-8.38 (m, 1H), 6.65-6.62 (m, 1H), 5.48 (d, 1H), 4.22 (t, 2H), 3.85-3.72 (m, 3H), 3.55-3.48 (m, 1H), 3.27 (t, 2H), 2.27-2.16 (m, 2H). LCMS (ESI) 418.2 m/z [M−H]⁺

### Synthetic Scheme for (S)-2-(2-fluoro-6-(3-fluoropiperidin-1-yl)pyridin-3-yl)-5-(isothiazol-4-yl)-6,7-dihydrothiazolo[5,4-c]pyridin-4(5H)-one (compound 27)

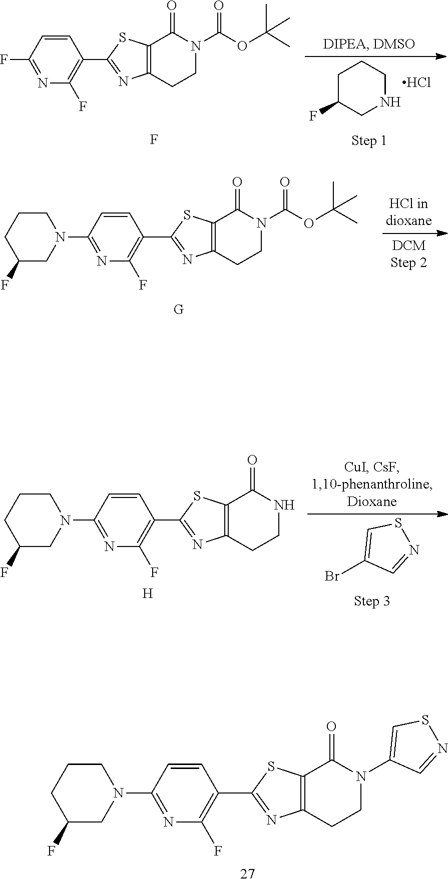

### Example 16 Synthesis of (S)-2-(2-fluoro-6-(3-fluoropiperidin-1-yl)pyridin-3-yl)-5-(isothiazol-4-yl)-6,7-dihydrothiazolo[5,4-c]pyridin-4(5H)-one

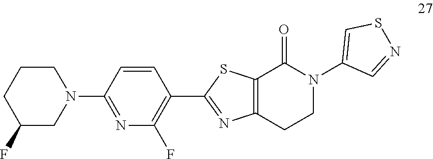 [SMILES: O=C1c2sc(-c3ccc(N4CCC[C@H](F)C4)nc3F)nc2CCN1c1cnsc1]

### Step-1: Synthesis of tert-butyl (S)-2-(2-fluoro-6-(3-fluoropiperidin-1-yl)pyridin-3-yl)-4-oxo-6,7-dihydrothiazolo[5,4-c]pyridine-5(4H)-carboxylate (G)

Title compound F (0.4 g, 1.0 mmol), (S)-3-fluoropiperidine hydrochloric acid salt (0.15 g, 1.0 mmol) and N,N-diisopropylethylamine (0.36 mL, 2.1 mmol) were suspended in DMSO (40 mL) and the reaction mixture was stirred at RT for 2 h. After completion, the reaction mixture was suspended in water (400 mL) and filtered over Whatmann filter paper. The solid was washed with water (20 mL) and dried under reduced pressure to afford the title compound G as a pale-yellow solid (0.47 g, 96%). ¹H NMR (500 MHz, DMSO-d₆): δ 8.37-8.33 (m, 1H), 6.97-6.95 (m, 1H), 4.85 (d, 1H), 4.22-4.16 (m, 1H), 4.06 (t, 2H), 4.01 (d, 1H), 3.67-3.57 (m, 1H), 3.38-3.35 (m, 1H), 3.10 (t, 2H), 1.95-1.87 (m, 2H), 1.78-1.70 (m, 1H), 1.61-1.56 (m, 1H), 1.49 (s, 9H), LCMS (ESI) 451.35 m/z [M+H]⁺.

### Step-2: Synthesis of (S)-2-(2-fluoro-6-(3-fluoropiperidin-1-yl)pyridin-3-yl)-6,7-dihydrothiazolo[5,4-c]pyridin-4(5H)-one (H)

Title compound G (0.47 g, 1.04 mmol) was dissolved in DCM (9 mL) and cooled to 0° C. in an ice bath with stirring. 4M HCl in 1,4-dioxane (4.7 mL) was added to the above solution and stirring was continued at RT for 2 h. After completion of the reaction, solvent was removed under reduced pressure. The residue obtained was dissolved in ice-cold water and basified with aqueous saturated sodium bicarbonate solution to pH 8-9. The precipitated solid was filtered and dried to afford the title compound H as a pale-yellow solid (0.36 g, 98%). 1H NMR (500 MHz DMSO-d6): δ 8.35-8.31 (m, 1H), 7.82 (s, 1H), 6.95-6.93 (m, 1H), 4.90-4.79 (m, 1H), 4.18-4.13 (m, 1H), 3.99-3.96 (m, 1H), 3.66-3.60 (m, 1H), 3.58-3.49 (m, 2H), 3.36-3.34 (m, 1H), 2.99 (t, 2H), 1.94-1.87 (m, 2H), 1.74-1.73 (m, 1H), 1.60-1.56 (m, 1H). LCMS (ESI) 351 [M+H]⁺.

### Step-3: Synthesis of (S)-2-(2-fluoro-6-(3-fluoropiperidin-1-yl)pyridin-3-yl)-5-(isothiazol-4-yl)-6,7-dihydrothiazolo[5,4-c]pyridin-4(5H)-one (28)

Compound H (0.05 g, 0.14 mmol) and 4-bromoisothiazole (0.07 g, 0.42 mmol) were dissolved in degassed 1,4-dioxane (5 mL). After the addition of CuI (0.013 g, 0.07 mmol), 1,10-phenanthroline (0.025 g, 0.14 mmol), and CsF (0.043 g, 0.28 mmol), the reaction mixture was heated at ˜100° C. in a sand bath for 48 h. The reaction mixture was concentrated under reduced pressure to afford the crude. The crude residue was purified by column chromatography on silica gel (100-200 mesh) using CH₂Cl₂/MeOH gradient (100/0->99.5/0.5->99/1->98.5/1.5) to afford the pure compound 27 as a yellow solid (0.047 g, 77%). ¹H NMR (400 MHz DMSO-d₆): δ 8.95 (s, 1H), 8.88 (s, 1H), 8.39-8.35 (m, 1H), 6.99-6.96 (m, 1H), 4.92-4.79 (m, 1H), 4.23-4.16 (m, 3H), 4.03-4.00 (m, 1H), 3.68-3.57 (m, 1H), 3.37-3.36 (m, 1H), 3.27 (t, 2H), 1.94-1.87 (m, 2H), 1.76-1.58 (m, 2H). LCMS (ESI) 433.9 m/z [M−H]⁺

### Synthetic Scheme (R)-2-(2-fluoro-6-(3-fluoropiperidin-1-yl)pyridin-3-yl)-5-(isothiazol-4-yl)-6,7-dihydrothiazolo[5,4-c]pyridin-4(5H)-one (Compound 24)

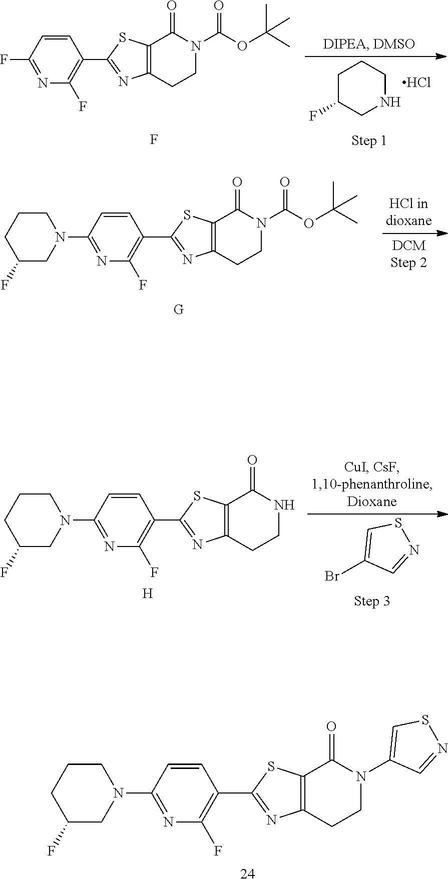

### Example 17 Synthesis of (R)-2-(2-fluoro-6-(3-fluoropiperidin-1-yl)pyridin-3-yl)-5-(isothiazol-4-yl)-6,7-dihydrothiazolo[5,4-c]pyridin-4(5H)-one

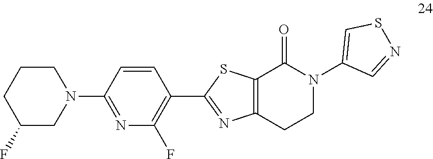 [SMILES: O=C1c2sc(-c3ccc(N4CCC[C@@H](F)C4)nc3F)nc2CCN1c1cnsc1]

### Step-1: Synthesis of tert-butyl (R)-2-(2-fluoro-6-(3-fluoropiperidin-1-yl)pyridin-3-yl)-4-oxo-6,7-dihydrothiazolo[5,4-c]pyridine-5(4H)-carboxylate (G)

Title compound F (0.6 g, 1.63 mmol), (R)-3-fluoropiperidine hydrochloric acid salt (0.22 g, 1.63 mmol) and N,N-diisopropylethylamine (0.55 mL, 3.27 mmol) were suspended in DMSO (60 mL) and stirring was continued at RT for 2 h. After completion of the reaction, the reaction mixture was suspended in water (600 mL) and filtered over Whatmann filter paper. The solid was washed with water (20 mL) and dried under reduced pressure to obtain the title compound G as a pale-yellow solid (0.63 g, 90%). ¹H NMR (500 MHz, DMSO-d₆): δ 8.35 (t, 1H), 6.97-6.95 (m, 1H), 4.85 (d, 1H), 4.21-4.16 (m, 1H), 4.06-3.99 (m, 3H), 3.67-3.57 (m, 1H), 3.38-3.34 (m, 1H), 3.11-3.08 (t, 2H), 1.93-1.87 (m, 2H), 1.76-1.73 (m, 1H), 1.61-1.56 (m, 1H), 1.49 (s, 9H), LCMS (ESI) 451.25 m/z [M+H]⁺.

### Step-2: Synthesis of (R)-2-(2-fluoro-6-(3-fluoropiperidin-1-yl)pyridin-3-yl)-6,7-dihydrothiazolo[5,4-c]pyridin-4(5H)-one (H)

Title compound G (0.63 g, 1.4 mmol) was dissolved in DCM (12 mL) and cooled to 0° C. in an ice bath with stirring. 4M HCl in 1,4-dioxane (6.3 mL) was added to above solution and stirring was continued at RT for 2 h. After completion of the reaction, solvent was removed under reduced pressure. The residue obtained was dissolved in ice-cold water and basified with aqueous saturated sodium bicarbonate solution to pH 8-9. The precipitated solid was filtered and dried to afford the title compound H as a pale-yellow solid (0.4 g, 85%). 1H NMR (500 MHz DMSO-d6): δ 8.36-8.32 (m, 1H), 7.83 (s, 1H), 6.96-6.94 (m, 1H), 4.89-4.79 (m, 1H), 4.18-4.13 (m, 1H), 3.99-3.96 (m, 1H), 3.66-3.57 (m, 1H), 3.51-3.48 (m, 2H), 3.36-3.34 (m, 1H), 2.99 (t, 2H), 1.95-1.87 (m, 2H), 1.77-1.71 (m, 1H), 1.60-1.56 (m, 1H). LCMS (ESI) 351 [M+H]⁺.

### Step-3: Synthesis of (R)-2-(2-fluoro-6-(3-fluoropiperidin-1-yl)pyridin-3-yl)-5-(isothiazol-4-yl)-6,7-dihydrothiazolo[5,4-c]pyridin-4(5H)-one (24)

Compound H (0.05 g, 0.14 mmol) and 4-bromoisothiazole (0.07 g, 0.42 mmol) were dissolved in degassed 1,4-dioxane (5 mL). After the addition of CuI (0.013 g, 0.07 mmol), 1,10-phenanthroline (0.025 g, 0.14 mmol), and CsF (0.043 g, 0.28 mmol), the reaction mixture was heated at ˜100° C. in a sand bath for 36 h. The reaction mixture was concentrated under reduced pressure to afford the crude product. The crude product thus obtained was purified by column chromatography on silica gel (100-200 mesh) using CH₂Cl₂/MeOH gradient (100/0->99.5/0.5->99/1) to afford the pure compound 24 as a yellow solid (0.03 g, 50%). ¹H NMR (400 MHz DMSO-d₆): δ 8.95 (s, 1H), 8.88 (s, 1H), 8.39-8.35 (m, 1H), 6.99-6.96 (m, 1H), 4.91-4.79 (m, 1H), 4.23-3.99 (m, 4H), 3.68-3.57 (m, 1H), 3.40-3.39 (m, 1H), 3.28-3.24 (m, 2H), 1.96-1.87 (m, 2H), 1.76-1.57 (m, 2H). LCMS (ESI) 433.8 m/z [M−H]⁺

### Synthetic Scheme for 2-(2-fluoro-6-(piperidin-1-yl)pyridin-3-yl)-5-(2-fluoropyridin-4-yl)-6,7-dihydrothiazolo[5,4-c]pyridin-4(5H)-one (Compound 23)

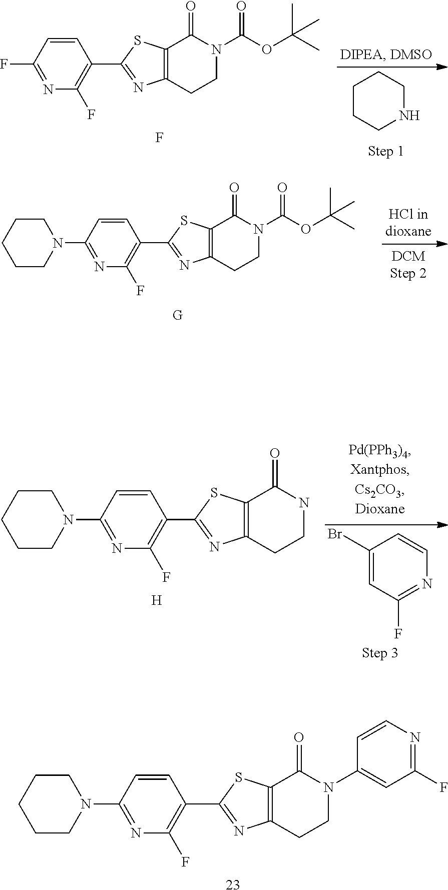

### Example 18 Synthesis of 2-(2-fluoro-6-(piperidin-1-yl)pyridin-3-yl)-5-(2-fluoropyridin-4-yl)-6,7-dihydrothiazolo[5,4-c]pyridin-4(5H)-one

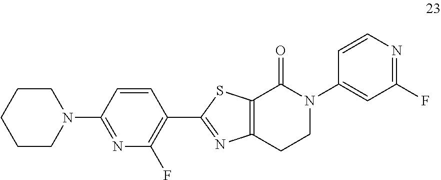 [SMILES: O=C1c2sc(-c3ccc(N4CCCCC4)nc3F)nc2CCN1c1ccnc(F)c1]

### Step-1: Synthesis of tert-butyl 2-(2-fluoro-6-(piperidin-1-yl)pyridin-3-yl)-4-oxo-6,7-dihydrothiazolo[5,4-c]pyridine-5(4H)-carboxylate (G)

Title compound F (0.3 g, 0.81 mmol), piperidine (0.069 g (0.08 mL), 0.81 mmol) and N,N-diisopropylethylamine (0.2 mL, 1.6 mmol) were suspended in DMSO (30 mL) and stirred at RT for 2 h. After completion, the reaction mixture was suspended in water (300 mL) and filtered over Whatmann filter paper. The solid was washed with water (20 mL) and dried under reduced pressure to obtain the title compound G as a pale-yellow solid (0.34 g, 98%). ¹H NMR (500 MHz, DMSO-d₆): δ 8.32 (t, 1H), 6.91-6.89 (m, 1H), 4.05 (t, 2H), 3.65 (t, 4H), 3.09 (t, 2H), 1.64 (t, 2H), 1.58-1.53 (m, 4H), 1.48 (s, 9H). LCMS (ESI) 432.90 m/z [M+H]⁺.

### Step-2: Synthesis of 2-(2-fluoro-6-(piperidin-1-yl)pyridin-3-yl)-6,7-dihydrothiazolo[5,4-c]pyridin-4(5H)-one (H)

Title compound G (0.34 g, 0.78 mmol) was dissolved in DCM (7 mL) and cooled to 0° C. in an ice bath with stirring. 4M HCl in 1,4-dioxane (3.4 mL) was added to the above solution and stirring was continued at RT for 2 h. After completion of the reaction, the solvent was removed under reduced pressure. The residue obtained was dissolved in ice-cold water and basified with aqueous saturated sodium bicarbonate solution to pH 8-9. The precipitated solid was filtered and dried to afford the title compound H as a yellow solid (0.255 g, 98%). 1H NMR (400 MHz DMSO-d6): δ 8.34-8.29 (m, 1H), 7.83 (s, 1H), 6.90-6.87 (m, 1H), 3.65-3.62 (m, 4H), 3.51-3.47 (m, 2H), 2.99 (t, 2H), 1.65-1.63 (m, 2H), 1.56-1.55 (m, 4H). LCMS (ESI) 333.2 [M+H]⁺.

### Step-3: Synthesis of 2-(2-fluoro-6-(piperidin-1-yl)pyridin-3-yl)-5-(2-fluoropyridin-4-yl)-6,7-dihydrothiazolo[5,4-c]pyridin-4(5H)-one (4)

Compound H (0.05 g, 0.15 mmol) and 4-bromo-2-fluoropyridine (0.039 g, 0.22 mmol) were dissolved in degassed 1,4-dioxane (5 mL). After the addition of Pd[PPh₃]₄ (0.008 g, 0.007 mmol), XantPhos (0.008 g, 0.015 mmol), and Cs₂CO₃ (0.073 g, 0.22 mmol), the reaction mixture was heated at ˜100° C. in a sand bath for 24 h. The reaction mixture was concentrated under reduced pressure to afford the crude residue. The residue was purified by column chromatography on silica gel (100-200 mesh) using CH₂Cl₂/MeOH gradient (100/0->99.5/0.5->99/1) to afford the impure compound. The impure compound was further purified by preparative TLC plates using CH₂Cl₂/MeOH (98.5/1.5) as a mobile phase to afford the title compound 23 as a pale-yellow solid (0.032 g, 50%). ¹H NMR (400 MHz DMSO-d₆): δ 8.37-8.32 (m, 1H), 8.22 (d, 1H), 7.47-7.45 (m, 1H), 7.25 (d, 1H), 6.93-6.90 (m, 1H), 4.22 (t, 2H), 3.66 (t, 4H), 3.24 (t, 2H), 1.65-1.63 (m, 2H), 1.57-1.56 (m, 4H). LCMS (ESI) 427.9 m/z [M+H]⁺

### Biological Assay Description

### 1. General Methods

Human brain material for these studies was obtained from Prof. William Seeley at the Neurodegenerative Disease Brain Bank UCSF (funding support from NIH grants P01AG019724 and P50AG023501, the Consortium for Frontotemporal Dementia Research, and the Tau Consortium), and also from Prof. Tammaryn Lashley at the Queen Square Brain Bank for Neurological Disorders, UCL. All material has been collected from donors from whom a written informed consent for brain autopsy and the use of the material and clinical information for research purposes has been obtained by the brain bank.

### 1.1 Radioligands

[³H]-Compound 1 which is described above having a specific activity of 48.5 Ci/mmol (1.0 mCi/mL), [³H]-Compound 7 which is described above having a specific activity of 50.8 Ci/mmol (1.0 mCi/mL), [³H]-Compound 8 which is described above having a specific activity of 55.1 Ci/mmol (1.0 mCi/mL), [³H]-Compound 9 which is described above having a specific activity of 57.6 Ci/mmol (1.0 mCi/mL), [³H]-Compound 15 which is described above having a specific activity of 53.3 Ci/mmol (1.0 mCi/mL), [³H]-Compound 16 which is described above having a specific activity of 52 Ci/mmol (1.0 mCi/mL), [³H]-Compound 17 which is described above having a specific activity of 55.4 Ci/mmol (1.0 mCi/mL), and/or [³H]-Reference Compound (which is the compound described as [³H]-Compound 16 in WO2023/285661) having a specific activity of 72.5 Ci/mmol (1.0 mCi/mL), were used in the assays below.

### 1.2. Preparation of Human Frontotemporal Dementia (FTD) Sarkosyl-Insoluble Brain Extracts

Human brain extracts were prepared as described in Laferriere et al., 2019, Nature Neurosc. A sample of brain tissue (frontal or temporal cortex) was homogenized at 1:4 (w/v) ratio in the homogenization-solubilization (HS) buffer at 4° C. using tissue homogenizer (Precellys) with CKmix homogenization tubes. The following sequence was used for homogenization: 3 cycles of 30 seconds at 5000 rpm (with 15 seconds pause between each cycle). Homogenized samples were aliquoted and stored at −80° C. in 1.5 mL low protein binding tubes.

Brain homogenates were thawed on ice and resuspended in HS buffer to obtain a final concentration of 2% sarkosyl, 1 unit/μL Benzonase and 1 mM MgCl₂. The samples were then incubated at 37° C. under constant shaking at 600 rpm on a thermomixer for 45 minutes (min). The supernatants were collected in a new tube (sarkosyl-soluble fraction, S1). The pellet was resuspended in 1000 μL of myelin floatation buffer and centrifuged at 20,000 g for 60 min at 4° C. The supernatant was carefully removed to remove all the floating lipids. This step was repeated if all the lipids could not be removed in a single step. The pellet was subsequently washed with Phosphate-Buffered Saline (PBS) and centrifuged for 30 min at 20,000 g at 4° C. The final pellet was resuspended in 200 μL PBS and stored at −80° C. (sarkosyl-insoluble fraction). The samples were analyzed by immunoblotting in denaturing conditions.

### 2. Biological Assays Description and Corresponding Results

### 2.1. Micro-Radiobinding Competition Assay for the Determination of Binding Affinity to TPD-43

Human FTD sarkosyl-insoluble brain extracts were spotted onto microarray slides. The slides were incubated with a tritiated reference ligand at 40 nM, and the example compounds (non-radiolabeled) at 2 μM and 250 nM. In some cases, the non-radiolabeled example compounds were further assessed for a range of different concentrations, varying from 0.24 nM to 2 μM for determination of the inhibition constant (Ki). After incubation, slides were washed and scanned by a real-time autoradiography system (BeaQuant, ai4R). Quantification of the signal was performed by using the Beamage image analysis software (ai4R). Non-specific signal was determined with an excess of non-radiolabeled reference compound (2 μM) and specific binding was calculated by subtracting the non-specific signal from the total signal. Competition was calculated as percent, where 0% was defined as the specific binding in the presence of vehicle and 100% as the values obtained in the presence of excess of the non-radiolabeled reference compound. Kᵢ values were calculated in GraphPad Prism8 by applying a nonlinear regression curve fit using a one site, specific binding model. Measurements were performed with at least two technical replicates in the two-concentration competition experiment and with one technical replicate in the experiments including a range of concentrations. For compounds tested in more than one experiment, the mean of the replicates or Kᵢ values in independent experiments is reported.

Results: Example compounds were assessed for their potency to compete with the binding of [³H]-reference ligand to FTD patient brain-derived TDP-43 aggregates. Results of the micro-radiobinding competition assay for the example compounds are shown in Table 5 below as: % competition at 2 μM and 250 nM. Kᵢ values are also shown in Table 5.

TABLE 5

| | | Human | | |
|---|---|---|---|---|
| | | brain | Human | |
| | | derived | brain | |
| | | TDP-43 | derived | Human |
| | | aggregates | TDP-43 | brain |
| | | Mean % | aggregates | derived |
| | | competition | Mean % | TDP-43 |
| | | at | competition | aggregates |
| Example | Product | 2 μM | at 250 nM | Ki [nM] |
| 1 | 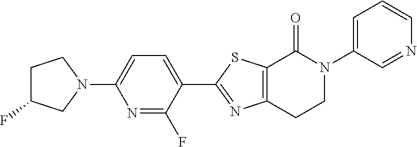 [SMILES: O=C1c2sc(-c3ccc(N4CC[C@@H](F)C4)nc3F)nc2CCN1c1cccnc1] | 78 | 65 | 22 |
| 6 | 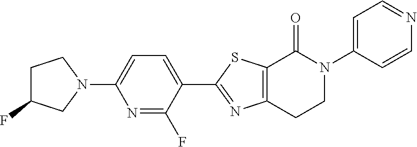 [SMILES: O=C1c2sc(-c3ccc(N4CC[C@H](F)C4)nc3F)nc2CCN1c1ccncc1] | 52 | 35 | n.d^{a} |
| 7 | 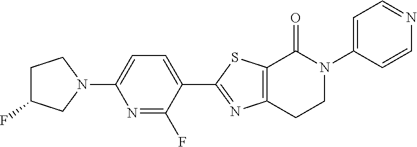 [SMILES: O=C1c2sc(-c3ccc(N4CC[C@@H](F)C4)nc3F)nc2CCN1c1ccncc1] | 73 | 51 | 51 |
| 8 | 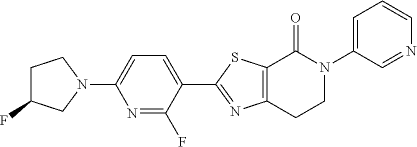 [SMILES: O=C1c2sc(-c3ccc(N4CC[C@H](F)C4)nc3F)nc2CCN1c1cccnc1] | 73 | 55 | 73 |
| 9 | 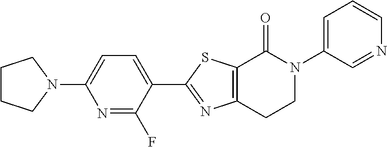 [SMILES: O=C1c2sc(-c3ccc(N4CCCC4)nc3F)nc2CCN1c1cccnc1] | 75 | 62 | 31 |
| 10 | 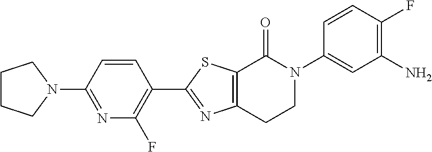 [SMILES: Nc1cc(N2CCc3nc(-c4ccc(N5CCCC5)nc4F)sc3C2=O)ccc1F] | 75 | 52 | 16 |
| 11 | 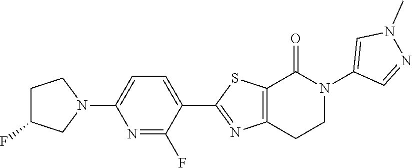 [SMILES: Cn1cc(N2CCc3nc(-c4ccc(N5CC[C@@H](F)C5)nc4F)sc3C2=O)cn1] | 102 | 73 | n.d^{a} |
| 12 | 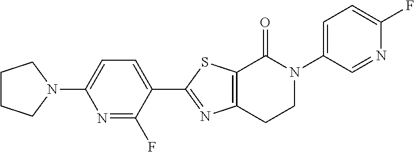 [SMILES: O=C1c2sc(-c3ccc(N4CCCC4)nc3F)nc2CCN1c1ccc(F)nc1] | 59 | 56 | n.d^{a} |
| 13 | 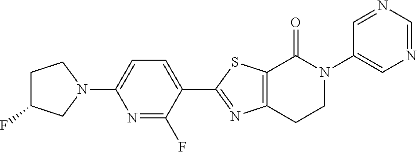 [SMILES: O=C1c2sc(-c3ccc(N4CC[C@@H](F)C4)nc3F)nc2CCN1c1cncnc1] | 84 | 57 | n.d^{a} |
| 14 | 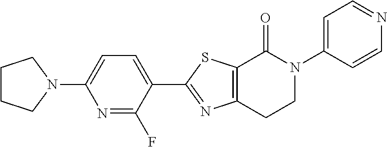 [SMILES: O=C1c2sc(-c3ccc(N4CCCC4)nc3F)nc2CCN1c1ccncc1] | 34 | 48 | n.d^{a} |
| 15 | 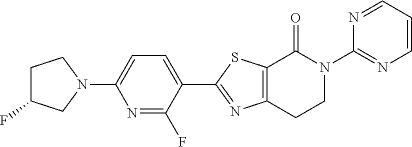 [SMILES: O=C1c2sc(-c3ccc(N4CC[C@@H](F)C4)nc3F)nc2CCN1c1ncccn1] | 86 | 62 | 11 |
| 16 | 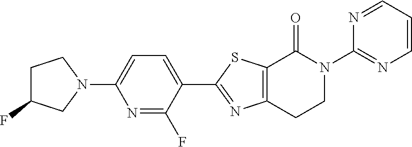 [SMILES: O=C1c2sc(-c3ccc(N4CC[C@H](F)C4)nc3F)nc2CCN1c1ncccn1] | 96 | 67 | 5 |
| 17 | 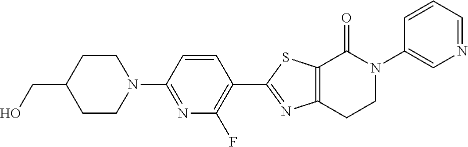 [SMILES: O=C1c2sc(-c3ccc(N4CCC(CO)CC4)nc3F)nc2CCN1c1cccnc1] | 85 | 73 | 41 |
| 21 | 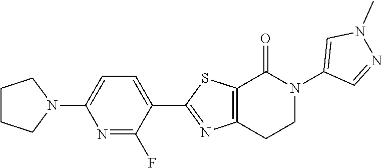 [SMILES: Cn1cc(N2CCc3nc(-c4ccc(N5CCCC5)nc4F)sc3C2=O)cn1] | 85 | 46 | 30 |
| 22 | 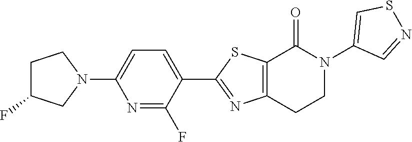 [SMILES: O=C1c2sc(-c3ccc(N4CC[C@@H](F)C4)nc3F)nc2CCN1c1cnsc1] | 100 | 68 | 54 |
| 23 | 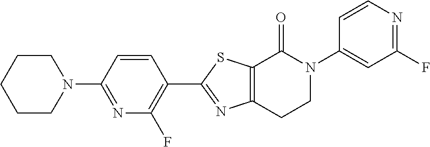 [SMILES: O=C1c2sc(-c3ccc(N4CCCCC4)nc3F)nc2CCN1c1ccnc(F)c1] | 104 | 73 | 25 |
| 24 | 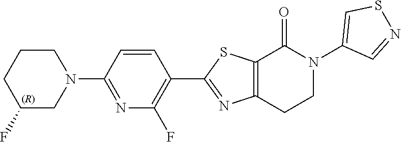 | 83 | 61 | 65 |
| 25 | 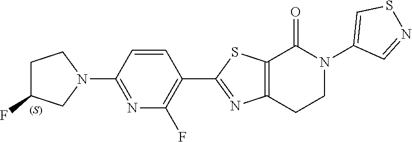 | 96 | 78 | 48 |
| 26 | 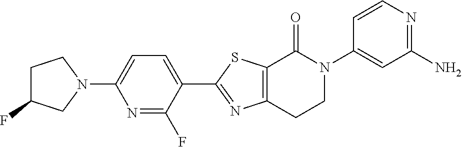 [SMILES: Nc1cc(N2CCc3nc(-c4ccc(N5CC[C@H](F)C5)nc4F)sc3C2=O)ccn1] | 91 | 55 | 13 |
| 27 | 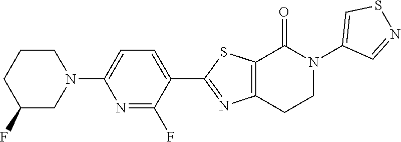 [SMILES: O=C1c2sc(-c3ccc(N4CCC[C@H](F)C4)nc3F)nc2CCN1c1cnsc1] | 94 | 57 | 48 |
| 28 | 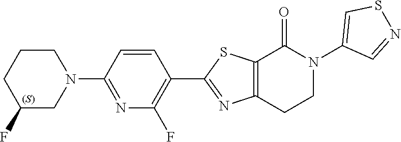 | 102 | 82 | 8 |
| 29 | 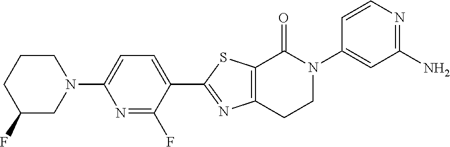 [SMILES: Nc1cc(N2CCc3nc(-c4ccc(N5CCC[C@H](F)C5)nc4F)sc3C2=O)ccn1] | 86 | 45 | 43 |
| 30 | 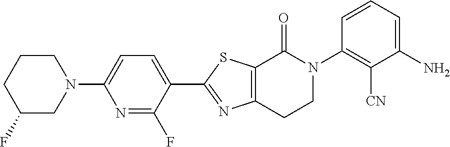 [SMILES: N#Cc1c(N)cccc1N1CCc2nc(-c3ccc(N4CCC[C@@H](F)C4)nc3F)sc2C1=O] | 93 | 66 | 7 |
| 31 | 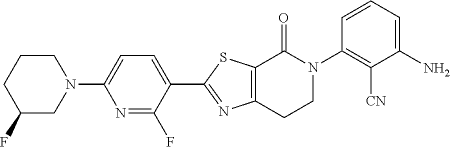 [SMILES: N#Cc1c(N)cccc1N1CCc2nc(-c3ccc(N4CCC[C@H](F)C4)nc3F)sc2C1=O] | 70 | 62 | 4 |
| 32 | 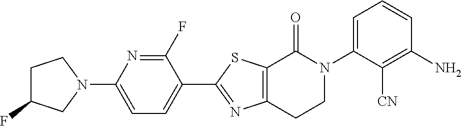 [SMILES: N#Cc1c(N)cccc1N1CCc2nc(-c3ccc(N4CC[C@H](F)C4)nc3F)sc2C1=O] | 80 | 65 | 4 |
| 33 | 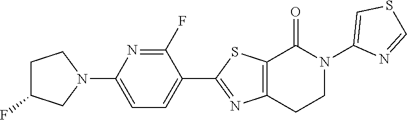 [SMILES: O=C1c2sc(-c3ccc(N4CC[C@@H](F)C4)nc3F)nc2CCN1c1cscn1] | 86 | 60 | 36 |
| 34 | 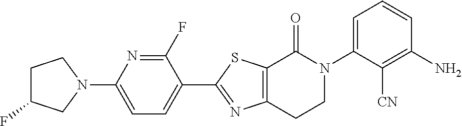 [SMILES: N#Cc1c(N)cccc1N1CCc2nc(-c3ccc(N4CC[C@@H](F)C4)nc3F)sc2C1=O] | 77 | 60 | 5 |
| 35 | 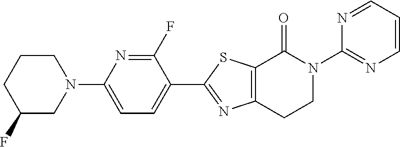 [SMILES: O=C1c2sc(-c3ccc(N4CCC[C@H](F)C4)nc3F)nc2CCN1c1ncccn1] | 70 | 55 | 49 |
| 36 | 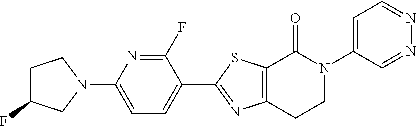 [SMILES: O=C1c2sc(-c3ccc(N4CC[C@H](F)C4)nc3F)nc2CCN1c1ccnnc1] | 75 | 66 | 7 |
| 37 | 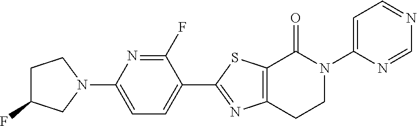 [SMILES: O=C1c2sc(-c3ccc(N4CC[C@H](F)C4)nc3F)nc2CCN1c1ccncn1] | 70 | 80 | 25 |

^{a}n.d. not determined

2.2 Kd Determination on Human FTD Sarkosyl-Insoluble Brain Extracts with Micro-Radiobinding Assay of [³H]-Compounds 1, 7, 8, 9, 15, 16 and 17

Human FTD sarkosyl-insoluble brain extracts were spotted onto microarray slides. The slides were incubated with [³H]-Compounds 1, 7, 8, 9, 15, 16 and 17 in a range of concentrations, varying from 1.29 to 300 nM. After incubation, slides were washed and scanned by a real-time autoradiography system (BeaQuant, ai4R). Quantification of the signal was performed by using the Beamage image analysis software (ai4R). Non-specific signal was determined with an excess of non-radiolabeled compound 1, 7, 8, 9, 15, 16 and 17 (1 μM), respectively, and specific binding was calculated by subtracting the non-specific signal from the total signal.

The Kd (Dissociation constant) and R² (parameter ranging between 0.0 and 1.0 that quantifies the goodness of fit, and the best curve fit obtained with value of 1.0) were obtained by fitting the specific binding data with non-linear regression analysis, using a one-site specific binding model in GraphPad Prism8.

### Results:

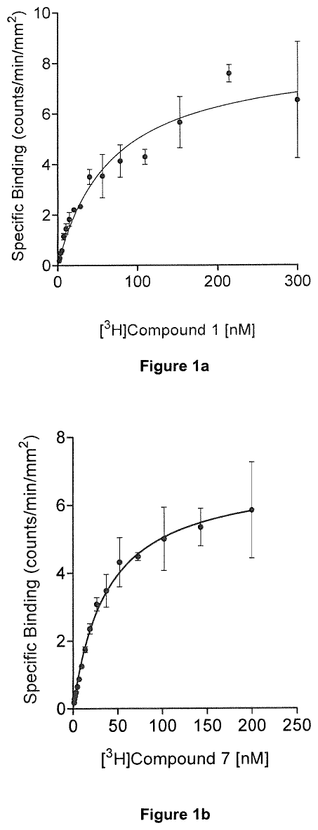

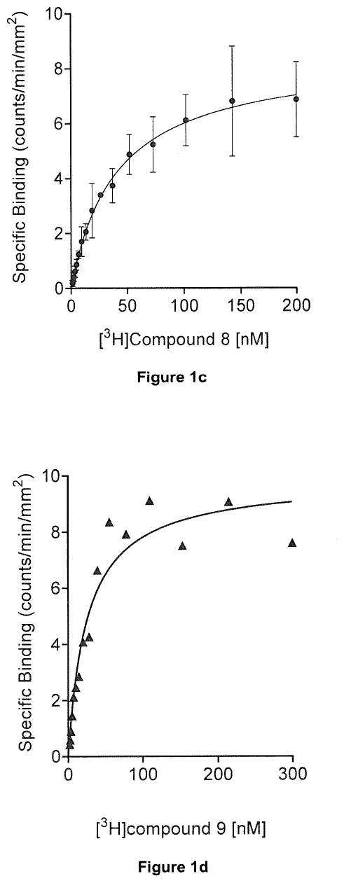

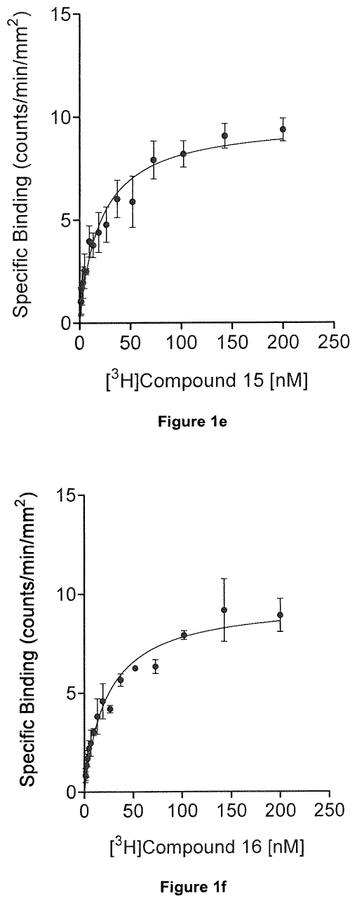

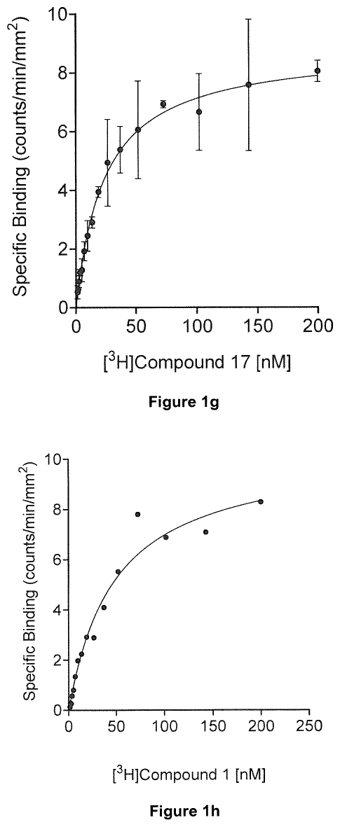

The dissociation constant (Kd) for [³H]-Compounds 1, 7, 8, 9, 15, 16 and 17 were determined on human FTD Type A sarkosyl-insoluble brain extracts in a micro-radiobinding assay. [³H]-Compounds 1, 7, 8, 9, 15, 16 and 17 had a high specific binding resulting in a high dynamic range. [³H]-Compound 1 showed a Kd value of 69 nM on human FTD sarkosyl insoluble brain extracts (FIG. 1a). Data from two independent experiments resulted in the average Kd of 69±21 nM. [³H]-Compound 7 showed a Kd value of 39 nM on human FTD sarkosyl insoluble brain extracts (FIG. 1b). Data from three independent experiments resulted in the average Kd of 39±11 nM. [³H]-Compound 8 showed a Kd value of 47 nM on human FTD sarkosyl insoluble brain extracts (FIG. 1c). Data from two independent experiments resulted in the average Kd of 47±8 nM. [³H]-Compound 9 showed a Kd value of 26 nM on human FTD sarkosyl insoluble brain extracts (FIG. 1d). [³H]-Compound 15 showed a Kd value of 21±7 nM on human FTD sarkosyl insoluble brain extracts (FIG. 1e). [³H]-Compound 16 showed a Kd value of 24±3 nM on human FTD sarkosyl insoluble brain extracts (FIG. 1f). [³H]-Compound 17 showed a Kd value of 25±6 nM on human FTD sarkosyl insoluble brain extracts (FIG. 1g). The dissociation constant (Kd) for [³H]-Compound 1 and [³H]-Compound 17 was also determined on human FTD Type B sarkosyl-insoluble brain extracts in a micro-radiobinding assay. [³H]-Compound 1 showed a Kd value of 49 nM on human FTD Type B sarkosyl insoluble brain extracts (FIG. 1h) and [³H]-Compound 17 a Kd value of 12 nM (FIG. 1i).

2.3. Micro-Autoradiography Staining with [³H]-Compound 1, [³H]-Compound 9 and [³H]-Compound 17 in Brain Sections from Patients with FTD

All tissues were collected from donors for or from whom a written informed consent for a brain autopsy and the use of the material and clinical information for research purposes had been obtained by the respective brain banks. All samples were anonymized and coded. Frozen brain tissue blocks with confirmed TDP-43 pathology upon autopsy were processed using a cryotome to generate sections of 10 μm in thickness and mounted on glass slides. Sections were kept at −80° C. until use.

Brain sections were immunostained using a commercially available antibody, specific for phosphorylated serine at amino acid 409/410 (anti-pTDP-43 pS409/410, Biolegend, 829901). Sections were fixed for 15 minutes at 4° C. with 4% formaldehyde (Sigma, 252549) and washed three times for height minutes with 1×PBS (Dulbecco's phosphate buffered saline, Sigma D1408) at room temperature. Next, sections were saturated and permeabilized in blocking buffer (PBS, 10% normal goat serum (NGS), 0.25% Triton X-100) for one hour at room temperature and incubated overnight at 4° C. with primary antibody against pTDP-43 (diluted at 1/500 in PBS, 5% NGS, 0.25% Triton X-100). The following day, sections were washed three times for height minutes with 1×PBS before incubation with a secondary, AlexaFluor633-labeled goat-anti-rat antibody (Invitrogen, A-21094, diluted at 1/500 in PBS) for 45 minutes at room temperature. Following incubation with primary antibodies the sections were washed three times in PBS before being processed further.

For micro-autoradiography, tritiated test compound ([³H]-Compound 1, [³H]-Compound 9 or [³H]-Compound 17 respectively) was incubated on the sections at 120 nM in 50 mM Tris buffer pH 7.4 for 45 minutes at room temperature. Sections were then washed as follows: once in ice-cold 50 mM Tris-HCl pH 7.4 buffer for one minute, twice in ice-cold 70% ethanol for one minute, once in ice-cold 50 mM Tris-HCl pH 7.4 buffer for one minute and finally rinsed briefly in ice-cold distilled water. Sections were subsequently dried for one hour under a stream of air and then exposed to Ilford Nuclear Emulsion Type K5 (Agar Scientific, AGP9281) for ten days at 4° C. in a light-proof slide storage box. The sections were developed by immersing them successively in the following solutions: 1.) Ilford Phenisol Developer (1:5 dilution in H₂O, Agar Scientific, AGP9106) for four minutes, 2.) Ilfostop solution (1:20 dilution in H₂O, Agar Scientific, AGP9104) for two minutes, 3.) Ilford Hypam Fixer (1:5 dilution in H₂O, Agar Scientific, AGP9183) for four minutes and finally rinsed with H₂O for 10 minutes.

For image acquisition, sections were mounted using ProLong Gold Antifade reagent (Invitrogen P36930) and imaged on a Panoramic250 Slide Scanner (3DHistech) with a 40× objective capturing brightfield and fluorescent images separately or on a Panoramic Scan II (3DHistech) with a 20× objective. The Visiopharm image analysis software suite was used to align the fluorescent image with the brightfield image.

### Results:

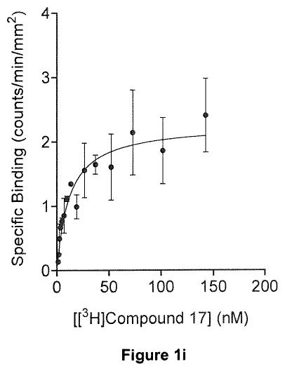

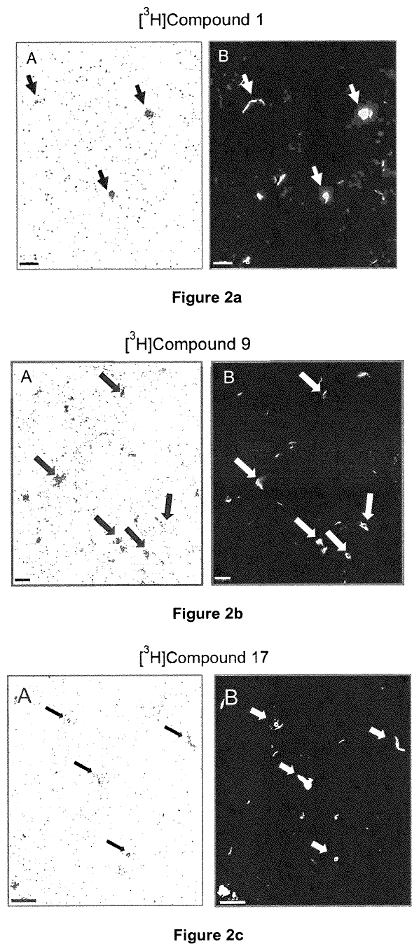

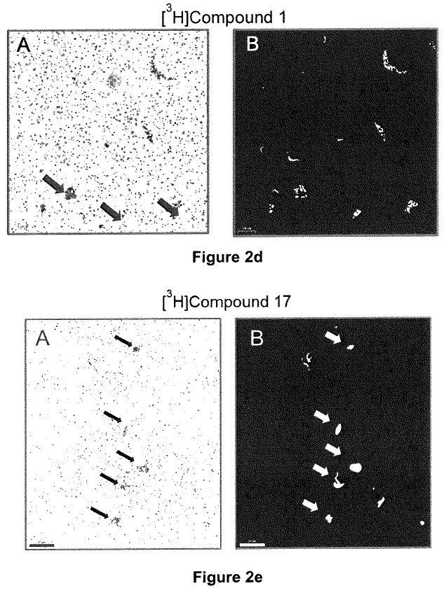

Micro-autoradiography signal from tritiated test compounds ([³H]-Compound 1, [³H]-Compound 9, [³H]-Compound 17) incubated on human brain sections, was detected in the form of accumulating silver grains that co-localize with immunofluorescence signal from pTDP-43-specific antibody suggesting target engagement of [³H]-Compound 1, [³H]-Compound 9 and [³H]-Compound 17 on TDP-43 aggregates. Incubation of brain sections from FTLD-TDP type A donors with [3H]-Compound 1 (120 nM) (FIG. 2a), [³H]-Compound 9 (FIG. 2b) and [³H]-Compound 17 (FIG. 2c) showed colocalization with pTDP-43 aggregates, further providing the evidence of target engagement. The same colocalization was observed with [³H]-Compound 1 and [³H]-Compound 17 on human brain sections from FTLD-TDP type B donors (FIG. 2d and FIG. 2e respectively).

2.4 Autoradiography with [³H]-Compound 1, [³H]-Compound 8 and [³H]-Compound 17 in Brain Sections from Patients with FTD

All tissues were collected from donors for or from whom a written informed consent for a brain autopsy and the use of the material and clinical information for research purposes had been obtained by the respective brain banks. All samples were anonymized and coded. Frozen brain tissue blocks with confirmed TDP-43 pathology upon autopsy were processed using a cryotome to generate sections of 10 μm in thickness and mounted on glass slides. Sections were kept at −80° C. until use.

Brain sections were fixed for 15 minutes at 4° C. with 4% formaldehyde on ice and washed three times for eight minutes with 1×PBS at RT. Sections were blocked in assay buffer for 30 minutes (50 mM Tris-HCl, 0.9% NaCl+0.1% BSA). Cold test compound (Compound 1, Compound 8 or Compound 17) was diluted in assay buffer to 4 μM and applied on half of the sections for 40 minutes at RT to determine non-specific binding. The remaining half of the sections were only incubated with assay buffer (total binding). Following this incubation, an equal volume of the corresponding tritiated compound (i.e. [³H]-Compound 1, [³H]-Compound 8 or [³H]-Compound 17, respectively), diluted to 20 nM (for [³H]-Compound 1 and [³H]-Compound 8) or 10 nM ([³H]-Compound 17) or increasing concentration from 0.1 to 200 nM (for [³H]-Compound 1 and [³H]-Compound 17), was added on sections and incubated for 2 hours at RT and then washed as follows: once in ice-cold 50 mM Tris-HCl pH 7.4 buffer for one minute, twice in ice-cold PBS for one minute, once in ice-cold 50 mM Tris-HCl pH 7.4 buffer for one minute and finally rinsed briefly in ice-cold distilled water.

Sections were subsequently dried under a stream of air and mounted with a copper-based adhesive strip. Bound radioactivity was quantified by exposing the slides in a real-time autoradiography system (Beaquant™ AI4R) for 2 hours, with measurement settings configured for tritium. Beavacq software (AI4R) was used to acquire the image which was subsequently quantified using Beamage image analysis software (AI4R). Specific binding (non-specific binding subtracted from total) and displacement (total binding divided by non-specific) were calculated. Bar graphs were generated using GraphPad Prism. For Kd determination on brain sections, the specific binding data were fitted with a non-linear regression analysis using a one-site specific binding model in GraphPad Prism.

### Results:

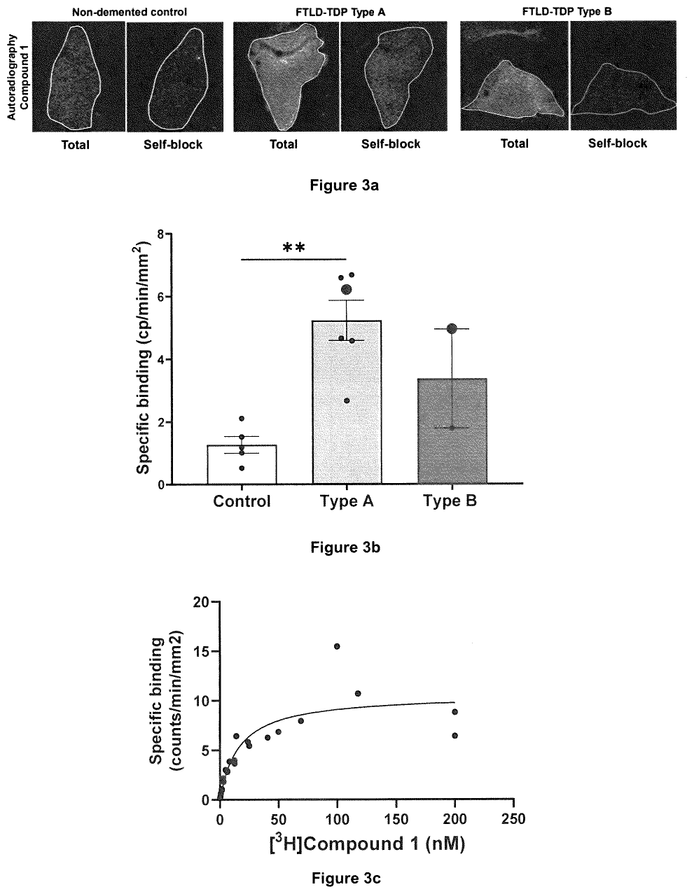

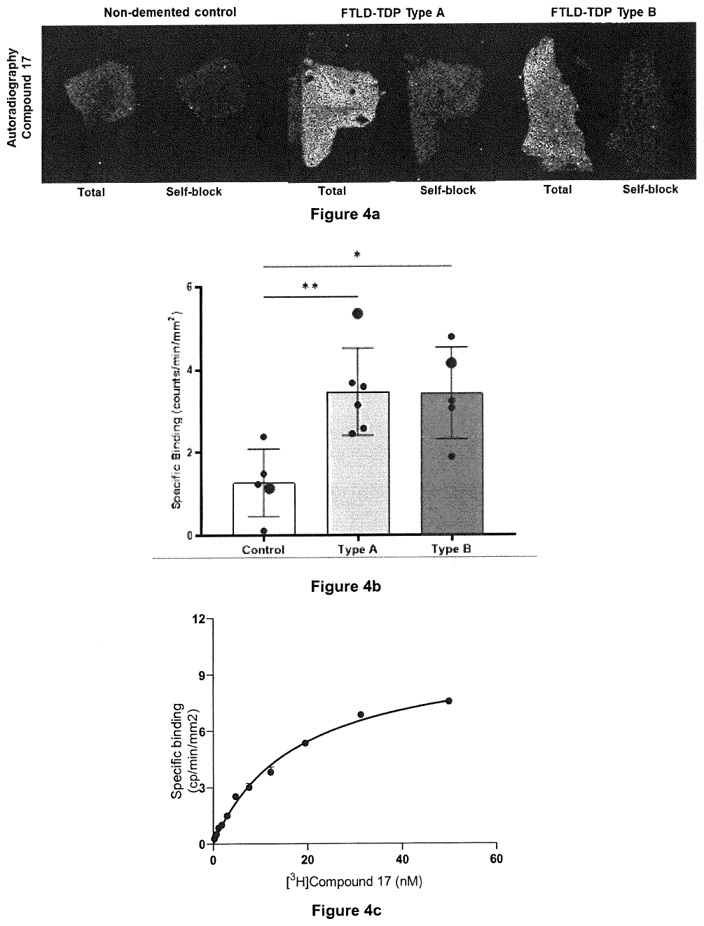

Example compounds were assessed for their binding on human tissue sections from non-demented control cases, FTLD-TDP type A or FTLD-TDP type B (FIGS. 3a and 4a). Displacement of the tritiated compound by the non-radiolabeled compound was calculated in the grey matter regions (total binding divided by non-specific binding) and is reported in Table 6 below for each case.

TABLE 6

| | cARG | | |
|---|---|---|---|
| | displacement | cARG | cARG |
| | on non- | displacement | displacement |
| Example | demented | on FTLD-TDP | on FTLD-TDP |
| Compound | control | Type A case | Type B case |
| 1 | 1.9 | 3.3 | 3.2 |
| 8 | 2.3 | 3.0 | 3.3 |
| 17 | 1.7 | 2.9 | 2.6 |

Compounds 1, 8 and 17 showed increased displacement on FTLD-TDP type A and B compared to on non-demented control, further providing the evidence of target engagement in pathological tissue. As seen from FIG. 3b and FIG. 4b, [³H]-Compound 1 and [³H]-Compound 17, respectively, displayed significantly higher specific binding in FTLD-TDP type A compared to control (larger plain circles correspond to the type A and type B images shown on FIGS. 3a and 4a). One-way ANOVA with Tukey's correction for multiple comparison, *p<0.05, **p<0.01). [³H]-Compound 1 and [³H]-Compound 17 were also assessed in saturation binding studies using FTLD-TDP type A tissue sections and Kd values of 16 nM and 18 nM respectively were measured suggesting that they bind with high affinity to TDP-43 aggregates (FIG. 3c and FIG. 4c).

### 2.5 Selectivity for TDP-43

### 2.5.1 Radiobinding Assay on Alzheimer's Disease (AD) Brain Homogenates

### 2.5.1.1 Preparation of Human AD Insoluble Fraction of Brain Homogenates

The procedure used was adapted from Bagchi et al., 2013, describing extraction of homogenized insoluble fraction containing protein aggregates from human brain tissue for in vitro binding and competition studies.

Approximately 4 g of frozen tissue block from frontal cortex brain region was used from an AD donor with confirmed burden of Tau and Abeta aggregates. The tissue was homogenized in three volumes (1:3 w/v) of high salt buffer (50 mM Tris-HCl pH 7.5, 750 mM NaCl, 5 mM EDTA) supplemented with protease inhibitors (Complete; Roche 11697498001) at 4° C. with a glass Dounce homogenizer. The homogenate was transferred into polycarbonate centrifuge bottles (16×76 mm; Beckman 355603) and centrifuged at 100,000×g (38,000 RPM) in an ultracentrifuge (Beckman, XL100K) for 60 minutes at 4° C. using a pre-cooled 70.1 rotor (Beckman, 342184). Pellets were resuspended in high salt buffer supplemented with 1% Triton X-100 and homogenized at 4° C. with a syringe. The homogenates were centrifuged again at 100,000×g (38,000 RPM, 70.1 rotor) for 60 minutes at 4° C. Pellets were resuspended in high salt buffer supplemented with 1% Triton X-100 and 1 M sucrose and homogenized at 4° C. with a syringe. The homogenates were centrifuged at 100,000×g (38,000 RPM, 70.1 rotor) for 60 minutes at 4° C. The resulting pellets containing insoluble fraction were resuspended in PBS (1:2 w/v relative to starting tissue weight), aliquoted and stored at −80° C. until use.

### 2.5.1.2 Radiobinding Competition Assay Using Abeta Reference Compound for Determination Of Inhibitor Constant (Ki) on AD Insoluble Fraction of Brain Homogenates

Fixed concentration of AD insoluble fraction was incubated with fixed concentration of radiolabeled Abeta reference compound (Kd=33 nM on AD brain homogenate) and increasing concentrations of a non-radiolabeled compound ranging from 0.41 nM to 2 μM. The reaction was performed in assay buffer (50 mM Tris pH 7.5 in 0.9% NaCl, 0.1% BSA) and incubated for two hours at room temperature (RT). Samples in duplicate were then filtered under vacuum in GF/C filter plates (PerkinElmer) to trap the aggregates with the bound radioligand and washed with 100 μL ice-cold 50 mM Tris pH 7.5. The GF/C filters were then dried and scintillation liquid (UltimateGold, PerkinElmer, 6013151) was added in each well. The filters were analyzed on a Microbeta2 scintillation counter (PerkinElmer). Values for the maximal signal were obtained in the absence of non-radiolabeled compound, while 100% displacement was obtained using 1 μM of non-radiolabeled reference compound. Ki values were calculated by nonlinear regression, using a one site—fit Ki model in Prism V7 (GraphPad). Average Ki for the non-radiolabeled Abeta reference compound is 32 nM.

### 2.5.1.3 Radiobinding Assay for Determination of Dissociation Constant (Kd) on AD Insoluble Fraction of Brain Homogenates

The AD insoluble fraction was incubated with [³H]Compound 1, [³H]Compound 17 or [³H]Abeta reference compound ([³H]Abeta ref) at different concentrations. To determine the non-specific signal, 1 μM of non-radiolabeled compound was added to all concentrations of tritiated compound in control wells. The reaction was performed in assay buffer (50 mM Tris pH 7.5 in 0.9% NaCl, 0.1% BSA) and incubated for two hours at RT. Samples were then filtered through GF/C filter plates (PerkinElmer) under vacuum to trap/immobilize the aggregates with the bound radioligand and washed five times with 50 mM Tris pH7.5. The GF/C filters were then dried and scintillation liquid (UltimateGold, PerkinElmer, 6013151) was added in each well. The filters were analyzed on a Microbeta scintillation counter (PerkinElmer). Specific binding was calculated by subtracting the non-specific signal from the total signal. The Kd and R² values were obtained by fitting the specific binding data with non-linear regression analysis using a one-site specific binding model in GraphPad Prism.

2.5.2. Micro-Radiobinding Assay in PD Brain-Derived a-Syn Aggregates and Tau PHF Derived from an AD Case
2.5.2.1 Isolation of PD Brain-Derived Pathological a-Syn Aggregates

The procedure was adapted from the protocol described in Spillantini et al., 1998. Frozen tissue blocks from PD donors were thawed on ice and homogenized using a glass dounce homogenizer. The homogenate was then centrifuged at 11,000×g (12,700 RPM) in an ultracentrifuge (Beckman, XL100K) for 20 minutes at 4° C. using a pre-cooled 70.1 rotor (Beckman, 342184). Pellets were resuspended in extraction buffer [10 mM Tris-HCl pH 7.4, 10% sucrose, 0.85 mM NaCl, 1% protease inhibitor (Calbiochem 539131), 1 mM EGTA, 1% phosphatase inhibitor (Sigma P5726 and P0044)] and centrifuged at 15,000×g (14,800 RPM, a 70.1 Ti rotor) for 20 minutes at 4° C. Pellets were discarded and sarkosyl (20% stock solution, Sigma L7414) was added to the supernatants to a final concentration of 1% at room temperature for one hour. This solution was then centrifuged at 100,000×g (38,000 RPM, 70.1 Ti rotor) for one hour at 4° C. Pellets containing enriched a-syn aggregates were resuspended in PBS and stored at −80° C. until use.

2.5.2.2 Preparation and Extraction of Paired Helical Filament (PHF)-Tau Aggregates from AD Brain

The enrichment procedure was modified from Jicha et al, 1997 and Rostagno and Ghiso, 2009. Briefly, around 15 g of the AD human brain were thawed on ice and homogenized with homogenization buffer (3 ml per gram of tissue) (0.75 M NaCl in RAB buffer (100 mM 2-(N-morpholino)ethanesulfonic acid (MES), 1 mM EGTA, 0.5 mM MgSO₄, 2 mM DTT, pH 6.8) supplemented with protease inhibitors (Complete; Roche 693124001)) in a glass Dounce homogenizer. The homogenate was then incubated at 4° C. for 20 min to let any residual microtubules depolymerize, before being transferred into polycarbonate centrifuge bottles (16×76 mm; Beckman 355603) and being centrifuged at 11,000 g (12,700 RPM) in an ultracentrifuge (Beckman, XL100K) for 20 min at 4° C. using the pre-cooled 70.1 rotor (Beckman, 342184). Pellets were kept on ice. Supernatants were pooled into polycarbonate bottles and centrifuged again at 100,000 g (38,000 RPM) for 1 h at 4° C. in the 70.1 Ti rotor. The pellets from the first and second centrifugations were resuspended in extraction buffer (10 ml per gram of tissue) (10 mM Tris-HCl pH 7.4, 10% sucrose, 0.85 M NaCl, 1 tablet/50 ml protease inhibitor (Roche, 4693124001), 1 mM EGTA, 1% phosphatase inhibitor (Sigma P5726 and P0044)). The solution was then transferred into polycarbonate centrifuge bottles (16×76 mm; Beckman 355603) and centrifuged at 15,000 g (14,800 RPM) in an ultracentrifuge (Beckman, XL100K) for 20 min at 4° C. using the 70.1 Ti rotor. The pellets were stored at −80° C. 30% Sarkosyl (Fluka analytical, 61747) was added to the supernatants to a final concentration of 1% and stirred at RT for 1 h. This solution was then centrifuged in polycarbonate bottles at 100,000 g (38,000 RPM) for 1 h at 4° C. in the 70.1 Ti rotor, and the pellets containing PHF-rich material were resuspended in 50 μl PBS/1 g of brain tissue. The resuspended PHF was then sonicated on ice for 2 min with 0.5 s-on/0.5 s-off cycles at 30% amplitude. Aliquots were snap frozen and stored at −80° C.

### 2.5.2.3 Compound Incubation and Readout

PD brain-derived a-synuclein aggregates and AD brain-derived Tau PHF aggregates were spotted onto microarray slides. The slides spotted with a-synuclein aggregates were incubated with [³H]-a-synuclein reference at 25 nM or 40 nM and the example compound (non-radiolabelled) at 100 nM and at a range of increasing concentrations, varying from 0.05 nM to 2 μM. Additionally, slides spotted with a-synuclein aggregates or with Tau PHF samples were incubated with [³H]Compound-1, [³H]Compound-17 and [³H]-a-synuclein reference or [³H]Tau reference compound, respectively, in a concentration range from 0.6 to 200 nM. After incubation, slides were washed and scanned by a real-time autoradiography system (BeaQuant, ai4R). Quantification of signal was performed by using the image analysis software Beamage (ai4R). Non-specific signal was determined with an excess of non-radiolabelled compound (2 μM) and specific binding was calculated by subtracting the non-specific signal from the total signal. Competition was calculated as percentage, where 0% was defined as the specific binding in the presence of vehicle and 100% as the values obtained in the presence of excess of the non-radiolabelled a-syn reference compound. Ki values were calculated in GraphPad Prism7 by applying a nonlinear regression curve fit using a one site, specific binding model. All measurements were performed with at least two technical replicates. The Kd and R² values were obtained by fitting the specific binding data with non-linear regression analysis using a one-site specific binding model in GraphPad Prism.

Results: Example compounds were assessed for selectivity to TDP-43 over Abeta and a-synuclein. To evaluate selectivity over Abeta, the inhibitor constant (Ki) values were measured on AD brain homogenates against a [³H]Abeta reference compound ([³H]Abeta ref) (Table 7). As seen from the high Ki values in Table 7, compounds of the invention show good selectivity for TDP-43 over Abeta.

TABLE 7

| | Ki (nM) on AD |
|---|---|
| Example | brain homogenates |
| 1 | >1 μM |
| 7 | >1 μM |
| 8 | >1 μM |
| 9 | >1 μM |
| 10 | 350 |
| 13 | >1 μM |
| 15 | >1 μM |
| 16 | >1 μM |
| 17 | >1 μM |
| 23 | >1 μM |
| 24 | >1 μM |
| 25 | >1 μM |
| 26 | 318 |
| 27 | >1 μM |
| 29 | >1 μM |
| 31 | >1 μM |
| 32 | >1 μM |
| 34 | >1 μM |
| 35 | >1 μM |
| 37 | >1 μM |

^{a}n.d^{:} not determined

To evaluate selectivity over a-synuclein, the potency of example Compound 1 to compete with the binding of a [3H]-reference a-syn compound on PD brain-derived insoluble fraction was measured Compound 1 showed a Ki of >1000 nM on AD brain homogenate and a Ki of >500 nM on PD brain-derived insoluble fraction.

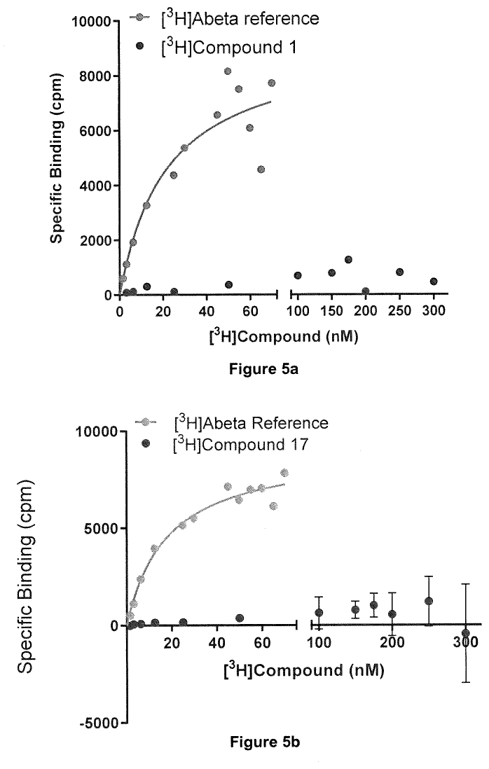

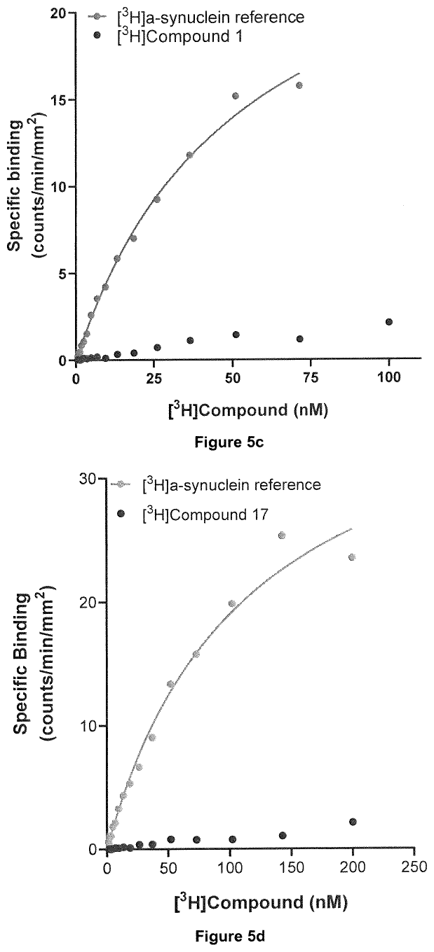

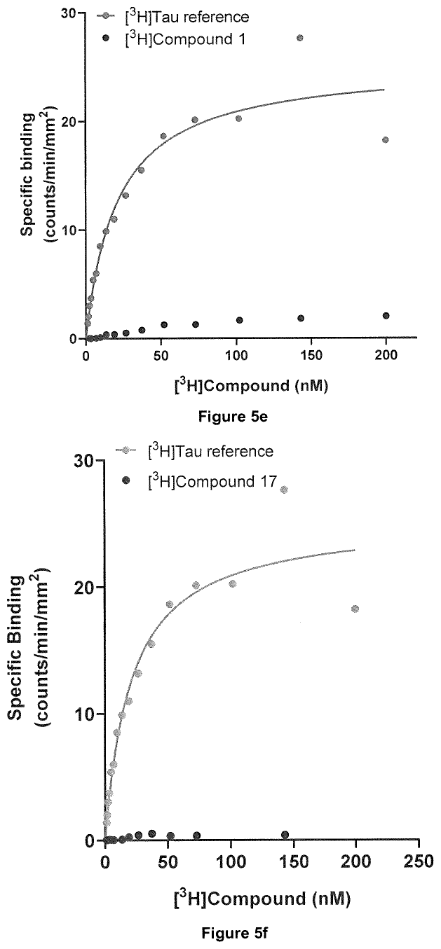

Furthermore, [³H]Compound-1 and [³H]Compound-17 were additionally assessed for TDP-43 selectivity over Abeta, a-synuclein, and Tau by direct binding in AD brain homogenates (containing Abeta and Tau, FIGS. 5a-5b), in PD brain-derived a-synuclein aggregates (FIGS. 5c-5d)) and in AD brain-derived Tau PHF aggregates (FIGS. 5e-5f). No significant binding of [³H]Compound-1 nor of [³H]Compound-17 was detected in any of these preparations as opposed to the respective reference ligands.

Results demonstrate that compounds according to the invention display good TDP-43 selectivity over Abeta, a-synuclein and Tau.

### 2.5.3. Assessment of Target Engagement of Example-1 and Example-17 in Abeta and Tau Aggregates in AD Tissue by Classical Autoradiography and High-Resolution Micro-Autoradiography

### 2.5.3.1 By Classical Autoradiography

All tissues were collected from donors for or from whom a written informed consent for a brain autopsy and the use of the material and clinical information for research purposes had been obtained by the respective brain banks. All samples were anonymized and coded. Frozen brain tissue blocks with confirmed Abeta and Tau pathology upon autopsy were processed using a cryotome to generate sections of 10 μm in thickness and mounted on glass slides. Sections were kept at −80° C. until use.

Brain sections were fixed for 15 minutes at 4° C. with 4% formaldehyde on ice and washed three times for eight minutes with 1×PBS at RT. Sections were blocked in assay buffer for 30 minutes (50 mM Tris-HCl, 0.9% NaCl+0.1% BSA). Cold test compound (Compound 1) was diluted in assay buffer to 4 μM and applied on half of the sections for 40 minutes at RT to determine non-specific binding. The remaining half of the sections were only incubated with assay buffer (total binding). Following this incubation, an equal volume of the corresponding tritiated compound (i.e. [³H]-Compound 1, [³H]-Compound 17, [³H]-Abeta reference compound or [³H]-tau reference compound), diluted to 10 nM, was added on sections and incubated for 2 hours at RT and then washed as follows: once in ice-cold 50 mM Tris-HCl pH 7.4 buffer for one minute, twice in ice-cold PBS for one minute, once in ice-cold 50 mM Tris-HCl pH 7.4 buffer for one minute and finally rinsed briefly in ice-cold distilled water.

Sections were subsequently dried under a stream of air and mounted with a copper-based adhesive strip. Bound radioactivity was quantified by exposing the slides in a real-time autoradiography system (Beaquant™ AI4R) for 2 hours with measurement settings configured for tritium. Beavacq software (AI4R) was used to acquire the image which was subsequently quantified using Beamage image analysis software (AI4R).

### Results:

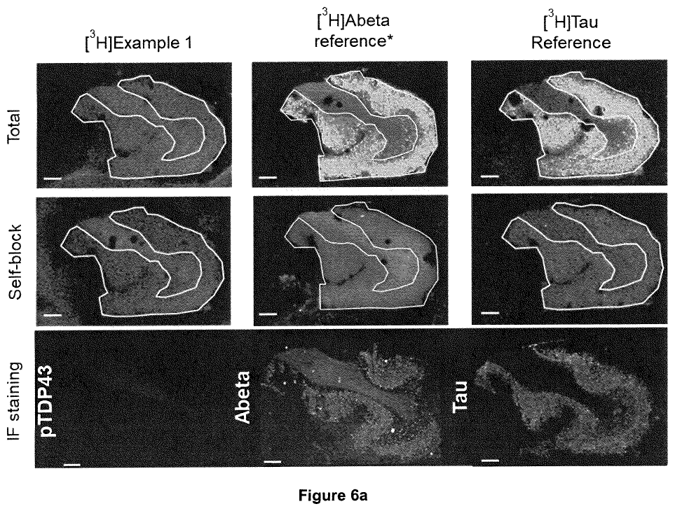

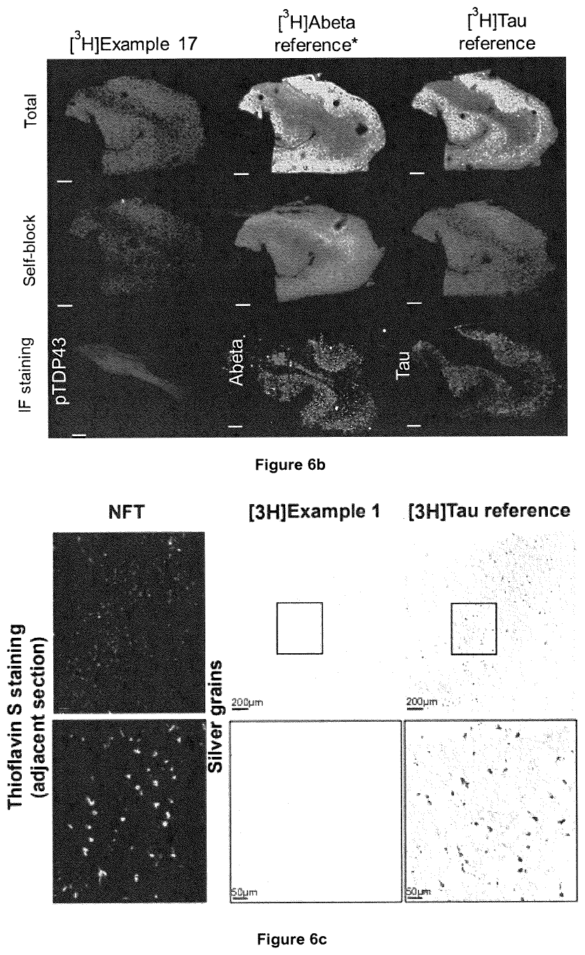

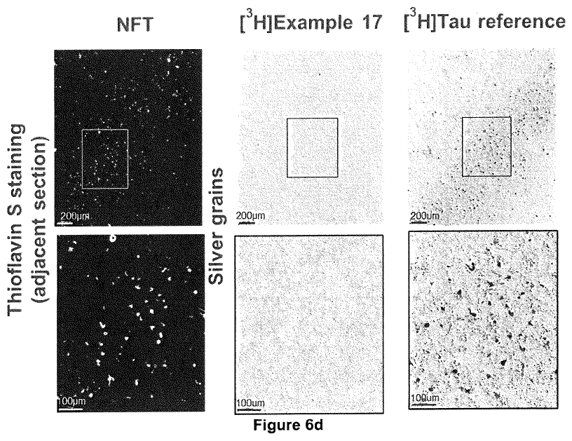

Example compounds 1 and 17 were assessed for their binding on human tissue sections from an AD case containing abundant Abeta and Tau pathology (FIGS. 6a and 6b, respectively). Images of total binding (first row) and non-specific binding (middle row) are shown for [³H]-Compounds 1 or 17 (first column), [³H]-Abeta reference compound (second column) or [³H]-Tau reference compound (last column). Immunofluorescence images (bottom row) of pTDP-43 (first column), Abeta (second column) or pTau (last column). Example compounds 1 and 17 showed no signal on the tissue with either of the conditions tested (radiolabeled compound alone or in self-competition), correlating with the absence of pTDP-43 immunolabelling on an adjacent section. Whereas both reference for Abeta and for Tau showed a total binding signal which was displaced under self-competition, correlating with the immunolabelling of pTau and Abeta on adjacent sections of the tissue. Results further demonstrate that compounds according to the invention display good TDP-43 selectivity over Abeta and Tau.

### 2.5.3.2 by High Resolution Micro-Autoradiography

The protocol was adapted from Marquie et al., 2015. AD sections were incubated with [³H]-Compound 1, [³H]-Compound 17 or [³H]-Tau reference compound at 20 nM for one hour at room temperature (RT). Sections were then washed as follows: One time in ice-cold 50 mM Tris-HCl pH 7.4 buffer for one minute, two times in 70% ice-cold ethanol for one minute, one time in ice-cold 50 mM Tris-HCl pH 7.4 buffer for one minute and finally rinsed briefly in ice-cold distilled water. Sections were subsequently dried and then exposed to Ilford Nuclear Emulsion Type K5 (Agar Scientific, AGP9281) in a light-proof slide storage box. After five days, the sections were developed by immersing them successively in the following solutions: 1.) Ilford Phenisol Developer (1:5 dilution in H₂O, Agar Scientific, AGP9106), 2.) Ilfostop solution (1:20 dilution in H₂O, Agar Scientific, AGP9104), 3.) Ilford Hypam Fixer (1:5 dilution in H₂O, Agar Scientific, AGP9183) and finally rinsed with H₂O.

Thioflavin S staining was performed on adjacent sections. For image acquisition, sections were mounted using ProLong Gold Antifade reagent (Invitrogen P36930) and imaged on a Panoramic150 Slide Scanner (3DHistech) with a 20× objective capturing separately brightfield and fluorescent images.

### Results:

Micro-autoradiography signals from [³H]-Compound 1, [³H]-Compound 17 and [H]-Tau reference incubated on human brain sections, were assessed in the form of accumulating silver grains in a region rich in Tau tangles. Results are shown in FIG. 6c ([³H]-Compound 1) and FIG. 6d ([³H]-Compound 17) Left, thioflavin S staining on the same tissue labelling Tau aggregates. Right, images of silver grain deposition for [³H]-Compound 1 or [³H]-Compound 17 and [³H]-Tau reference compound on the same tissue. No accumulation of silver grains on Tau tangles with [³H]-Compound 1 or [³H]-Compound 17, as compared to [³H]-Tau reference. Bottom row images are zoomed-in images from the region indicated by a square on the top row image. *NFT: Neurofibrillary tangles. Scale bar is 200 μm (top row) and 50 μm (bottom row, FIG. 6c) or 100 μm (bottom row, FIG. 6d). As shown in FIG. 6c and FIG. 6d, incubation of brain sections from an AD case with [³H]-Compound 1 or [³H]-Compound 17 showed absence of accumulating silver grains, when on adjacent sections [³H]-Tau reference compound showed target engagement in the region with Tau tangles, as determined by Thioflavin S staining. Results further demonstrate that compounds according to the invention display good TDP-43 selectivity over Tau.

### 2.6 PK Studies in Healthy Monkey

Non-Human Primate (NHP) was injected intravenously (iv) with the [¹⁸F]-labeled Compound 1 ([¹⁸F]Compound 1) (4.2 mCi) or with the [¹⁸F]-labeled Compound 17 ([¹⁸F]-Compound 17) (5.3 mCi). Monkey PET scans were performed using a Siemens Focus 220. PET acquisition started immediately before the radioactive dose was injected. Images were generated as dynamic scans for 120 minutes with head focused. [¹⁸F]-Compound 1 and [¹⁸F]-Compound 17 entered the brain quickly (5.5 min and 4.5 min post injection, respectively) and showed robust uptake with 2.7 and 1.1 SUVmax whole brain, respectively (FIGS. 7a-7b). In addition, [¹⁸F]-Compound 1 and [¹⁸F]-Compound 17 had a quick washout with peak to half peak of <17 min and of <13 min, respectively. These data demonstrate PK profiles of [¹⁸F]-Compound 1 and [¹⁸F]-Compound 17 of the invention in non-human primates suitable for their use as brain PET agent in humans.

1. A compound having the formula (I) 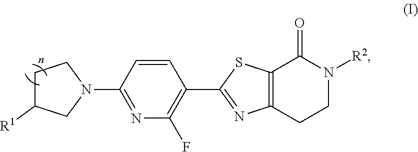
or a detectably labelled compound, stereoisomer, polymorph, racemic mixture, tautomer, pharmaceutically acceptable salt, prodrug, hydrate, or solvate thereof;
wherein
n is 1 or 2;
R¹ is H, hydroxy(C₁-C₄)alkyl, or F; and
R² is a 5- or 6-membered carbocyclic or heterocyclic ring which can be optionally substituted with F, NH₂, CN and/or CH₃, wherein the heterocyclic ring contains one or more heteroatoms selected from N, O and S.

2. A compound having the formula (I) according to claim 1, wherein
R¹ is H or F.

3. The compound according to claim 1, wherein
R² is 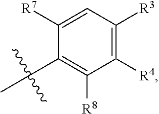
wherein

| | | | |
|---|---|---|---|
| R³ is F, | R⁴ is NH₂, | R⁷ is H, and | R⁸ is H; |
| R³ is NH₂, | R⁴ is F, | R⁷ is H, and | R⁸ is H, |
| R³ is CN, | R⁴ is NH₂, | R⁷ is H, and | R⁸ is H, |
| R³ is H, | R⁴ is NH₂, | R⁷ is H, and | R⁸ is CN, |
| R³ is H, | R⁴ is NH₂, | R⁷ is H, and | R⁸ is F, or |
| R³ is H, | R⁴ is NH₂, | R⁷ is H, and | R⁸ is CN; |

or R² is 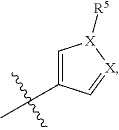
wherein X is N and R⁵ is CH₃ or H;
or R² is 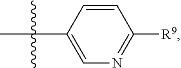
wherein R⁹ is selected from H, F, CH₃ and NH₂, preferably H;
or R² is 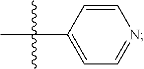 [SMILES: *c1ccncc1]
or R² is 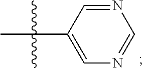 [SMILES: *c1cncnc1]
or R² is 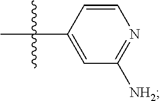 [SMILES: *c1ccnc(N)c1]
or R² is 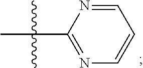 [SMILES: *c1ncccn1]
or R² is 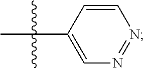 [SMILES: *c1ccnnc1]
or R² is 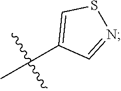 [SMILES: *c1cnsc1]
or R² is 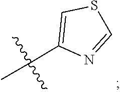 [SMILES: *c1cscn1]
or R² is

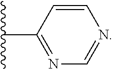 [SMILES: *c1ccncn1]

4. The compound according to claim 1, wherein
R² is 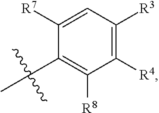
wherein R³ is F, R⁴ is NH₂, R⁷ is H, and R⁸ is H; 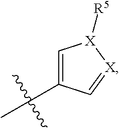
wherein X is N and R⁵ is CH₃; 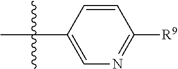
wherein R⁹ is selected from H and F;

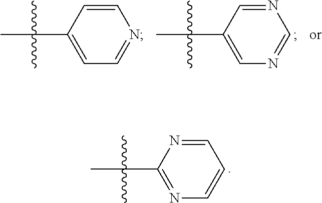 [SMILES: *c1ccncc1 ; *c1cncnc1 ; *c1ncccn1]

5. The compound according to claim 1, which is selected from

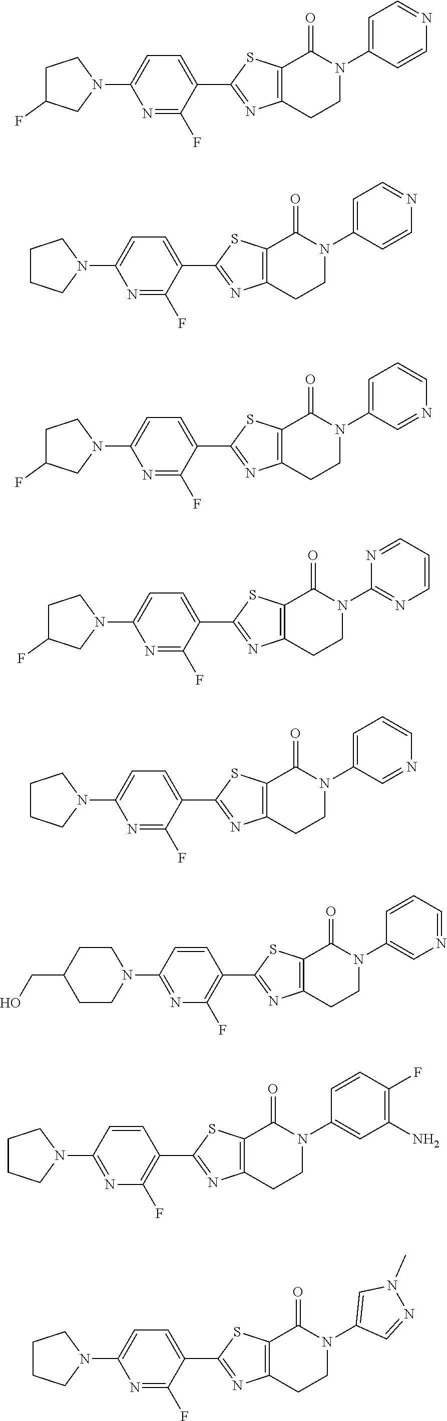

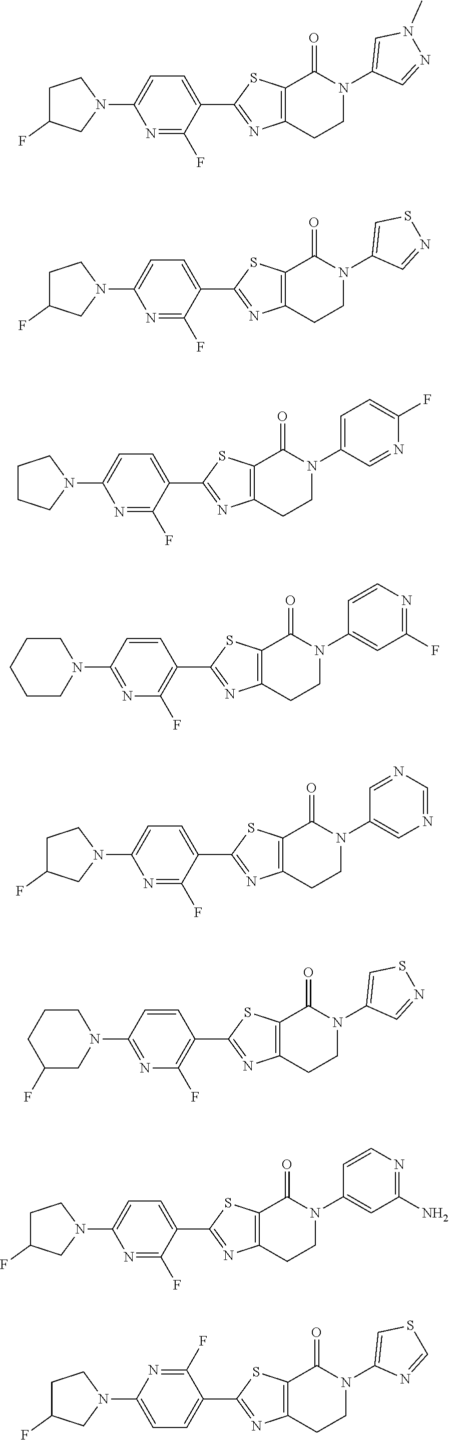

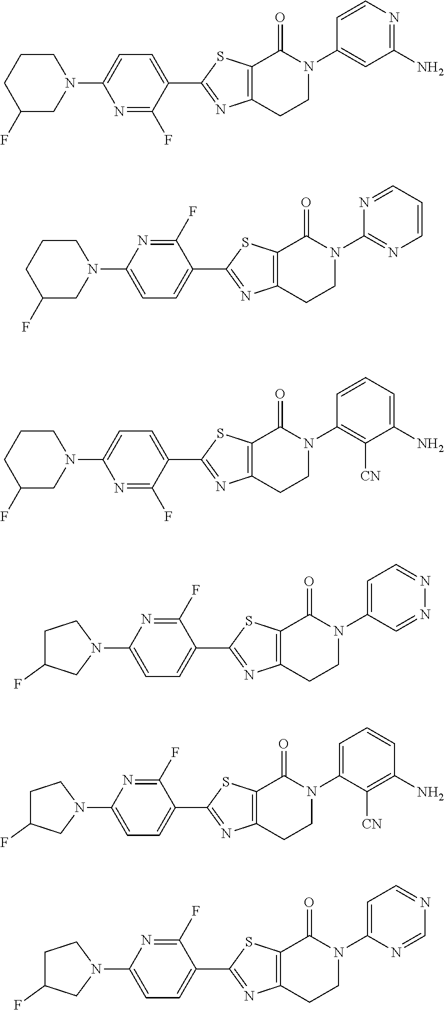 [SMILES: Nc1cc(N2CCc3nc(-c4ccc(N5CCCC(F)C5)nc4F)sc3C2=O)ccn1 ; O=C1c2sc(-c3ccc(N4CCCC(F)C4)nc3F)nc2CCN1c1ncccn1 ; N#Cc1c(N)cccc1N1CCc2nc(-c3ccc(N4CCCC(F)C4)nc3F)sc2C1=O ; O=C1c2sc(-c3ccc(N4CCC(F)C4)nc3F)nc2CCN1c1ccnnc1 ; N#Cc1c(N)cccc1N1CCc2nc(-c3ccc(N4CCC(F)C4)nc3F)sc2C1=O ; O=C1c2sc(-c3ccc(N4CCC(F)C4)nc3F)nc2CCN1c1ccncn1]

6. The compound according to claim 1, which comprises a detectable label, which is optionally ³H or ¹⁸F.

7. (canceled)

8. The compound according to claim 6, having the formula (I-T) 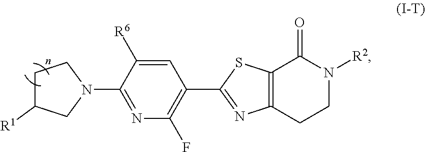
or a stereoisomer, polymorph, racemic mixture, tautomer, pharmaceutically acceptable salt, prodrug, hydrate, or solvate thereof;
wherein
n is 1 or 2;
R¹ is H, hydroxy(C₁-C₄)alkyl, or F;
T is ³H; and
wherein
R² is 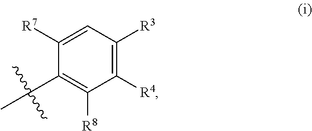
wherein R³ is F, R⁴ is —NH₂, and at least one of R⁷ and R⁸ is T and, if applicable, the other is H; preferably R⁷ is T and R⁸ is T; and
R⁶ is T;
or wherein
R² is 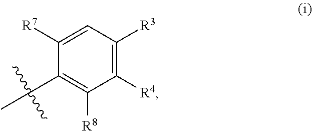
wherein R⁴ is —NH₂, R⁸ is CN, and at least one of R³ or R⁷ is T and and, if applicable, the other is H; preferably R⁷ is T and R³ is T; or R⁷ is T and R³ is H and
R⁶ is T; or
wherein
R² is 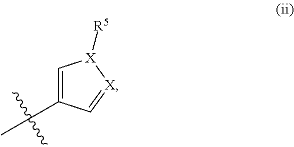
wherein X is N and R⁵ is CT₃; and
R⁶ is H; or wherein
R² is 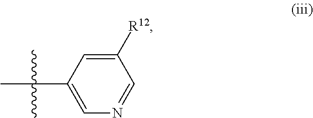
wherein R¹² is T;
or wherein
R² is 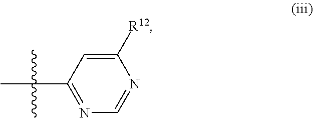
wherein R¹² is T;
or wherein
R² is 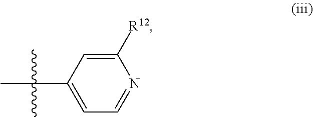
wherein R¹² is T;
or wherein
R² is 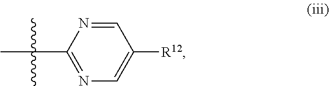
wherein R¹² is T.

9. The compound according to claim 6, having the formula (I-T′) 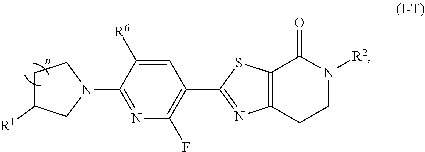
or a stereoisomer, polymorph, racemic mixture, tautomer, pharmaceutically acceptable salt, prodrug, hydrate, or solvate thereof;
wherein
n is 1 or 2;
R¹ is hydroxy(C₁-C₄)alkyl substituted by one or two, preferably two, T,
wherein T is ³H; and
R² is a 5- or 6-membered carbocyclic or heterocyclic ring which can be optionally substituted with F, NH₂, CN and/or CH₃, wherein the heterocyclic ring contains one or more heteroatoms selected from N, O and S.

10. The compound according to claim 8, wherein the compound is selected from 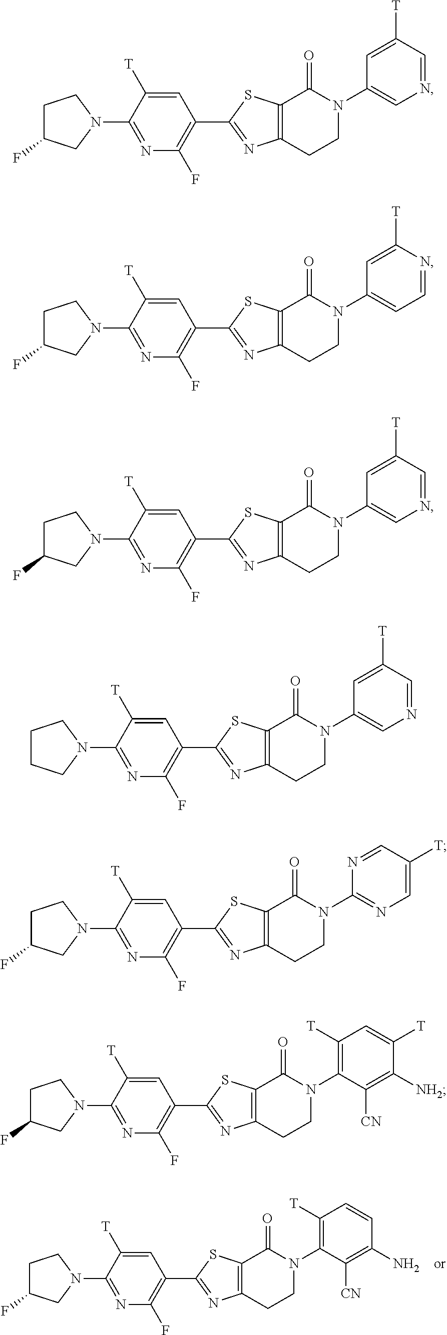 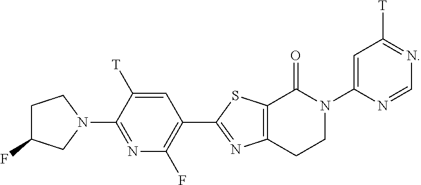
wherein T is ³H.

11. The compound according to claim 9, wherein the compound is selected from 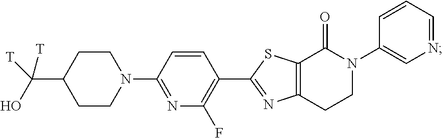
wherein T is ³H.

12. The compound according to claim 6, having the formula (I-F) 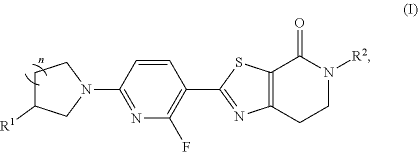
or a stereoisomer, polymorph, racemic mixture, tautomer, pharmaceutically acceptable salt, prodrug, hydrate, or solvate thereof;
wherein
n is 1 or 2;
R¹ is ¹⁸F;
R² is 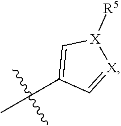
wherein X is N and R⁵ is CH₃; or 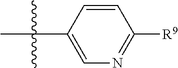
wherein R⁹ is H.

13. The compound according to claim 6, having the formula (I-F) 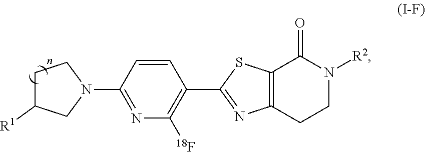
or a stereoisomer, polymorph, racemic mixture, tautomer, pharmaceutically acceptable salt, prodrug, hydrate, or solvate thereof;
wherein
n is 1 or 2;
R¹ is H or hydroxy(C₁-C₄)alkyl, and
R² is 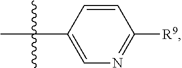
wherein R⁹ is H.

14. The compound according to claim 12, which is

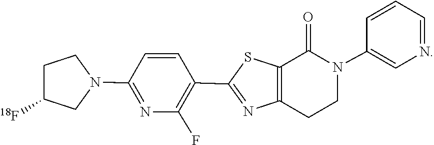 [SMILES: O=C1c2sc(-c3ccc(N4CC[C@@H]([18F])C4)nc3F)nc2CCN1c1cccnc1]

15. The compound according to claim 13, which is

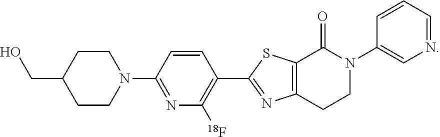 [SMILES: O=C1c2sc(-c3ccc(N4CCC(CO)CC4)nc3[18F])nc2CCN1c1cccnc1]

16. A diagnostic composition comprising a compound according to claim 1, and optionally at least one physiologically acceptable carrier, diluent, adjuvant and/or excipient.

17. (canceled)

18. A method of imaging of TDP-43 aggregates, particularly wherein the imaging is conducted by positron emission tomography, wherein a compound according to claim 1 is employed.

19. A method of diagnosing a disease, disorder or abnormality associated with TDP-43 aggregates, wherein a compound according to claim 1 is employed, particularly wherein the diagnosing is conducted by positron emission tomography.

20. A method which is selected from the group consisting of:
(i) A method of diagnosing a disease, disorder or abnormality associated with TDP-43 aggregates or a predisposition thereto in a subject, the method comprising the steps:
(a) Administering a compound according to claim 1 to the subject;
(b) Allowing the compound to bind to the TDP-43 aggregates; and
(c) Detecting the compound bound to the TDP-43 aggregates;
(ii) A method of positron emission tomography (PET) imaging of TDP-43 aggregates in a tissue of a subject, the method comprising the steps:
(a) Administering a compound according to claim 1 to the subject;
(b) Allowing the compound to bind to the TDP-43 aggregates; and
(c) Detecting the compound bound to the TDP-43 aggregates by collecting a positron emission tomography (PET) image of the tissue of the subject:
(iii) A method for the detection and optionally quantification of TDP-43 aggregates in a tissue of a subject, the method comprising the steps:
(a) Bringing a sample or a specific body part or body area suspected to contain TDP-43 aggregates into contact with a compound according to claim;
(b) Allowing the compound to bind to the TDP-43 aggregates;
(c) Detecting the compound bound to the TDP-43 aggregates using positron emission tomography; and
(d) Optionally quantifying the amount of the compound bound to the TDP-43 aggregates;
(iv) A method of collecting data for the diagnosis of a disease, disorder or abnormality associated with TDP-43 aggregates or a predisposition thereto, the method comprising the steps:
(a) Bringing a sample or a specific body part or body area suspected to contain TDP-43 aggregates into contact with a compound according to claim 1;
(b) Allowing the compound to bind to the TDP-43 aggregates;
(c) Detecting the compound bound to the TDP-43 aggregates; and
(d) Optionally correlating the presence or absence of the compound bound with the TDP-43 aggregates with the presence or absence of TDP-43 aggregates in the sample or specific body part or body area;
(v) A method of collecting data for monitoring the progression of a disease, disorder or abnormality associated with TDP-43 aggregates in a patient, the method comprising the steps:
(a) Bringing a sample, a specific body part or body area suspected to contain TDP-43 aggregates into contact with the compound according to claim 1;
(b) Allowing the compound to bind to the TDP-43 aggregates;
(c) Detecting the compound bound to the TDP-43 aggregates;
(d) Optionally correlating the presence or absence of the compound bound with the TDP-43 aggregates with the presence or absence of TDP-43 aggregates in the sample or specific body part or body area; and
(e) Optionally repeating steps (a) to (c) and, if present, optional step (d) at least one time; and
(vi) A method of collecting data for predicting responsiveness of a patient suffering from a disease, disorder or abnormality associated with TDP-43 aggregates to a treatment with a medicament, the method comprising the steps:
(a) Bringing a sample, a specific body part or body area suspected to contain TDP-43 aggregates into contact with a compound according to claim 1;
(b) Allowing the compound to bind to the TDP-43 aggregates;
(c) Detecting the compound bound to the TDP-43 aggregates;
(d) Optionally correlating the presence or absence of the compound bound with the TDP-43 aggregates with the presence or absence of TDP-43 aggregates in the sample or specific body part or body area; and
(e) Optionally repeating steps (a) to (c) and, if present, optional step (d) at least one time.

21. (canceled)

22. (canceled)

23. (canceled)

24. (canceled)

25. (canceled)

26. (canceled)

27. (canceled)

28. (canceled)

29. A TDP-43 aggregates' biomarker, a TDP-43 proteinopathy biomarker, a TDP-43 proteinopathy diagnostic agent or TDP-43 proteinopathy diagnostic tool comprising a compound according to claim 1.

30. (canceled)

31. An in vitro analytical reference or an in vitro screening tool comprising a compound according to claim 1.

32. The method according to claim 19, to wherein the disease, disorder or abnormality associated with TDP-43 aggregates or the TDP-43 proteinopathy is selected from Frontotemporal dementia (FTD, such as Sporadic or familial with or without motor-neuron disease (MND), with progranulin (GRN) mutation, with C9orf72 mutations, with TARDBP mutation, with valosine-containing protein (VCP) mutation, linked to chromosome 9p, corticobasal degeneration, frontotemporal lobar degeneration (FTLD) including Frontotemporal lobar dementia TDP-43 or Frontotemporal lobar degeneration with ubiquitin-positive TDP-43 inclusions (FTLD-TDP), Argyrophilic grain disease, Pick's disease, semantic variant primary progressive aphasia (svPPA), behavioural variant FTD (bvFTD), Nonfluent Variant Primary Progressive Aphasia (such as nfvPPA), Amyotrophic lateral sclerosis (ALS, such as Sporadic ALS, with TARDBP mutation, with angiogenin (ANG) mutation), Alexander disease (AxD), limbic-predominant age-related TDP-43 encephalopathy (LATE), Chronic Traumatic Encephalopathy, Perry syndrome, Alzheimer's disease (AD, including sporadic and familial forms of AD), Down syndrome, Familial British dementia, Polyglutamine diseases (Huntington's disease and spinocerebellar ataxia type 3 (SCA3; also known under Machado Joseph Disease)), Hippocampal sclerosis dementia and Myopathies (Sporadic inclusion body myositis, Inclusion body myopathy with a mutation in the valosin-containing protein (VCP); also Paget disease of bone and frontotemporal dementia), Oculo-pharyngeal muscular dystrophy with rimmed vacuoles, Myofibrillar myopathies with mutations in the myotilin (MYOT) gene or mutations in the gene coding for desmin (DES), Traumatic Brain Injury (TBI), Dementia with Lewy Bodies (DLB) and Parkinson's disease (PD), preferably, the disease, the disorder or the abnormality associated with TDP-43 aggregates or the TDP-43 proteinopathy is selected from Frontotemporal dementia (FTD), amyotrophic lateral sclerosis (ALS), Alzheimer's disease (AD), Parkinson's disease (PD), Chronic Traumatic Encephalopathy (CTE), and limbic-predominant age-related TDP-43 encephalopathy (LATE).

33. The method according to claim 32, wherein the disease, disorder or abnormality associated with TDP-43 aggregates or the TDP-43 proteinopathy is amyotrophic lateral sclerosis (ALS), Alzheimer's disease (AD), Frontotemporal dementia (FTD) including Frontotemporal lobar dementia TDP-43, Frontotemporal lobar degeneration with TDP-43 inclusions (FTLD-TDP), limbic-predominant age-related TDP-43 encephalopathy (LATE), Frontotemporal dementia (FTD) with progranulin (GRN) mutation, or Frontotemporal dementia (FTD) with C9orf72 mutations.

34. (canceled)

35. (canceled)

36. (canceled)

37. (canceled)

38. A compound having the formula (II) or (III), or a stereoisomer, polymorph, racemic mixture, tautomer, pharmaceutically acceptable salt, prodrug, hydrate, or solvate thereof, or mixtures thereof 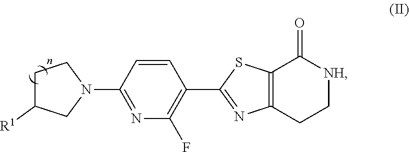
wherein
n is 1 or 2; and
R¹ is H, hydroxy(C₁-C₄)alkyl or F; 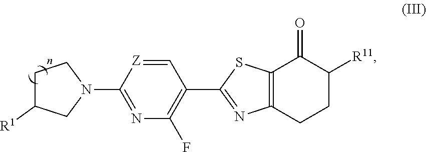
wherein
Z is selected from C—Br, C—I, and C—H, wherein
n is 1 or 2;
R¹ is H, hydroxy(C₁-C₄)alkyl or F;
R¹¹ is a 5- or 6-membered carbocyclic or heterocyclic ring which can be optionally substituted with Br, I, F, NH₂, CN and/or CH₃, wherein the heterocyclic ring contains one or more heteroatoms selected from N, O and S;
and at least one of Z and/or R¹¹ comprises Br or I.

39. (canceled)

40. A compound having the formula (IV) or (V) 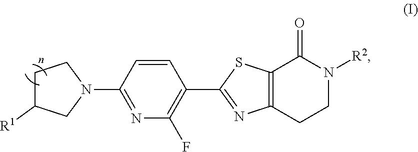
wherein
n is 1 or 2;
R² is 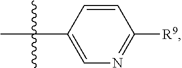
wherein R⁹ is H; and
R¹⁴ is a leaving group (LG); 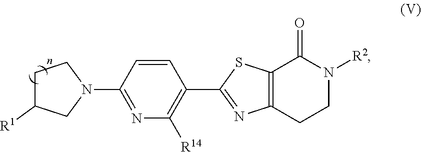
wherein
n is 1 or 2;
R¹ is H or hydroxy(C₁-C₄)alkyl;
R² is 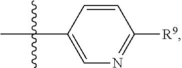
wherein R⁹ is H; and
R¹⁴ is a leaving group (LG).

41. (canceled)

42. A compound having the formula (III) 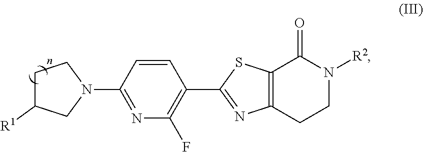
or a stereoisomer, polymorph, racemic mixture, tautomer, pharmaceutically acceptable salt, prodrug, hydrate, or solvate thereof;
wherein
n is 1 or 2;
R¹⁵ is selected from —COOR′ or —C₁-C₃alkylCOOR′; wherein R′ is C₁-C₃ alkyl; and
R² is a 5- or 6-membered carbocyclic or heterocyclic ring which can be optionally substituted with F, NH₂, CN and/or CH₃, wherein the heterocyclic ring contains one or more heteroatoms selected from N, O and S.

43. A kit for preparing a radiopharmaceutical preparation comprising a compound of formula (II) as defined in claim 38.

44. A kit for preparing a radiopharmaceutical preparation comprising a compound of formula (II) or (III) as defined in claim 38.

45. A kit for preparing a radiopharmaceutical preparation comprising a compound of formula (IV) or (V) as defined in claim 40.

46. The method according to claim 20, wherein the disease, disorder or abnormality associated with TDP-43 aggregates or the TDP-43 proteinopathy is selected from Frontotemporal dementia (FTD, such as Sporadic or familial with or without motor-neuron disease (MND), with progranulin (GRN) mutation, with C9orf72 mutations, with TARDBP mutation, with valosine-containing protein (VCP) mutation, linked to chromosome 9p, corticobasal degeneration, frontotemporal lobar degeneration (FTLD) including Frontotemporal lobar dementia TDP-43 or Frontotemporal lobar degeneration with ubiquitin-positive TDP-43 inclusions (FTLD-TDP), Argyrophilic grain disease, Pick's disease, semantic variant primary progressive aphasia (svPPA), behavioural variant FTD (bvFTD), Nonfluent Variant Primary Progressive Aphasia (such as nfvPPA), Amyotrophic lateral sclerosis (ALS, such as Sporadic ALS, with TARDBP mutation, with angiogenin (ANG) mutation), Alexander disease (AxD), limbic-predominant age-related TDP-43 encephalopathy (LATE), Chronic Traumatic Encephalopathy, Perry syndrome, Alzheimer's disease (AD, including sporadic and familial forms of AD), Down syndrome, Familial British dementia, Polyglutamine diseases (Huntington's disease and spinocerebellar ataxia type 3 (SCA3; also known under Machado Joseph Disease)), Hippocampal sclerosis dementia and Myopathies (Sporadic inclusion body myositis, Inclusion body myopathy with a mutation in the valosin-containing protein (VCP); also Paget disease of bone and frontotemporal dementia), Oculo-pharyngeal muscular dystrophy with rimmed vacuoles, Myofibrillar myopathies with mutations in the myotilin (MYOT) gene or mutations in the gene coding for desmin (DES), Traumatic Brain Injury (TBI), Dementia with Lewy Bodies (DLB) and Parkinson's disease (PD), preferably, the disease, the disorder or the abnormality associated with TDP-43 aggregates or the TDP-43 proteinopathy is selected from Frontotemporal dementia (FTD), amyotrophic lateral sclerosis (ALS), Alzheimer's disease (AD), Parkinson's disease (PD), Chronic Traumatic Encephalopathy (CTE), and limbic-predominant age-related TDP-43 encephalopathy (LATE).

47. The method according to claim 46, wherein the disease, disorder or abnormality associated with TDP-43 aggregates or the TDP-43 proteinopathy is amyotrophic lateral sclerosis (ALS), Alzheimer's disease (AD), Frontotemporal dementia (FTD) including Frontotemporal lobar dementia TDP-43, Frontotemporal lobar degeneration with TDP-43 inclusions (FTLD-TDP), limbic-predominant age-related TDP-43 encephalopathy (LATE), Frontotemporal dementia (FTD) with progranulin (GRN) mutation, or Frontotemporal dementia (FTD) with C9orf72 mutations.
